(19) United States
(12) Patent Application Publication
(10) Pub. No.: US 2025/0388568 A1
(43) Pub. Date: Dec. 25, 2025

(54) NLRP3 INHIBITORS

(71) Applicant: Kodiak Sciences Inc., Palo Alto, CA (US)

(72) Inventors: Jianhua Chao, Palo Alto, CA (US); Michael Joseph Bishop, San Diego, CA (US); Daniel Victor Perlroth, Palo Alto, CA (US)

(21) Appl. No.: 18/881,231

(22) PCT Filed: Jul. 3, 2023
(86) PCT No.: PCT/US2023/026859

§ 371 Date: Jan. 3, 2025

Related U.S. Application Data
(60) Provisional application No. 63/358,639, filed on Jul. 6, 2022.

(51) Int. Cl. C07D 405/06; A61K 31/341; A61K 31/351; A61K 31/397; A61K 31/4025; A61K 31/415; A61K 31/4155; A61K 31/4355; A61K 31/496; C07D 231/18; C07D 307/64; C07D 307/68; C07D 403/06; C07D 405/12; C07D 407/12; C07D 491/048

(52) U.S. Cl. CPC C07D 405/06; A61K 31/341; A61K 31/351; A61K 31/397; A61K 31/4025; A61K 31/415; A61K 31/4155; A61K 31/4355; A61K 31/496; C07D 231/18; C07D 307/64; C07D 307/68; C07D 403/06; C07D 405/12; C07D 407/12; C07D 491/048

(57) ABSTRACT

Disclosed herein are small molecule compounds that are capable of inhibiting NLRP3 family proteins and NLRP3 inflammasome function in various disease settings. The disease is characterized by a disease progression pathology that comprises the activity of NLRP3 inflammasome. Disclosed herein also include pharmaceutical compositions comprising a therapeutically effective amount of the NLRP3 inhibitor compound, the use and preparation thereof.

### BACKGROUND OF THE INVENTION

### Field of the Invention

The present disclosure relates generally to the field of NLRP3 (NOD-like receptor-, LRR- and pyrin domain-containing 3) family proteins and NLRP3 inflammasome function. More specifically, the present disclosure relates to the field of small molecule compounds that are capable of inhibiting NLRP3 family proteins and NLRP3 inflammasome function in various disease settings.

### Description of the Related Art

Inflammasomes are large dynamic multimeric protein complexes formed in the cytosol, upon activation by either pathogen-associated molecular patterns (PAMPs) due to bacterial infection or by endogenous danger signals, danger-associated molecular patterns (DAMPs), released from damaged or dying cells. Tschopp and Martinon in 2002 established a connection between the inflammasomes and the activation of pro-inflammatory protease caspase-1. Caspase-1 is a proteolytic enzyme responsible for converting the inactive precursors, Pro-IL1β and Pro-IL18, to their active form IL1β and IL18 respectively. Both IL-1β and IL-18 belong to the IL-1 family of cytokines that play key roles in innate and adaptive immune responses impacting a wide range of disease conditions. Tschopp's discovery positioned inflammasomes at the center of immune regulation and homeostasis. Since then, the field has expanded tremendously with new insights garnered on assembly, regulation, and function of inflammasome complexes, as well as their roles in the inflammatory associated diseases.

The inflammasome complexes are composed of three components, a sensor, an adaptor, and an effector enzyme. The sensor is usually a nucleotide-binding domain and leucine-rich-repeat-containing (NLR) protein or an AIM2-like receptor (ALR) protein. The majority of the inflammasomes contains a sensor from the NLR family, and importantly, each inflammasome has its own unique sensor protein. The adaptor protein is called ASC (apoptosis-associated speck-like protein containing a CARD), which recruits and links the effector enzyme to the sensor. ASC is a common component in many inflammasome complexes. Finally, the effector enzyme typically is pro-caspase-1. Upon assembly and activation of the inflammasomes, pro-caspase-1 will be converted to active caspase-1 which will proceed with the activation of key cytokines such as IL-1β and IL-18.

### SUMMARY OF THE INVENTION

The present disclosure provides a compound represented by the structure of Formula (I):

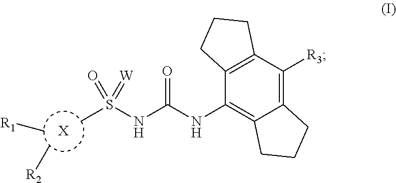

or a pharmaceutically acceptable salt, solvate, isomer, or tautomer thereof, wherein W is selected from O, NH, and N—CN; R₁ is selected from:

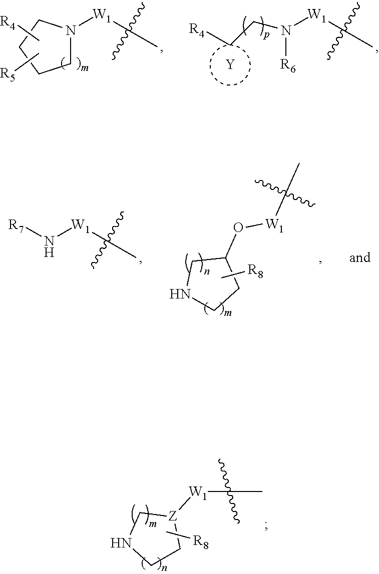

W₁ is (CH₂)_{q} or C(═O), and q is 1, 2, or 3; R₄ is selected from halogen, CF₃, C1-C8 alkyl, and C3-C8 cycloalkyl, or absent; R₅ is hydroxyl or hydroxyalkyl; R₆ is selected from H, optionally substituted C1-C8 alkyl, and optionally substituted C3-C8 cycloalkyl; or R₆ and R₂ together form a 5-10 member optionally substituted heterocyclic ring, the heterocyclic ring having one or more heteroatoms selected from O, N, and S; R₇ is H or C1-C8 alkyl; R₈ is selected from halogen, hydroxyalkyl, and optionally substituted C1-C8 alkyl, or absent;

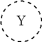

is C3-C8 cycloalkyl or 4-8 member heterocycloalkyl with one heteroatom selected from N, O, and S; m is selected from 0, 1, 2, 3, and 4; n is selected from 1, 2, 3, and 4; p is selected from 1, 2, and 3; and Z is selected from N, CH, and C—CH₃; R₂ is selected from C1-C8 alkyl, CF₃, C3-C8 cycloalkyl, and optionally substituted hydroxyalkyl, or absent; R₃ is selected from H, halogen, or C1-C8 alkyl; and

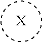

is a 5-member heteroaryl having 1 or 2 heteroatoms, each of the heteroatoms is selected from O, N, and S.

In several embodiments, [symbol] is selected from the group consisting of furan, thiophene, pyrrole, pyrazole, imidazole.

In several embodiments,

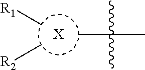

is selected from the group consisting of

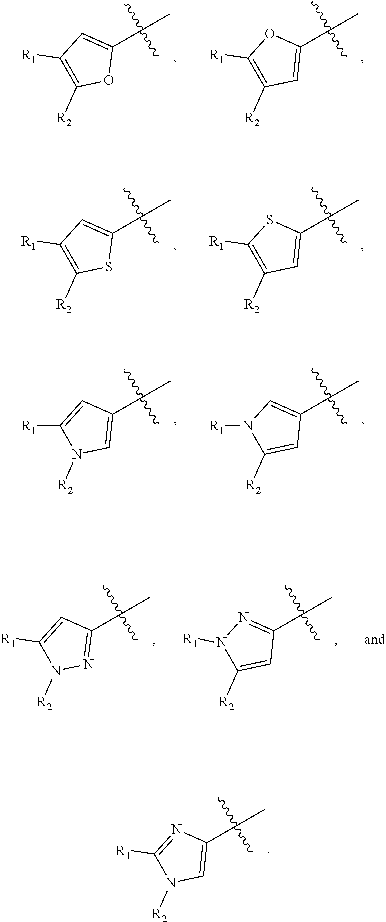

In several embodiments, R₁ is

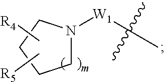

R₄ is selected from halogen, CF₃, C1-C8 alkyl, and C3-C8 cycloalkyl, or absent; R₅ is hydroxyl or hydroxy(C1-C3 alkyl); and m is 0, 1, 2, or 3.

In several embodiments, R₁ is

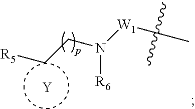

R₅ is hydroxyl or hydroxy(C1-C3 alkyl); R₆ is H or optionally substituted C1-C8 alkyl; or R₆ and R₂ together form a 5-10-member optionally substituted heterocyclic ring, the heterocyclic ring having one or more heteroatom selected from O, N, and S; and

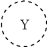

is C3-C6 cycloalkyl or 4-6-member heterocycloalkyl with one heteroatom selected from the group consisting of N, O, and S.

In several embodiments, R₁ is H

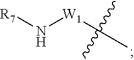

and R₇ is H or C1-C8 alkyl.

In several embodiments, R₁ is

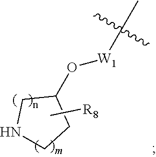

R₈ is selected from halogen, hydroxy, and optionally substituted alkyl, or absent; m is selected from 0, 1, 2, 3, and 4; and n is selected from 1, 2, 3, and 4.

In several embodiments, R₁ is

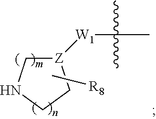

R₈ is selected from halogen, hydroxy, and optionally substituted alkyl, or absent; m is selected from 0, 1, 2, 3, and 4; and n is selected from 1, 2, 3, and 4.

In several embodiments, the compound is further represented by the structure of Formula (Ia):

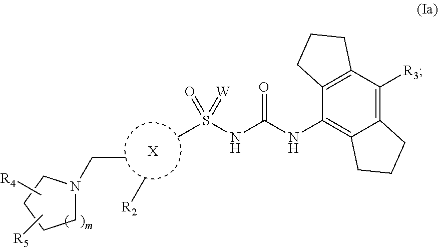

or a pharmaceutically acceptable salt, solvate, isomer, or tautomer thereof. In several embodiments, W is O or NH; R₄ is halogen, CF₃, or absent; R₅ is OH or CH₂OH; m is 0, 1, or 3; R₂ is H or C1-C3 alkyl; R₃ is H, F, or Cl; and

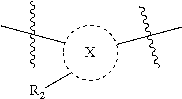

is selected from

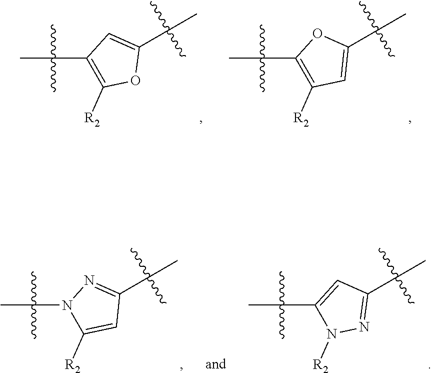

In several embodiments, the compound is further represented by the structure of Formula (Ib):

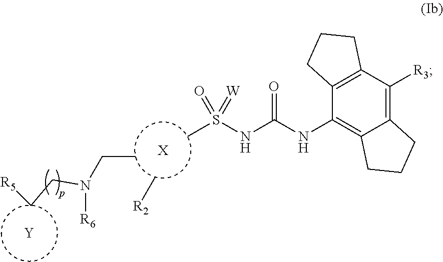

or a pharmaceutically acceptable salt, solvate, isomer, or tautomer thereof. In several embodiments,

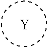

is C3-C6 cycloalkyl or a 4-6-member heterocycloalkyl having one O; W is O or NH; R₅ is OH or CH₂OH; R₆ is H or C1-C3 alkyl; p is 1 or 2; R₂ is H or C1-C3 alkyl; R₃ is H or halogen; and

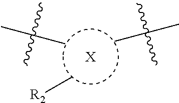

is selected from

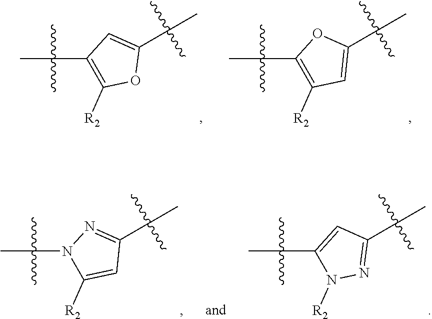

In several embodiments, the compound is further represented by the structure of Formula (Ic):

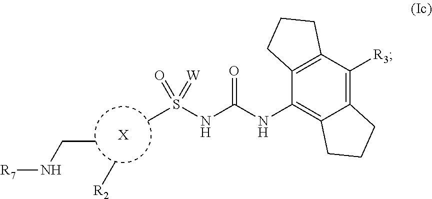

or a pharmaceutically acceptable salt, solvate, isomer, or tautomer thereof. In several embodiments, W is O or NH, R₇ is H or C1-C3 alkyl; R₂ is H or C1-C3 alkyl; R₃ is H or halogen; and

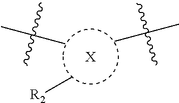

is selected from the group consisting of

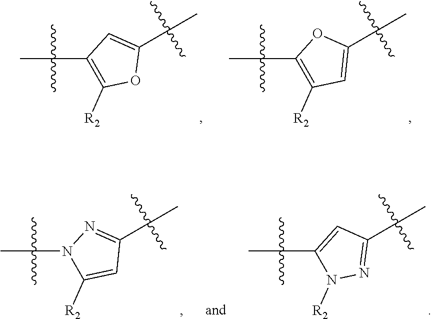

In several embodiments, the compound is further represented by the structure of Formula (Id):

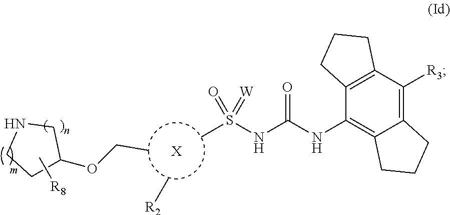

or a pharmaceutically acceptable salt, solvate, isomer, or tautomer thereof. In several embodiments, W is O or NH; Re is absent; m is 0, 1, or 2; n is 1 or 2; R₂ is hydroxy (C1-C3 alkyl); R₃ is H or halogen; and

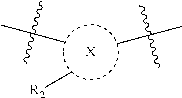

is selected from the group consisting of

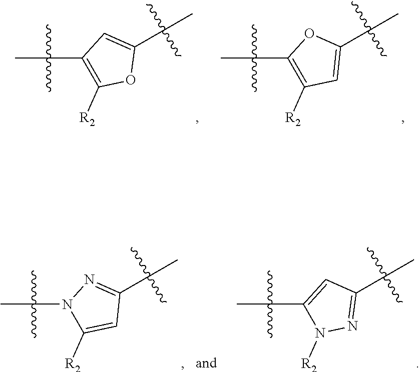

In several embodiments, the compound is further represented by the structure of Formula (Ie):

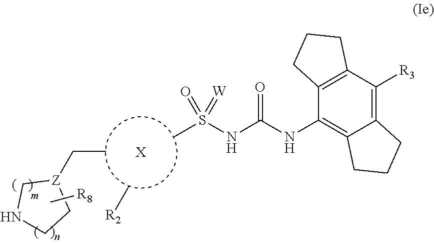

or a pharmaceutically acceptable salt, solvate, isomer, or tautomer thereof. In several embodiments, W is O or NH; R₈ is absent, m is 0, 1, or 2; n is 1, 2, or 3; Z is N or CH; R₂ is C1-C3 alkyl or hydroxy (C1-C3 alkyl); R₈ is H or halogen; and

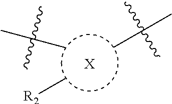

is selected from the group consisting of

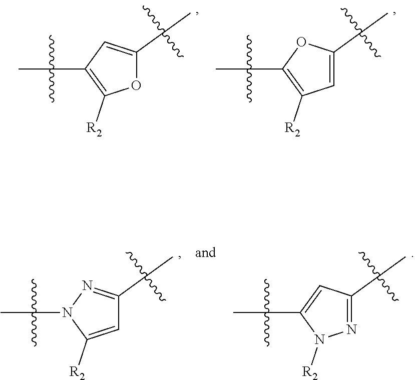

The present disclosure also provides a pharmaceutical composition comprising a therapeutically effective amount of a compound and a pharmaceutically acceptable excipient as described herein.

The present disclosure further provides a method of preventing, treating, or ameliorating one or more diseases in a subject, comprising administering a compound or a pharmaceutically acceptable excipient as described herein, to a subject in need thereof. In some embodiments, the method is a monotherapy. In some embodiments, the method includes administering at least one another form of treatment. In some embodiments, the subject is human

In several embodiments, the disease is characterized by a disease progression that comprises the activity of IL-1β, IL-18, or both.

In several embodiments, the disease is selected from the group consisting of atherosclerosis, gout, acute gouty arthritis, rheumatoid arthritis, nonalcoholic steatoheptitis, inflammatory bowel disease, Parkinson's disease, Alzheimer's disease, multiple sclerosis, glaucoma, age related macula degeneration, diabetic retinopathy, and dry eye.

In some embodiments, the disease is at least one chronic inflammatory disorder. In some embodiments, the disease is characterized by a disease progression pathology that comprises the activity of NLRP3 inflammasome. In some embodiments, the NLRP3 inflammasome comprises at least one mutation.

### DETAILED DESCRIPTION

The compounds disclosed herein are potent inhibitors of NLRP3 inflammasome. Such NLRP3 inhibitory compounds may be useful in the treatment or prevention of inflammatory disorders, and diseases with underlying pathology of inflammation, associated with NLRP3 inflammasome. Lack of high-resolution crystal structures of ligand bound NLRP3 protein complex makes it a challenge to apply structure-based design approaches in the discovery of NLRP3 inhibitors. We have used a pharmacophore/ligand-based approach to identify a structural region where incorporation of a polar hydroxyl or amino group is tolerated for activity while allowing us to modulate physicochemical properties. Surprisingly, incorporating hydroxyl and amino groups at strategic positions of the molecular structure is not only tolerated but also delivered robust inhibitory potency, while also facilitating a solubility increase and a lipophilicity reduction for an enhanced safety profile. As such, the compounds of Formula (I) not only are potent inhibitors of NLRP3 inflammasome, but also have structural component that can contribute to the enhanced safety profile of a drug.

### Definitions

For the purpose of the present invention the following terminology will be used in accordance with the definitions set forth below.

“A” and “an” are used herein to refer to one or more than one (e.g., to at least one) of the grammatical object of the article. By way of example, “an element” means one element or more than one element.

“And/or” is used herein to mean either “and” or “or” unless indicated otherwise.

“About” as used herein means variation one might see in measurements taken among different instruments, samples, and sample preparations.

“Administer”, “administering”, or “administration” as used herein refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

“Alkyl” refers to a straight or branched, saturated, aliphatic radical. The number of carbon atoms present in the alkyl group may be specified by indicating the number of carbon in the group (e.g., C3 alkyl contains three carbon atoms). The size range of an alkyl group can be specified by indicating a range of the numbers of carbon atoms (e.g., C1-C3 alkyl or (C₁-C₃)alkyl for a one to three carbon atom containing alkyl group). For example, C1-C6 alkyl includes, but is not limited to, methyl, ethyl, propyl, isopropyl, butyl, isobutyl, sec-butyl, tert-butyl, pentyl, isopentyl, hexyl, etc. Non-limiting examples of alkyl groups include methyl, ethyl, propyl, butyl, pentyl, 1-methylbutyl (i.e., 2-pentyl), 1-ethylpropyl (i.e., 3-pentyl), 3-methylpentyl, and the like. Alkyl can include any number of carbons, such as 1-2, 1-3, 1-4, 1-5, 1-6, 1-7, 1-8, 1-9, 1-10, 2-3, 2-4, 2-5, 2-6, 3-4, 3-5, 3-6, 4-5, 4-6 and 5-6. The alkyl group is typically monovalent, but can be divalent, such as when the alkyl group links two moieties together, and it is understood that “alkyl” includes alkylene when two functionalities are appended.

“Heteroalkyl” refers to an alkyl group having from 1 to 3 heteroatoms such as N, O and S. The heteroatoms can also be oxidized, such as, but not limited to, —S(O)— and —S(O)₂—. For example, heteroalkyl can include ethers, thioethers, alkyl-amines and alkyl-thiols. The heteroalkyl group is typically monovalent, but can be divalent or multivalent, such as when the heteroalkyl group links two moieties together, and it is understood that “heteroalkyl” includes heteroalkylene when two functionalities are appended.

“Cycloalkyl” or “carbocyclyl” refers to a cyclic hydrocarbon group (saturated or partially unsaturated) that contains from about 3 to 12, from 3 to 10, from 3 to 8, or from 3 to 7 endocyclic carbon atoms. Cycloalkyl groups include fused, bridged and spiro ring structures. Where cycloalkyl groups may have a range of sizes, that size range may be specified by indicating the number of carbon atoms present in the cycloalkyl group (e.g., C3-C10 cycloalkyl for a three to ten carbon atom containing cycloalkyl group).

“Heterocycloalkyl” or “heterocyclic” refers to a ring system having from 3 ring members to about 20 ring members and from 1 to about 5 heteroatoms such as N, O and S. The heteroatoms can also be oxidized, such as, but not limited to, —S(O)— and —S(O)₂—. For example, heterocycle includes, but is not limited to, tetrahydrofuranyl, tetrahydropyranyl, oxepanyl, tetrahydrothiophenyl, morpholino, pyrrolidinyl, imidazolidinyl, imidazolinyl, pyrazolidinyl, pyrazolinyl, piperazinyl, piperidinyl, azepanyl, indolinyl, quinuclidinyl and 1,4-dioxa-8-aza-spiro[4.5]dec-8-yl.

“Alkoxy” refers to a straight or branched chain saturated or unsaturated (fully or partially) hydrocarbon containing 1-12 carbon atoms containing a terminal “0” in the chain, e.g., —O(alkyl). Examples of alkoxy groups include without limitation, methoxy, ethoxy, propoxy, butoxy, t-butoxy, or pentoxy groups. In an embodiment, “alkoxy” is fully saturated.

“Alkoxyalkoxy” refers to an alkoxy group as defined herein which is substituted with an alkoxy group e.g., —O(alkyl)-O-(alkyl). Examples of alkoxyalkoxy groups include without limitation, methoxymethoxy, ethoxyethoxy, propoxymethoxy, or ethoxymethoxy.

“Alkenyl” refers to a straight or branched chain unsaturated hydrocarbon containing 2-12 carbon atoms. The “alkenyl” group contains at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl. An alkenyl group can be unsubstituted or substituted. Alkenyl, as herein defined, may be straight or branched.

“Alkynyl” refers to a straight or branched chain unsaturated hydrocarbon containing 2-12 carbon atoms. The “alkynyl” group contains at least one triple bond in the chain. Examples of alkynyl groups include ethynyl, propynyl, n-butynyl, iso-butynyl, pentynyl, or hexynyl. An alkynyl group can be unsubstituted or substituted.

“Aryl” refers to a monocyclic or fused bicyclic, tricyclic or greater, aromatic ring assembly containing 6 to 16 ring carbon atoms. For example, aryl may be phenyl, benzyl or naphthyl, preferably phenyl. “Arylene” means a divalent radical derived from an aryl group. Aryl groups can be mono-, di- or tri-substituted by one, two or three radicals selected from alkyl, alkoxy, aryl, hydroxy, halogen, cyano, amino, amino-alkyl, trifluoromethyl, alkylenedioxy and oxy-C2-C3-alkylene; all of which are optionally further substituted, for instance as hereinbefore defined; or 1- or 2-naphthyl; or 1- or 2-phenanthrenyl.

“Heteroaryl” refers to an atom that is not a carbon atom and is part of the contiguous cyclic structure of a cyclic compound.

“Heteroaryl” refers to a monocyclic or fused bicyclic or tricyclic aromatic ring assembly containing 5 to 16 ring atoms, where from 1 to 4 of the ring atoms are each a heteroatom independently selected from N, O and S. Non-limiting examples of heteroaryl includes pyridyl, indolyl, indazolyl, quinoxalinyl, quinolinyl, isoquinolinyl, benzothienyl, benzofuranyl, furanyl, pyrrolyl, thiazolyl, benzothiazolyl, oxazolyl, isoxazolyl, triazolyl, tetrazolyl, pyrazolyl, imidazolyl, thienyl, or any other radicals substituted, especially mono- or di-substituted, by e.g. alkyl, nitro or halogen. Pyridyl represents 2-, 3- or 4-pyridyl, advantageously 2- or 3-pyridyl. Thienyl represents 2- or 3-thienyl. Quinolinyl represents preferably 2-, 3- or 4-quinolinyl. Isoquinolinyl represents preferably 1-, 3- or 4-isoquinolinyl. Benzopyranyl, benzothiopyranyl represents preferably 3-benzopyranyl or 3-benzothiopyranyl, respectively. Thiazolyl represents preferably 2- or 4-thiazolyl, and most preferred, 4-thiazolyl. Triazolyl is preferably 1-, 2- or 5-(1,2,4-triazolyl). Tetrazolyl is preferably 5-tetrazolyl.

Substituents for the aryl and heteroaryl groups are varied and are selected from: halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —Ni, —CH(Ph)₂, perfluoro(C1-C4)alkoxy, and perfluoro(C1-C₄)alkyl, in a number ranging from zero to the total number of open valences on the aromatic ring system; and where R′, R″ and R′″ are independently selected from hydrogen, (C1-C8)alkyl and heteroalkyl, unsubstituted aryl and heteroaryl, (unsubstituted aryl)-(C1-C4)alkyl, and (unsubstituted aryl)oxy-(C1-C4)alkyl.

“Carrier”, as used herein, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

“Compound” as used herein is intended to encompass not only the specified molecular entity but also its pharmaceutically acceptable, pharmacologically active derivatives, including, but not limited to, salts, prodrug, metabolites, hydrates, solvates and the like.

“Cyano” as used herein means a substituent having a carbon atom joined to a nitrogen atom by a triple bond, e.g., C≡N.

“Disorder” is used herein to mean, and is used interchangeably with, the terms disease, condition, or illness, unless otherwise indicated.

“Halogen” or “halo” refers to fluorine, chlorine, bromine, or iodine.

“Haloalkyl” as used herein refers to an alkyl group, as defined herein, which is substituted by one or more halogen. Examples of haloalkyl groups include, but are not limited to, trifluoromethyl, difluoromethyl, pentafluoroethyl, trichloromethyl, etc. In an embodiment, “haloalkyl” is fully saturated.

“Haloalkoxy” as used herein refers to an alkoxy group, as defined herein, which is substituted one or more halogen. Examples of haloalkyl groups include, but are not limited to, trifluoromethoxy, difluoromethoxy, pentafluoroethoxy, trichloromethoxy, etc. In an embodiment, “haloalkoxy” is fully saturated.

“Hydroxyalkyl” means an alkyl group as defined above, where the alkyl group is substituted with one or more —OH groups. Examples of hydroxyalkyl groups include HOCH₂—, HO—CH₂—CH₂— and CH₃—CH(OH)—. In an embodiment, “hydroxyalkyl” is fully saturated.

“Isomer” refers to certain compound of the present invention which possess asymmetric carbon atoms (optical centers) or double bonds; the racemate, diastereomer, geometric isomer and individual isomer (e.g., separate enantiomers). All of these are encompassed by the term “isomer” within the scope of the present disclosure.

“Optionally substituted” is understood to mean that a given chemical moiety (e.g., an alkyl group) can (but is not required to) be bonded to other substituents (e.g., heteroatoms). For instance, an alkyl group that is optionally substituted can be a fully saturated alkyl chain (e.g., a pure hydrocarbon). Alternatively, the same optionally substituted alkyl group can have substituents different from hydrogen. For instance, it can, at any point along the chain be bounded to a halogen atom, a hydroxyl group, or any other substituent described herein. Thus the term “optionally substituted” means that a given chemical moiety has the potential to contain other functional groups, but does not necessarily have any further functional groups. Suitable substituents used in the optional substitution of the described groups include, without limitation, halogen, oxo, —OH, —CN, —COOH, —CH₂CN, —O—(C₁-C₆) alkyl, (C₁-C6) alkyl, (C₁-C₆) alkenyl, (C₁-C₆) alkynyl, (C₁-C₆) hydroxyalkyl, (C₁-C₆) alkoxy, (C₁-C₆) haloalkyl, (C₁-C₆) haloalkoxy, (C₃-C₇) cycloalkyl, aryl, heterocycloalkyl, heteroaryl, —O—(C₂-C₆) alkenyl, —O—(C₂-C₆) alkynyl, (C₂-C₆) alkenyl, (C₂-C₆) alkynyl, —OP(O)(OH)₂, —OC(O)(C₁-C₆) alkyl, —C(O)(C₁-C₆) alkyl, —OC(O)O(C₁-C₆) alkyl, —NH₂, —NH((C₁-C₆) alkyl), —N((C₁-C₆) alkyl)₂, —NHC(O)(C₁-C₆) alkyl, —C(O)NH(C₁-C₆) alkyl, —S(O)₂(C₁-C₆) alkyl, —S(O)₂NH(C₁-C₆) alkyl, and S(O)₂N((C₁-C₆) alkyl)₂. “Optionally substituted” as used herein also refers to substituted or unsubstituted whose meaning is described below.

“Substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

“Unsubstituted” means that the specified group bears no substituents.

“Oxo” as used herein refers to an “═O” group.

“Patient” or “subject in need thereof” refers to a living organism suffering from or prone to a condition that can be prevented or treated by administration of a pharmaceutical composition as provided herein. Non-limiting examples include humans, other mammals and other non-mammalian animals.

“Pharmaceutically acceptable” composition or “pharmaceutical composition” refers to a composition comprising a compound of the invention and a pharmaceutically acceptable excipient or pharmaceutically acceptable excipients.

“Pharmaceutically acceptable excipient” and “pharmaceutically acceptable carrier” refer to an excipient that can be included in the compositions of the invention and that causes no significant adverse toxicological effect on the patient. Non-limiting examples of pharmaceutically acceptable excipients include water, NaCl, normal saline solutions, lactated Ringer's, normal sucrose, normal glucose and the like.

“Salt” includes, without limitation, acid addition salts including hydrochlorides, hydrobromides, phosphates, sulphates, hydrogen sulphates, alkylsulphonates, arylsulphonates, acetates, benzoates, citrates, maleates, fumarates, succinates, lactates, and tartrates; salts of alkali metal cations such as Na⁺, K⁺, Li⁺ (e.g., NaCl, KCl) organic amine salts or alkali earth metal salts such as Mg or Ca salts.

“Solvate” refers to a complex of variable stoichiometry formed by a solute and solvent. Such solvents for the purpose of the invention may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

“Spirocycloalkyl” or “spirocyclyl” means carbogenic bicyclic ring systems with both rings connected through a single atom. The ring can be different in size and nature, or identical in size and nature. Examples include spiropentane, spirihexane, spiroheptane, spirooctane, spirononane, or spirodecane. One or both of the rings in a spirocycle can be fused to another ring carbocyclic, heterocyclic, aromatic, or heteroaromatic ring. One or more of the carbon atoms in the spirocycle can be substituted with a heteroatom (e.g., O, N, S, or P). A (C₃-C₁₂) spirocycloalkyl is a spirocycle containing between 3 and 12 carbon atoms. One or more of the carbon atoms can be substituted with a heteroatom. In an embodiment, “spirocycloalkyl” or “spirocyclyl” is fully saturated. The term “spiroheterocycloalkyl” or “spiroheterocyclyl” is understood to mean a spirocycle wherein at least one of the rings is a heterocycle (e.g., at least one of the rings is furanyl, morpholinyl, or piperadinyl). In an embodiment, “spiroheterocycloalkyl” or “spiroheterocyclyl is fully saturated.

“Therapeutically effective amount” refers to an amount of a biologically active agent or of a pharmaceutical composition useful for treating, ameliorating, or preventing an identified disease or condition, or for exhibiting a detectable therapeutic or inhibitory effect. The effect can be detected by any assay method known in the art.

“Treating” with regard to a subject, refers to improving at least one symptom of the subject's disorder. Treating includes curing, improving, or at least partially ameliorating the disorder.

### Compounds

Provided herein are compounds that are capable of inhibiting NLRP3 family proteins and NLRP3 inflammasome function. Various embodiments of these compounds include compounds having the structure of formula (I) as described herein or pharmaceutically acceptable salts, solvates, or tautomers thereof. The structure of formula (I) encompasses all stereoisomers and racemic mixtures, including the structures described herein.

The present disclosure provides a compound represented by the structure of Formula (I):

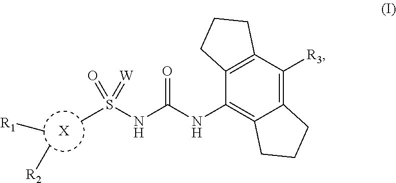

or a pharmaceutically acceptable salt, solvate, isomer, or tautomer thereof, wherein W is O, NH, or N—CN;

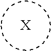

is a 5-member heteroaryl ring having 1 or 2 heteroatoms independently selected from N, O, and S; R₁ is selected from one of the following moieties:

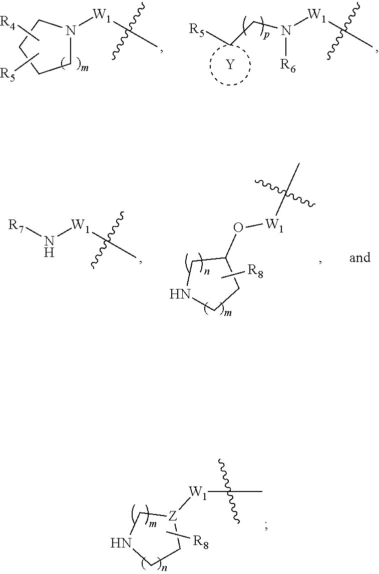

wherein W₁ is (CH₂)_{q} or C(═O), wherein q is 1, 2, or 3; R₄ is halogen, CF₃, C1-C8 alkyl, or C3-C8 cycloalkyl, or R₄ is absent; R₅ is hydroxyl or hydroxyalkyl; R₆ is H, optionally substituted C1-C8 alkyl, or optionally substituted C3-C8 cycloalkyl; or R and R₂ together form a 5-10 member optionally substituted heterocyclic ring, the heterocyclic ring having one or more heteroatom selected from O, N, and S; R₇ is H or C1-C8 alkyl; R₈ is halogen, hydroxyalkyl, or optionally substituted alkyl, or R₈ is absent;

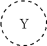

is C3-C8 cycloalkyl or 4-8-member heterocycloalkyl with one heteroatom selected from N, O, and S; m is 0, 1, 2, 3, or 4; n is 1, 2, 3, or 4; p is 1, 2, or 3; Z is N, CH, or C—CH₃; R₂ is C1-C8 alkyl, CF₃, C3-C8 cycloalkyl, or optionally substituted hydroxyalkyl, or R₂ is absent; and R₃ is H, halogen, or C1-C8 alkyl.

In some embodiments of Formula (I),

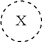

is selected from furan, thiophene, pyrrole, pyrazole, and imidazole. In some embodiments,

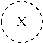

is selected from the group consisting of 2,3,5-furan, 2,3,5-thiophene, 1,2,4-pyrrole, 1,3,5-pyrazole, 1,2,4-imidazole. In some embodiments,

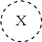

may have one of the following structures:

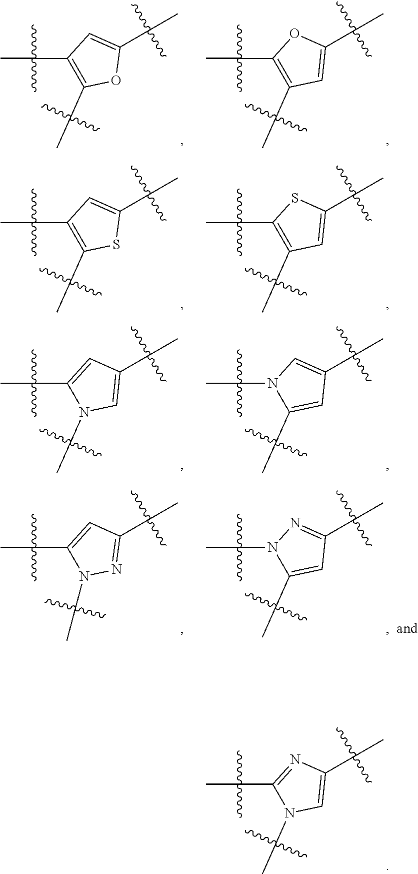

In some embodiments,

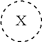

is

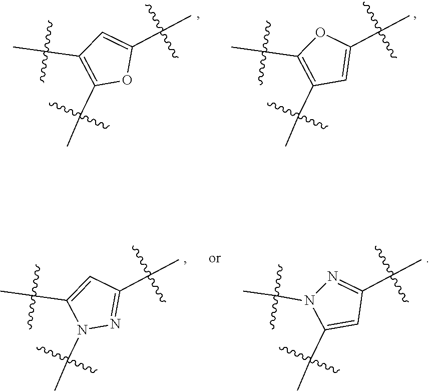 [SMILES: *c1cc(*)c(*)o1 ; *c1cc(*)c(*)o1 ; *c1cc(*)n(*)n1 ; *c1cc(*)n(*)n1]

In some embodiments,

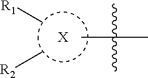

has one of the following structures:

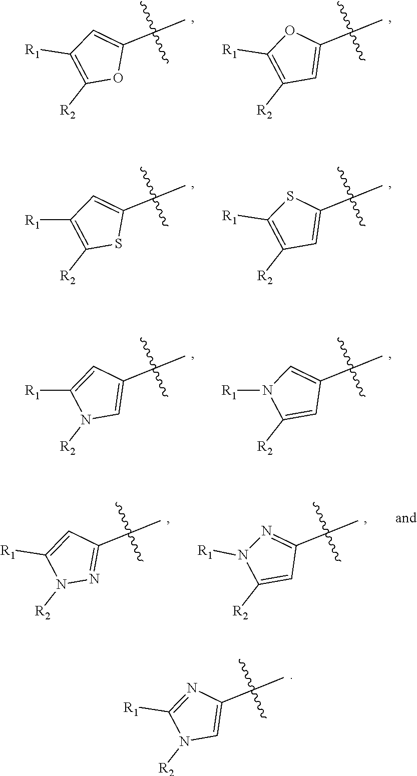

In some embodiments,

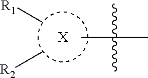

is

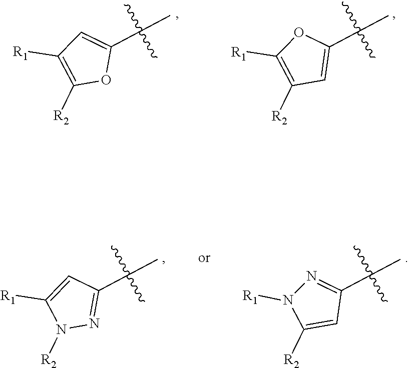

In some embodiments of Formula (I), R₁ is

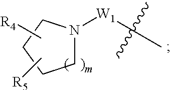

R₄ is selected from halogen, CF₃, C1-C8 alkyl, and C3-C8 cycloalkyl, or absent; R₅ is hydroxyl or hydroxy(C1-C3 alkyl); and m is 0, 1, 2, or 3. In some embodiments, R₁ is

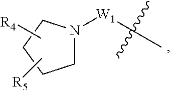

In some embodiments, R₄ is halogen. In some embodiments, R₄ is F. In some embodiments, R₅ is OH. In some embodiments, R₅ is hydroxy(C1-C3)alkyl. In some embodiments, R₅ is CH₂OH. In some embodiments, m is 0, 1, or 3.

In some embodiments of Formula (I), R₁ is

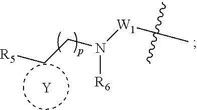

R₅ is hydroxyl or hydroxy(C1-C3)alkyl; R₆ is H or optionally substituted C1-C8 alkyl; or R₆ and R₂ together form a 5-10-member optionally substituted heterocyclic ring, the heterocyclic ring having one or more heteroatom selected from O, N. and S; and

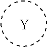

is C3-C6 cycloalkyl or 4-6-member heterocycloalkyl with one heteroatom selected from the group consisting of N, O, and S; and p is 1, 2, or 3. In some embodiments, R₅ is OH. In some embodiments, R₅ is hydroxy(C1-C3)alkyl. In some embodiments, R₅ is CH₂OH. In some embodiments, R₆ is H. In some embodiments, R₆ is optionally substituted C1-C3 alkyl. In some embodiments, R₆ is methyl. In some embodiments,

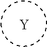

is C3-C6 cycloalkyl. In some embodiments,

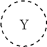

is

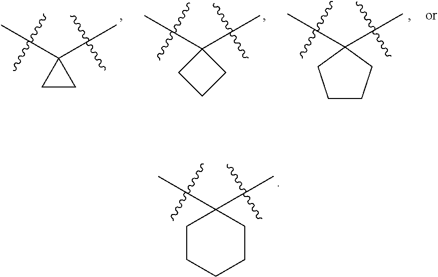 [SMILES: *C1(*)CC1 ; *C1(*)CCC1 ; *C1(*)CCCC1 ; *C1(*)CCCCC1]

In some embodiments,

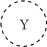

is a 4-6-member heterocycloalkyl having one O. In some embodiments,

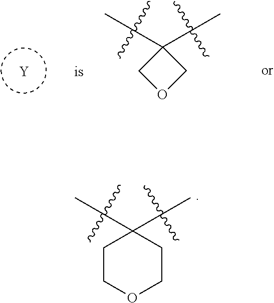

In some embodiments, p is 1 or 2.

In some embodiments of Formula (I), R₁ is

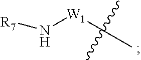

and R₇ is H or C1-C8 alkyl. In some embodiments, R₇ is H or C1-C3 alkyl. In some embodiments, R₇ is methyl. In some embodiments, R₇ is ethyl. In some embodiments, R₇ is propyl. In some embodiments, R₇ is isopropyl.

In some embodiments of Formula (I), R₁ is

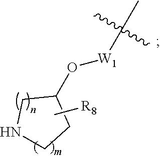

R₈ is selected from halogen, hydroxy, and optionally substituted alkyl, or R₈ is absent; m is selected from 0, 1, 2, 3, and 4; and n is selected from 1, 2, 3, and 4. Tn some embodiments, R₈ is selected from F, OH, methyl, ethyl, propyl, or isopropyl. In some embodiments, R₈ is absent. In some embodiments, m is 0 or 1. In some embodiments, n is 1 or 2. In some embodiments, R₁ is

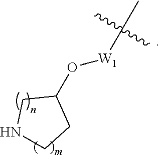

In some embodiments, R₁ is

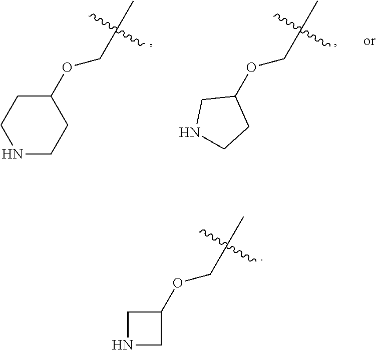 [SMILES: *COC1CCNCC1 ; *COC1CCNC1 ; *COC1CNC1]

In some embodiments of Formula (I), R₁ is

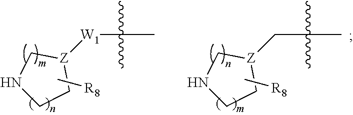

R₈ is selected from halogen, hydroxy, and optionally substituted alkyl, or R₈ is absent; m is selected from 0, 1, 2, 3, and 4; n is selected from 1, 2, 3, and 4; and Z is selected from N, CH, and C—CH₃. In some embodiments, R is selected from F, OH, methyl, ethyl, propyl, and isopropyl. In some embodiments, Ra is absent. In some embodiments, m is 1. In some embodiments, n is 2. In some embodiments, Z is N. In some embodiments, Z is CH. In some embodiments, Z is C—CH₃. In some embodiments, R₁ is

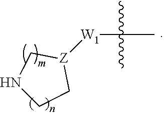

In some embodiments, R₁ is

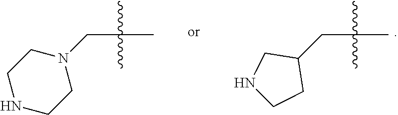 [SMILES: *CN1CCNCC1 ; *CC1CCNC1]

In some embodiments, the compound is represented by the structure of Formula (Ia):

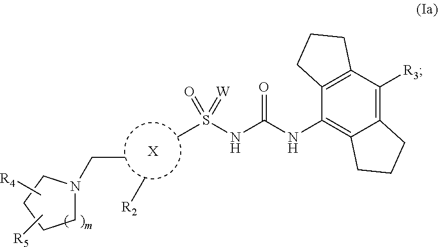

or a pharmaceutically acceptable salt, solvate, isomer, or tautomer thereof. In several embodiments of Formula (Ia), W is O or NH. In several embodiments, W is O. In several embodiments, W is NH. In several embodiments, R₄ is halogen, CF₃, or R₄ is absent. In several embodiments, R₄ is halogen. In several embodiments, R₄ is F. In several embodiments, R₄ is CF₃. In several embodiments, R₄ is absent. In several embodiments, R₅ is OH or CH₂OH. In several embodiments, R₅ is OH. In several embodiments, R₅ is CH₂OH. In several embodiments, m is 0, 1, 2, 3, or 4. In several embodiments, m is 0. In several embodiments, m is 1. In several embodiments, m is 3. In several embodiments, R₂ is H, C1-C8 alkyl, CF₃, C3-C8 cycloalkyl, or optionally substituted hydroxyalkyl. In several embodiments, R₂ is H or C1-C3 alkyl. In several embodiments, R₂ is H. In several embodiments, R₂ is C1-C3 alkyl. In some embodiments, R₂ is CH₃. In some embodiments, R₂ is CH(CH₃)₂. In several embodiments, R₃ is H, halogen, or C1-C8 alkyl. In several embodiments, R₃ is H, F, or Cl. In several embodiments, R₃ is H. In several embodiments, R₃ is F. In several embodiments, R₃ is Cl. In several embodiments,

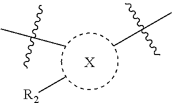

is selected from

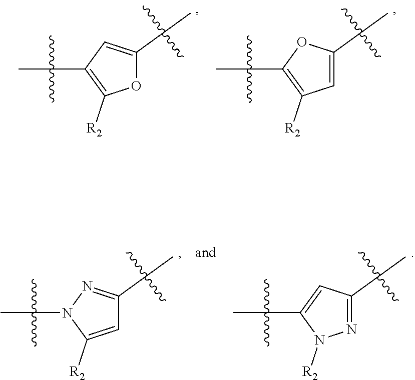

In several embodiments,

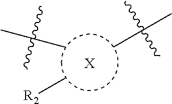

is

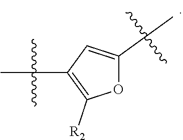

In several embodiments,

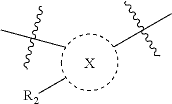

is

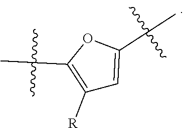

In several embodiments,

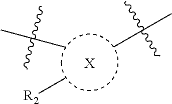

is

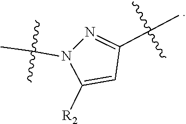

In several embodiments,

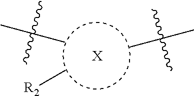

is

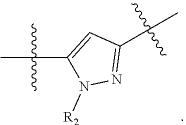

In some embodiments, the compound is represented by the structure of Formula (Ib):

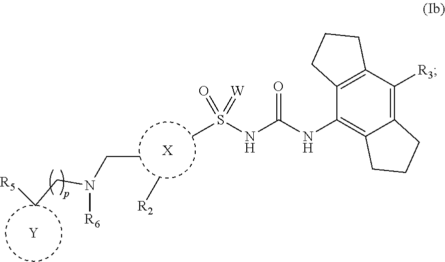

or a pharmaceutically acceptable salt, solvate, isomer, or tautomer thereof. In several embodiments of Formula (Ib),

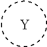

is C3-C6 cycloalkyl or a 4-6-member heterocycloalkyl having one O. In several embodiments,

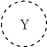

is C3-C6 cycloalkyl. In some embodiments,

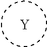

is

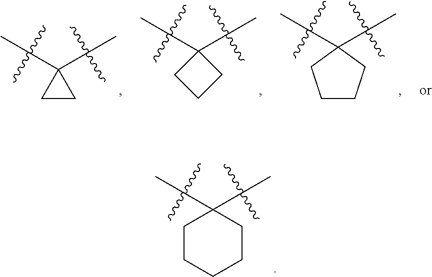 [SMILES: *C1(*)CC1 ; *C1(*)CCC1 ; *C1(*)CCCC1 ; *C1(*)CCCCC1]

In some embodiments,

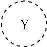

is

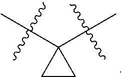 [SMILES: *C1(*)CC1]

In some embodiments,

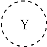

is

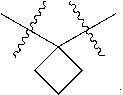 [SMILES: *C1(*)CCC1]

In some embodiments,

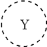

is

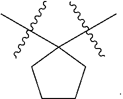 [SMILES: *C1(*)CCCC1]

In some embodiments,

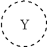

is

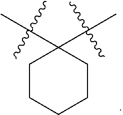 [SMILES: *C1(*)CCCCC1]

In several embodiments,

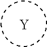

is a 4-6-member heterocycloalkyl having one O. In some embodiments,

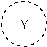

is

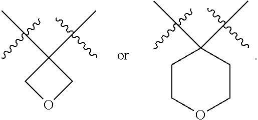 [SMILES: *C1(*)COC1 ; *C1(*)CCOCC1]

In some embodiments,

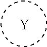

is

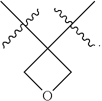 [SMILES: *C1(*)COC1]

In some embodiments,

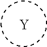

is

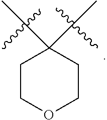 [SMILES: *C1(*)CCOCC1]

In several embodiments, W is O or NH. In several embodiments, W is O. In several embodiments, W is NH. In several embodiments, R₅ is OH or CH₂OH. In several embodiments, R₅ is OH. In several embodiments, R₅ is CH₂OH. In several embodiments, R₆ is H or C1-C3 alkyl. In several embodiments, R₆ is H. In several embodiments, R₆ is C1-C3 alkyl. In some embodiments, R₆ is CH₃. In several embodiments, p is 1 or 2. In several embodiments, p is 1. In several embodiments, p is 2. In several embodiments, R₂ is H or C1-C3 alkyl. In several embodiments, R₂ is H. In several embodiments, R₂ is C1-C3 alkyl. In some embodiments, R₂ is CH₃. In some embodiments, R₂ is CH(CH₃)₂. In several embodiments, R₃ is H or halogen. In several embodiments, R₃ is H. In several embodiments, R₃ is halogen. In some embodiments, R₃ is F. In some embodiments, R₃ is Cl. In several embodiments,

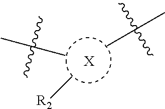

is selected from

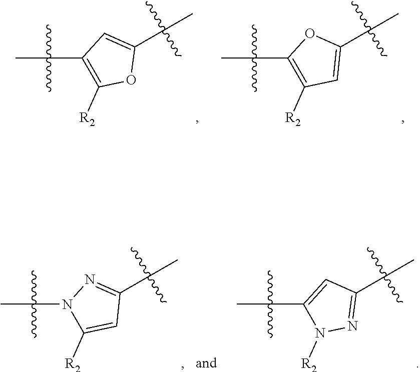

In several embodiments,

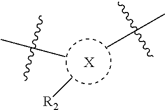

is

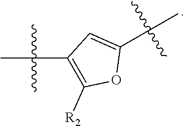

In several embodiments,

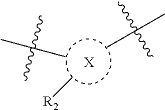

is

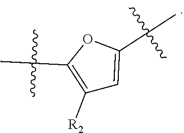

In several embodiments,

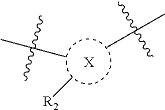

is

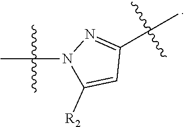

In several embodiments,

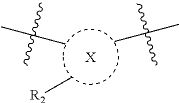

is

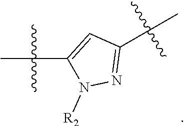

In some embodiments, the compound is, represented by the structure of Formula (Ic).

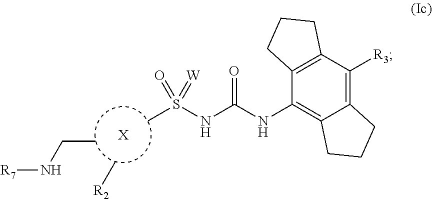

or a pharmaceutically acceptable salt, solvate, isomer, or tautomer thereof. In several embodiments of Formula (Ic), W is O or NH. In several embodiments, W is O. In several embodiments, W is NH. In several embodiments, R₇ is H or C1-C3 alkyl. In several embodiments, R₇ is H. In several embodiments, R₇ is C1-C3 alkyl. In some embodiments, R₇ is methyl. In some embodiments, R₇ is ethyl. In some embodiments, R₇ is propyl. In some embodiments, R₇ is isopropyl. In several embodiments, R₂ is H or C1-C3 alkyl. In several embodiments, R₂ is H. In several embodiments, R₂ is C1-C3 alkyl. In some embodiments, R₂ is CH₃. In some embodiments, R₂ is CH(CH₃)₂. In several embodiments, R₃ is H or halogen. In several embodiments, R₃ is H. In several embodiments, R₃ is F. In several embodiments, R₃ is Cl. In several embodiments,

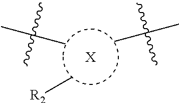

is selected from

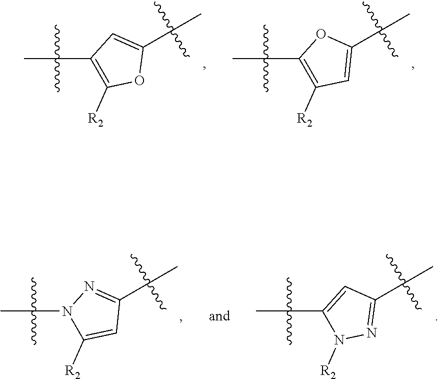

In several embodiments,

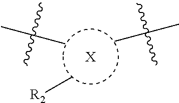

is

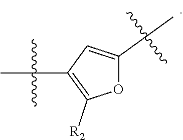

In several embodiments,

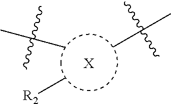

is

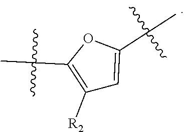

In several embodiments,

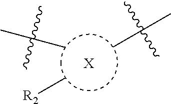

is

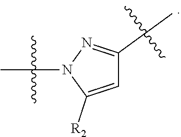

In several embodiments,

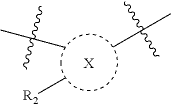

is

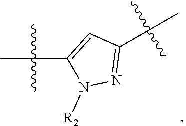

In some embodiments, the compound is represented by the structure of Formula (Id):

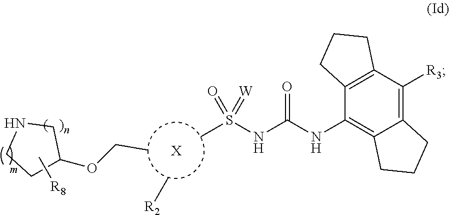

or a pharmaceutically acceptable salt, solvate, isomer, or tautomer thereof. In some embodiments of Formula (Id), W is O or NH. In some embodiments, W is O. In some embodiments, W is NH. In some embodiments, R₈ is absent. In some embodiments, m is 0, 1, 2, 3, or 4. In some embodiments, m is 0. In some embodiments, m is 1. In some embodiments, m is 2. In some embodiments, n is 1, 2, 3, or 4. In some embodiments, n is 1. In some embodiments, n is 2. In some embodiments, R₂ is hydroxy (C1-C3). In some embodiments, R₂ is

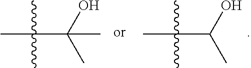

In some embodiments, R₂ is

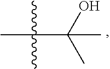 [SMILES: *C(C)(C)O]

In some embodiments, R₂ is

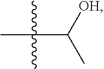 [SMILES: *C(C)O]

In some embodiments, R₃ is H or halogen. In some embodiments, R₃ is H. In some embodiments, R₃ is F. In several embodiments,

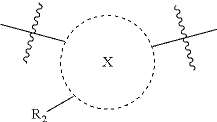

is selected from

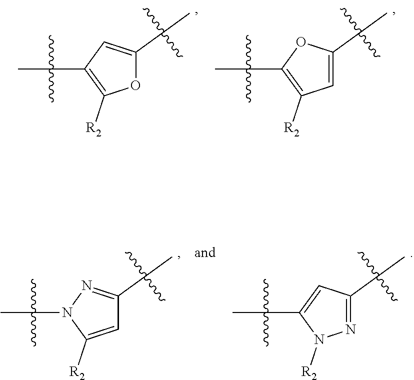

In several embodiments,

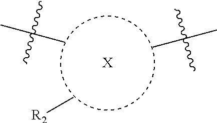

is

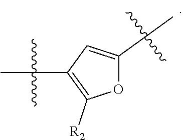

In several embodiments,

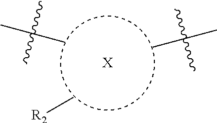

is

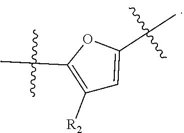

In several embodiments,

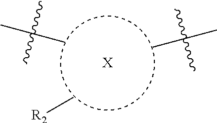

is

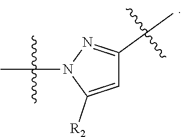

In several embodiments,

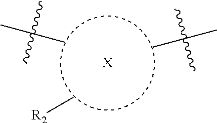

is

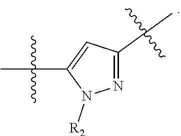

In some embodiments, the compound is represented by the structure of Formula (Ie):

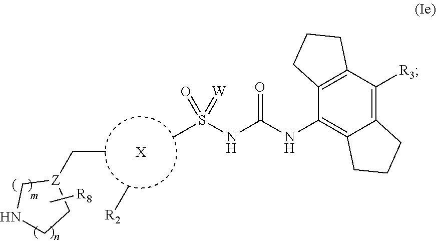

or a pharmaceutically acceptable salt, solvate, isomer, or tautomer thereof. In several embodiments of Formula (Ie), W is O or NH. In some embodiments, W is O. In some embodiments, W is NH. In some embodiments, R₈ is halogen, hydroxy, optionally substituted alkyl. In some embodiments, R₈ is absent. In some embodiments, m is 0, 1, 2, 3, or 4. In some embodiments, m is 0. In some embodiments, m is 1. In some embodiments, m is 2. In some embodiments, n is 1, 2, 3, or 4. In some embodiments, n is 1. In some embodiments, n is 2. In some embodiments, n is 3. In some embodiments, Z is N, CH, or C—CH₃. In some embodiments, Z is N. In some embodiments, Z is CH. In some embodiments, Z is C—CH₃. In some embodiments, R₂ is H, C1-C8 alkyl, CF₃, C3-C8 cycloalkyl, or optionally substituted hydroxyalkyl. In some embodiments, R₂ is C1-C3 alkyl or hydroxy (C1-C3 alkyl). In some embodiments, R₂ is C1-C3 alkyl. In some embodiments, R₂ is hydroxy (C1-C3 alkyl). In some embodiments, R₂ is

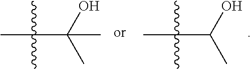

In some embodiments, R₂ is

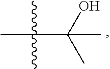 [SMILES: *C(C)(C)O]

In some embodiments, R₂ is

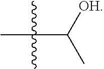 [SMILES: *C(C)O]

In some embodiments, R₃ is H, halogen, or C1-C8 alkyl. In some embodiments, R₃ is H. In some embodiments, R₃ is F. In several embodiments,

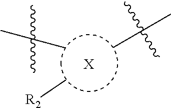

is selected from

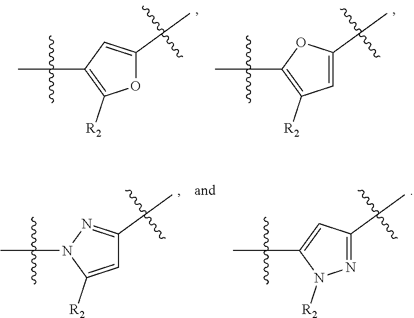

In several embodiments,

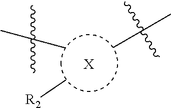

is

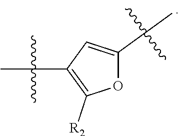

In several embodiments,

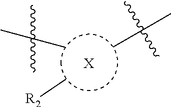

is

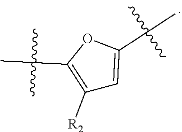

In several embodiments,

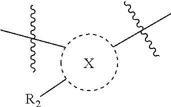

is

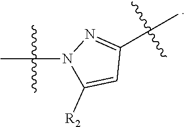

In several embodiments,

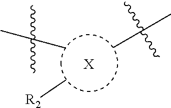

is

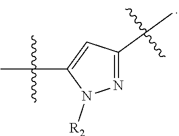

In several embodiments, the compound is further represented by any one of the following:

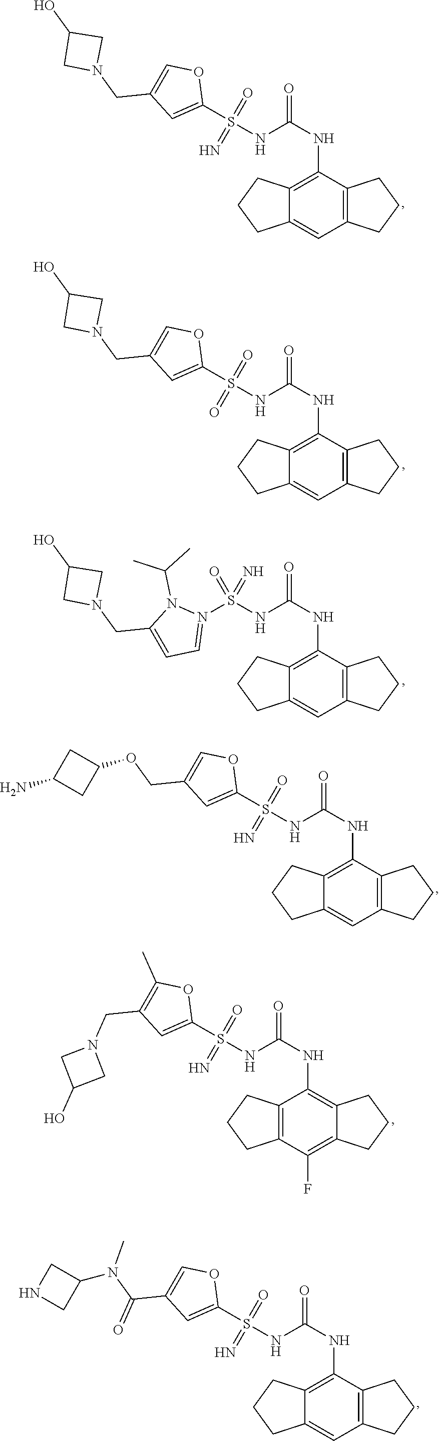

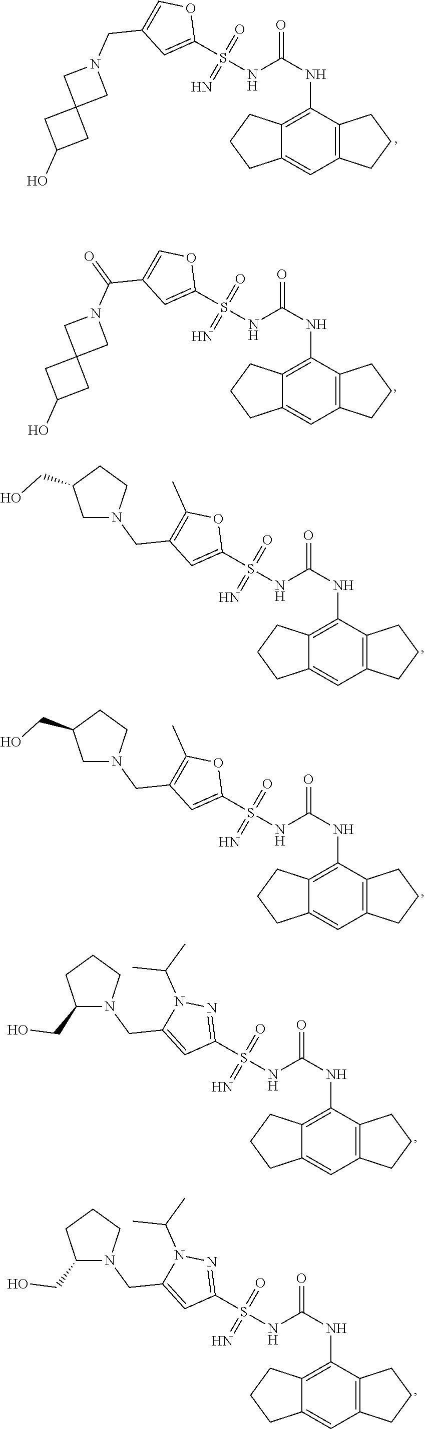

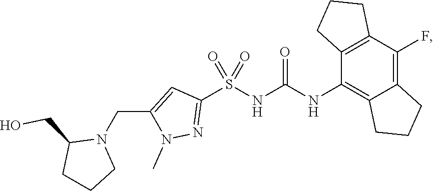 [SMILES: Cn1nc(S(=O)(=O)NC(=O)Nc2c3c(c(F)c4c2CCC4)CCC3)cc1CN1CCC[C@H]1CO]

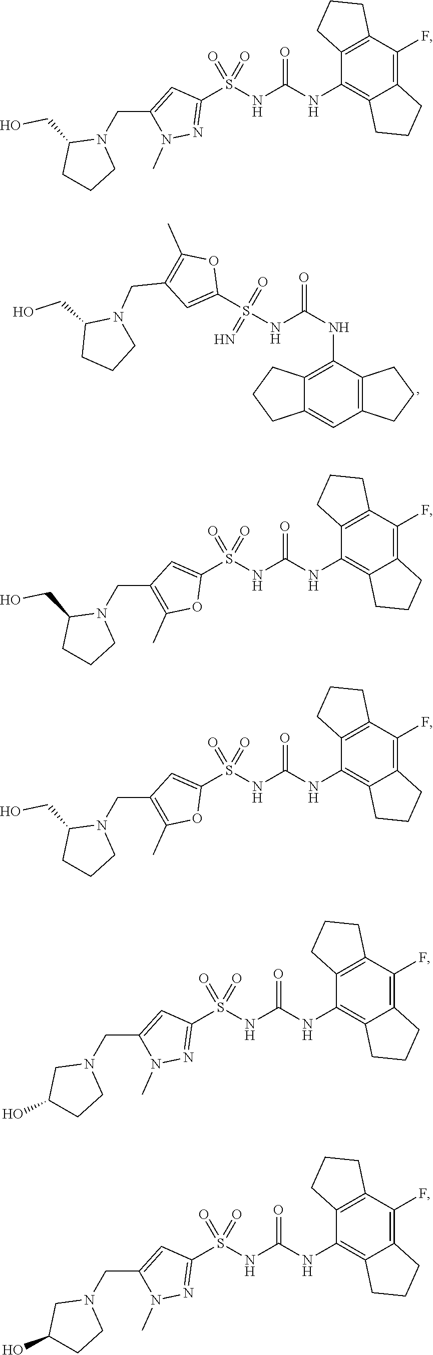 [SMILES: Cn1nc(S(=O)(=O)NC(=O)Nc2c3c(c(F)c4c2CCC4)CCC3)cc1CN1CCC[C@@H]1CO ; Cc1oc(S(=N)(=O)NC(=O)Nc2c3c(cc4c2CCC4)CCC3)cc1CN1CCC[C@@H]1CO ; Cc1oc(S(=O)(=O)NC(=O)Nc2c3c(c(F)c4c2CCC4)CCC3)cc1CN1CCC[C@H]1CO ; Cc1oc(S(=O)(=O)NC(=O)Nc2c3c(c(F)c4c2CCC4)CCC3)cc1CN1CCC[C@@H]1CO ; Cn1nc(S(=O)(=O)NC(=O)Nc2c3c(c(F)c4c2CCC4)CCC3)cc1CN1CC[C@H](O)C1 ; Cn1nc(S(=O)(=O)NC(=O)Nc2c3c(c(F)c4c2CCC4)CCC3)cc1CN1CC[C@@H](O)C1]

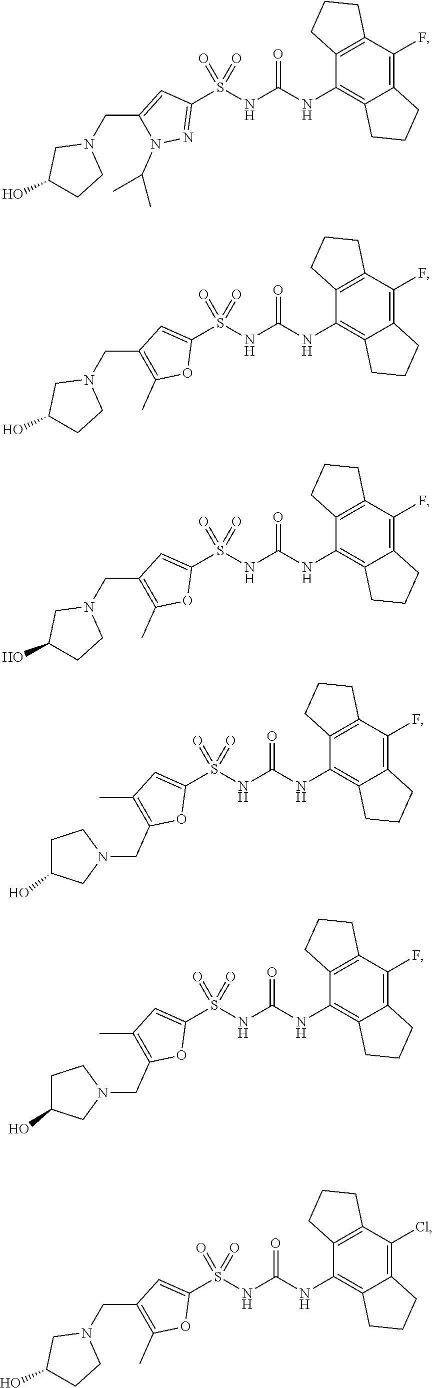 [SMILES: CC(C)n1nc(S(=O)(=O)NC(=O)Nc2c3c(c(F)c4c2CCC4)CCC3)cc1CN1CC[C@H](O)C1 ; Cc1oc(S(=O)(=O)NC(=O)Nc2c3c(c(F)c4c2CCC4)CCC3)cc1CN1CC[C@H](O)C1 ; Cc1oc(S(=O)(=O)NC(=O)Nc2c3c(c(F)c4c2CCC4)CCC3)cc1CN1CC[C@@H](O)C1 ; Cc1cc(S(=O)(=O)NC(=O)Nc2c3c(c(F)c4c2CCC4)CCC3)oc1CN1CC[C@@H](O)C1 ; Cc1cc(S(=O)(=O)NC(=O)Nc2c3c(c(F)c4c2CCC4)CCC3)oc1CN1CC[C@H](O)C1 ; Cc1oc(S(=O)(=O)NC(=O)Nc2c3c(c(Cl)c4c2CCC4)CCC3)cc1CN1CC[C@H](O)C1]

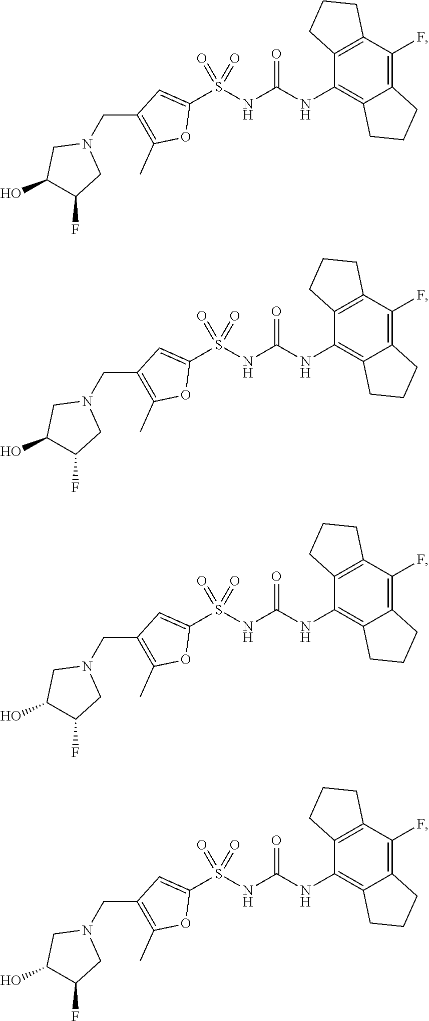 [SMILES: Cc1oc(S(=O)(=O)NC(=O)Nc2c3c(c(F)c4c2CCC4)CCC3)cc1CN1C[C@@H](F)[C@@H](O)C1 ; Cc1oc(S(=O)(=O)NC(=O)Nc2c3c(c(F)c4c2CCC4)CCC3)cc1CN1C[C@H](O)[C@@H](F)C1 ; Cc1oc(S(=O)(=O)NC(=O)Nc2c3c(c(F)c4c2CCC4)CCC3)cc1CN1C[C@@H](O)[C@@H](F)C1 ; Cc1oc(S(=O)(=O)NC(=O)Nc2c3c(c(F)c4c2CCC4)CCC3)cc1CN1C[C@@H](O)[C@H](F)C1]

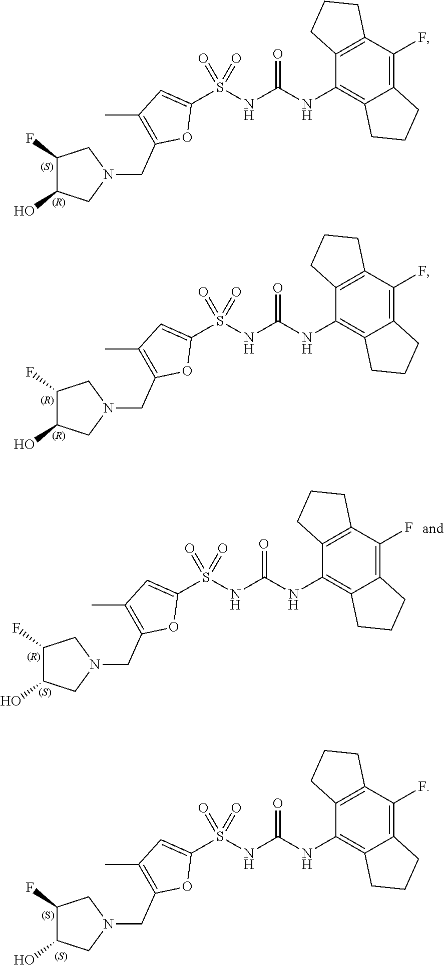

In several embodiments, the compound is further represented by any one of the following:

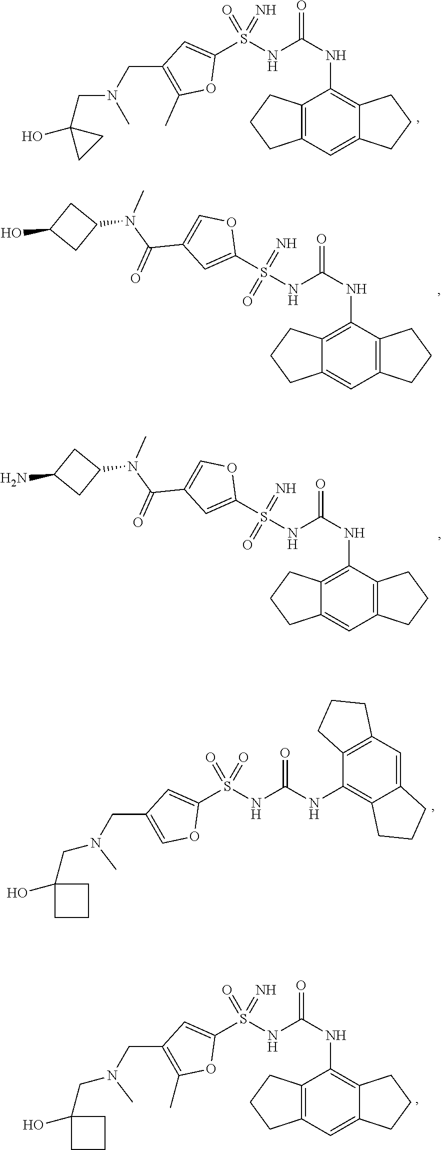

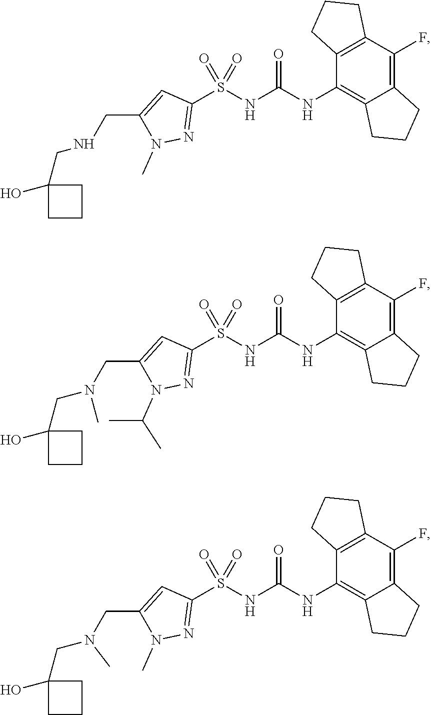 [SMILES: Cn1nc(S(=O)(=O)NC(=O)Nc2c3c(c(F)c4c2CCC4)CCC3)cc1CNCC1(O)CCC1 ; CC(C)n1nc(S(=O)(=O)NC(=O)Nc2c3c(c(F)c4c2CCC4)CCC3)cc1CN(C)CC1(O)CCC1 ; CN(Cc1cc(S(=O)(=O)NC(=O)Nc2c3c(c(F)c4c2CCC4)CCC3)nn1C)CC1(O)CCC1]

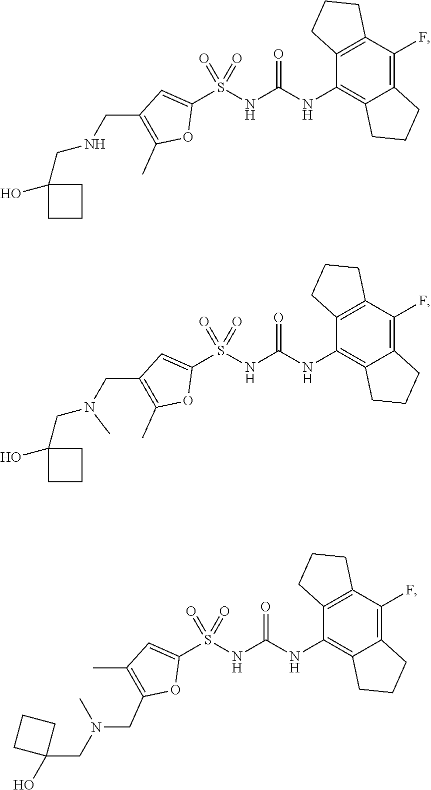 [SMILES: Cc1oc(S(=O)(=O)NC(=O)Nc2c3c(c(F)c4c2CCC4)CCC3)cc1CNCC1(O)CCC1 ; Cc1oc(S(=O)(=O)NC(=O)Nc2c3c(c(F)c4c2CCC4)CCC3)cc1CN(C)CC1(O)CCC1 ; Cc1cc(S(=O)(=O)NC(=O)Nc2c3c(c(F)c4c2CCC4)CCC3)oc1CN(C)CC1(O)CCC1]

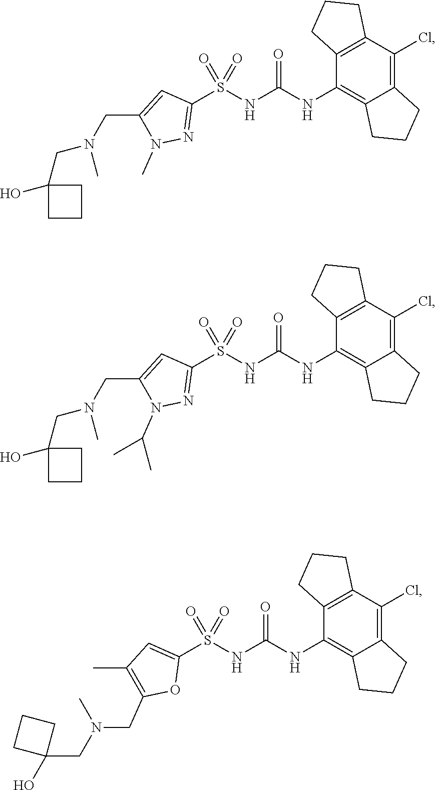 [SMILES: CN(Cc1cc(S(=O)(=O)NC(=O)Nc2c3c(c(Cl)c4c2CCC4)CCC3)nn1C)CC1(O)CCC1 ; CC(C)n1nc(S(=O)(=O)NC(=O)Nc2c3c(c(Cl)c4c2CCC4)CCC3)cc1CN(C)CC1(O)CCC1 ; Cc1cc(S(=O)(=O)NC(=O)Nc2c3c(c(Cl)c4c2CCC4)CCC3)oc1CN(C)CC1(O)CCC1]

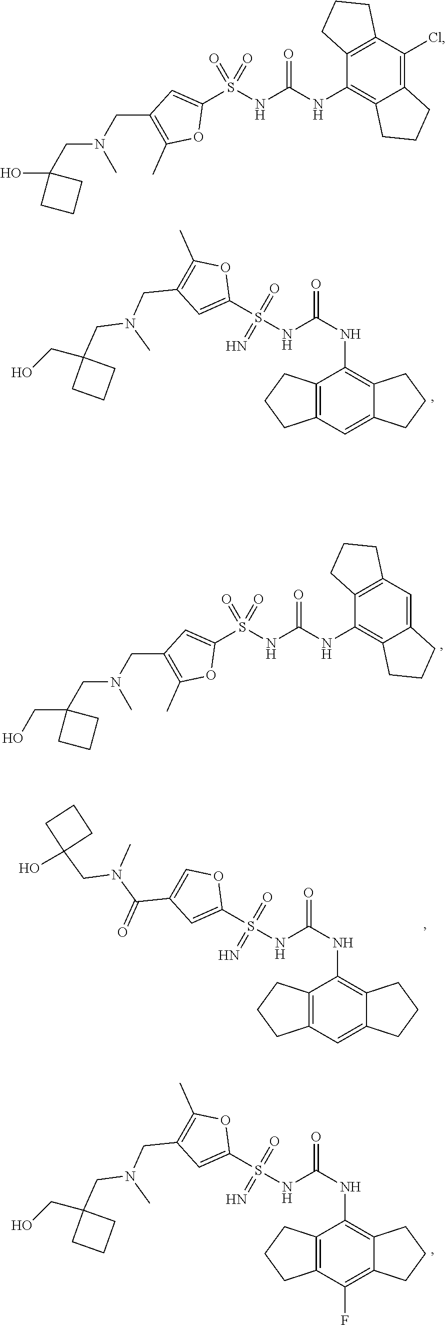 [SMILES: Cc1oc(S(=O)(=O)NC(=O)Nc2c3c(c(Cl)c4c2CCC4)CCC3)cc1CN(C)CC1(O)CCC1 ; Cc1oc(S(=N)(=O)NC(=O)Nc2c3c(cc4c2CCC4)CCC3)cc1CN(C)CC1(CO)CCC1 ; Cc1oc(S(=O)(=O)NC(=O)Nc2c3c(cc4c2CCC4)CCC3)cc1CN(C)CC1(CO)CCC1 ; CN(CC1(O)CCC1)C(=O)c1coc(S(=N)(=O)NC(=O)Nc2c3c(cc4c2CCC4)CCC3)c1 ; Cc1oc(S(=N)(=O)NC(=O)Nc2c3c(c(F)c4c2CCC4)CCC3)cc1CN(C)CC1(CO)CCC1]

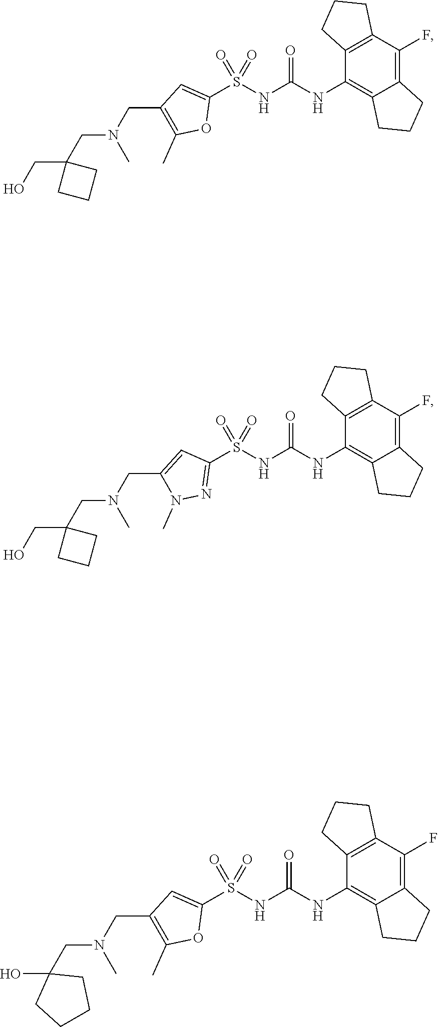 [SMILES: Cc1oc(S(=O)(=O)NC(=O)Nc2c3c(c(F)c4c2CCC4)CCC3)cc1CN(C)CC1(CO)CCC1 ; CN(Cc1cc(S(=O)(=O)NC(=O)Nc2c3c(c(F)c4c2CCC4)CCC3)nn1C)CC1(CO)CCC1 ; Cc1oc(S(=O)(=O)NC(=O)Nc2c3c(c(F)c4c2CCC4)CCC3)cc1CN(C)CC1(O)CCCC1]

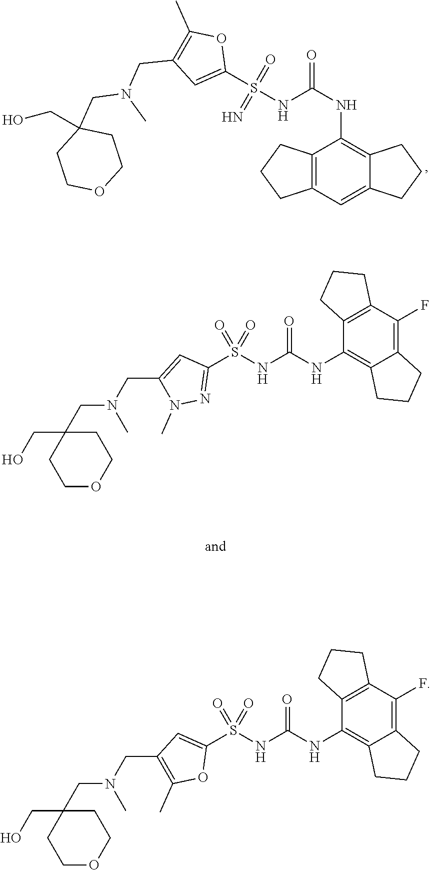 [SMILES: Cc1oc(S(=N)(=O)NC(=O)Nc2c3c(cc4c2CCC4)CCC3)cc1CN(C)CC1(CO)CCOCC1 ; CN(Cc1cc(S(=O)(=O)NC(=O)Nc2c3c(c(F)c4c2CCC4)CCC3)nn1C)CC1(CO)CCOCC1 ; Cc1oc(S(=O)(=O)NC(=O)Nc2c3c(c(F)c4c2CCC4)CCC3)cc1CN(C)CC1(CO)CCOCC1]

In several embodiments, the compound is further represented by any one of the following:

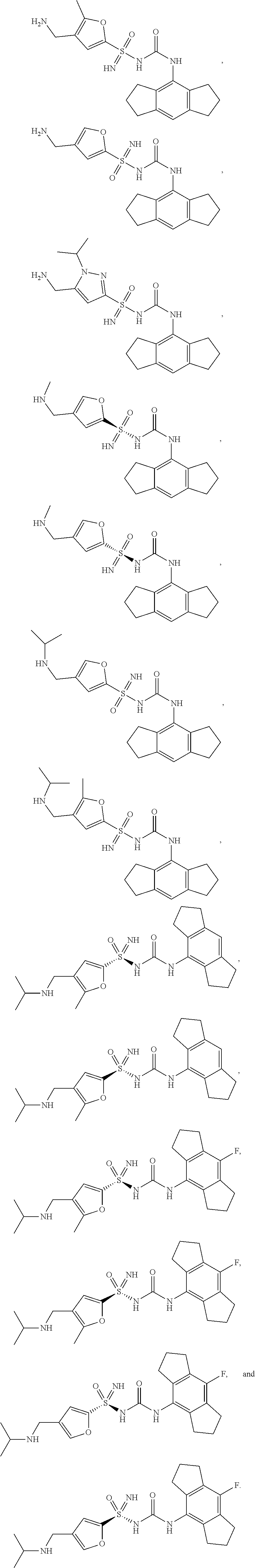

In several embodiments, the compound is further represented by the following:

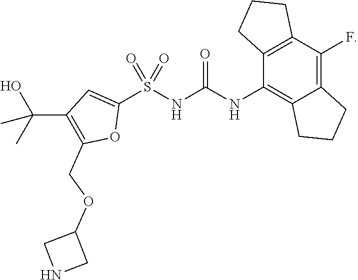 [SMILES: CC(C)(O)c1cc(S(=O)(=O)NC(=O)Nc2c3c(c(F)c4c2CCC4)CCC3)oc1COC1CNC1]

In several embodiments, the compound is further represented by any one of the following:

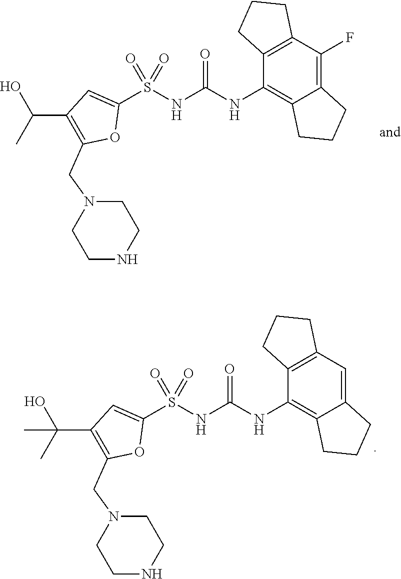 [SMILES: CC(O)c1cc(S(=O)(=O)NC(=O)Nc2c3c(c(F)c4c2CCC4)CCC3)oc1CN1CCNCC1 ; CC(C)(O)c1cc(S(=O)(=O)NC(=O)Nc2c3c(cc4c2CCC4)CCC3)oc1CN1CCNCC1]

In several embodiments, the compound is further represented by any one of the following:

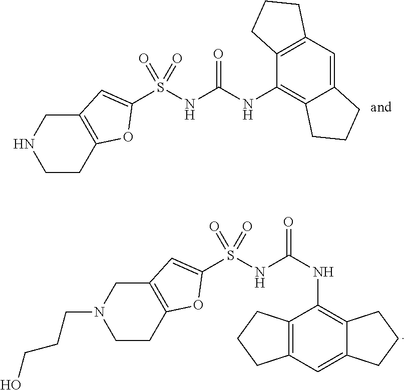 [SMILES: O=C(Nc1c2c(cc3c1CCC3)CCC2)NS(=O)(=O)c1cc2c(o1)CCNC2 ; O=C(Nc1c2c(cc3c1CCC3)CCC2)NS(=O)(=O)c1cc2c(o1)CCN(CCCO)C2]

### Methods of Preparation

The compounds disclosed herein may be synthesized by methods described below, or by modification of these methods. Ways of modifying the methodology include, among others, temperature, solvent, reagents etc., known to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

In the following schemes, protecting groups for oxygen atoms are selected for their compatibility with the requisite synthetic steps as well as compatibility of the introduction and deprotection steps with the overall synthetic schemes (P. G. M. Green, T. W. Wutts, Protecting Groups in Organic Synthesis (3rd ed.) Wiley, New York (1999)).

If the compounds of the present technology contain one or more chiral centers, such compounds can be prepared or isolated as pure stereoisomers, i.e., as individual enantiomers or d(1) stereoisomers, or as stereoisomer-enriched mixtures. All such stereoisomers (and enriched mixtures) are included within the scope of the present technology, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Emka-Chemce or Sigma (St. Louis, Missouri, USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

The methods disclosed herein may include using standard organic synthesis techniques to construct compounds of the general formula (A). In one embodiment, the method includes a coupling reaction between a silyl-protected ethyl-S-aminosulfonimidoyl carboxylate (B) and an appropriately substituted aromatic isocyanate (C) to provide an intermediate (D). In one embodiment, the method includes a series of synthetic steps to transform intermediate (D) into an electrophile (E) that includes a leaving group (LG). In one embodiment, the method includes a substitution reaction that displaces LG with an amine followed by purification to yield the final product (A). In one embodiment, the leaving group is a halide (e.g., Cl, Br, I) or sulfonate. (Scheme 1).

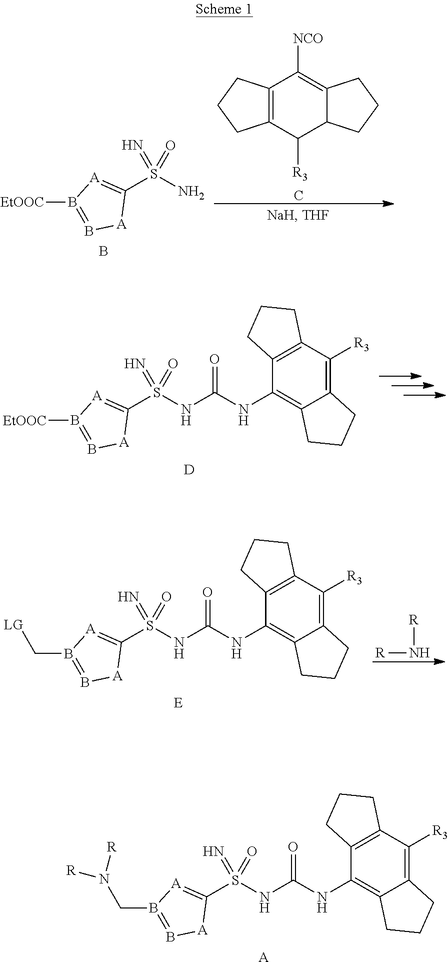

In one embodiment, the method includes a series of synthetic steps to transform intermediate (D) into a carboxylic acid (F) which reacts with an amine, for example, under oxidative catalysis to achieve direct decarboxylative amination followed by purification to yield the final product (A). (Scheme 2).

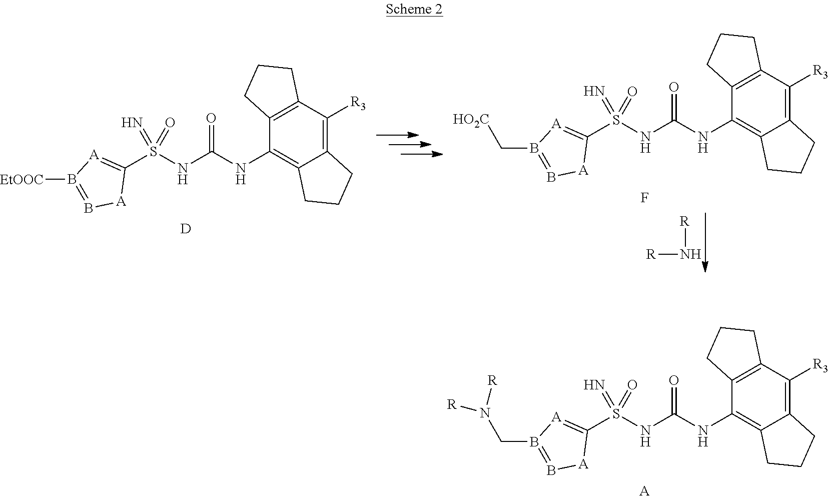

The above example schemes are provided for the guidance of the reader, and collectively represent an example method for making the compounds encompassed herein. Furthermore, other methods for preparing compounds described herein will be readily apparent to the person of ordinary skill in the art in light of the following reaction schemes and examples. Unless otherwise indicated, all variables are as defined above.

### Methods of Treatment

Provided herein are methods that are capable of inhibiting NLRP3 family proteins and NLRP3 inflammasome function. Various embodiments of these methods include compounds having the structures described herein.

The present disclosure provide a method of preventing, treating, or ameliorating one or more diseases in a subject. In several embodiments, the method includes administering to a subject in need thereof at least one compound having a structure as described elsewhere herein. In several embodiments, the method includes administering to a subject in need thereof a pharmaceutically acceptable salt of at least one compound having a structure as described elsewhere herein.

In several embodiments, the disease is characterized by a disease progression that comprises the activity of at least one member of the IL-1 family of cytokines. In several embodiments, the disease progression comprises the activity of at least one IL-1 cytokine with agonistic activity, antagonistic activity, anti-inflammatory activity, and any combination of the foregoing. In several embodiments, the disease progression comprises the activity of at least one IL-1 cytokine with agonistic activity. In several embodiments, the disease progression comprises the activity of at least one of IL-1α, IL-1β, IL-18, IL-33, IL-36α, IL-36β, IL-36γ, IL-1Ra, IL-36Ra, IL-38, and IL-37. In several embodiments, the disease progression comprises the activity of at least one of IL-1α, IL-1β, IL-18, IL-33, IL-36α, IL-36β, IL-36γ. In several embodiments, the disease progression comprises the activity of IL-1β. In several embodiments, the disease progression comprises the activity of IL-18. In several embodiments, the disease progression comprises the activity of IL-1β and IL-18.

In several embodiments, the disease is selected from the group consisting of atherosclerosis, gout, acute gouty arthritis, rheumatoid arthritis, nonalcoholic steatoheptitis (NASH), inflammatory bowel disease, Parkinson's disease, Alzheimer's disease, multiple sclerosis, glaucoma, age related macula degeneration, diabetic retinopathy, and dry eye.

In several embodiments, the disease is at least one chronic inflammatory disorder. In several embodiments, the disease is characterized by a disease progression pathology that comprises the activity of NLRP3 inflammasome. In several embodiments, the NLRP3 inflammasome comprises at least one mutation. In several embodiments, the disease is selected from the group consisting of cryopyrin-associated periodic syndrome, Behcet's syndrome, neonatal onset multisystem inflammatory disease, and Schnitzler's syndrome.

In several embodiments, the method is a monotherapy. In several embodiments, the method includes administering at least one another form of treatment.

In several embodiments, the subject is a mammal. In several embodiments, the subject is human.

### Administration and Pharmaceutical Compositions

Some embodiments of the present disclosure relate to a pharmaceutical composition comprising a therapeutically effective amount of at least one compound having the structure described herein and a pharmaceutically acceptable excipient.

The compounds are administered at a therapeutically effective dosage. While human dosage levels have yet to be optimized for the compounds described herein, generally, a daily dose may be from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. Thus, for administration to a 70 kg person, the dosage range would be from about 17 mg per day to about 8000 mg per day, from about 35 mg per day or less to about 7000 mg per day or more, from about 70 mg per day to about 6000 mg per day, from about 100 mg per day to about 5000 mg per day, or from about 200 mg to about 3000 mg per day. The amount of active compound administered will, of course, be dependent on the subject and disease state being treated, the severity of the affliction, the manner and schedule of administration and the judgment of the prescribing physician.

Administration of the compounds disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

The compounds useful as described above can be formulated into pharmaceutical compositions for use in treatment of these conditions. Standard pharmaceutical formulation techniques are used, such as those disclosed in Remington's The Science and Practice of Pharmacy, 21st Ed., Lippincott Williams & Wilkins (2005), incorporated by reference in its entirety. Accordingly, some embodiments include pharmaceutical compositions comprising: (a) a safe and therapeutically effective amount of a compound described herein (including enantiomers, diastereoisomers, tautomers, polymorphs, and solvates thereof), or pharmaceutically acceptable salts thereof; and (b) a pharmaceutically acceptable carrier, diluent, excipient or combination thereof.

In addition to the selected compound useful as described above, some embodiments include compositions containing a pharmaceutically-acceptable carrier. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, intravitreous, subcutaneous, or other parental routes of administration. In some embodiments, the compositions may be in a form suitable for subcutaneous administration. The skilled artisan will appreciate that oral and nasal compositions comprise compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropes, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carrier suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use, or contain a preservative to prevent contamination over multiple uses.

For ophthalmic application, solutions or medicaments are often prepared using a physiological saline solution as a major vehicle. Ophthalmic solutions should preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations. PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan.

The compounds and compositions described herein, if desired, may be presented in a pack or dispenser device containing one or more unit dosage forms containing the active ingredient. Such a pack or device may, for example, comprise metal or plastic foil, such as a blister pack, or glass, and rubber stoppers such as in vials. The pack or dispenser device may be accompanied by instructions for administration. Compounds and compositions described herein are formulated in a compatible pharmaceutical carrier may also be prepared, placed in an appropriate container, and labeled for treatment of an indicated condition.

The amount of the compound in a formulation can vary within the full range employed by those skilled in the art. Typically, the formulation will contain, on a weight percent (wt %) basis, from about 0.01 to about 99.99 wt % of a compound of the present technology based on the total formulation, with the balance being one or more suitable pharmaceutical excipients. Preferably, the compound is present at a level of about 1 to about 80 wt %. Representative pharmaceutical formulations are described below.

### FORMULATION EXAMPLES

The following are representative pharmaceutical formulations containing a compound of Formula I.

### Formulation Example 1—Tablet Formulation

The following ingredients are mixed intimately and pressed into single scored tablets.

| Ingredient | Quantity per tablet, mg |
|---|---|
| compounds disclosed herein | 400 |
| cornstarch | 50 |
| croscarmellose sodium | 25 |
| lactose | 120 |
| magnesium stearate | 5 |

### Formulation Example 2—Capsule Formulation

The following ingredients are mixed intimately and loaded into a hard-shell gelatin capsule.

| Ingredient | Quantity per tablet, mg |
|---|---|
| compounds disclosed herein | 200 |
| lactose, spray-dried | 148 |
| magnesium stearate | 2 |

### Formulation Example 3—Suspension Formulation

The following ingredients are mixed to form a suspension for oral administration.

| Ingredient | Amount | |
|---|---|---|
| compounds disclosed herein | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.0 | g |
| Veegum K (Vanderbilt Co.) | 1.0 | g |
| flavoring | 0.035 | mL |
| Colorings | 0.5 | mg |

| | |
|---|---|
| distilled water | q.s. to 100 mL |

### Formulation Example 4—Injectable Formulation

The following ingredients are mixed to form an injectable formulation.

| Ingredient | Amount |
|---|---|
| compounds disclosed herein | 0.2 mg-20 mg |
| sodium acetate buffer solution, 0.4M | 2.0 mL |
| HCl (1N) or NaOH (1N) | q.s. to suitable pH |
| water (distilled, sterile) | q.s. to 20 mL |

### Formulation Example 5—Suppository Formulation

A suppository of total weight 2.5 g is prepared by mixing the compound of the present technology with Witepsol® H-15 (triglycerides of saturated vegetable fatty acid; Riches-Nelson, Inc., New York), and has the following composition:

| Ingredient | Amount |
|---|---|
| compounds disclosed herein | 500 mg |
| Witepsol ® H-15 | balance |

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples. The following examples will further describe the present invention, and are used for the purposes of illustration only, and should not be considered as limiting.

### EXAMPLES

### General Procedures

It will be apparent to the skilled artisan that methods for preparing precursors and functionality related to the compounds claimed herein are generally described in the literature. In these reactions, it is also possible to make use of variants which are themselves known to those of ordinary skill in this art, but are not mentioned in greater detail. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March Advanced Organic Chemistry (Wiley), Carey and Sundberg, Advanced Organic Chemistry (incorporated herein by reference in their entirety) and the like. All the intermediate compounds of the present invention were used without further purification unless otherwise specified.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene and P. Wuts Protecting Groups in Organic Synthesis, 4th Ed., John Wiley & Sons (2007), incorporated herein by reference in its entirety.

The following example schemes are provided for the guidance of the reader, and represent preferred methods for making the compounds exemplified herein. These methods are not limiting, and it will be apparent that other routes may be employed to prepare these compounds. Such methods specifically include solid phase based chemistries, including combinatorial chemistry. The skilled artisan is thoroughly equipped to prepare these compounds by those methods given the literature and this disclosure. The compound numberings used in the synthetic schemes depicted below are meant for those specific schemes only, and should not be construed as or confused with same numberings in other sections of the application.

Trademarks used herein are examples only and reflect illustrative materials used at the time of the invention. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the invention.

The following abbreviations have the indicated meanings:
2N=2 normality solution
ACN=acetonitrile
AIBN=azobisisobutyronitrile
aq.=aqueous
Bn=benzyl
Boc=tert-butoxycarbonyl
Bu=butyl
CD₃OD=deuterated methanol
CHCl₃=chloroform
CH₂Cl₂=methylene chloride, or dichloromethane
Cs₂CO₃=cesium carbonate
DIEA=N,N-diisopropylethylamine
DMF=dimethylformamide
DMSO=dimethylsulfoxide
EDCl=1-ethyl-3-(3-dimethylaminopropyl)carbodiimide
Et=ethyl
EtOAc=ethyl acetate
g=gram
FA=formic acid
h=hour
¹H=proton
H=hydrogen
H₂O=water
HCl=hydrochloric acid
HOBt=hydroxybenzotriazole
HPLC=high-performance liquid chromatography
iPrOH=isopropyl alcohol
LCMS=liquid chromatographic mass spectroscopy
Me=methyl
M=molarity
m=multiplet
[M+H]⁺=molecular ion plus one proton
MeOH=methanol
min=minute
mL=milliliter
mmol=millimole
MHz=megahertz
m/z=mass to charge ratio
N₂=nitrogen
NaCl=sodium chloride
Na₂SO₄=sodium sulfate
NH₃=ammonia
NBS=N-bromo succinimide
NH₄HCO₃=ammonium hydrogen carbonate
NMR=nuclear magnetic resonance
PBr₃=phosphorous tribromide
PCl₅=phosphorous pentachloride
PEG=polyethylene glycol
Ph=phenyl
pH=negative logarithm of hydrogen ion concentration
PPh₃Cl₂=triphenylphosphine dichloride
ppm=parts per million
q=quartet
rt=room temperature
s=singlet
sat.=saturated
t=triplet
SFC=supercritical fluid chromatography
TBSCl=tert-butyldimethylsilyl chloride
tBu=tert-butyl
TFA=trifluoroacetic acid
THF=tetrahydrofuran
TLC=thin layer chromatography
TMS=trimethylsilyl

The following example schemes are provided for the guidance of the reader, and collectively represent an example method for making the compounds provided herein. Furthermore, other methods for preparing compounds described herein will be readily apparent to the person of ordinary skill in the art in light of the following reaction schemes and examples. Unless otherwise indicated, all variables are as defined above.

### Example 1: Preparation of N-((1,2,3,5,6,7-hexahydro-s-indacen-4-yl)carbamoyl)-4-((3-hydroxyazetidin-1-yl)methyl)furan-2-sulfonimidamide

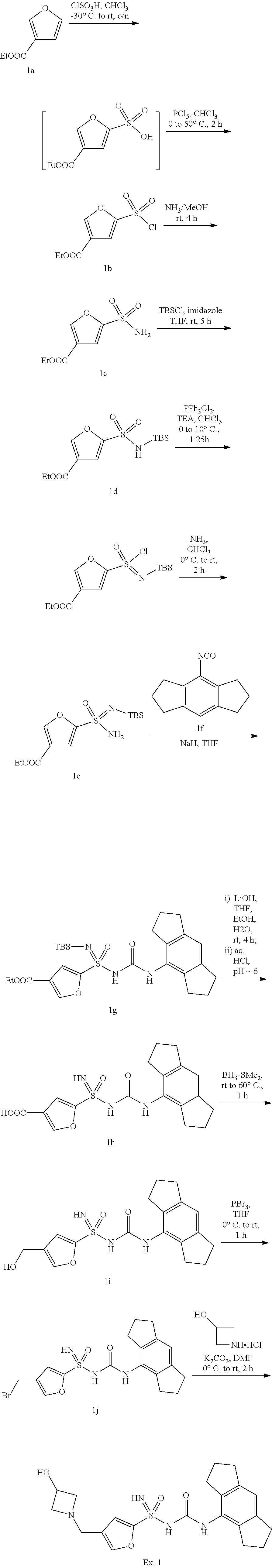

### Step 1:

To a pale yellow solution of ethyl furan-3-carboxylate 1a (3.00 g, 21.407 mmol) in CHCl₃ (80 mL) at −30° C. was added a colorless solution of chlorosulfonic acid (4.99 g, 42.815 mmol) in CHCl₃ (5 mL) dropwise with stirring. The resulting dark mixture was warmed up slowly to rt and then stirred overnight. The reaction progress was monitored by TLC and LCMS. After completion, the black reaction mixture was cooled to −20° C., and PCl_{5 (}11.14 g, 53.519 mmol) was added to give a black suspension. The suspension was warmed to rt and stirred at 50° C. for 2 h to generate a brown suspension, which was poured onto crushed ice (200 g) with stirring. The mixture was separated, the aqueous phase was extracted with CH₂Cl₂ (50 mL×2), and the combined organic phases were diluted with EtOAc (120 mL), washed with a cold sat. aq. NaCl solution(50 mL×2), and dried over Na₂SO₄. Removal of solvents afforded ethyl 5-(chlorosulfonyl)furan-3-carboxylate 1b as a brown oil (3.9 g, 75.7%). This crude product was used in the next step without further purification. 1b: ¹HNMR (300 MHz, DMSO-d6) δ (ppm): 8.26 (s, 1H), 6.58 (s, 1H), 4.23 (q, J=7.14 Hz, 2H), 1.25 (t, J=7.14 Hz, 3H).

### Step 2:

A 7 M solution of NH₃ in MeOH (30 mL, 1056.93 mmol) was added to ethyl 5-(chlorosulfonyl)furan-3-carboxylate 1b (3.5 g, 14.666 mmol). The reaction mixture was stirred at room temperature for 4 h. N₂ gas was bubbled into the reaction mixture for 0.5 h to purge excess NH₃. The mixture was concentrated to a residue and purified by silica gel flash chromatography eluting with EtOAc in petroleum ether (0 to 34%) to provide ethyl 5-sulfamoylfuran-3-carboxylate 1c as a light yellow solid (1.4 g, 43.55%), ¹HNMR (300 MHz, DMSO-d₆) δ (ppm): 8.64 (s, 1H), 7.13 (s, 1H), 4.26 (q, J=7.10 Hz, 2H), 1.28 (t, J=7.10 Hz, 3H), LC-MS: m/z [M+H]⁺=218.0.

### Step 3:

A colorless suspension of ethyl 5-sulfamoylfuran-3-carboxylate 1c (1.20 g, 5.474 mmol), TBSCl (4.13 g, 27.381 mmol) and 1H-imidazole (0.75 g, 10.948 mmol) in DCM (120 mL) was stirred at room temperature overnight. The suspension was washed with H₂O (100 mL×2) and brine (100 mL×2). The organic phase was dried over Na₂SO₄, filtered and concentrated to a residue, purified by silica gel flash chromatography eluting with EtOAc/petroleum ether (0-17%) to give ethyl 5-[(tert-butyldimethylsilyl)sulfamoyl]furan-3-carboxylate 1d as a white solid (1.6 g, 80.43%). ¹HNMR (300 MHz, DMSO-d6) δ (ppm): 8.64 (s, 1H), 8.14 (s, 1H), 7.10 (s, 1H), 4.28 (q, J=7.10 Hz, 2H), 1.28 (t, J=7.10 Hz, 3H), 0.89 (s, 9H), 0.17 (s, 6H); LC-MS: m/z [M+H]⁺=332.0.

### Step 4:

A colorless mixture of triphenylphosphine (3.80 g, 14.488 mmol) and hexachloroethane (3.43 g, 14.488 mmol) in CHCl₃ (72 mL) was refluxed at 70° C. overnight to provide a colorless suspension of PPh₃Cl₂, used directly without a workup. The suspension was cooled at 0° C., and added dropwise an anhydrous Et₃N (3.75 mL, 37.073 mmol) to generate a yellow suspension. After stirred at 0° C. for 10 min, a solution of ethyl 5-[(tert-butyldimethylsilyl)sulfamoyl]furan-3-carboxylate Id (3.00 g, 8.996 mmol) in CHCl₃ (8 mL) was added dropwise 0° C. resulting a light brown suspension. Reaction mixture was stirred at 0° C. for 1 h, and a saturated solution of NH₃ in CHCl₃ (120 mL) was added dropwise. Cooling bath was removed, the suspension was stirred at room temperature for 2 h. N₂ gas was bubbled into the mixture to purge excess NH Solvents were removed under vacuo and the residue was purified by silica gel flash chromatography affording ethyl 5-[(tert-butyldimethylsilyl)-S-aminosulfonimidoyl]furan-3-carboxylate 1e as a white solid (1.0 g, 33%), ¹HNMR (400 MHz, DMSO-d6) δ (ppm): 8.54 (s, 1H), 7.14 (s, 1H), 6.90 (s, 1H), 4.28 (q, J=7.10 Hz, 2H), 1.28 (t, J=7.10 Hz, 3H), 0.86 (s, 9H), 0.01 (s, 6H).

### Step 5:

Sodium hydride 60% dispersion in oil (106 mg, 4.448 mmol) was added to a colorless solution of ethyl 5-[amino[(tert-butyldimethylsilyl)imino]methylidene-lambda6-sulfanyl]furan-3-carboxylate 1e (1.40 g, 4.236 mmol) in THF at 0° C. The resulting mixture was stirred at 0° C. for 10 min. 4-isocyanato-1,2,3,5,6,7-hexahydro-s-indacene if (843 mg, 4.236 mmol) was added. Cooling bath was removed, and the reaction mixture was stirred at room temperature for 2 h. DCM (50 mL) was added, and the mixture was washed with an aqueous solution of NH₄Cl (50 mL×2). The aqueous washings were combined and re-extracted with DCM (20 mL×2). The combined organic extracts were washed with brine (50 mL×2), dried over Na₂SO₄, filtered and concentrated to a residue, which was purified by silica gel flash chromatography (0-20% EtOAc/petroleum ether) to give ethyl 5-(N′-(tert-butyldimethylsilyl)-N-((1,2,3,5,6,7-hexahydro-s-indacen-4-yl)carbamoyl)sulfamidimidoyl)furan-3-carboxylate 1g as a yellow solid (1.6 g, 67.97%), ¹HNMR (300 MHz, DMSO-d6) δ (ppm): 8.64 (s, 1H), 8.12 (s, 1H), 7.88 (s, 1H), 7.16 (s, 1H), 7.10 (s, 1H), 4.24 (q, J=7.10 Hz, 2H), 2.58-2.79 (m, 8H), 1.89-1.99 (m, 4H), 1.28 (t, J=7.10 Hz, 3H), 0.85 (s, 9H), 0.07 (s, 6H); LC-MS: m/z [M+H]⁺=532.4.

### Step 6:

A solution of LiOH (190 mg, 7.899 mmol) in H₂O (8 mL) was added to a slight yellow solution of ethyl 5-[[(tert-butyldimethylsilyl)imino]([[(1,2,3,5,6,7-hexahydro-s-indacen-4-yl)carbamoyl]amino])oxo-lambda6-sulfanyl]furan-3-carboxylate 1g (1.40 g, 2.633 mmol) in THE (16 mL) and EtOH (4 mL) at room temperature. The mixture was stirred at room temperature for 4 h and concentrated in vacuo to remove volatile solvents. The resulting colorless suspension was treated dropwise with a 2N HCl aqueous solution until its pH value reached approximately 6 and colorless solids precipitated. The precipitate was filtered, rinsed with MeCN, and dried under reduced vacuum to afford 5-([[(1,2,3,5,6,7-hexahydro-s-indacen-4-yl)carbamoyl]amino](imino)oxo-lambda6-sulfanyl)furan-3-carboxylic acid 1 h as a white solid (0.9 g). 150 mg of the solid was purified by Prep-HPLC using the following conditions: Column: XBridge Shield RP18 OBD Column, 19*250 mm, 10 μm; Mobile Phase A: Water (10 mM NH₄HCO₃), Mobile Phase B: ACN; Flow rate: 25 mL/min; Gradient: 12% B to 45% B in 7 min, 45% B to B; 220 nm; RTL: 6.32 min. After lyophilization, 5-(N-((1,2,3,5,6,7-hexahydro-s-indacen-4-yl)carbamoyl)sulfamidimidoyl)furan-3-carboxylic acid 1h was obtained as a white solid (42.5 mg, 4.12%); ¹HNMR (300 MHz, Methanol-d₄) δ (ppm): 8.13 (s, 1H), 7.31 (s, 1H), 6.92 (s, 1H), 2.76-2.87 (m, 8H), 1.98-2.08 (m, 4H); LC-MS: m/z [M+H]⁺=388.0.

### Step 7:

To a stirred solution of 5-([[(1,2,3,5,6,7-hexahydro-s-indacen-4-yl)carbamoyl]amino](imino)oxo-lambda6-sulfanyl)furan-3-carboxylic acid 1h (900 mg, 2.311 mmol) in THE (18 mL) was added dimethyl sulfide borane (18 ml) dropwise at rt under a nitrogen atmosphere. The resulting colorless suspension was heated at 60° C. for 1 h. After cooling to room temperature, the reaction was quenched carefully with the addition of MeOH (150 mL), and the mixture was filtered and concentrated. The crude product was purified by Prep-HPLC using the following conditions: Column: YMC-Actus Triart C18, 30*250 mm, 5 um; Mobile Phase A: Water (10 mmol/L NH₄HCO₃+0.1% NH₃·H₂O), Mobile Phase B: ACN; Flow rate: 60 mL/min; Gradient: 26% B to 46% B in 7 min, 46% B to B. After lyophilization, N-((1,2,3,5,6,7-hexahydro-s-indacen-4-yl)carbamoyl)-4-(hydroxymethyl)furan-2-sulfonimidamide 1i was obtained as a white solid (26.1 mg, 3%), ¹H NMR (300 MHz, CD₃OD) δ (ppm): 7.67 (s, 1H), 7.07 (s, 1H), 6.90 (s, 1H), 4.48 (d, J=1.00 Hz, 2H), 2.62-3.02 (m, 8H), 1.89-2.16 (m, 4H); LC-MS: m/z [M+H]⁺=376.1.

### Step 8:

PBr₃ (793 mg, 2.930 mmol) was added to a stirred solution of 3-(1,2,3,5,6,7-hexahydro-s-indacen-4-yl)-1-[[4-(hydroxymethyl)furan-2-yl](imino)oxo-lambda6-sulfanyl]urea 1i (1.1 g, 2.930 mmol) in THE (20 mL) at 0° C. The mixture was stirred for 10 min at 0° C. and continued at room temperature for 1 h. The reaction was quenched by addition of water (10 mL). The aqueous mixture was extracted with 2-propanol/CHCl₃ (v/v, 1/3, 20 mL×3), the combined organic extracts were washed with brine (20 mL), dried over Na₂SO₄ and filtered. Removal of solvents provided obtained a solid which was further recrystallized from PE to give 1-[[4-(bromomethyl)furan-2-yl](imino)oxo-lambda6-sulfanyl]-3-(1,2,3,5,6,7-hexahydro-s-indacen-4-yl)urea 1j as a white solid (1.05 g, 70.72%), LC-MS: m/z [M]⁺, [M+2]⁺=438.05, 440.05.

### Step 9:

A colorless suspension of 3-hydroxyazetidin-1-ium chloride (60.0 mg, 0.548 mmol) and Cs₂CO₃ (356.8 mg, 1.096 mmol) in DMF (2 mL) was stirred at room temperature for 20 min. 3-[[4-(bromomethyl)furan-2-yl](imino)oxo-lambda6-sulfanyl]-1-(1,2,3,5,6,7-hexahydro-s-indacen-4-yl)urea 1j (120.0 mg, 0.274 mmol) was added. The resulting brown suspension was stirred at room temperature for 1.5 h. Reaction was quenched by the addition of water (5 mL), the aqueous mixture was extracted with CHCl₃/iPrOH (1/3, v/v, 20 mL×2). The combined organic phases were dried over Na₂SO₄ and concentrated under vacuo to a yellow oil, which was purified by Prep-HPLC using the following conditions: Column: XBridge BEH C18 OBD Prep Column, 19*250 mm; Mobile Phase A: Water (10 mmol/L NH₄HCO₃+0.1% NH₃·H₂O), Mobile Phase B: ACN; Flow rate: 25 mL/min; Gradient: 25% B to 30% B in 8 min, 30% B to B; 254 nm; RT1:7.07 min. After lyophilization, the titled compound 1-(1,2,3,5,6,7-hexahydro-s-indacen-4-yl)-3-([4-[(3-hydroxyazetidin-1-yl)methyl]furan-2-yl](imino)oxo-lambda6-sulfanyl)urea (1) was obtained as a white solid (13.3 mg, 11.18%), ¹HNMR (400 MHz, CD₃OD) δ (ppm): 7.67 (s, 1H), 7.04 (s, 1H), 6.90 (s, 1H), 4.33 (m, 1H), 3.61 (m, 2H), 3.53 (s, 2H), 2.83 (m, 2H), 2.75-2.81 (m, 8H), 2.00-2.03 (m, 4H); LC-MS: m/z [M+H]⁺=431.2.

### Example 2: Preparation of N-((1,2,3,5,6,7-hexahydro-s-indacen-4-yl)carbamoyl)-4-((6-hydroxy-2-azaspiro[3.3]heptan-2-yl)methyl)furan-2-sulfonimidamide

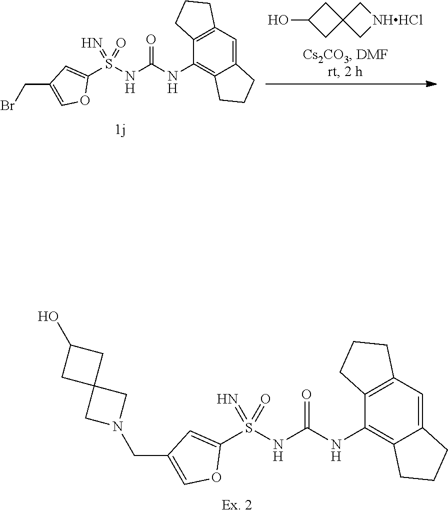

A colorless suspension of 6-hydroxy-2-azaspiro[3.3]heptan-2-ium chloride (88.8 mg, 0.593 mmol) and Cs₂CO₃ (386.5 mg, 1.186 mmol) in DMF (2 mL) was stirred at room temperature for 20 min. 3-[[4-(bromomethyl)furan-2-yl](imino)oxo-lambda6-sulfanyl]-1-(1,2,3,5,6,7-hexahydro-s-indacen-4-yl)urea 1j (130.0 mg, 0.297 mmol) was added. The resulting brown suspension was stirred at room temperature for an additional 1.5 h. Water (5 mL) was added and the aqueous mixture was extracted with CHCl₃/iPrOH (1/3, 20 mL×2). The combined organic extracts were dried over Na₂SO₄ and concentrated to a yellow oil, which was purified by Prep-HPLC using the following conditions: Column: YMC-Triart Diol Hilic, 20*150 mm 5 um; Mobile Phase A: Water (10 mmol/L NH₄HCO₃+0.1% NH₃·H₂O), Mobile Phase B: ACN; Flow rate: 60 mL/min; Gradient: 21% B to 41% B in 7 min, 41% B to B; 254 nm; RTL: 6.47 min. After lyophilization, the titled compound 1-(1,2,3,5,6,7-hexahydro-s-indacen-4-yl)-3-[[4-([6-hydroxy-2-azaspiro[3.3]heptan-2-yl]methyl)furan-2-yl](imino)oxo-lambda6-sulfanyl]urea Ex. 2 was obtained as a white solid (31.8 mg, 22.57%), ¹HNMR (400 MHz, CD₃OD) δ (ppm): 7.65 (s, 1H), 7.02 (s, 1H), 6.90 (s, 1H), 4.08 (m, 1H), 3.48 (s, 2H), 3.25 (s, 4H), 2.74-2.85 (m, 8H), 2.43-2.48 (m, 2H), 1.97-2.05 (m, 6H); LC-MS: m/z [M+H]⁺=470.59.

### Example 3: Preparation of N-((1,2,3,5,6,7-hexahydro-s-indacen-4-yl)carbamoyl)-4-(6-hydroxy-2-azaspiro[3.3]heptane-2-carbonyl)furan-2-sulfonimidamide

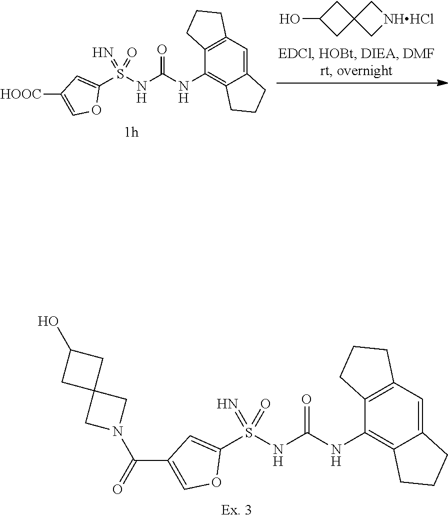

To a stirred colorless solution of 5-([[(1,2,3,5,6,7-hexahydro-s-indacen-4-yl)carbamoyl]amino](imino)oxo-lambda6-sulfanyl)furan-3-carboxylic acid 1h (130 mg, 0.334 mmol), 6-hydroxy-2-azaspiro[3.3]heptan-2-ium chloride (100 mg, 0.668 mmol) and DIEA (216 mg, 1.669 mmol) in DMF (5 mL) was added 1-ethyl-3-(3-dimethylaminopropyl)carbodiimide (EDCl, 96 mg, 0.501 mmol) and hydroxybenzotriazole (HOBt, 90 mg, 0.668 mmol). The reaction mixture was stirred at room temperature overnight. Water (10 mL) was added to quench the reaction. The aqueous mixture was extracted with EtOAc (30 mL×3), and the combined organic phases were washed with sat. NaCl (10 mL×3), dried over Na₂SO₄ and concentrated under vacuo. The residue was purified by Prep-HPLC using the following conditions: Column: Column: XBridge Shield RP18 OBD Column, 19*250 mm, 10 μm; Mobile Phase A: Water (10 mmol/L NH₄HCO₃+0.1% NH₃·H₂O), Mobile Phase B: ACN; Flow rate: 25 mL/min; Gradient: 21% B to 37% B in 7 min, 37% B to B; 254 nm; RT1: 6.38 min. After lyophilization, the titled compound 1-(1,2,3,5,6,7-hexahydro-s-indacen-4-yl)-3-[(4-[6-hydroxy-2-azaspiro[3.3]heptane-2-carbonyl]furan-2-yl)(imino)oxo-lambda6-sulfanyl]urea Ex. 3 was obtained as a white solid (32.8 mg, 20.25%), ¹HNMR (400 MHz, CD₃OD) δ (ppm): 8.17 (s, 1H), 7.26 (s, 1H), 6.91 (s, 1H), 4.37-4.40 (m, 2H), 4.00-4.14 (m, 3H), 2.76-2.85 (m, 8H), 2.56-2.59 (m, 2H), 1.97-2.14 (m, 6H); LC-MS: m/z [M+H]⁺=483.20.

### Example 4: Preparation of 5-(N-((1,2,3,5,6,7-hexahydro-s-indacen-4-yl)carbamoyl)sulfamidimidoyl)-N-((1-hydroxycyclobutyl)methyl)-N-methylfuran-3-carboxamide

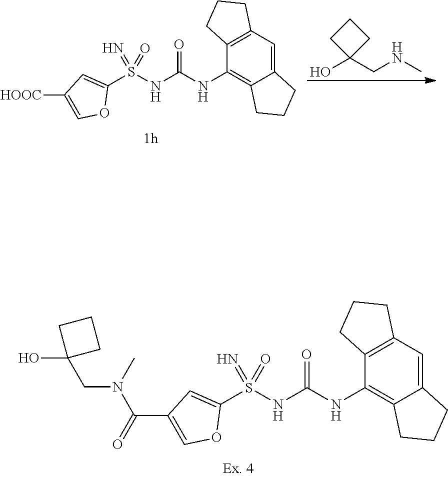

Following a similar procedure described in preparative example 3, the titled compound 5-([[(1,2,3,5,6,7-hexahydro-s-indacen-4-yl)carbamoyl]amino](imino)oxo-lambda6-sulfanyl)-N-[(1-hydroxycyclobutyl)methyl]-N-methylfuran-3-carboxamide Ex. 4 was obtained as a white solid (28.0 mg, 27.82%), ¹HNMR (400 MHz, CD₃OD) δ ppm: 8.14 (s, 1H), 7.29 (s, 1H), 6.90 (s, 1H), 3.63 (s, 2H), 3.10 (s, 1H), 2.75-2.84 (m, 9H), 1.90-2.14 (m, 9H), 1.67-1.79 (m, 2H); LC-MS: m/z [M+H]⁺=487.2.

### Example 5: Preparation of N-((1,2,3,5,6,7-hexahydro-s-indacen-4-yl)carbamoyl)-4-(((R)-3-(hydroxymethyl)pyrrolidin-1-yl)methyl)-5-methylfuran-2-sulfonimidamide

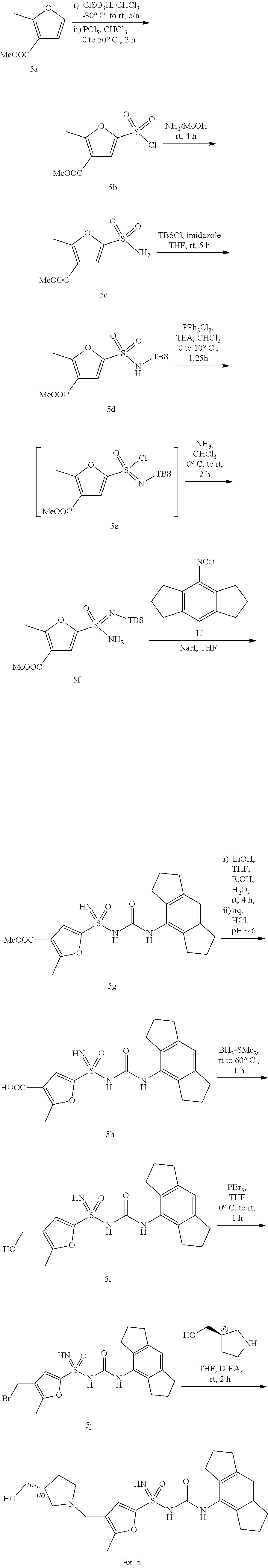

### Steps 1 and 2:

To a stirred light yellow solution of methyl 2-methylfuran-3-carboxylate 5a (85 g, 606.54 mmol) in CHCl₃ (2000 mL) at −30° C. was added chlorosulfonic acid (141 g, 1213 mmol) dropwise. The resulting light brown mixture was allowed to warm up slowly to rt and stirred overnight. The reaction progress was monitored by TLC. After completion, the mixture was cooled down to −20° C., and added PCl₅ (316 g, 1516 mmol) to give a black suspension. The suspension was warmed to rt and then heated at 50° C. for 2 h. After cooling to rt, the suspension was poured onto ice (2000 g) under stirring. The mixture was separated, the aqueous phase was extracted with CH₂Cl₂ (1000 mL×2), and the combined organic phases were diluted with EtOAc (4000 mL), washed with cold brine (2000 mL×2), dried with Na₂SO₄, filtered and concentrated in vacuo to give methyl 5-(chlorosulfonyl)-2-methylfuran-3-carboxylate 5b as a brown oil (95 g, 65.63%).

The crude product 5b was dissolved in a solution of NH₃ in MeOH (300 mL, 7 M). The resulting brown solution was stirred at room temperature overnight. N₂ was bubbled into the reaction for 0.5 h to purge excess NH₃. The mixture was concentrated and the residue was purified by silica gel flash chromatography (0-34% EtOAc/petroleum ether) to give methyl 2-methyl-5-sulfamoylfuran-3-carboxylate 5c as a white solid (40 g, 30.07%), ¹HNMR (400 MHz, CD₃OD) δ (ppm): 7.10 (s, 1H), 3.84 (s, 3H), 2.79 (s, 3H); LC-MS: m/z [M+H]⁺=218.0.

### Step 3:

A colorless suspension of methyl 5-sulfamoylfuran-3-carboxylate 5c (40 g, 182.47 mmol), TBSCl (137.5 g, 912.36 mmol) and 1H-imidazole (24.8 g, 364.94 mmol) in DCM (1500 mL) was stirred at room temperature overnight. The suspension was washed with H₂O (1000 mL×2) and sat. NaCl (1000 mL×2). The organic phase was dried over Na₂SO₄, filtered, and concentrated. The residue was purified by silica gel flash chromatography (0-17% EtOAc in petroleum ether) to give methyl 5-[(tert-butyldimethylsilyl)sulfamoyl]furan-3-carboxylate 5d as a white solid (50 g, 82.17%), ¹HNMR (400 MHz, CD₃OD) δ (ppm): 7.00 (s, 1H), 3.79 (s, 3H), 2.57 (s, 3H), 0.89 (s, 9H), 0.17 (s, 6H); LC-MS: m/z [M+H]⁺=332.0.

### Step 4:

To a colorless suspension of freshly prepared dichlorotriphenylphosphorane (60 g, 179.92 mmol) in CHCl₃ (700 mL) at 0° C. was added dropwise anhydrous triethyl amine (50 mL, 359.85 mmol). The resulting light yellow suspension was stirred at 0° C. for 10 min. A colorless solution of methyl 5-(N-(tert-butyldimethylsilyl)sulfamoyl)-2-methylfuran-3-carboxylate 5d (40 g, 119.95 mmol) in CHCl₃ (30 mL) was added dropwise leading to a light brown suspension. The mixture was stirred at 0° C. for 1 h. Then, a saturated solution of NH₃ in CHCl₃ (800 mL) was added slowly. Cooling bath was removed, and the reaction mixture was stirred at room temperature for 2 h. N₂ was bubbled into the mixture to remove excess NH₃. Removal of solvents provided a residue, which was purified by silica gel flash chromatography to afford methyl 5-(N′-(tert-butyldimethylsilyl)sulfamidimidoyl)-2-methylfuran-3-carboxylate 5f as a white solid (15 g, 37.61%), ¹HNMR (400 MHz, DMSO-d&) δ (ppm): 6.88 (s, 1H), 3.78 (s, 3H), 2.56 (s, 3H), 0.86 (s, 9H), 0.02 (s, 6H); LC-MS: m/z [M+H]⁺=331.0.

### Step 5:

NaH (60% dispersion in oil, 216 mg, 9.023 mmol) was added to a colorless solution of methyl 5-[amino[(tert-butyldimethylsilyl)imino]methylidene-lambda6-sulfanyl]furan-3-carboxylate 5f (3 g, 9.023 mmol) in THF (30 mL) at 0° C. The resulting mixture was stirred at 0° C. for 10 min. 4-isocyanato-1,2,3,5,6,7-hexahydro-s-indacene 1f (1.8 g, 9.023 mmol) was added. Cooling bath was removed, and the reaction mixture was stirred at room temperature for 2 h. The mixture was diluted with CHCl₃/iPr-OH (v/v, 3/1, 50 mL) and washed with a sat. aqueous solution of NH₄Cl (50 mL×2). The aqueous phases were combined and re-extracted with CHCl₃/iPr-OH (v/v, 3/1, 40 mL×2). The combined organic phases were washed with brine (50 mL×2), dried with Na₂SO₄, filtered and concentrated. The residue was purified by silica gel flash chromatography to provide methyl 5-N-((1,2,3,5,6,7-hexahydro-s-indacen-4-yl)carbamoyl)sulfamidimidoyl)furan-3-carboxylate 5g as a white solid (3.2 g, 84.95%), LC-MS: m/z [M+H]⁺=418.20.

### Step 6:

An aqueous solution of LiOH (3.10 g, 129.348 mmol) in H₂O (35 mL) was added to a slight brown suspension of methyl 5-([[(1,2,3,5,6,7-hexahydro-s-indacen-4-yl)carbamoyl]amino](imino)oxo-lambda6-sulfanyl)-2-methylfuran-3-carboxylate 5g (18 g, 43.116 mmol) in THF (70 mL) and MeOH (12.5 mL). The resulting suspension was stirred at room temperature for 4 h. An aqueous solution of HCl (2 N) was added to the mixture until pH 6. Solvents were removed in vacuo to give a slight brown solid, which was rinsed with MeCN (60 mL) to afford 5-([[(1,2,3,5,6,7-hexahydro-s-indacen-4-yl)carbamoyl]amino](imino)oxo-lambda6-sulfanyl)-2-methylfuran-3-carboxylic acid 5h as a slightly brown solid (15 g). 100 mg of the crude 5h was purified by Prep-HPLC using the following conditions: Column: Xselect CSH OBD Column 30*150 mm 5 um; Mobile Phase A: Water(0.1% FA), Mobile Phase B: ACN; Flow rate: 60 mL/min; Gradient: 25% B to 53% B in 7 min, 53% B to B; 220 nm; RT1: 6.42 min. After lyophilization, 5-([[(1,2,3,5,6,7-hexahydro-s-indacen-4-yl)carbamoyl]amino](imino)oxo-lambda6-sulfanyl)-2-methylfuran-3-carboxylic acid 5h was obtained as a white solid (40.1 mg). ¹HNMR (400 MHz, CD₃OD) δ (ppm): 7.23 (s, 1H), 6.91 (s, 1H), 2.76-2.85 (m, 8H), 2.64 (s, 3H), 1.98-2.06 (m, 4H); LC-MS: m/z [M+H]⁺=404.30.

### Step 7:

Borane dimethyl sulfide complex (17.9 mL, 10M, 179 mmol) was added dropwise to a stirred colorless suspension of 5-([[(1,2,3,5,6,7-hexahydro-s-indacen-4-yl)carbamoyl]amino](imino)oxo-lambda6-sulfanyl)-2-methylfuran-3-carboxylic acid 5h (8 g, 19.829 mmol) in THF (150 mL) at 0° C. under N2. Cooling was stopped, and the reaction mixture was heated up at 60° C. for 1 h. After cooled to room temperature, the reaction was quenched with an aqueous solution of NH₄Cl (20 mL) and further diluted with water (50 mL). The aqueous mixture was extracted with CHCl₃/iPrOH (3v/1v, 300 mL×3). The combined organic phases were dried over Na₂SO₄ and concentrated in vacuo to give N-((1,2,3,5,6,7-hexahydro-s-indacen-4-yl)carbamoyl)-4-(hydroxymethyl)-5-methylfuran-2-sulfonimidamide Si as a white solid (6.2 g). 100 mg of the crude Si was purified by Prep-HPLC using the following conditions: Column: Xselect CSH OBD Column 30*150 mm 5 μm; Mobile Phase A: Water(0.1% FA), Mobile Phase B: ACN; Flow rate: 60 mL/min: Gradient: 25% B to 53% B in 7 min, 53% B to B; 220 nm; RT1: 6.42 min; after lyophilization, N-((1,2,3,5,6,7-hexahydro-s-indacen-4-yl)carbamoyl)-4-(hydroxymethyl)-5-methylfuran-2-sulfonimidamide Si was obtained as a white solid (44.2 mg), ¹HNMR (400 MHz, CD₃OD) δ (ppm): 7.02 (s, 1H), 6.91 (s, 1H), 4.41 (s, 2H), 2.77-2.85 (m, 8H), 2.36 (s, 3H), 1.98-2.06 (m, 4H); LC-MS: m/z [M+H]⁺=390.15.

### Step 8:

PBr₃ (2.78 g, 10.270 mmol) was added dropwise to a stirred colorless suspension of N-((1,2,3,5,6,7-hexahydro-s-indacen-4-yl)carbamoyl)-4-(hydroxymethyl)-5-methylfuran-2-sulfonimidamide Si (4 g, 10.270 mmol) in THF (50 mL) at 0° C. Cooling was stopped, and the resulting suspension was stirred at room temperature for 1 h. The reaction was quenched with the addition of water (30 mL). The aqueous mixture was extracted with iPrOH/CHCl₃ (v/v, 1/3, 50 mL×3). The combined organic phases were washed with brine (50 mL), dried with Na₂SO₄, filtered and concentrated to a solid. The crude solid product was recrystallized from petroleum ether to afford 4-(bromomethyl)-N-((1,2,3,5,6,7-hexahydro-s-indacen-4-yl)carbamoyl)-5-methylfuran-2-sulfonimidamide 5j as a white solid (4 g, 86.10%), LC-MS: m/z [M]⁺=450.05, [M+2]⁺=452.05.

### Step 9:

A brown suspension of 4-(bromomethyl)-N-((1,2,3,5,6,7-hexahydro-s-indacen-4-yl)carbamoyl)-5-methylfuran-2-sulfonimidamide 5j (150 mg, 0.332 mmol), (3R)-pyrrolidin-3-ylmethanol (67 mg, 0.663 mmol) and DIEA (129 mg, 0.996 mmol) in THF (2 mL) was stirred at room temperature for 2 h. Water (5 mL) was added, and the aqueous mixture was extracted with iPrOH/CHCl₃ (v/v, 1/3, 10 mL×3). The combined organic phases were concentrated under vacuo to a residue which was purified by reverse phase column (spherical C18, 120 g, MeCN/water (0.05% NH₄HCO₃)). The combined elutes were extracted with iPrOH/CHCl₃ (v/v, 1/3, 100 mL×3), and the organic phase was concentrated to a residue. This residue was purified by Prep-HPLC using the following conditions: Column: XBridge Shield RP1 8 OBD Column, 30*150 mm, 5 μm; Mobile Phase A: Water(0.1% FA), Mobile Phase B: ACN; Flow rate: 60 mL/min; 254 nm; RT1:6.92 min. After lyophilization, the titled compound N-((1,2,3,5,6,7-hexahydro-s-indacen-4-yl)carbamoyl)-4-(((R)-3-(hydroxymethyl)pyrrolidin-1-yl)methyl)-5-methylfuran-2-sulfonimidamide (Ex. 5) formic acid salt was obtained as a white solid (33.3 mg, 18.76%). ¹HNMR (400 MHz, CD₃OD) δ (ppm): 7.09 (s, 1H), 6.91 (s, 1H), 3.89 (m, 2H), 3.47-3.53 (m, 2H), 3.04-3.18 (m, 3H), 2.75-2.85 (m, 9H), 2.50 (m, 1H), 2.41 (s, 3H), 1.98-2.06 (m, 5H), 1.71-1.74 (m, 1H); LC-MS: m/z [M+H]⁺=473.25.

### Example 6: Preparation of N-((1,2,3,5,6,7-hexahydro-s-indacen-4-yl)carbamoyl)-4-(((S)-3-(hydroxymethyl)pyrrolidin-1-yl)methyl)-5-methylfuran-2-sulfonimidamide

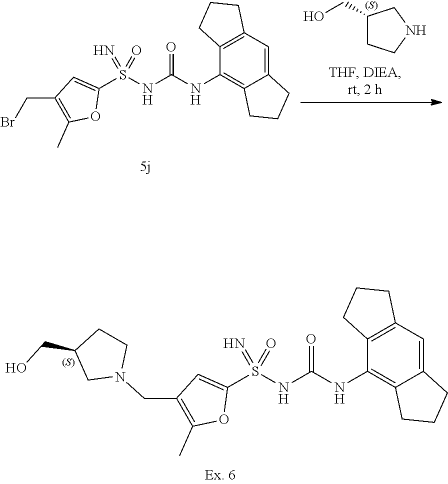

A brown suspension of 1-[[4-(bromomethyl)-5-methylfuran-2-yl](imino)oxo-lambda6-sulfanyl]-3-(1,2,3,5,6,7-hexahydro-s-indacen-4-yl)urea 5j (150 mg, 0.332 mmol), (3S)-pyrrolidin-3-ylmethanol (67 mg, 0.663 mmol) and DIEA (129 mg, 0.996 mmol) in THF (2 mL) was stirred at room temperature for 2 h. Water (5 mL) was added and the aqueous mixture was extracted with iPrOH/CHCl₃ (v/v, 1/3, 10 mL×3). The combined organic extracts were concentrated under vacuo to a residue, which was purified by reverse phase column chromatography (spherical C18, 120 g, MeCN/water (0.05% NH₄HCO₃)). The combined elutes were extracted with iPrOH/CHCl₃ (v/v, 1/3, 100 mL×3). Removal of solvents gave a residue, which was further purified by Prep-HPLC using the following conditions: Column: Xselect CSH OBD Column 30*150 mm 5 μm; Mobile Phase A: Water(0.1% formic acid), Mobile Phase B: ACN; Flow rate: 60 mL/min; Gradient: 14% B to 34% B in 9 min, 34% B; Wavelength: 254 nm; RT1(min): 6.92. After lyophilization, the titled compound 3-(1,2,3,5,6,7-hexahydro-s-indacen-4-yl)-1-[(4-[[(3S)-3-(hydroxymethyl)pyrrolidin-1-yl]methyl]-5-methylfuran-2-yl)(imino)oxo-lambda6-sulfanyl]urea Ex. 6 formic acid salt was obtained as a white solid (35.8 mg, 20.21%). ¹HNMR (400 MHz, CD₃OD) δ (ppm): 7.07 (s, 1H), 6.91 (s, 1H), 3.89 (m, 2H), 3.47-3.53 (m, 2H), 3.04-3.18 (m, 3H), 2.75-2.85 (m, 9H), 2.50 (m, 1H), 2.41 (s, 3H), 1.98-2.06 (m, 5H), 1.71-1.74 (m, 1H); LC-MS: m/z [M+H]⁺=473.10.

### Examples 7 to 12

Following the procedures described in Preparative Example 6, by using suitable amine precursor R₁R₂NH under condition A or R₁R₂NH—HCl salt under condition B, Examples 7 to 12 were prepared from bromo intermediate 5j, available from Preparative Example 5.

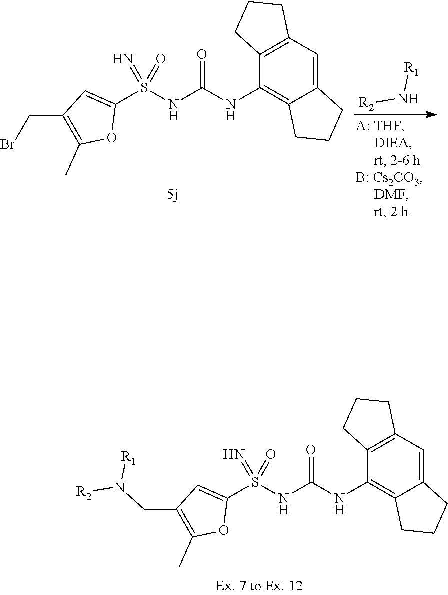

TABLE 1

| Ex. | | | |
|---|---|---|---|
| No. | R₁R₂NH | Structure/Name | LC-MS, ¹H NMR |
| 7 | 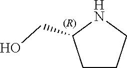 | 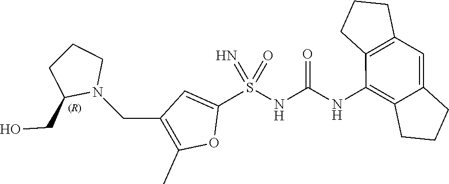 | LC-MS: m/z [M + H]⁺ = 473.15; ¹H NMR (400 MHz, CD₃OD) δ (ppm): 7.09 (s, 1H), 6.91 (s, 1H), 4.08 − 4.13 (m, 1H), 3.59 − 3.67 (m, 3H), 3.13 − 3.19 (m, 1H), 2.95 − 3.02 (m, 1H), 2.61 − 2.85 (m, 9H), 2.40 (s, 3H), 1.98 − 2.06 (m, 5H), 1.71 − 1.90 (m, 3H). |
| | | N-((1,2,3,5,6,7-hexahydro-s- | |
| | | indacen-4-yl)carbamoyl)-4-(((R)-2- | |
| | | (hydroxymethyl)pyrrolidin-1- | |
| | | yl)methyl)-5-methylfuran-2- | |
| | | sulfonimidamide | |
| 8 | 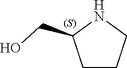 | 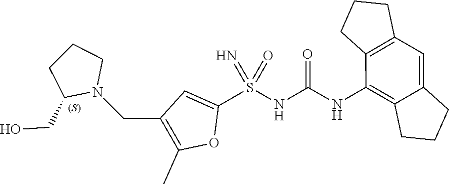 | LC-MS: m/z [M + H]⁺ = 473.25; ¹H NMR (400 MHz, CD₃OD) δ (ppm): 7.10 (s, 1H), 6.91 (s, 1H), 4.19 − 4.23 (m, 1H), 3.61 − 3.78 (m, 3H), 3.12 − 3.26 (m, 2H), 2.76 − 2.85 (m, 9H), 2.41 (s, 3H), 1.96 − 2.10 (m, 5H), 1.71 − 1.97 (m, 3H). |
| | | N-((1,2,3,5,6,7-hexahydro-s- | |
| | | indacen-4-yl)carbamoyl)-4-(((S)-2- | |
| | | (hydroxymethyl)pyrrolidin-1- | |
| | | yl)methyl)-5-methylfuran-2- | |
| | | sulfonimidamide | |
| 9 | 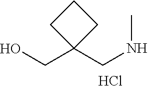 | 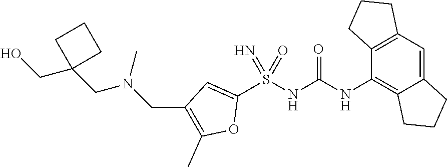 [SMILES: Cc1oc(S(=N)(=O)NC(=O)Nc2c3c(cc4c2CCC4)CCC3)cc1CN(C)CC1(CO)CCC1] | LC-MS: m/z [M + H]⁺ = 501.20; ¹H NMR (400 MHz, CD₃OD) δ (ppm): 7.05 (s, 1H), 6.91 (s, 1H), 3.77 (m, 4H), 2.93 (s, 2H), 2.75 − 2.85 (m, 8H), 2.49 (s, 3H), 2.39 (s, 3H), 1.87 − 2.06 (m, 10H). |
| | | N-((1,2,3,5,6,7-hexahydro-s- | |
| | | indacen-4-yl)carbamoyl)-4-((((1- | |
| | | (hydroxymethyl)cyclobutyl)methyl) | |
| | | (methyl)amino)methyl)-5- | |
| | | methylfuran-2-sulfonimidamide | |
| 10 | 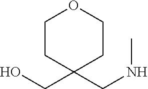 | 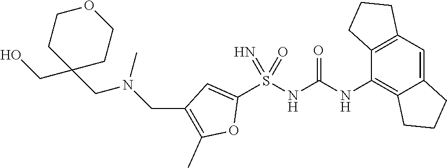 [SMILES: Cc1oc(S(=N)(=O)NC(=O)Nc2c3c(cc4c2CCC4)CCC3)cc1CN(C)CC1(CO)CCOCC1] | LC-MS: m/z [M + H]⁺ = 531.20; ¹H NMR (400 MHz, CD;OD) δ (ppm): 7.05 (s, 1H), 6.91 (s, 1H), 3.68 (s, 2H), 3.54 − 3.67 (m, 6H), 2.79 − 2.87 (m, 4H), 2.63 − 2.79 (m, 6H), 2.47 (s, 3H), 2.39 (s, 3H), 1.96 − 2.08 (m, 4H), 1.46 − 1.54 (m, 4H). |
| | | N-((1,2,3,5,6,7-hexahydro-s- | |
| | | indacen-4-yl)carbamoyl)-4-((((4- | |
| | | (hydroxymethyl)tetrahydro-2H- | |
| | | pyran-4- | |
| | | yl)methyl)(methyl)amino)methyl)- | |
| | | 5-methylfuran-2-sulfonimidamide | |
| 11 | 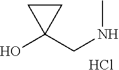 | 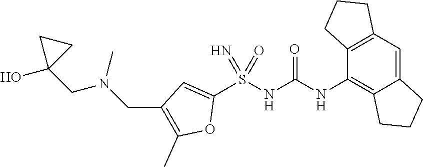 [SMILES: Cc1oc(S(=N)(=O)NC(=O)Nc2c3c(cc4c2CCC4)CCC3)cc1CN(C)CC1(O)CC1] | LC-MS: m/z [M + H]⁺ = 473.25; ¹H NMR (300 MHz, CD₃OD) δ (ppm): 7.07 (s, 1H), 6.93 (s, 1H), 3.51 (s, 2H), 2.68 − 2.95 (m, 8H), 2.55 (s, 2H), 2.27 − 2.41 (m, 6H), 1.92 − 2.14 (m, 4H), 0.69 − 0.77 (m, 2H), 0.47 − 0.56 (m, 2H). |
| | | N-((1,2,3,5,6,7-hexahydro-s- | |
| | | indacen-4-yl)carbamoyl)-4-((((1- | |
| | | hydroxycyclopropyl)methyl)(methyl) | |
| | | amino)methyl)-5-methylfuran-2- | |
| | | sulfonimidamide | |
| 12 | 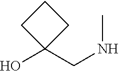 | 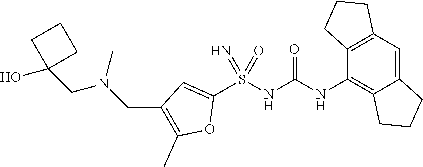 [SMILES: Cc1oc(S(=N)(=O)NC(=O)Nc2c3c(cc4c2CCC4)CCC3)cc1CN(C)CC1(O)CCC1] | LC-MS: m/z [M + H]⁺ = 487.25; ¹H NMR (300 MHz, CD₃OD) δ (ppm): 7.00 (s, 1H), 6.81 (s, 1H), 3.84 (s, 2H), 2.92 (s, 2H), 2.59 − 2.83 (m, 8H), 2.54 (s, 3H), 2.32 (s, 3H), 1.98 − 2.13 (m, 4H), 1.80 − 1.98 (m, 4H), 1.64 − 1.78 (m, 1H), 1.42 − 1.56 (m, 1H). |
| | | N-((1,2,3,5,6,7-hexahydro-s- | |
| | | indacen-4-yl)carbamoyl)-4-((((1- | |
| | | hydroxycyclobutyl)methyl)(methyl) | |
| | | amino)methyl)-5-methylfuran-2- | |
| | | sulfonimidamide | |

### Example 13: Preparation of N-((8-fluoro-1,2,3,5,6,7-hexahydro-s-indacen-4-yl)carbamoyl)-4-((((1-(hydroxymethyl)cyclobutyl)methyl)(methyl)amino)methyl)-5-methylfuran-2-sulfonimidamide

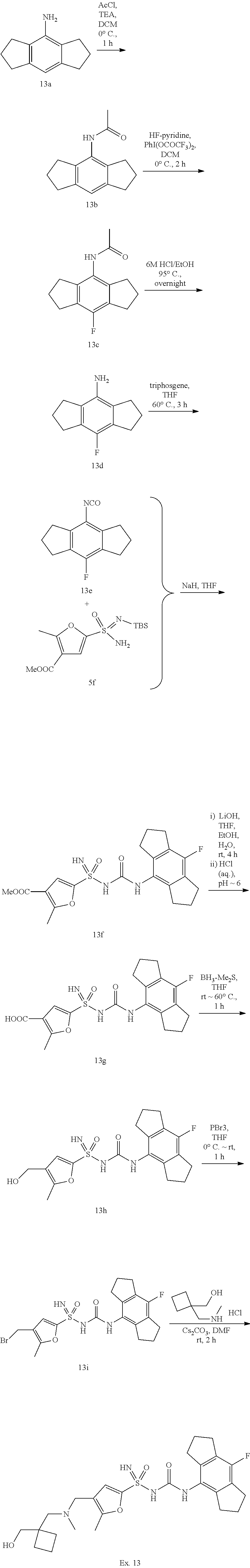

### Step 1:

To a stirred colorless solution of 1,2,3,5,6,7-hexahydro-s-indacen-4-amine 13a (20 g, 115.610 mmol) and TEA (25 mL, 173.410 mmol) in CH₂Cl₂ (2000 mL) at 0° C., AcCl (10.89 g, 138.732 mmol) was added dropwise to give a colorless suspension. The suspension was stirred at rt for 1 h. Solvents were evaporated under vacuo to yield a colorless solid. The solid was suspended in water (500 mL) and filtered, this process was repeated three times, and resulting solid was dried in oven to afford N-(1,2,3,5,6,7-hexahydro-s-indacen-4-yl)acetamide 13b as a white solid (20 g, 90.90%). LC-MS: m/z [M+H]⁺=216.15.

### Step 2:

To a colorless suspension of N-(1,2,3,5,6,7-hexahydro-s-indacen-4-yl)acetamide 13b (5 g, 23.256 mmol) in DCM (25 mL) at −15° C. was added HF-pyridine (25 mL, 60% wt) dropwise. The mixture turned to a brown solution within 5 min. To this solution, a colorless suspension of PhI(OCOCF₃)₂ (15 g, 34.884 mmol) was added giving a black solution. The reaction mixture was stirred at −15° C. for 2 h. The reaction was quenched with the addition of an aqueous solution of Ca(OH)₂. The aqueous mixture was extracted with DCM (200 mL×3). The combined organic extracts were washed with brine (300 mL×2), dried with Na₂SO₄, filtered and concentrated to give a black solid, which was recrystallized with EtOAc and PE to afford N-(8-fluoro-1,2,3,5,6,7-hexahydro-s-indacen-4-yl)acetamide 13c as a brown solid (2.5 g, 46.30%). LC-MS: m/z [M+H]⁺=234.05.

### Step 3:

A dark brown solution of N-(8-fluoro-1,2,3,5,6,7-hexahydro-s-indacen-4-yl)acetamide 13c (2.5 g, 10.716 mmol) in EtOH (10 mL) and HCl (con., 10 mL) was heated at 95° C. with stirring overnight. After cooling to rt, NaOH (aq., 10 M) was added to adjust the pH to ˜10. The aqueous mixture was extracted with DCM (100 mL×3). The combined organic extracts were washed with brine (100 mL), dried with Na₂SO₄, filtered and concentrated to give a crude solid, which was purified by silica gel column chromatography yielding 8-fluoro-1,2,3,5,6,7-hexahydro-s-indacen-4-amine 13d as a light brown solid (1.2 g, 58.55%). ¹HNMR (400 MHz, CDCl₃) δ (ppm): 3.54 (s, b, 2H), 2.89-2.92 (m, 4H), 2.71-2.73 (m, 4H), 2.11-2.18 (m, 4H); ¹⁹FNMR (376 MHz, CDCl₃) δ (ppm). −134.75; LC-MS: m/z [M+H]⁺=192.05.

### Step 4:

A colorless suspension of 8-fluoro-1,2,3,5,6,7-hexahydro-s-indacen-4-amine 13d (600 mg, 3.317 mmol) and triphosgene (307 mg, 1.035 mmol) in THF (50 mL) was stirred at 60° C. for 3 h. The reaction was monitored by TLC. After completion, the solvent was removed under vacuo to give 4-fluoro-8-isocyanato-1,2,3,5,6,7-hexahydro-s-indacene 13e as a grey solid (640 mg, 93.90%). This crude product was used in the next step without any purification.

### Step 5:

NaH (70.1 mg, 2.946 mmol) was added to a colorless solution of methyl 5-[(tert-butyldimethylsilyl)-S-aminosulfonimidoyl]-2-methylfuran-3-carboxylate 5f (0.98 g, 2.946 mmol), available from Preparative Example 5, in THF at 0° C. with stirring. The resulting mixture was stirred at this temperature for 10 min. 4-Fluoro-8-isocyanato-1,2,3,5,6,7-hexahydro-s-indacene 13e (640 mg, 2.946 mmol) was added. Cooling bath was removed, and the reaction mixture was stirred at room temperature for 2 h. A 2M aqueous solution of HCl (10 mL) was added and stirring was continued for 5 min. The aqueous mixture was extracted with iPrOH/CHCl₃ (v/v, 1/3, 50 mL×3). The combined organic extract were washed with brine (50 mL), dried with Na₂SO₄, filtered and evaporated to dryness to provide methyl 5-([[(8-fluoro-1,2,3,5,6,7-hexahydro-s-indacen-4-yl)carbamoyl]amino](imino)oxo-lambda6-sulfanyl)-2-methylfuran-3-carboxylate 13f as a white solid (1.0 g, 70.23%). LC-MS: m/z [M+H]⁺=435.13.

### Step 6:

A solution of LiOH (165 mg, 6.889 mmol) in H₂O (8 mL) was added to a colorless solution of methyl 5-([[(8-fluoro-1,2,3,5,6,7-hexahydro-s-indacen-4-yl)carbamoyl]amino](imino)oxo-lambda6-sulfanyl)-2-methylfuran-3-carboxylate 13f (1 g, 2.296 mmol) in THF (16 mL) and EtOH (4 mL) at room temperature. The mixture was stirred for 4 h and concentrated in vacuo. The remaining aqueous mixture was acidified to pH ˜6 using a 2M aqueous HCl solution. The resulting suspension was filtered, and the solid collected was dried in oven to provide 5-([[(8-fluoro-1,2,3,5,6,7-hexahydro-s-indacen-4-yl)carbamoyl]amino](imino)oxo-lambda6-sulfanyl)-2-methylfuran-3-carboxylic acid 13g as a white solid (800 mg, 76.13%). LC-MS: m/z [M+H]⁺=422.05.

### Step 7:

BH₃-Me₂S complex (0.95 mL, 10 M, 9.5 mmol) was added dropwise to a colorless suspension of 5-([[(8-fluoro-1,2,3,5,6,7-hexahydro-s-indacen-4-yl)carbamoyl]amino](imino)oxo-lambda6-sulfanyl)-2-methylfuran-3-carboxylic acid 13g (800 mg, 1.898 mmol) in THF (20 mL) at 0° C. under N₂. Cooling bath was removed, and the mixture was heated up at 60° C. and stirred for 2 h. After cooling to room temperature, the reaction was quenched by the addition of a saturated aqueous NH₄Cl solution (5 mL) and further diluted with water (10 mL). The aqueous mixture was extracted with CHCl₃/iPrOH (v/v, 3/1, 40 mL×3). The combined organic extracts were concentrated to give a white solid (0.7 g). 70 mg of this solid was purified by Prep-HPLC using the following conditions: Column: Xselect CSH OBD Column 30*150 mm 5 μm; Mobile Phase A: Water (0.1% FA), Mobile Phase B: ACN; Flow rate: 60 mL/min; Gradient: 25% B to 53% B in 7 min, 53% B to B; 220 nm; RT1: 6.42 min. After lyophilization, 3-(8-fluoro-1,2,3,5,6,7-hexahydro-s-indacen-4-yl)-1-[[4-(hydroxymethyl)-5-methylfuran-2-yl](imino)oxo-lambda6-sulfanyl]urea 13h was obtained as a white solid (25.8 mg). ¹HNMR (400 MHz, DMSO-d6) δ (ppm): 8.42 (s, b, 1H), 7.60 (m, 2H), 6.87 (s, 1H), 5.03 (t, J=5.60 Hz, 1H), 4.28 (d, J=5.60 Hz, 2H), 2.67-2.83 (m, 8H), 2.29 (s, 3H), 1.98-2.01 (m, 4H); ¹⁹F NMR (376 MHz, DMSO-d₆) δ (ppm): −126.54; LC-MS: m/z [M+H]⁺=408.10.

### Step 8:

PBr₃ (418.5 mg, 1.546 mmol) was added dropwise to a colorless suspension of 3-(8-fluoro-1,2,3,5,6,7-hexahydro-s-indacen-4-yl)-1-[[4-(hydroxymethyl)-5-methylfuran-2-yl](imino)oxo-lambda6-sulfanyl]urea 13h (630 mg, 1.546 mmol) in THF (10 mL) at 0° C. The resulting suspension was stirred at room temperature for 1 h. The mixture was diluted with water (5 mL) and extracted with iPrOH/CHCl₃ (v/v, 1/3, 30 mL×3). The combined organic extracts were washed with brine (30 mL), dried over Na₂SO₄, filtered and concentrated to a solid, which was recrystallized from petroleum ether to give 1-[[4-(bromomethyl)-5-methylfuran-2-yl](imino)oxo-lambda6-sulfanyl]-3-(8-fluoro-1,2,3,5,6,7-hexahydro-s-indacen-4-yl)urea 13i as a white solid (630 mg, 76.67%). LC-MS: m/z [M]⁺, [M+2]⁺=469.95, 471.95.

### Step 9:

A colorless suspension of [[1-(hydroxymethyl)cyclobutyl]methyl](methyl)azanium hydrochloride (70 mg, 0.425 mmol) and Cs₂CO₃ (346 mg, 1.063 mmol) in DMF (1 mL) was stirred at room temperature for 0.5 h. 3-[[4-(bromomethyl)-5-methylfuran-2-yl](imino)oxo-lambda6-sulfanyl]-1-(8-fluoro-1,2,3,5,6,7-hexahydro-s-indacen-4-yl)urea 13i (100 mg, 0.213 mmol) was added. The resulting suspension was stirred at room temperature for 1.5 h. The mixture was diluted with iPrOH/CHCl₃ (v/v, 1/3, 10 mL) and water (3 mL), and separated. The aqueous phase was extracted with iPrOH/CHCl₃ (v/v, 1/3, 5 mL×2). The combined organic extracts were concentrated in vacuo to a residue, which was purified by Prep-HPLC using the following conditions: Column: XSelect CSH Prep C18 OBD Column, 19*250 mm, 5 μm; Mobile Phase A: Water (0.1% FA), Mobile Phase B: ACN; Flow rate: 25 mL/min; Gradient: 25% B to 52% B in 7 min; 52% B to B; 254 nm; RTL: 5.35 min. After lyophilization, the titled compound 1-(8-fluoro-1,2,3,5,6,7-hexahydro-s-indacen-4-yl)-3-([4-[([[1-(hydroxymethyl)cyclobutyl]methyl](methyl)amino)methyl]-5-methylfuran-2-yl](imino)oxo-lambda6-sulfanyl)urea (Ex. 13) was obtained as a white solid (24.5 mg, 20.29%). ¹HNMR (400 MHz, CD₃OD) δ (ppm): 7.00 (s, 1H), 3.72 (s, 2H), 3.49 (s, 2H), 2.70-2.87 (m, 10H), 2.36 (s, 3H), 2.30 (s, 3H), 1.82-2.10 (m, 10H); ¹⁹FNMR (376 MHz, CD₃OD) δ (ppm): −128.17; LC-MS: m/z [M+H]⁺=519.25.

### Example 14: Preparation of N-((8-fluoro-1,2,3,5,6,7-hexahydro-s-indacen-4-yl)carbamoyl)-4-((3-hydroxyazetidin-1-yl)methyl)-5-methylfuran-2-sulfonimidamide

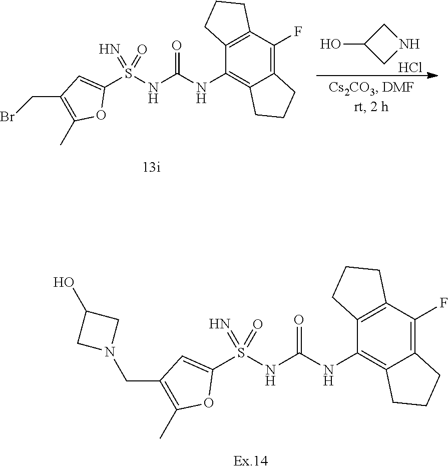

A colorless suspension of 3-hydroxyazetidin-1-ium chloride (46.6 mg, 0.425 mmol) and Cs₂CO₃ (346 mg, 1.063 mmol) in DMF (1 mL) was stirred at room temperature for 0.5 h. To this suspension, 3-[[4-(bromomethyl)-5-methylfuran-2-yl](imino)oxo-lambda6-sulfanyl]-1-(8-fluoro-1,2,3,5,6,7-hexahydro-s-indacen-4-yl)urea 13i (100 mg, 0.213 mmol) was added and the resulting brown suspension was stirred at room temperature for 1.5 h. The mixture was diluted with iPrOH/CHCl₃ (v/v, 1/3, 10 mL) and water (3 mL), and separated. The aqueous phase was extracted with iPrOH/CHCl₃ (v/v, 1/3, 5 mL×2). The combined organic extracts were concentrated in vacuo to a residue which was purified by Prep-HPLC using the following conditions: Column: Kinetex EVO C18 Column, 30*150 mm, 5 μm; Mobile Phase A: Water (0.05% FA), Mobile Phase B: ACN; Flow rate: 60 mL/min; Gradient: 15% B to 35% B in 7 min; 35% B to B: 254 nm; RT1: 5.58 min. After lyophilization, the titled compound 1-(8-fluoro-1,2,3,5,6,7-hexahydro-s-indacen-4-yl)-3-([4-[(3-hydroxyazetidin-1-yl)methyl]-5-methylfuran-2-yl](imino)oxo-lambda6-sulfanyl)urea (Ex. 14) was obtained as a white solid (23.3 mg, 21.14%). ¹HNMR (400 MHz, CD₃OD) δ (ppm): 7.06 (s, 1H), 4.50 (m, 1H), 3.99-4.00 (m, 2H), 3.90-3.93 (m, 2H), 3.47-3.50 (m, 2H), 2.80-2.92 (m, 8H), 2.43 (s, 3H), 2.00-2.10 (m, 4H); ¹⁹FNMR (376 MHz, CD₃OD) δ (ppm): −128.07; LC-MS: m/z [M+H]⁺=463.10.

### Example 15: Preparation of 5-(N-((1,2,3,5,6,7-hexahydro-s-indacen-4-yl)carbamoyl)sulfamidimidoyl)-N-((1r,3r)-3-hydroxycyclobutyl)-N-methylfuran-3-carboxamide

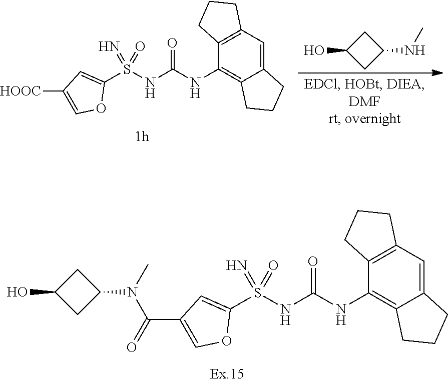

To a stirred solution of 5-([[(1,2,3,5,6,7-hexahydro-s-indacen-4-yl)carbamoyl]amino](imino)oxo-lambda6-sulfanyl)furan-3-carboxylic acid 1h (150 mg, 0.385 mmol), available from Preparative Example 1, in DMF (5 mL) was added (1r,3r)-3-(methylamino)cyclobutan-1-ol (78 mg, 0.770 mmol) and DIEA (149 mg, 1.156 mmol). EDCl (111 mg, 0.578 mmol) and HOBt (104 mg, 0.770 mmol) were added resulting a slight yellow solution. The reaction mixture was stirred at room temperature overnight. Water (10 mL) was added. The aqueous mixture was extracted with EtOAc (15 mL×3). The combined organic extracts were washed with sat. NaCl (10 mL×3), dried with Na₂SO₄ and concentrated under vacuo to a residue. The residue was purified by Prep-HPLC using the following conditions: Column: YMC-Actus Triart C18, 30*250 mm, 5 μm; Mobile Phase A: Water (10 mmol/L NH₄HCO₃+0.1% NH₃·H₂O), Mobile Phase B: ACN; Flow rate: 60 mL/min, Gradient: 21% B to 36% B in 7 min; 36% B to B; 254 nm; RT1: 5.22 min. After lyophilization, the titled compound 5-([[(1,2,3,5,6,7-hexahydro-s-indacen-4-yl)carbamoyl]amino](imino)oxo-lambda6-sulfanyl)-N-methyl-N-[(1r,3r)-3-hydroxycyclobutyl]furan-3-carboxamide (Ex. 15) was obtained as a white solid (37.0 mg, 20.44%). ¹HNMR (400 MHz, CD₃OD) δ (ppm): 8.08 (s, 1H), 7.22 (s, 1H), 6.93 (s, 1H), 5.03 (m, 1H), 4.37 (m, 1H), 3.10 (s, 3H), 2.75-2.87 (m, 8H), 2.60-2.69 (m, 2H), 2.22-2.26 (m, 2H), 2.00-2.08 (m, 4H); LC-MS: m/z [M+H]⁺=471.

### Example 16: Preparation of N-((1r,3r)-3-aminocyclobutyl)-5-(N-((1,2,3,5,6,7-hexahydro-s-indacen-4-yl)carbamoyl)sulfinimidoyl)-N-methylfuran-3-carboxamide)

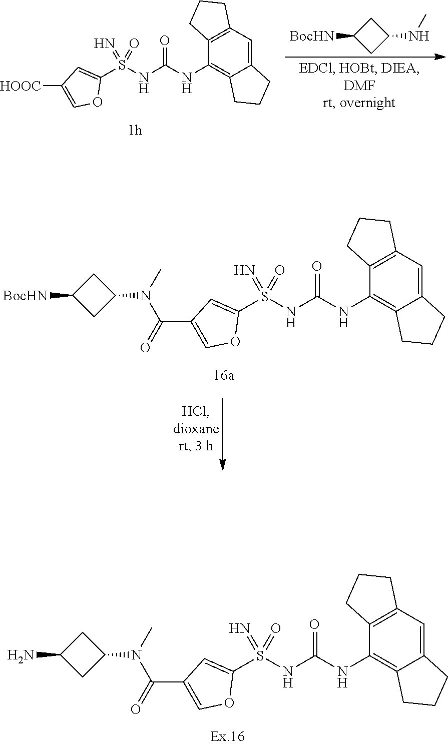 [SMILES: N=S(=O)(NC(=O)Nc1c2c(cc3c1CCC3)CCC2)c1cc(C(=O)O)co1 ; CN[C@H]1C[C@H](NC(=O)OC(C)(C)C)C1 ; CN(C(=O)c1coc(S(=N)(=O)NC(=O)Nc2c3c(cc4c2CCC4)CCC3)c1)[C@H]1C[C@H](NC(=O)OC(C)(C)C)C1 ; CN(C(=O)c1coc(S(=N)(=O)NC(=O)Nc2c3c(cc4c2CCC4)CCC3)c1)[C@H]1C[C@H](N)C1]

### Step 1:

A brown suspension of 5-([[(1,2,3,5,6,7-hexahydro-s-indacen-4-yl)carbamoyl]amino](imino)oxo-lambda6-sulfanyl)furan-3-carboxylic acid 1h (150 mg, 0.385 mmol), tert-butyl N-[(1r,3r)-3-(methylamino)cyclobutyl]carbamate (154 mg, 0.770 mmol), EDCl (111 mg, 0.578 mmol), HOBt (104 mg, 0.770 mmol) and DIEA (149 mmol, 1.156 mmol) in DMF (5 mL) was stirred at room temperature overnight. A solution of CHCl₃/iPrOH (v/v, 3/1, 10 mL) was added followed by water (5 mL). The mixture was separated, the aqueous phase was extracted with CHCl₃/iPrOH (v/v, 3/1, 10 mL×2), and the combined organic phases were washed with brine (5 mL×2), dried with Na₂SO₄, filtered and concentrated in vacuo to give tert-butyl N-[(1r,3r)-3-[N-methyl5-([[(1,2,3,5,6,7-hexahydro-s-indacen-4-yl)carbamoyl]amino](imino)oxo-lambda6-sulfanyl)furan-3-amido]cyclobutyl]carbamate 16a as a brown oil (150 mg, 54.56%). LC-MS: m/z [M+H]⁺=570.20.

### Step 2:

To a yellow solution of tert-butyl N-[(1r,3r)-3-[N-methyl5-([[(1,2,3,5,6,7-hexahydro-s-indacen-4-yl)carbamoyl]amino](imino)oxo-lambda6-sulfanyl)furan-3-amido]cyclobutyl]carbamate 16a (150 mg, 0.262 mmol) in DCM (3 mL) was added a solution of HCl in 1,4-dioxane (3 mL, 4 M, 12 mmol). The resulting suspension was stirred at room temperature for 3 h. Solvents were removed through evaporation in vacuo to give a yellow oil, which was purified by Prep-HPLC using the following conditions: Column. XBridge Prep OBD C18 Column, 30*150 mm 5 μm; Mobile Phase A: Water (10 mmol/L NH₄HCO₃+0.1% NH₃·H₂O), Mobile Phase B: ACN; Flow rate: 60 mL/min; Gradient: 10% B to 35% B in 7 min; 35% B to B: 220 nm; RT1: 6.32 min. After lyophilization, the titled compound 5-([[(1,2,3,5,6,7-hexahydro-s-indacen-4-yl)carbamoyl]amino](imino)oxo-lambda6-sulfanyl)-N-methyl-N-[(1r,3r)-3-aminocyclobutyl]furan-3-carboxamide (Ex. 16) was obtained as a white solid (56.9 mg, 45.73%). ¹HNMR (400 MHz, CD₃OD) δ (ppm): 8.03 (s, 1H), 7.12 (s, 1H), 6.90 (s, 1H), 4.95 (m, 1H), 3.57 (s, 1H), 3.09 (s, 1H), 2.70-2.87 (m, 10H), 2.12-2.23 (m, 2H), 2.00-2.05 (m, 4H); LC-MS: m/z [M+H]⁺=472.30.

### Example 17: Preparation of N-(azetidin-3-yl)-5-(N-((1,2,3,5,6,7-hexahydro-s-indacen-4-yl)carbamoyl)sulfamidimidoyl)-N-methylfuran-3-carboxamide

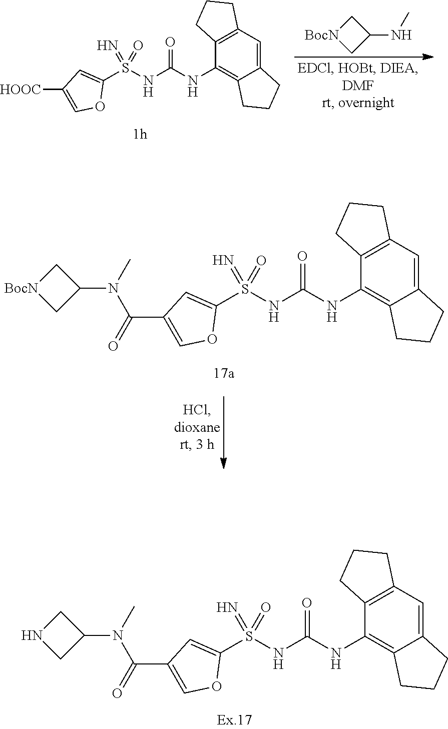 [SMILES: N=S(=O)(NC(=O)Nc1c2c(cc3c1CCC3)CCC2)c1cc(C(=O)O)co1 ; CNC1CN(C(=O)OC(C)(C)C)C1 ; CN(C(=O)c1coc(S(=N)(=O)NC(=O)Nc2c3c(cc4c2CCC4)CCC3)c1)C1CN(C(=O)OC(C)(C)C)C1 ; CN(C(=O)c1coc(S(=N)(=O)NC(=O)Nc2c3c(cc4c2CCC4)CCC3)c1)C1CNC1]

### Step 1:

A brown suspension of 5-([[(1,2,3,5,6,7-hexahydro-s-indacen-4-yl)carbamoyl]amino](imino)oxo-lambda6-sulfanyl)furan-3-carboxylic acid 1h (150 mg, 0.385 mmol), tert-butyl 3-(methylamino)azetidine-1-carboxylate (186 mg, 0.770 mmol), EDCl (111 mg, 0.578 mmol), HOBt (104 mg, 0.770 mmol) and DIEA (149 mmol, 1.156 mmol) in DMF (5 mL) was stirred at room temperature overnight. A solution of CHCl₃/iPrOH (v/v, 3/1, 10 mL) was added followed by water (5 mL). The mixture was separated, the aqueous phase was extracted with CHCl₃/iPrOH(v/v, 3/1, 10 mL×2), and the combined organic phases were washed with brine (5 mL×2), dried with Na₂SO₄, filtered and concentrated. The residue was purified by silica gel column (0 to 7% MeOH/DCM) providing tert-butyl 3-[N-methyl5-([[(1,2,3,5,6,7-hexahydro-s-indacen-4-yl)carbamoyl]amino](imino)oxo-lambda6-sulfanyl)furan-3-amido]azetidine-1-carboxylate 17a as an off-white solid (150 mg, 69.62%). LC-MS: m/z [M+H]⁺=558.25.

### Step 2:

A yellow solution of tert-butyl 3-[N-methyl5-([[(1,2,3,5,6,7-hexahydro-s-indacen-4-yl)carbamoyl]amino](imino)oxo-lambda6-sulfanyl)furan-3-amido]azetidine-1-carboxylate 17a (130 mg, 0.233 mmol) in DCM (2 mL) and TFA (0.5 mL) was stirred at room temperature for 1 h. Solvent was removed in vacuo to give a yellow oil, which was re-dissolved with TEA in DCM and concentrated again to a yellow oily crude product. The crude oil was purified by Prep-HPLC using the following conditions: Column: XBridge BEH C18 OBD Prep Column, 19*250 mm; Mobile Phase A: Water (10 mmol/L NH₄HCO₃+0.1% NH₃·H₂O), Mobile Phase B: ACN; Flow rate: 25 mL/min; Gradient: 25% B to 30% B in 8 min; 30% B to B; 254 nm; RT1: 7.07 min. After lyophilization, the titled compound N-(azetidin-3-yl)-5-(N-((1,2,3,5,6,7-hexahydro-s-indacen-4-yl)carbamoyl)sulfamidimidoyl)-N-methylfuran-3-carboxamide Ex. 17 was obtained as a white solid (22.8 mg, 21.19%). ¹HNMR (400 MHz, CD₃OD) δ (ppm): 8.02 (s, 1H), 7.13 (s, 1H), 6.90 (s, 1H), 4.26 (m, 1H), 3.50 (m, 2H), 2.70-2.97 (m, 10H), 1.99-2.05 (m, 4H), 1.23 (d, J=6.20 Hz, 6H); LC-MS: m/z [M+H]⁺=458.20.

### Example 18: Preparation of 4-(((1r,3r)-3-aminocyclobutoxy)methyl)-N-((1,2,3,5,6,7-hexahydro-s-indacen-4-yl)carbamoyl)furan-2-sulfonimidamide

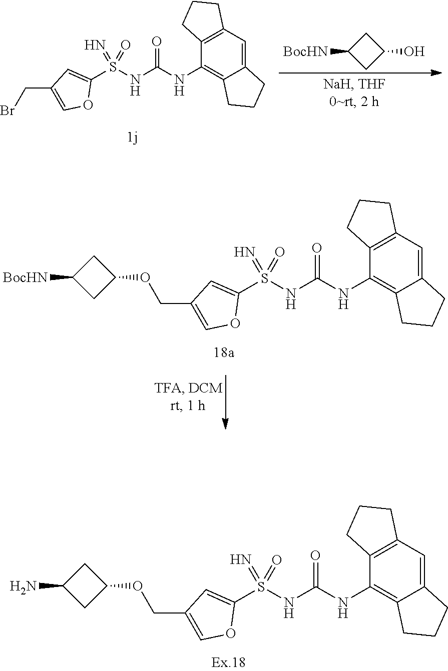 [SMILES: N=S(=O)(NC(=O)Nc1c2c(cc3c1CCC3)CCC2)c1cc(CBr)co1 ; CC(C)(C)OC(=O)N[C@H]1C[C@H](O)C1 ; CC(C)(C)OC(=O)N[C@H]1C[C@H](OCc2coc(S(=N)(=O)NC(=O)Nc3c4c(cc5c3CCC5)CCC4)c2)C1 ; N=S(=O)(NC(=O)Nc1c2c(cc3c1CCC3)CCC2)c1cc(CO[C@H]2C[C@H](N)C2)co1]

Step 1:

NaH (60% dispersion in oil, 55 mg, 2.282 mmol) was added to a solution of tert-butyl ((1r,3r)-3-hydroxycyclobutyl)carbamate (427 mg, 2.282 mmol) in THF (5 mL) at 0° C. and the resulting suspension was stirred at this temperature for 0.5 h. 4-(Bromomethyl)-N-((1,2,3,5,6,7-hexahydro-s-indacen-4-yl)carbamoyl)furan-2-sulfonimidamide 1j (500 mg, 1.141 mmol), available from Preparative Example 1, was added. The reaction mixture was then stirred at rt for 2 h. The reaction was quenched with water and extracted with CHCl₃/iPrOH. The combined organic extracts were washed with brine, dried over Na₂SO₄, and concentrated to a residue, which was purified by reverse phase column (0.05% FA) to provide tert-butyl ((1r,3r)-3-((5-(N-((1,2,3,5,6,7-hexahydro-s-indacen-4-yl)carbamoyl)sulfamidimidoyl)furan-3-yl)methoxy)cyclobutyl)carbamate 18a as a white solid (80 mg, 12.88%).

### Step 2:

A paly yellow solution of tert-butyl N-[(1r,3r)-3-[[5-([[(1,2,3,5,6,7-hexahydro-s-indacen-4-yl)carbamoyl]amino](imino)oxo-lambda6-sulfanyl)furan-3-yl]methoxy]cyclobutyl]carbamate 18a (76 mg, 0.140 mmol) in TFA (1 mL) and DCM (4 mL) was stirred at room temperature for 1 h. Solvents were removed in vacuo, and the solid residue was dissolved in DMF. The solution was adjusted to pH ˜8 using a solution of LiOH in MeOH. The solution was purified by Prep-HPLC using the following conditions: Column: Xselect CSH OBD Column 30*150 mm 5 μm; Mobile Phase A: Water(0.1% FA), Mobile Phase B: ACN; Flow rate: 60 mL/min; Gradient: 14% B to 34% B in 7 min, 34% B to B; 220 nm; RT1: 5.23 min. After lyophilization, the titled compound 3-(1,2,3,5,6,7-hexahydro-s-indacen-4-yl)-1-[imino(oxo)(4-[[(1r,3r)-3-aminocyclobutoxy]methyl]furan-2-yl)-lambda6-sulfanyl]urea Ex. 18 was obtained as a white solid (26.2 mg, 37.89%). ¹HNMR (400 MHz, CD₃OD) δ (ppm): 7.72 (s, 1H), 7.07 (s, 1H), 6.91 (s, 1H), 4.35 (m, 2H), 4.25-4.31 (m, 1H), 3.77-3.84 (m, 1H), 2.72-2.85 (m, 8H), 2.28-2.44 (m, 4H), 1.97-2.04 (m, 4H); LC-MS: m/z [M+H]⁺=445.20.

### Example 19: Preparation of N-((1,2,3,5,6,7-hexahydro-s-indacen-4-yl)carbamoyl)-5-((3-hydroxyazetidin-1-vi)methyl)-1-isopropyl-1H-pyrazole-3-sulfonimidamide

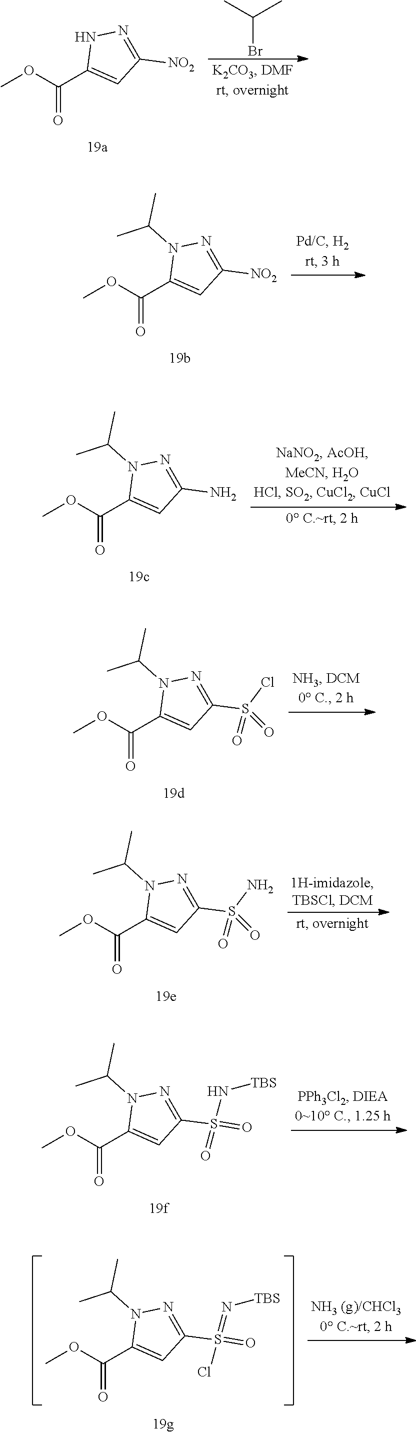

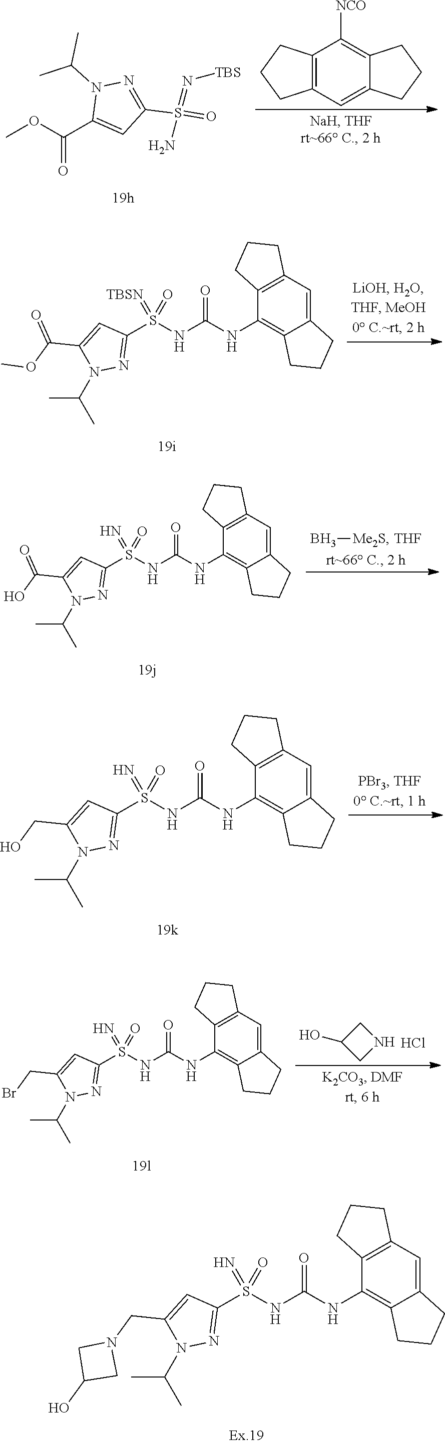

### Step 1:

To a stirred solution of methyl 5-nitro-2H-pyrazole-3-carboxylate 19a(50.00 g, 292.206 mmol) in DMF (500 ml) was added K₂CO₃ (121.15 g, 876.619 mmol) and 2-bromopropane (71.88 g, 584.413 mmol) to give a colorless suspension. The mixture was stirred at rt overnight and poured into water (600 ml). The aqueous mixture was extracted with EtOAc (500 mL×3). The combined extracts were washed with brine (500 mL×2), dried over anhydrous Na₂SO₄, filtered and concentrated. The residue was purified by silica gel column chromatography to afford methyl 1-isopropyl-3-nitro-1H-pyrazole-5-carboxylate 19b as a yellow oil (330 mg, 31.055%).

### Step 2:

To a solution of methyl 2-isopropyl-5-nitropyrazole-3-carboxylate 19b (53.30 g, 250.008 mmol) in MeOH (533 ml) was added 10% Pd/C (133.03 g, 1250.047 mmol) under N₂ giving a black suspension. The mixture was then stirred at rt for 3 h under H2. The suspension was filtered and concentrated, and the residue was purified by silica gel column chromatography to give methyl 3-amino-1-isopropyl-1H-pyrazole-5-carboxylate 19c as a light yellow solid (35 g, 76.41%). LC-MS: m/z [M+H]⁺=184.1.

### Step 3:

A colorless solution of NaNO₂ (11.75 g, 170.29 mmol) in water (710 mL) was added dropwise to a colorless solution of methyl 3-amino-1-isopropyl-1H-pyrazole-5-carboxylate 19c (26.0 g, 141.91 mmol) in MeCN (71 mL) and a 7.5 M HCl aqueous solution (117.86 mL) at 0° C. The resulting yellow suspension was stirred at this temperature for 1 h. To this suspension, a saturated solution of SO₂ in AcOH (710 mL) was added and followed by a green solution of CuCl₂ (9.54 g, 70.975 mmol) and CuCl (0.70 g, 7.096 mmol) in water (8 mL) to give a dark green solution. The reaction mixture was stirred at 0° C. for another 3 h. EtOAc (1500 mL) and water (500 mL) were added, and the mixture was separated. The aqueous phase was extracted with EtOAc (1000 mL×2). The combined organic phases were washed with brine (500 mL), dried over Na₂SO₄ and filtered. Removal of solvents afforded methyl 3-(chlorosulfonyl)-1-isopropyl-1H-pyrazole-5-carboxylate 19d as a yellow oil (12.5 g, 26.62%).

### Step 4:

Methyl 3-(chlorosulfonyl)-1-isopropyl-1H-pyrazole-5-carboxylate 19d (30 g, 112.48 mmol) was treated with a saturated solution of NH₃(g) in DCM (500 mL) at 0° C. to give a yellow suspension. The mixture was stirred at rt overnight. After concentration, the residue was purified by silica gel column chromatography to give methyl 1-isopropyl-3-sulfamoyl-1H-pyrazole-5-carboxylate 19e as a yellow solid (24.6 g, 80.22%). LC-MS: m/z [M+H]⁺=248.0.

### Step 5:

A colorless suspension of methyl 1-isopropyl-3-sulfamoyl-1H-pyrazole-5-carboxylate 19e (20.0 g, 80.883 mmol), TBSCl (60.95 g, 404.41 mmol) and 1H-imidazole (11.01 g, 161.76 mmol) in DCM (300 mL) was stirred at room temperature overnight. The suspension was washed with H₂O (200 mL×2) and sat. NaCl (200 mL×2). The organic phase was dried with Na₂SO₄, filtered and concentrated. The residue was purified by flash silica gel column chromatography (0-17% EtOAc/petroleum ether) to afford methyl 3-(N-(tert-butyldimethylsilyl)sulfamoyl)-1-isopropyl-1H-pyrazole-5-carboxylate 19f as a white solid (24 g, 99.0%). ¹HNMR (400 MHz, CDCl₃) δ ppm: 7.16 (s, 1H), 5.56 (m, 1H), 3.90 (s, 3H), 1.51 (d, J=6.60 Hz, 6H), 0.91 (s, 9H), 0.21 (s, 6H); LC-MS: m/z [M+H]⁺=362.0.

### Steps 6 and 7:

Anhydrous Et₂N (4.61 mL, 33.192 mmol) was added dropwise to a colorless suspension of freshly prepared dichlorotriphenylphosphorane (5.53 g, 16.596 mmol) in CHCl₃ (150 mL) at 0° C. to yield a light yellow suspension. The mixture was stirred at 0° C. for 10 min and was added a colorless solution of methyl 3-(N-(tert-butyldimethylsilyl)sulfamoyl)-1-isopropyl-1H-pyrazole-5-carboxylate 19f (4 g, 11.064 mmol) in CHCl₃ (8 mL) dropwise. The resulting light brown suspension was stirred at 0° C. for 1 h. Then, this suspension was added dropwise to a saturated solution of NH₃ in CHCl₃ (350 mL) at 0° C. After addition, cooling was stopped, the mixture was stirred at room temperature for 2 h. N₂ was bubbled into the reaction mixture to remove excess NH₃. Solvents were removed in vacuo and the residue was purified by silica gel flash column chromatography to provide methyl 3-(N′-(tert-butyldimethylsilyl)sulfamidimidoyl)-1-isopropyl-1H-pyrazole-5-carboxylate 19h as a white solid (2 g, 50.14%). LC-MS: m/z [M+H]⁺=360.17.

### Step 8:

A solution of methyl 5-[(tert-butyldimethylsilyl)-S-aminosulfonimidoyl]-2-isopropylpyrazole-3-carboxylate 19h (3.4 g, 9.430 mmol) in THF (47 mL) was cooled at 0° C. NaH (0.27 g, 11.316 mmol) was added with stirring. The mixture was stirred at 0° C. for 0.25 h. 4-isocyanato-1,2,3,5,6,7-hexahydro-s-indacene (2.07 g, 10.373 mmol) was added. Cooling was stopped, and the reaction mixture was stirred at rt for 1.5 h. Water (1 mL) was added to quenched the reaction. Solvents were removed under vacuo to give methyl 3-(N′-(tert-butyldimethylsilyl)-N-((1,2,3,5,6,7-hexahydro-s-indacen-4-yl)carbamoyl)sulfamidimidoyl)-1-isopropyl-1H-pyrazole-5-carboxylate 19i as a brown solid (2.8 g, 51.85%). LC-MS: m/z [M+H]⁺=560.20.

### Step 9:

An aqueous solution of LiOH (0.24 g, 10.004 mmol) in H₂O (7.5 mL) was added to a slightly brown suspension of methyl 3-(N′-(tert-butyldimethylsilyl)-N-((1,2,3,5,6,7-hexahydro-s-indacen-4-yl)carbamoyl)sulfamidimidoyl)-1-isopropyl-1H-pyrazole-5-carboxylate 19i (2.8 g, 5.002 mmol) in THF (30 mL) and MeOH (7.5 mL). The resulting suspension was stirred at room temperature for 4 h. An aqueous solution of HCl (2 N) was added to the suspension to adjust to pH 4. Solvents were removed under vacuo to give a slight brown solid. The solid was washed with MeCN (60 mL) and collected to give 3-(N-((1,2,3,5,6,7-hexahydro-s-indacen-4-yl)carbamoyl)sulfamidimidoyl)-1-isopropyl-1H-pyrazole-5-carboxylic acid 19j as a light brown solid (2 g, 90.90%). LC-MS: m/z [M+H]⁺=432.10.

### Step 10:

To a solution of 5-([[(1,2,3,5,6,7-hexahydro-s-indacen-4-yl)carbamoyl]amino](imino)oxo-lambda6-sulfanyl)-2-isopropylpyrazole-3-carboxylic acid 19j (200 mg, 0.463 mmol) in THF (10 mL) was added BH₃-Me₂S complex (0.1 ml, 10 M, 1 mmol) dropwise at rt under a nitrogen atmosphere. The resulting mixture was stirred at 60° C. for 2 h. After cooling, the mixture was poured into 25 ml of water and extracted with EtOAc (20 mL×3). The combined extracts were washed with brine (30 mL×2), dried over anhydrous Na₂SO₄, filtered and concentrated. The residue was purified by Prep-HPLC using the following conditions: Column: XBridge Prep OBD C18 Column, 30*150 mm 5 μm; Mobile Phase A: Water(0.05% FA), Mobile Phase B: ACN; Flow rate: 60 mL/min; Gradient: 28% B to 48% B in 7 min, 48% B; 254 nm; RT1: 6.63 min. After lyophilization, N-((1,2,3,5,6,7-hexahydro-s-indacen-4-yl)carbamoyl)-5-(hydroxymethyl)-1-isopropyl-1H-pyrazole-3-sulfonimidamide 19k was obtained as a white solid (35.1 mg, 18.03%). ¹HNMR (300 MHz, CD₃OD) δ ppm: 6.90 (s, 1H), 6.66 (s, 1H), 4.73-4.79 (m, 1H), 4.71 (s, 2H), 2.74-2.85 (m, 8H), 1.98-2.06 (m, 4H), 1.49 (d, J=6.60 Hz, 6H); LC-MS: m/z [M+H]⁺=418.1.

### Step 11:

PBr₃ (538.09 mg, 1.988 mmol) was added to a colorless suspension of N-((1,2,3,5,6,7-hexahydro-s-indacen-4-yl)carbamoyl)-5-(hydroxymethyl)-1-isopropyl-1H-pyrazole-3-sulfonimidamide 19k (830 mg, 1.988 mmol) in THF (40 mL) at 0° C. The resulting suspension was stirred at room temperature for 1 h. The mixture was diluted with water (20 mL) and extracted with iPrOH/CHCl₃ (v/v, 1/3, 40 mL×3). The combined organic phases were washed with brine (30 mL), dried with Na₂SO₄, filtered and concentrated. The crude solid was recrystallized from petroleum ether to afford 5-(bromomethyl)-N-((1,2,3,5,6,7-hexahydro-s-indacen-4-yl)carbamoyl)-1-isopropyl-1H-pyrazole-3-sulfonimidamide 191 as a yellow solid (530 mg, 44.67%). LC-MS: m/z [M+H]⁺=480.1.

### Step 12:

A colorless suspension of azetidin-3-ol hydrochloride (41.04 mg, 0.375 mmol) and K₂CO₃ (129.45 mg, 0.937 mmol) in DMF (4 mL) was stirred at rt for 0.5 h. To this suspension, 1-[[5-(bromomethyl)-1-isopropylpyrazol-3-yl](imino)oxo-lambda6-sulfanyl]-3-(1,2,3,5,6,7-hexahydro-s-indacen-4-yl)urea 191 (150.00 mg, 0.312 mmol) was added. The resulting brown suspension was stirred at rt for 5.5 h. The suspension was filtered and the filtrate was purified by Prep-HPLC using the following Column: XBridge Prep OBD C18 Column, 30*150 mm 5 μm; Mobile Phase A: Water (10 MMOL/L NH₄HCO₃), Mobile Phase B: ACN; Flow rate: 60 m/min; Gradient: 37% B to 57% B in 7 min, 57% B to B; 254 nm; RT1: 6.67 min. After lyophilization, the titled compound N-((1,2,3,5,6,7-hexahydro-s-indacen-4-yl)carbamoyl)-5-((3-hydroxyazetidin-1-yl)methyl)-1-isopropyl-1H-pyrazole-3-sulfonimidamide Ex. 19 was obtained as a white solid (46.9 mg, 31.50%). ¹HNMR (300 MHz, Methanol-d4) δ (ppm): 6.90 (s, 1H), 6.63 (s, 1H), 4.65-4.80 (m, 1H), 4.26-4.40 (m, 1H), 3.71 (s, 2H), 3.56-3.67 (m, 2H), 2.89-3.00 (m, 2H), 2.70-2.88 (m, 8H), 1.93-2.09 (m, 4H), 1.51-1.42 (m, 6H); LC-MS: m/z [M+H]⁺=473.2.

### Example 20: Preparation of N-((1,2,3,5,6,7-hexahydro-s-indacen-4-yl)carbamoyl)-5-(((R)-2-(hydroxymethyl)pyrrolidin-1-yl)methyl)-1-isopropyl-1H-pyrazole-3-sulfonimidamide

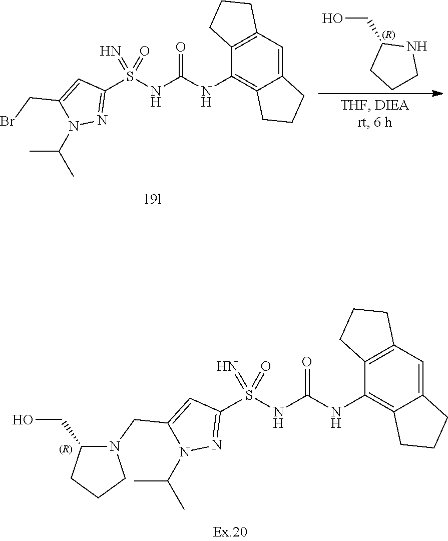

To a mixture of (R)-pyrrolidin-2-ylmethanol (67 mg, 0.663 mmol) in THF (3 mL) was added DIEA (129 mg, 0.995 mmol) and 5-(bromomethyl)-N-((1,2,3,5,6,7-hexahydro-s-indacen-4-yl)carbamoyl)-1-isopropyl-1H-pyrazole-3-sulfonimidamide 191 (150 mg, 0.332 mmol). The reaction mixture was stirred at rt for 6 h. After filtration, the filtrate was purified by Prep-HPLC using the following conditions: Column: XBridge Prep OBD C18 Column, 30*150 mm 5 μm; Mobile Phase A: Water (10 mmol/L NH₄HCOs), Mobile Phase B: ACN; Flow rate: 60 mL/min; Gradient: 37% B to 57% B in 7 min, 57% B to B; 254 nm; RT1: 6.67 min. After lyophilization, the titled compound N-((1,2,3,5,6,7-hexahydro-s-indacen-4-yl)carbamoyl)-5-(((R)-2-(hydroxymethyl)pyrrolidin-1-yl)methyl)-1-isopropyl-1H-pyrazole-3-sulfonimidamide Ex. 20 was obtained as a white solid (47.7 mg, 28.68%). ¹HNMR (300 MHz, Methanol-d4) δ 6.90 (s, 1H), 6.64 (s, 1H), 4.88-5.00 (m, 1H), 4.14-4.26 (m, 1H), 3.40-3.57 (m, 3H), 2.59-2.93 (m, 10H), 2.21-2.35 (m, 1H), 1.86-2.08 (m, 5H), 1.57-1.75 (m, 3H), 1.42-1.51 (m, 6H); LC-MS: m/z [M+H]⁺=501.2.

### Example 21: Preparation of N-((1,2,3,5,6,7-hexahydro-s-indacen-4-yl)carbamoyl)-5-(((S)-2-(hydroxymethyl)pyrrolidin-1-yl)methyl)-1-isopropyl-1H-pyrazole-3-sulfonimidamide

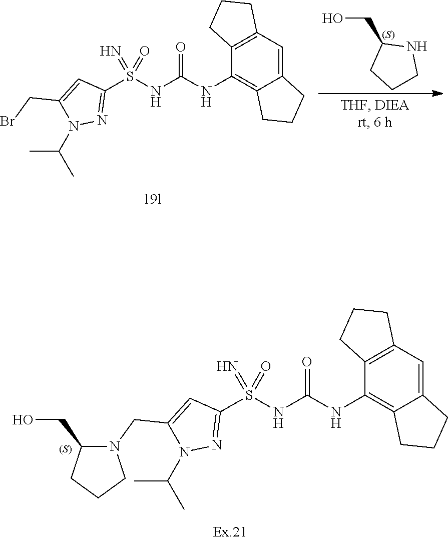

To a solution of (S)-pyrrolidin-2-ylmethanol (67 mg, 0.663 mmol) in THF (3 mL) was added DIEA (128.57 mg, 0.995 mmol) and 5-(bromomethyl)-N-((1,2,3,5,6,7-hexahydro-s-indacen-4-yl)carbamoyl)-1-isopropyl-1H-pyrazole-3-sulfonimidamide 191 (150.00 mg, 0.332 mmol). The resulting brown suspension was stirred at rt for 6 h. Water (5 mL) was added and the aqueous mixture was extracted with iPrOH/CHCl₃ (v/v, 1/3, 10 mL×3). The combined organic phases were evaporated under vacuo and the residue was purified by Prep-HPLC using the following conditions: Column: YMC-Actus Triart C18, 30 mm×150 mm, 5 μm; Mobile Phase A: Water (10 mmol/L NH₄HCO₃+0.1% NH₃·H₂O), Mobile Phase B: ACN; Flow rate: 60 mL/min; Gradient: 37% B to 57% B in 7 min, 57% B; 254 nm; RT1:6.87 min. After lyophilization, the titled compound N-((1,2,3,5,6,7-hexahydro-s-indacen-4-yl)carbamoyl)-5-(((S)-2-(hydroxymethyl)pyrrolidin-1-yl)methyl)-1-isopropyl-1H-pyrazole-3-sulfonimidamide Ex. 21 was obtained as a white solid (52.4 mg, 31.28%). ¹HNMR (300 MHz, CD₃OD) δ (ppm): 6.90 (s, 1H), 6.64 (s, 1H), 4.88-5.00 (m, 1H), 4.14-4.26 (m, 1H), 3.42-3.57 (m, 3H), 2.58-2.97 (m, 10H), 2.20-2.35 (m, 1H), 1.86-2.08 (m, 5H), 1.58-1.79 (m, 3H), 1.42-1.51 (m, 6H); LC-MS: m/z [M+H]⁺=501.2.

### Example 22: Preparation of 4-(aminomethyl)-N-((1,2,3,5,6,7-hexahydro-s-indacen-4-yl)carbamoyl)furan-2-sulfonimidamide

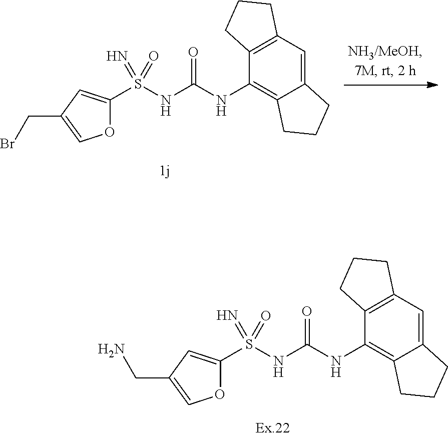 [SMILES: N=S(=O)(NC(=O)Nc1c2c(cc3c1CCC3)CCC2)c1cc(CBr)co1 ; N=S(=O)(NC(=O)Nc1c2c(cc3c1CCC3)CCC2)c1cc(CN)co1]

To a solution of 1-[[4-(bromomethyl)furan-2-yl](imino)oxo-lambda6-sulfanyl]-3-(1,2,3,5,6,7-hexahydro-s-indacen-4-yl)urea 1j (150 mg, 0.342 mmol) in MeOH (10 ml) was added NH₃(g) (7 M/in MeOH) (10 ml). The reaction mixture was stirred at rt for 2 h and concentrated. The residue was purified by Prep-HPLC using the following conditions Column: YMC-Actus Triart C18 ExRS, 30*150 mm, 5 μm; Mobile Phase A: Water (10 mmol/L NH₄HCO₃), Mobile Phase B: ACN; Flow rate: 60 mL/min; Gradient: 21% B to 33% B in 7 min; 254/220 nm; RT1: 6.18 min. After lyophilization, the titled compound 4-(aminomethyl)-N-((1,2,3,5,6,7-hexahydro-s-indacen-4-yl)carbamoyl)furan-2-sulfonimidamide Ex. 22 was obtained as a white solid (22.1 mg, 16.83%). ¹HNMR (300 MHz, CD₃OD) δ (ppm): 7.64-7.71 (m, 1H), 7.04-7.11 (m, 1H), 6.90 (s, 1H), 3.71-3.78 (m, 2H), 2.80-2.88 (m, 4H), 2.72-2.79 (m, 4H), 1.94-2.09 (m, 4H); LC-MS: m/z [M+H]⁺=375.1.

### Example 23: Preparation of N-((1,2,3,5,6,7-hexahydro-s-indacen-4-yl)carbamoyl)-4-((isopropylamino)methyl)furan-2-sulfonimidamide

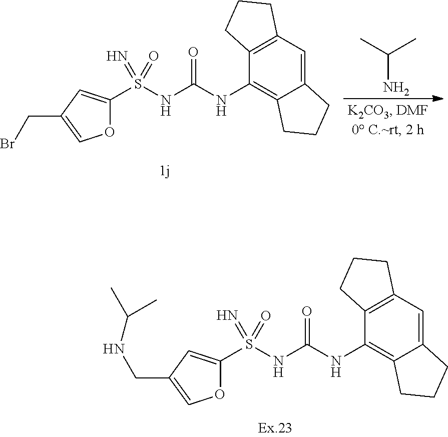

To a solution of isopropylamine (107.88 mg, 1.825 mmol) in DMF (3 ml) at 0° C. was added K₂CO₃ (100.89 mg, 0.730 mmol) and 1-[[4-(bromomethyl)furan-2-yl](imino)oxo-lambda6-sulfanyl]-3-(1,2,3,5,6,7-hexahydro-s-indacen-4-yl)urea 1j (160.00 mg, 0.365 mmol). Cooling was stopped, and the reaction mixture was stirred at rt for 2 h, filtered and concentrated. The crude product was purified by Prep-HPLC using the following conditions: YMC-Actus Triart C18, 30*250 mm, 5 μm; Mobile Phase A: Water(10 mmol/L NH₄HCO₃+0.1% NH₃·H₂O), Mobile Phase B: ACN; Flow rate: 60 mL/min; Gradient: 28% B to 36% B in 8 min, 36% B; 254 nm; RT1: 6.95 min. After lyophilization, the titled compound N-((1,2,3,5,6,7-hexahydro-s-indacen-4-yl)carbamoyl)-4-((isopropylamino)methyl)furan-2-sulfonimidamide Ex. 23 was obtained as a white solid (26.2 mg, 17.06%). ¹HNMR (400 MHz, DMSO-d6) δ (ppm): 7.78 (s, 1H), 7.03 (s, 1H), 6.87 (s, 1H), 3.49-3.64 (m, 2H), 2.71-2.81 (m, 51), 2.57-2.74 (m, 4H), 1.86-1.98 (m, 4H), 0.98-1.04 (m, 6H); LC-MS: m/z [M+H]⁺=417.3.

### Example 24: Preparation of 4-(aminomethyl)-N-((1,2,3,5,6,7-hexahydro-s-indacen-4-yl)carbamoyl)-5-methylfuran-2-sulfonimidamide

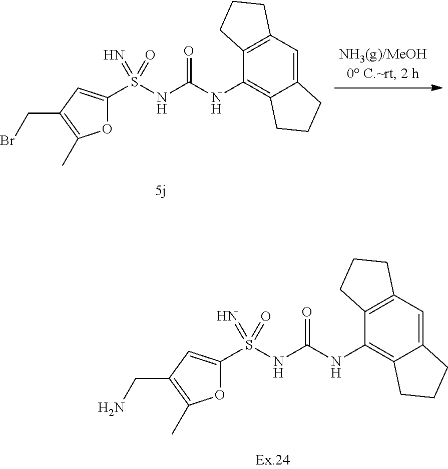 [SMILES: Cc1oc(S(=N)(=O)NC(=O)Nc2c3c(cc4c2CCC4)CCC3)cc1CBr ; Cc1oc(S(=N)(=O)NC(=O)Nc2c3c(cc4c2CCC4)CCC3)cc1CN]

1-[[4-(bromomethyl)-5-methylfuran-2-yl](imino)oxo-lambda6-sulfanyl]-3-(1,2,3,5,6,7-hexahydro-s-indacen-4-yl)urea 5j (3.5 g, 7.737 mmol) was added to a solution of NH₃ in MeOH (200 mL, 7 M) with stirring. The mixture was stirred at room temperature for 2 h. Solvent was evaporated in vacuo and the residue was purified by reverse phase column (spherical C18, 330 g, 5 to 45% MeCN in water(0.05% NH₄HCO₃)). After lyophilization, the titled compound 1-[[4-(aminomethyl)-5-methylfuran-2-yl](imino)oxo-lambda6-sulfanyl]-3-(1,2,3,5,6,7-hexahydro-s-indacen-4-yl)urea Ex. 24 was obtained as a white solid (1.155 g, 37.97%). ¹HNMR (400 MHz, CD₃OD) δ (ppm): 7.99 (s, 1H), 6.87 (s, 1H), 3.90 (s, 2H), 2.61-2.79 (m, 8H), 2.29 (s, 3H), 1.90-1.97 (m, 4H); LC-MS: m/z [M+H]⁺=389.35.

### Example 25: Preparation of N-((1,2,3,5,6,7-hexahydro-s-indacen-4-yl)carbamoyl)-4-((isopropylamino)methyl)-5-methylfuran-2-sulfonimidamide

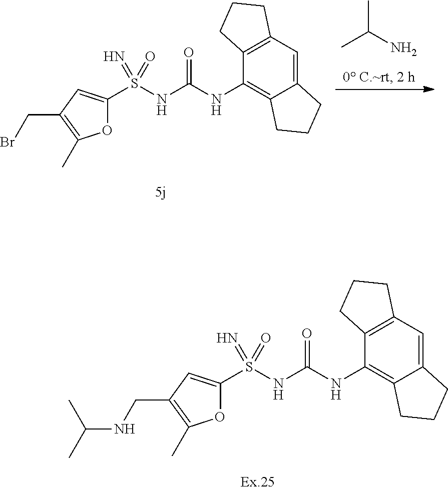

A pale brown solution of 1-[[4-(bromomethyl)-5-methylfuran-2-yl](imino)oxo-lambda6-sulfanyl]-3-(1,2,3,5,6,7-hexahydro-s-indacen-4-yl)urea 5j (150 mg, 0.332 mmol), isopropylamine (98 mg, 1.658 mmol) and DIEA (0.17 mL, 1.341 mmol) in THF (3 mL) was stirred at room temperature for 2 h. Then, the reaction solution was diluted with water (5 mL). The aqueous mixture was extracted with CHCl₃/iPrOH (15 mL×3). The combined organic phases were concentrated and the residue was purified by Prep-HPLC using the following conditions: Column; XSelect CSH Prep C18 OBD Column, 19*250 mm, 5 μm; Mobile Phase A: Water(50 mmol/L NH₄HCO₃), Mobile Phase B: ACN; Flow rate: 60 mL/min; Gradient: 17% B to 37% B in 9 min, 37% B; 254 nm; RT1: 6.52 min. After lyophilization, the titled compound 3-(1,2,3,5,6,7-hexahydro-s-indacen-4-yl)-1-[imino([4-[(isopropylamino)methyl]-5-methylfuran-2-yl])oxo-lambda6-sulfanyl]urea Ex. 25 was obtained as a yellow solid (37.0 mg, 25.70%). ¹HNMR (400 MHz, CD₃OD) δ (ppm): 7.10 (s, 1H), 6.91 (s, 1H), 3.99 (s, 2H), 3.34-3.36 (m, 1H), 2.75-2.85 (m, 8H), 2.41 (s, 3H), 1.98-2.06 (m, 4H), 1.32 (d, J=6.38 Hz, 6H); LC-MS: m/z [M+H]⁺=429.15.

### Examples 26 and 27: Preparation of N-((1,2,3,5,6,7-hexahydro-s-indacen-4-yl)carbamoyl)-4-((isopropylamino)methyl)-5-methylfuran-2-sulfonimidamide (Ex. 26) and N-((1,2,3,5,6,7-hexahydro-s-indacen-4-yl)carbamoyl)-4-((isopropylamino)methyl)-5-methylfuran-2-sulfonimidamide (Ex. 27)

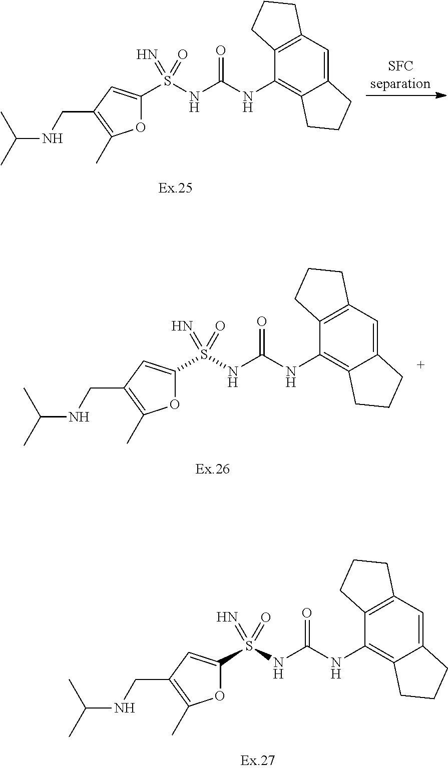

3-(1,2,3,5,6,7-Hexahydro-s-indacen-4-yl)-1-[imino([4-[(isopropylamino)methyl]-5-methylfuran-2-yl])oxo-lambda6-sulfanyl]urea Ex. 25 (200 mg), prepared from Preparative Example 25, was further purified and separated by under the following conditions: Column: CHIRALPAK IE, 2*25 cm, 5 μm; Mobile Phase A: MTBE (0.2% MSA), Mobile Phase B: EtOH; Flow rate: 17 mL/min; Gradient: 20% B to 20% B in 29 min; 254/220 nm; RT1:19.838 min; RT2: 23.948 min; Injection Volume: 0.4 ml; Number of runs: 13.

The first fraction collected at RT1 (19.838 min) was neutralized with TEA and concentrated through evaporation. The residue was purified by Prep-HPLC using the following conditions: Column: XSelect CSH Prep C18 OBD Column, 19*250 mm, 5 μm; Mobile Phase A: Water(0.05% FA), Mobile Phase B: ACN; Flow rate: 25 mL/min; Gradient: 21% B to 51% B in 7 min, 51% B; Wavelength: 254 nm; RT1(min): 5.9. After lyophilization, the titled compound Ex. 26 3-(1,2,3,5,6,7-hexahydro-s-indacen-4-yl)-1-[(S)-imino([4-[(isopropylamino)methyl]-5-methylfuran-2-yl])oxo-lambda6-sulfanyl]urea; formic acid was obtained as a white solid (30.6 mg, 4.46%). ¹HNMR (400 MHz, CD₃OD) δ (ppm): 7.10 (s, 1H), 6.91 (s, 1H), 3.96 (s, 2H), 3.34-3.36 (m, 1H), 2.69-2.85 (m, 8H), 2.42 (s, 3H), 1.99-2.06 (m, 4H), 1.31 (d, J=6.60 Hz, 6H); LC-MS: m/z [M+H]⁺=431.20. The stereo chemistry at the S atom was arbitrarily assigned.

The second fraction collected at RT2 (23.948 min) was neutralized with TEA. Solvents were removed solvents through evaporation. The residue was purified by Prep-HPLC using the following conditions: Column: XSelect CSH Prep C18 OBD Column, 19*250 mm, 5 μm; Mobile Phase A: Water(0.05% FA), Mobile Phase B: ACN; Flow rate: 25 mL/min; Gradient: 21% B to 51% B in 7 min, 51% B; Wavelength: 254 nm; RT1(min): 5.9. After lyophilization, the titled compound 3-(1,2,3,5,6,7-hexahydro-s-indacen-4-yl)-1-[(S)-imino([4-[(isopropylamino)methyl]-5-methylfuran-2-yl])oxo-lambda6-sulfanyl]urea Ex. 27 was obtained as a white solid (28.3 mg, 12.77%). ¹HNMR (400 MHz, CD₃OD) δ (ppm): 7.10 (s, 1H), 6.91 (s, 1H), 3.96 (s, 2H), 3.34-3.36 (m, 1H), 2.69-2.85 (m, 8H), 2.42 (s, 3H), 1.99-2.06 (m, 4H), 1.31 (d, J=6.60 Hz, 6H); LC-MS: m/z [M+H]⁺=431.20. The stereo chemistry at the S atom was arbitrarily assigned.

### Examples 28 and 29: Preparation of N-((8-fluoro-1,2,3,5,6,7-hexahydro-s-indacen-4-yl)carbamoyl)-4-((isopropylamino)methyl)-5-methylfuran-2-sulfonimidamide (Ex. 28) and N-((8-fluoro-1,2,3,5,6,7-hexahydro-s-indacen-4-yl)carbamoyl)-4-((isopropylamino)methyl)-5-methylfuran-2-sulfonimidamide (Ex. 29)

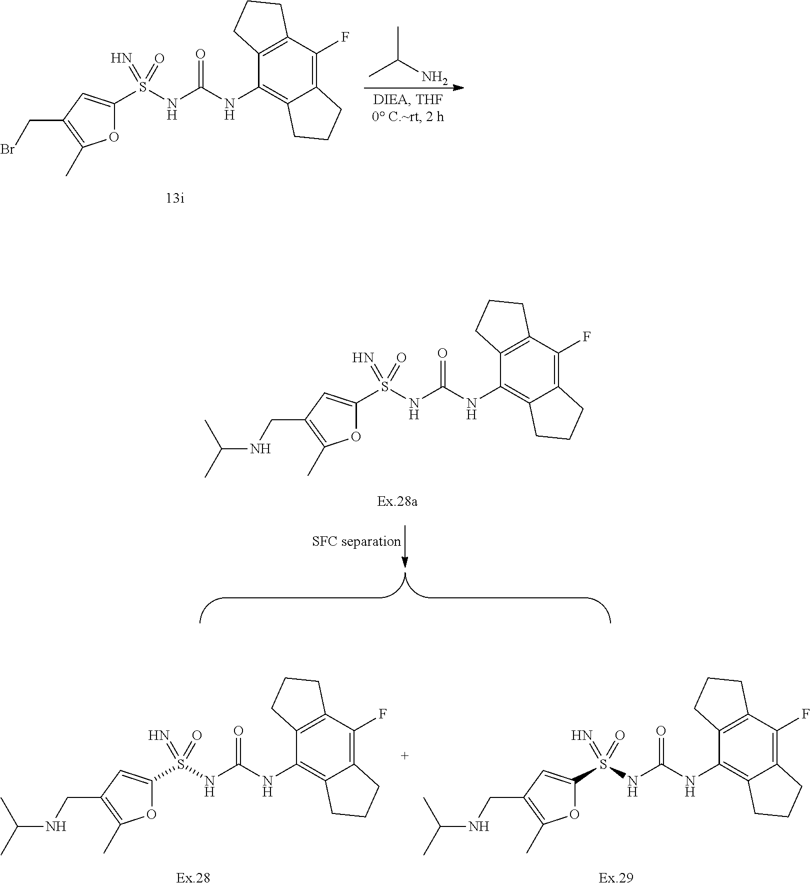

### Step 1:

To a solution of propan-2-amine (170.92 mg, 2.891 mmol) in THF (10 mL) was added DIEA (0.76 mL, 4.363 mmol) and 4-(bromomethyl)-N-((8-fluoro-1,2,3,5,6,7-hexahydro-s-indacen-4-yl)carbamoyl)-5-methylfuran-2-sulfonimidamide 13i (680 mg, 1.446 mmol). The resulting light brown suspension was stirred at room temperature for 2 h. Water (5 mL) was added, and the aqueous mixture was extracted with iPrOH/CHCl₃ (v/v, 1:3, 3×10 mL). The organic extracts were combined, dried over anhydrous sodium sulfate, filtered and concentrated. The crude product was purified by Prep-HPLC using the following conditions: Column, Cat No: SO₂₃₀₁₂₀-2, C18, 120 g, 20-45 μm, 100 Å, Lot: BP0002P2503; mobile phase A: H₂O (0.05% FA), mobile phase B: CH₃CN; Gradient: 15% B to 35% B in 40 min, 35% B in 10 min, 35% to 95% B in 2 min, 95% B in 10 min; Detector, UV 220 nm & 254 nm. After lyophilization, N-((8-fluoro-1,2,3,5,6,7-hexahydro-s-indacen-4-yl)carbamoyl)-4-((isopropylamino)methyl)-5-methylfuran-2-sulfonimidamide 28a was obtained as a white solid (150 mg, 22.08%). LC-MS: m/z [M+H]⁺=449.20.

### Step 2:

3-(8-Fluoro-1,2,3,5,6,7-hexahydro-s-indacen-4-yl)-1-[imino([4-[(isopropylamino)methyl]-5-methylfuran-2-yl])oxo-lambda6-sulfanyl]urea 28a (150.00 mg, 0.334 mmol) was subjected to SFC separation using following conditions: Column: CHIRALPAK IE, 2*25 cm, 5 μm; Mobile Phase A: MTBE (0.2% MSA), Mobile Phase B: EtOH; Flow rate: 20 mL/min: Gradient: 20% B to 20% B in 21 min; 254/220 nm; RT1: 15.53 min; RT2: 19.02 min: Sample Solvent: EtOH; Injection Volume: 0.5 ml; Number of runs: 8.

The first fraction collected at RT1 (15.53 min) from SFC separation was neutralized with TEA and concentrated. The residue was further purified by Prep-HPLC using the following conditions: Column: Xselect CSH OBD Column 30*150 mm 5 μm; Mobile Phase A: Water (0.05% FA), Mobile Phase B: ACN; Flow rate: 60 mL/min; Gradient: 10% B to 40% B in 9 min, 40% B; Wavelength: 254 nm; RT1(min): 7.23. After lyophilization, the titled compound 3-(8-fluoro-1,2,3,5,6,7-hexahydro-s-indacen-4-yl)-1-[(S)-imino([4-[(isopropylamino)methyl]-5-methylfuran-2-yl])oxo-lambda6-sulfanyl]urea Ex. 28 was obtained as a white solid (21.5 mg, 12.99%). ¹HNMR (300 MHz, Methanol-d₄) δ (ppm): 7.13 (s, 1H), 3.98 (s, 2H), 3.27-3.32 (m, 1H), 2.70-2.98 (m, 8H), 2.43 (s, 3H), 2.00-2.20 (m, 4H), 1.35 (s, 3H), 1.32 (s, 3H); ¹⁹FNMR (282 MHz, Methanol-d₄) δ (ppm): −128.06; LC-MS: m/z [M+H]⁺=449.20. The stereo chemistry at the S atom was arbitrarily assigned.

The second fraction collected at RT2 (19.02 min) from SFC separation was neutralized with TEA and solvents were removed in vacuo. The residue was further purified by Prep-HPLC using the following conditions: Column: Xselect CSH OBD Column 30*150 mm 5 μm; Mobile Phase A: Water(0.1% FA), Mobile Phase B: ACN; Flow rate: 60 mL/min; Gradient: 10% B to 40% B in 9 min, 40% B; Wavelength; 254 nm; RT1(min): 7.23. After lyophilization, the titled compound 3-(8-fluoro-1,2,3,5,6,7-hexahydro-s-indacen-4-yl)-1-[(R)-imino([4-[(isopropylamino)methyl]-5-methylfuran-2-yl])oxo-lambda6-sulfanyl]urea Ex. 29 was obtained as a white solid (21.7 mg, 13.11%). ¹HNMR (300 MHz, Methanol-d₄) δ (ppm): 7.12 (s, 1H), 3.96 (s, 2H), 3.24-3.31 (m, 1H), 2.71-2.95 (m, 8H), 2.43 (s, 3H), 2.01-2.19 (m, 4H), 1.33 (s, 3H), 1.31 (s, 3H); ¹⁹FNMR (282 MHz, Methanol-d₄) δ (ppm): −128.07; LC-MS: m/z [M+H]⁺=449.20. The stereo chemistry at the S atom was arbitrarily assigned.

### Examples 30 and 31: Preparation of N-((8-fluoro-1,2,3,5,6,7-hexahydro-s-indacen-4-yl)carbamoyl)-4-((isopropylamino)methyl)furan-2-sulfonimidamide (Ex. 30) and N-((8-fluoro-1,2,3,5,6,7-hexahydro-s-indacen-4-yl)carbamoyl)-4-((isopropylamino)methyl)furan-2-sulfonimidamide (Ex. 31)

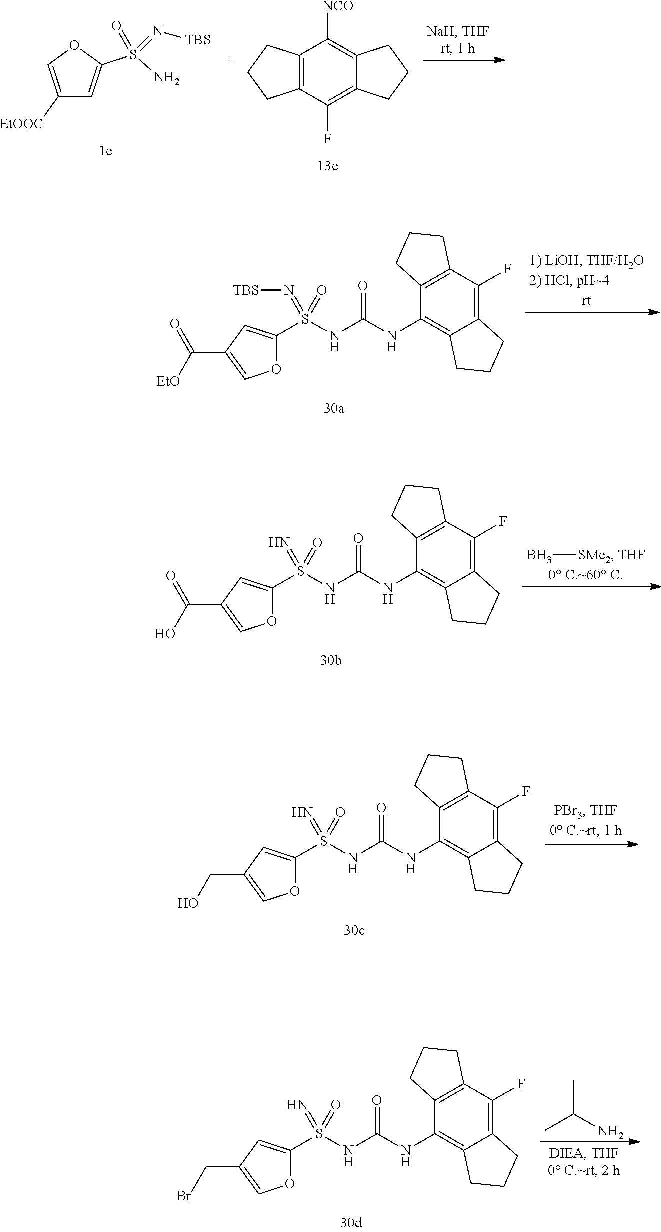

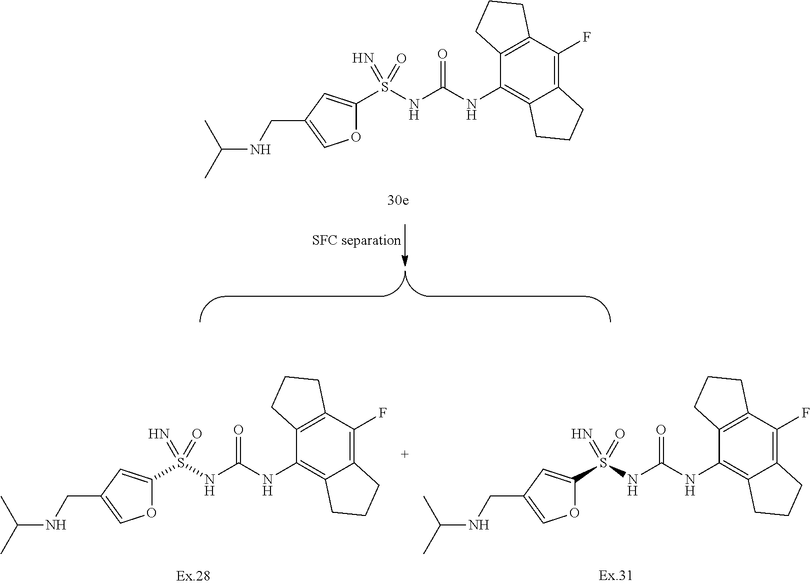

### Step 1:

NaH (73 mg, 3.038 mmol) was added to a stirred solution of ethyl 5-[(tert-butyldimethylsilyl)-S-aminosulfonimidoyl]furan-3-carboxylate 1e (1010 mg, 3.038 mmol) in THF (20 mL) at 0° C. The suspension was stirred at this temperature for 15 min. 4-fluoro-8-isocyanato-1,2,3,5,6,7-hexahydro-s-indacene 13e (660 mg, 3.038 mmol), available from preparative example 13, was added. Cooling was stopped, and the resulting suspension was stirred at rt for 1 h. A solution of CHCl₃/iPrOH (v/v, 3/1, 30 mL) was added followed by water (15 mL). The mixture was separated, the aqueous phase was extracted with CHCl₃/iPrOH (v/v, 3/1, 30 mL×2). The combined organic phases were washed with brine (20 mL×2), dried with Na₂SO₄, filtered and concentrated in vacuo to give ethyl 5-([[(8-fluoro-1,2,3,5,6,7-hexahydro-s-indacen-4-yl)carbamoyl]amino](imino)oxo-lambda6-sulfanyl)furan-3-carboxylate 30a as a white solid (1.0 g, 75.59%). LC-MS: m/z [M+H]⁺=549.21.

### Step 2:

A solution of LiOH (165 mg, 6.889 mmol) in H₂O (12 ML) was added to a solution of ethyl 5-([[(8-fluoro-1,2,3,5,6,7-hexahydro-s-indacen-4-yl)carbamoyl]amino](imino)oxo-lambda6-sulfanyl)furan-3-carboxylate 30a (1 g, 2.296 mmol) in THF (20 mL) and EtOH (8 mL). The resulting brown solution was stirred at room temperature for 3 h. The mixture was concentrated in vacuo to remove the volatiles and the remaining aqueous mixture was acidified to pH ˜4 using a 2M HCl aqueous solution to afford a suspension. The white solids were collected by filtration and further dried in oven to give 5-([[(8-fluoro-1,2,3,5,6,7-hexahydro-s-indacen-4-yl)carbamoyl]amino](imino)oxo-lambda6-sulfanyl)furan-3-carboxylic acid 30b as a white solid (700 g, 74.82%). LC-MS: m/z [M+H]⁺=408.15.

### Step 3:

BH₃-Me₂S complex (0.86 mL, 10M, 8.6 mmol) was added dropwise into a stirred colorless suspension of 5-([[(8-fluoro-1,2,3,5,6,7-hexahydro-s-indacen-4-yl)carbamoyl]amino](imino)oxo-lambda6-sulfanyl)furan-3-carboxylic acid 30b (700 mg, 1.718 mmol) in THF (20 mL) at 0° C. under N₂. Cooling was stopped and the resulting mixture was heated at 60° C. for 1 h to generate a colorless suspension. After cooled to room temperature, the reaction mixture was quenched with NH₄Cl (aq, 5 mL) and diluted with water 10 mL. The aqueous mixture was extracted with CHCl3/iPrOH (v/v, 3/1, 40 mL×3), and the combined organic extracts were concentrated to afford 3-(8-fluoro-1,2,3,5,6,7-hexahydro-s-indacen-4-yl)-1-[[4-(hydroxymethyl)furan-2-yl](imino)oxo-lambda6-sulfanyl]urea 30c as a white solid (550 mg, 81.37%). LC-MS: m/z [M+H]⁺=394.15.

### Step 4:

PBr₃ (378 mg, 1.398 mmol) was added dropwise to a colorless suspension of 3-(8-fluoro-1,2,3,5,6,7-hexahydro-s-indacen-4-yl)-1-[[4-(hydroxymethyl)furan-2-yl](imino)oxo-lambda6-sulfanyl]urea 30c (550 mg, 1.398 mmol) in THF (10 mL) at 0° C. The resulting suspension was stirred at room temperature for 1 h. The mixture was diluted with water (5 mL) and extracted with iPrOH/CHCl₃ (v/v, 1/3, 30 mL×3). The combined organic extracts were washed with brine (30 mL), dried with Na₂SO₄, filtered and concentrated. The crude solid was recrystallized from petroleum ether to give 1-[[4-(bromomethyl)furan-2-yl](imino)oxo-lambda6-sulfanyl]-3-(8-fluoro-1,2,3,5,6,7-hexahydro-s-indacen-4-yl)urea 30d as a white solid (500 mg, 78.38%). LC-MS: m/z [M]+, [M+2]⁺=456.05, 458.05.

### Step 5:

A colorless suspension of isopropylamine (129 mg, 2.191 mmol), DIEA (424 mg, 3.287 mmol) and 1-[[4-(bromomethyl)furan-2-yl](imino)oxo-lambda6-sulfanyl]-3-(8-fluoro-1,2,3,5,6,7-hexahydro-s-indacen-4-yl)urea 30d (500 mg, 1.096 mmol) in THF (4 mL) was stirred at room temperature for 2 h. The suspension was diluted with iPrOH/CHCl₃ (v/v, 1/3, 10 mL) and water (3 mL). The mixture was separated, the aqueous phase was extracted with iPrOH/CHCl₃ (v/v, 1/3, 10 mL×2), and the combined organic phases were concentrated. The residue was purified by reverse phase column (spherical C18, 80 g, 0 to 45% MeCN in water(0.05% FA)). After lyophilization, N-((8-fluoro-1,2,3,5,6,7-hexahydro-s-indacen-4-yl)carbamoyl)-4-((isopropylamino)methyl)furan-2-sulfonimidamide 30e was obtained as a white solid (250 mg),

### Step 6:

N-((8-fluoro-1,2,3,5,6,7-hexahydro-s-indacen-4-yl)carbamoyl)-4-((isopropylamino)methyl)furan-2-sulfonimidamide 30e (250 mg) was subjected to SFC separation under the following conditions: Column: CHIRALPAK IE, 2*25 cm, 5 μm; Mobile Phase A: MTBE (0.2% MSA), Mobile Phase B: EtOH; Flow rate: 17 mL/min; Gradient: 20% B to 20% B in 29 min; 254/220 nm; RT1:19.838 min; RT2: 23.948 min; Injection Volumne: 0.4 ml; Number of runs: 13.

The first fraction obtained at RT1 (19.838 min) from SFC separation was neutralized with TEA and solvents were removed under vacuo. The residue was purified further by Prep-HPLC using the following conditions: Column: XBridge Shield RP18 OBD Column, 30*150 mm, 5 μm; Mobile Phase A: Water(10 mmol/L NH₄HCO₃+0.1% NH₃·H₂O), Mobile Phase B: ACN; Flow rate: 60 mL/min; Gradient: 21% B to 51% B in 7 min, 51% B; Wavelength: 254 nm; RT1(min): 5.15. After lyophilization, the titled compound 3-(8-fluoro-1,2,3,5,6,7-hexahydro-s-indacen4-yl)-1-[(S)-imino([4-[(isopropylamino)methyl]furan-2-yl])oxo-lambda6-sulfanyl]urea Ex. 30 was obtained as a white solid (19.2 mg, 4.02%). ¹HNMR (400 MHz, CD₃OD) δ (ppm): 7.70 (s, 1H), 7.09 (s, 1H), 3.72 (s, 2H), 2.79-2.93 (m, 9H), 2.03-2.11 (m, 4H), 1.13 (d, J=6.50 Hz, 6H); ¹⁹FNMR (376 MHz, CD₃OD) δ (ppm): −128.27; LC-MS: m/z [M+H]⁺=435.20. The stereo chemistry at the S atom was arbitrarily assigned.

The second fraction obtained at RT2 (23.948 min) from SFC separation was neutralized with TEA and solvents were removed through evaporation. The residue was further purified by Prep-HPLC using the following conditions: Column: XBridge Shield RP18 OBD Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 20% B to 50% B in 7 min, 50% B; Wavelength: 254 nm; RT1(min): 6.20. After lyophilization, the titled compound 3-(8-fluoro-1,2,3,5,6,7-hexahydro-s-indacen-4-yl)-1-[(R)-imino([4-[(isopropylamino)methyl]furan-2-yl])oxo-lambda6-sulfanyl]urea Ex. 31 was obtained as a white solid (19.1 mg, 7.62%). ¹HNMR (400 MHz, CD₃OD) δ (ppm): 7.69 (s, 1H), 7.08 (s, 1H), 3.72 (s, 2H), 2.79-2.96 (m, 9H), 2.03-2.11 (m, 4H), 1.13 (d, J=6.50 Hz, 6H); ¹⁹FNMR (376 MHz, CD₃OD) δ (ppm): −128.27; LC-MS: m/z [M+H]⁺=435.20. The stereo chemistry at the S atom was arbitrarily assigned.

### Example 32: Preparation of N-((1,2,3,5,6,7-hexahydro-s-indacen-4-yl)carbamoyl)-4-((3-hydroxyazetidin-1-yl)methyl)furan-2-sulfonamide

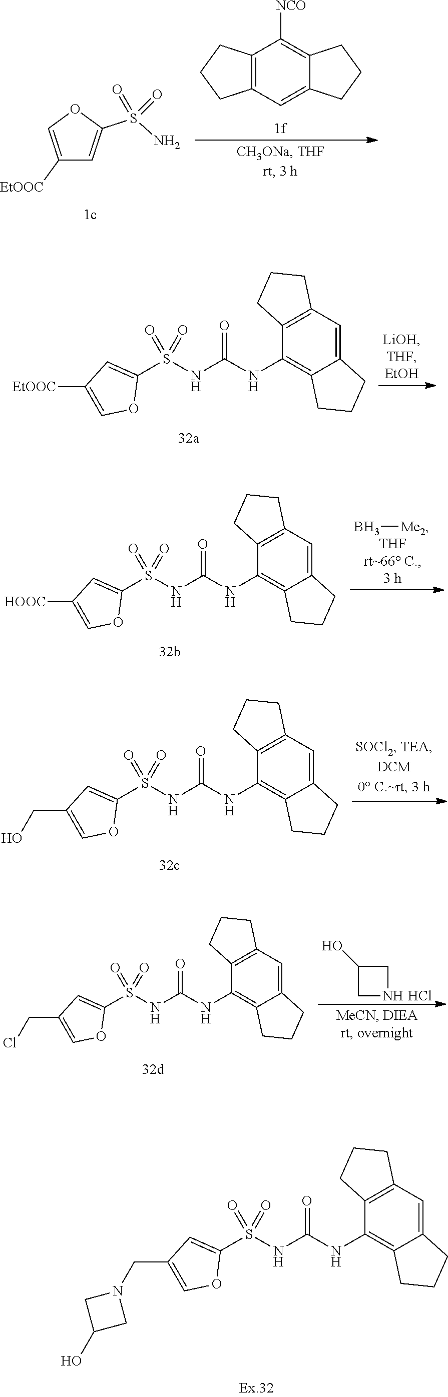

### Step 1:

Sodium methoxide (596.49 mg, 11.041 mmol) was added to a solution of ethyl 5-sulfamoylfuran-3-carboxylate 1c (2 g, 10.037 mmol) in THF (30 mL) at 0(C. The resulting suspension was stirred this temperature for 0.5 h. 4-isocyanato-1,2,3,5,6,7-hexahydro-s-indacene 1f (2.2 g, 10.037 mmol) was added. The reaction mixture was stirred at rt for another 2.5 h. A solution of CHCl₃/iPrOH (v/v, 3/1, 100 mL) was added followed by water (100 mL). The mixture was separated, the aqueous phase was extracted with CHCl₃/iPrOH (v/v, 3/1, 100 mL×2), and the combined organic phases were washed with brine (60 mL×2), dried with Na₂SO₄, filtered and concentrated in vacuo to give ethyl 5-(N-((1,2,3,5,6,7-hexahydro-s-indacen-4-yl)carbamoyl)sulfamoyl)furan-3-carboxylate 32a as a white solid (1.0 g, 42.50%). LC-MS: m z [M+H]⁺=419.10.

### Step 2:

A colorless solution of LIOH (0.17 g, 7.170 mmol) in H₂O (5 mL) was added to a slight yellow solution of ethyl 5-(N-((1,2,3,5,6,7-hexahydro-s-indacen-4-yl)carbamoyl)sulfamoyl)furan-3-carboxylate 32a (1 g, 0.239 mmol) in THF (20 mL) and EtOH (5 mL) at room temperature. The resulting mixture was stirred at room temperature for 4 h. Solvents were removed in vacuo to give a colorless suspension. The pH value of this suspension was adjusted to about 6 by addition of an aqueous solution of HCl (2 N) to generate a colorless precipitate. The precipitate was filtered and dried under reduced vacuum providing 5-(N-((1,2,3,5,6,7-hexahydro-s-indacen-4-yl)carbamoyl)sulfamoyl)furan-3-carboxylic acid 32b as a white solid (800 mg, 73.31). LC-MS: m/z [M+H]⁺=391.15.

### Step 3:

To a solution of 5-[[(1,2,3,5,6,7-hexahydro-s-indacen-4-yl)carbamoyl]aminosulfonyl]furan-3-carboxylic acid 32b (200 mg, 0.512 mmol) in THF (4 mL) was added BH₂-Me₂S complex (4 ml, 10 M, 1 mmol) dropwise at rt under a nitrogen atmosphere. The resulting mixture was stirred at 65° C. for 3 h. The mixture was poured into 25 ml of water and extracted with EtOAc (20 mL×3). The combined extracts were washed with brine (30 mL×2), dried over anhydrous Na₂SO₄, filtered and concentrated. The residue was purified by Prep-HPLC using the following conditions: Column: XBridge Prep OBD C18 Column, 30*150 mm, 5 μm; Mobile Phase A: Water(0.05% FA), Mobile Phase B: ACN; Flow rate; 60 mL/min; Gradient: 28% B to 48% B in 7 min, 48% B to B; 254 nm; RT1: 6.63 min. After lyophilization, N-((1,2,3,5,6,7-hexahydro-s-indacen-4-yl)carbamoyl)-4-(hydroxymethyl)furan-2-sulfonamide 32c as a white solid (28.0 mg, 13.81%). ¹HNMR (300 MHz, Methanol-d4) δ (ppm): 7.56-7.77 (m, 1H), 7.15 (s, 1H), 6.89-7.07 (m, 1H), 4.45-4.53 (m, 21), 2.80-2.94 (m, 4H), 2.63-2.76 (m, 4H), 1.95-2.15 (m, 4H); LC-MS: m/z [M+H]⁺=377.1.

### Step 4:

To a solution of N-((1,2,3,5,6,7-hexahydro-s-indacen-4-yl)carbamoyl)-4-(hydroxymethyl)furan-2-sulfonamide 32c (400 mg, 1.063 mmol) in DCM (3 ml) was added TEA (161.29 mg, 1.595 mmol) and SOCl₂ (151.70 mg, 1.276 mmol) at 0° C. The resulting suspension was stirred at room temperature for 3 h. Water (20 mL) was added, and the aqueous mixture was extracted with EtOAc (40 mL×3). The combined organic extracts were washed with brine (30 mL), dried with Na₂SO₄, filtered and concentrated. The crude solid was recrystallized from petroleum ether to afford 4-(chloromethyl)-N-((1,2,3,5,6,7-hexahydro-s-indacen-4-yl)carbamoyl)furan-2-sulfonamide 32d as a yellow solid (170 mg, 26.42%). LC-MS: m/z [M+H]⁺=395.0.

### Step 5:

A colorless suspension of azetidin-3-ol hydrochloride (44.39 mg, 0.405 mmol) and DIEA (78.55 mg, 0.609 mmol) in MeCN (2 mL) was stirred at rt for 0.5 h. To this suspension, 1-[4-(chloromethyl)furan-2-ylsulfonyl]-3-(1,2,3,5,6,7-hexahydro-s-indacen-4-yl)urea 32d (80.00 mg, 0.203 mmol) was added. The reaction mixture was stirred at rt overnight, filtered and concentrated. The residue was purified by Prep-HPLC using the following conditions: Column: XBridge Shield RP18 OBD Column, 19*250 mm, 10 μm; Mobile Phase A: Water(10 mmol/L NH₄HCO₃+0.1% NH₃·H₂O), Mobile Phase B: ACN; Flow rate: 25 mL/min; Gradient: 12% B to 32% B in 7 min; 220 nm; RT1:5.60 min. After lyophilization, the titled compound N-((1,2,3,5,6,7-hexahydro-s-indacen-4-yl)carbamoyl)-4-((3-hydroxyazetidin-1-yl)methyl)furan-2-sulfonamide Ex. 32 was obtained as a white solid (13.0 mg, 14.28%). ¹HNMR (300 MHz, DMSO-d6) δ (ppm): 7.80 (s, 1H), 6.77-6.83 (m, 2H), 4.38-4.49 (m, 1H), 4.04-4.16 (m, 4H), 3.67-3.79 (m, 2H), 2.68-2.79 (m, 4H), 2.56-2.67 (m, 4H), 1.81-1.96 (m, 4H); LC-MS: m/z [M+H]⁺=431.15.

### Example 33: Preparation of N-((1,2,3,5,6,7-hexahydro-s-indacen-4-yl)carbamoyl)-4-((((1-hydroxycyclobutyl)methyl)(methyl)amino)methyl)furan-2-sulfonamide

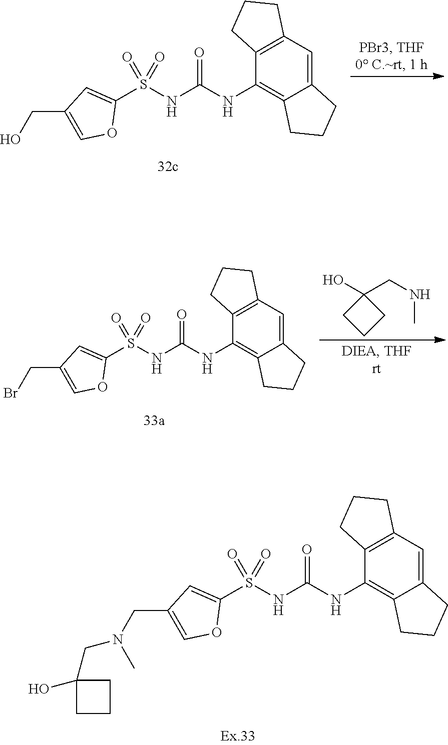

Step 1:

PBr₃ (418.5 mg, 1.546 mmol) was added dropwise to a colorless suspension of N-((1,2,3,5,6,7-hexahydro-s-indacen-4-yl)carbamoyl)-4-(hydroxymethyl)furan-2-sulfonamide 32c (580 mg, 1.546 mmol) in THF (10 mL) at 0° C. The resulting suspension was stirred at room temperature for 1 h. The reaction mixture was diluted with water (5 mL), and extracted with iPrOH/CHCl₃ (v/v, 1/3, 30 mL×3). The combined organic extracts were washed with brine (30 mL), dried with Na₂SO₄, filtered and concentrated. The crude solid was recrystallized from petroleum ether providing 4-(bromomethyl)-N-((1,2,3,5,6,7-hexahydro-s-indacen-4-yl)carbamoyl)furan-2-sulfonamide 33a as a white solid (570 mg, 84.19%). LC-MS: ma [M]⁺, [M+2]=439.00, 441.00.

### Step 2:

A brown suspension of 1-[4-(bromomethyl)furan-2-ylsulfonyl]-3-(1,2,3,5,6,7-hexahydro-s-indacen-4-yl)urea 33a (150.0 mg, 0.341 mmol), 1-[(methylamino)methyl]cyclobutan-1-ol (78.7 mg, 0.682 mmol) and DIEA (132.4 mg, 1.023 mmol) in THF (3 mL) was stirred at room temperature for 2 h. The reaction mixture was diluted with water (5 mL) and extracted with iPrOH/CHCl₃ (v/v, 1/3, 10 mL×3). The combined organic extracts were washed with brine (10 mL), dried with Na₂SO₄, filtered and concentrated. The residue was purified by Pre-HPLC using the following conditions: Column: YMC-Actus Triart C18 ExRS, 30*150 mm, 5 μm; Mobile Phase A: Water(0.05% FA), Mobile Phase B: ACN; Flow rate: 60 mL/min; Gradient: 22% B to 30% B in 11 min; 254 nm; RTL: 5.6 min. After lyophilization, the titled compound 3-(1,2,3,5,6,7-hexahydro-s-indacen-4-yl)-1-[4-([[(1-hydroxycyclobutyl)methyl](methyl)amino]methyl)furan-2-ylsulfonyl]urea Ex. 33 was obtained as a white solid (20.0 mg, 11.89%). ¹HNMR (300 MHz, CD₃OD) δ (ppm): 7.87 (s, 1H), 7.10 (s, 1H), 6.89 (s, 1H), 4.22 (s, 2H), 3.25 (s, 2H), 2.72-2.87 (m, 11H), 2.13-2.18 (m, 4H), 1.96-2.08 (m, 4H), 1.77-1.84 (m, 1H), 1.56-1.66 (m, 1H); LC-MS: m/z [M+H]⁺=474.15.

### Example 34: Preparation of N-((1,2,3,5,6,7-hexahydro-s-indacen-4-yl)carbamoyl)-4-((((1-(hydroxymethyl)cyclobutyl)methyl)(methyl)amino)methyl)-5-methylfuran-2-sulfonamide

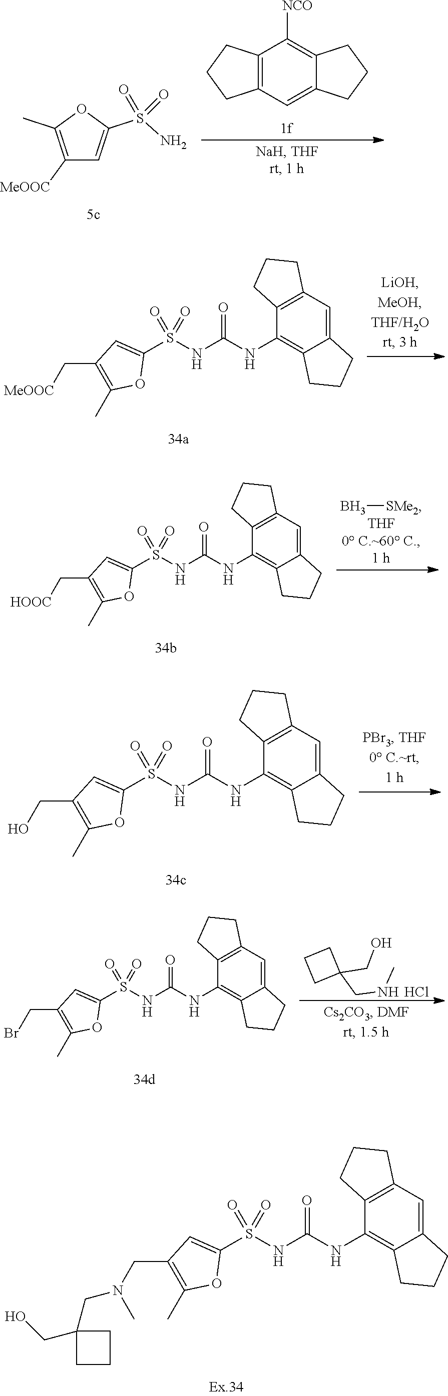

### Step 1:

NaH (0.23 g, 9.635 mmol) was added to a stirred solution of methyl 2-methyl-5-sulfamoylfuran-3-carboxylate 5c (1.92 g, 8.759 mmol) in THF (30 mL) at 0° C. The resulting suspension was stirred at this temperature for 15 min. 4-isocyanato-1,2,3,5,6,7-hexahydro-s-indacene 1f (2.09 g, 10.511 mmol) was added. Cooling was stopped, and the reaction mixture was stirred at rt for 1 h. A solution of CHCl₃/iPrOH (v/v, 3/1, 70 mL) was added followed by water (50 mL). The mixture was separated, the aqueous phase was extracted with CHCl₃/iPrOH (v/v, 3/1, 50 mL×2), and the combined organic phases were washed with brine (80 mL×2), dried with Na₂SO₄, filtered and concentrated in vacuo to give methyl 5-(N-((1,2,3,5,6,7-hexahydro-s-indacen-4-yl)carbamoyl)sulfamoyl)-2-methylfuran-3-carboxylate 34a as a white solid (3.5 g, 95.23%). LC-MS: m/z [M+H]⁺=419.1.

### Step 2:

A solution of LiOH (0.60 g, 25.092 mmol) in H₂O (40 mL) was added to a stirred solution of methyl 5-(N-((1,2,3,5,6,7-hexahydro-s-indacen-4-yl)carbamoyl)sulfamoyl)-2-methylfuran-3-carboxylate 34a (3.5 g, 8.364 mmol) in THF (80 mL) and MeOH (20 mL) to give a brown solution. The reaction mixture was stirred at room temperature for 3 h. The volatile solvents were removed in vacuo and the remainder aqueous mixture was acidified to pH ˜4 using a 2M aqueous HCl solution to form a suspension. The white solids were collected through filtration and further dried in oven to provide 5-(N-((1,2,3,5,6,7-hexahydro-s-indacen-4-yl)carbamoyl)sulfamoyl)-2-methylfuran-3-carboxylic acid 34b as a white solid (3.0 g, 95.23%). LC-MS: m/z [M+H]⁺=405.0.

### Step 3:

BH₃-Me₂S complex (1.24 mL, 10 M, 12.365 mmol) was added dropwise to a stirred colorless suspension of 5-(N-((1,2,3,5,6,7-hexahydro-s-indacen-4-yl)carbamoyl)sulfamoyl)-2-methylfuran-3-carboxylic acid 34b (1 g, 2.473 mmol) in THF (50 mL) at 0° C. under N₂ to give a colorless solution. Cooling was stopped, the reaction mixture was heated up to 60° C. and stirred at this temperature for 1 h. After cooled to room temperature, the reaction was quenched with NH₄Cl (aq, 10 mL) and diluted with water 30 mL. The aqueous mixture was extracted with iPrOH/CHCl₃ (3/1, 40 mL×3). The combined organic phases were concentrated in vacuo to provide N-((1,2,3,5,6,7-hexahydro-s-indacen-4-yl)carbamoyl)-4-(hydroxymethyl)-5-methylfuran-2-sulfonamide 34c as a white solid (620 mg, 64.22%). LC-MS: m/z [M+H]⁺=391.1.

### Step 4:

PBr₃ (429.83 mg, 1.588 mmol) was added dropwise to a colorless suspension of N-((1,2,3,5,6,7-hexahydro-s-indacen-4-yl)carbamoyl)-4-(hydroxymethyl)-5-methylfuran-2-sulfonamide 34c (620 mg, 1.588 mmol) in THF (10 mL) at 0° C. The resulting suspension was stirred at room temperature for 1 h. The mixture was diluted with water (5 mL) and extracted with iPrOH/CHCl₃ (v/v, 1/3, 10 mL×3). The combined organic extracts were washed with brine (10 mL), dried with Na₂SO₄, filtered and concentrated. The crude solid was recrystallized from petroleum ether to provide 4-(bromomethyl)-N-((1,2,3,5,6,7-hexahydro-s-indacen-4-yl)carbamoyl)-5-methylfuran-2-sulfonamide 34d as a white solid (680 mg, 94.46%). LC-MS: m/z [M+H]⁺=453.0.

### Step 5:

A colorless suspension of (1-((methylamino)methyl)cyclobutyl)methanol hydrochloride (171.00 mg, 1.324 mmol) and Cs₂CO₃ (1078.04 mg, 3.310 mmol) in DMF (2 mL) was stirred at rt for 0.5 h. To this suspension, 4-(bromomethyl)-N-((1,2,3,5,6,7-hexahydro-s-indacen-4-yl)carbamoyl)-5-methylfuran-2-sulfonamide 34d (300 mg, 0.662 mmol) was added. The resulting light brown suspension was stirred at rt for 1 h and filtered. The filtrate was purified by Prep-HPLC using the following conditions: Column: XBridge Prep OBD C18 Column, 30*150 mm, 5 μm; Mobile Phase A: Water(10 mmol/L FA), Mobile Phase B: ACN; Flow rate: 60 mL/min: Gradient: 37% B to 67% B in 7 min; Wavelength: 254 nm; RT1(min): 6.5. After lyophilization, The titled compound N-((1,2,3,5,6,7-hexahydro-s-indacen-4-yl)carbamoyl)-4-((((1-(hydroxymethyl)cyclobutyl)methyl)methyl)amino)methyl)-5-methylfuran-2-sulfonamide Ex. 34 was obtained as a white solid (22.6 mg, 6.74%). ¹HNMR (400 MHz, CD₃OD) δ (ppm): 6.95 (s, 1H), 6.90 (s, 1H), 4.09 (s, 2H), 3.81 (s, 2H), 3.23 (s, 2H), 2.81-2.88 (m, 4H), 273-2.81 (m, 7H), 2.41 (s, 3H), 1.86-2.08 (m, 10H); LC-MS: m/z [M+H]⁺=502.2.

### Example 35: Preparation of N-((8-fluoro-1,2,3,5,6,7-hexahydro-s-indacen-4-yl)carbamoyl)-4-((((1-hydroxycyclobutyl)methyl)(methyl)amino)methyl)-5-methylfuran-2-sulfonamide

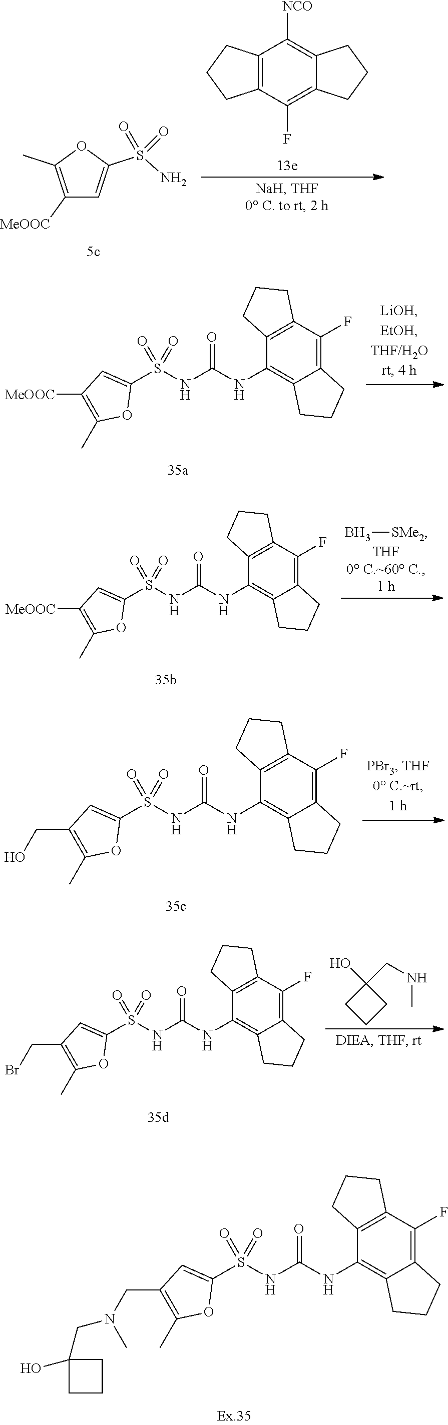

### Step 1:

NaH (181 mg, 7.527 mmol) was added to a stirred solution of methyl 2-methyl-5-sulfamoylfuran-3-carboxylate 5c(1.5 g, 6.843 mmol) in THF (30 mL) at 0° C. The resulting suspension was stirred at this temperature for 15 min. Then, 4-fluoro-8-isocyanato-1,2,3,5,6,7-hexahydro-s-indacene 13e (1.64 g, 7.527 mmol) was added. Cooling was stopped and the reaction mixture was stirred at rt for 2 h. A solution of CHCl₃/iPrOH (v/v, 3/1, 100 mL) was added followed by water (50 mL). The mixture was separated, the aqueous phase was extracted with CHCl₃/iPrOH (v/v, 3/1, 100 mL×2), and the combined organic phases were washed with brine (50 mL×2), dried with Na₂SO₄, filtered and concentrated in vacuo to give methyl 5-[[(8-fluoro-1,2,3,5,6,7-hexahydro-s-indacen-4-yl)carbamoyl]aminosulfonyl]-2-methylfuran-3-carboxylate 35a as a white solid (2.5 g, 83.71%). LC-MS: m/z [M+H]⁺=437.05.

### Step 2:

A solution of LiOH (412 mg, 17.184 mmol) in H₂O (20 mL) was added to a solution of methyl 5-[[(8-fluoro-1,2,3,5,6,7-hexahydro-s-indacen-4-yl)carbamoyl]aminosulfonyl]-2-methylfuran-3-carboxylate 35a (2.5 g, 5.728 mmol) in THF (50 mL) and MeOH (10 mL). The resulting brown solution was stirred at room temperature for 4 h. The mixture was concentrated, and the aqueous residue was acidified to pH ˜6 using a 2M aqueous solution of HCl to afford a suspension. The white solids were filtered and dried in oven to give 5-[[(8-fluoro-1,2,3,5,6,7-hexahydro-s-indacen-4-yl)carbamoyl]aminosulfonyl]-2-methylfuran-3-carboxylic acid 35b as a white solid (2.1 g, 86.79%). LC-MS: m/z [M+H]⁺=423.10.

### Step 3:

BH₃-Me₂S complex (2.5 mL, 10 M, 25 mmol) was added dropwise to a stirred colorless suspension of 5-(N-((8-fluoro-1,2,3,5,6,7-hexahydro-s-indacen-4-yl)carbamoyl)sulfamoyl)-2-methylfuran-3-carboxylic acid 35b (2.1 g, 4.976 mmol) in THF (50 mL) at 0° C. under N₂ to generate a colorless solution. The reaction mixture was then heated up to 60° C. and continued for 1 h. After cooled to room temperature, the reaction was quenched with NH₄Cl (aq, 10 mL) and diluted with water (20 mL). The aqueous mixture was extracted with CHCl₃/iPrOH (3/1, 100 mL×3). The combined organic extracts were concentrated in vacuo to give N-((8-fluoro-1,2,3,5,6,7-hexahydro-s-indacen-4-yl)carbamoyl)-4-(hydroxymethyl)-5-methylfuran-2-sulfonamide 35c as a white solid (1.7 g, 80.95%). LC-MS: m/z [M+H]⁺=409.30.

### Step 4:

PBr₃ (1.06 g, 3.917 mmol) was added dropwise to a colorless suspension of N-((8-fluoro-1,2,3,5,6,7-hexahydro-s-indacen-4-yl)carbamoyl)-4-(hydroxymethyl)-5-methylfuran-2-sulfonamide 35c (1.6 g, 3.917 mmol) in THF (30 mL) at 0° C. The resulting suspension was stirred at room temperature for 1 h. The reaction mixture was diluted with water (5 mL) and extracted with iPrOH/CHCl₃ (v/v, 1/3, 80 mL×3). The combined organic extracts were washed with brine (80 mL), dried with Na₂SO₄, filtered and concentrated. The crude solid was recrystallized from petroleum ether providing 4-(bromomethyl)-N-((8-fluoro-1,2,3,5,6,7-hexahydro-s-indacen-4-yl)carbamoyl)-5-methylfuran-2-sulfonamide 35d as a white solid (1.6 g, 86.66%). LC-MS: m/z [M]⁺, [M+2]=471.20, 473.20.

### Step 5:

To a solution of 1-[(methylamino)methyl]cyclobutan-1-ol (48.87 mg, 0.424 mmol) in THF (2 mL) was added DIEA (82.26 mg, 0.636 mmol) and 1-[4-(bromomethyl)-5-methylfuran-2-ylsulfonyl]-3-(8-fluoro-1,2,3,5,6,7-hexahydro-s-indacen-4-yl)urea 35d (100.00 mg, 0.212 mmol). The resulting brown suspension was stirred at rt for 6 hours. Water (5 mL) was added, and the aqueous mixture was extracted with iPrOH/CHCl₃ (v/v, 1/3, 10 mL×3). The combined organic extracts were concentrated in vacuo to a residue, which was purified by Prep-HPLC using the following Column: Xselect CSH OBD Column 30*150 mm 5 μm; Mobile Phase A: Water (50 mmol/L NH₄HCO₃), Mobile Phase B: ACN; Flow rate: 60 mL/min; Gradient: 25% B to 45% B in 7 min; 254 nm; RT1: 6.35 min. After lyophilization, the titled compound N-((8-fluoro-1,2,3,5,6,7-hexahydro-s-indacen-4-yl)carbamoyl)-4-((((1-hydroxycyclobutyl)methyl)(methyl)amino)methyl)-5-methylfuran-2-sulfonamide Ex. 35 was obtained as a white solid (26.2 mg, 23.94%). ¹HNMR (300 MHz, Methanol-d4) δ (ppm): 6.91 (s, 1H), 4.15 (s, 2H), 3.25 (s, 2H), 2.74-2.90 (m, 11H), 2.40 (s, 3H), 2.14-2.24 (m, 4H), 2.00-2.13 (m, 4H), 1.76-1.90 (m, 1H), 1.55-1.74 (m, 1H); LC-MS: m/z [M+H]⁺=506.1.

### Example 36: Preparation of N-((8-fluoro-1,2,3,5,6,7-hexahydro-s-indacen-4-yl)carbamoyl)-4-((((1-hydroxycyclobutyl)methyl)amino)methyl)-5-methylfuran-2-sulfonamide

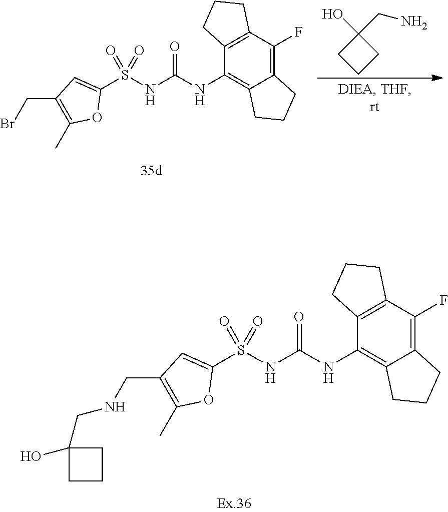 [SMILES: Cc1oc(S(=O)(=O)NC(=O)Nc2c3c(c(F)c4c2CCC4)CCC3)cc1CBr ; NCC1(O)CCC1 ; Cc1oc(S(=O)(=O)NC(=O)Nc2c3c(c(F)c4c2CCC4)CCC3)cc1CNCC1(O)CCC1]

To a mixture of 4-(bromomethyl)-N-((8-fluoro-1,2,3,5,6,7-hexahydro-s-indacen-4-yl)carbamoyl)-5-methylfuran-2-sulfonamide 35d (150 mg, 0.319 mmol) in THF (2 mL) was added 1-(aminomethyl)cyclobutan-1-ol (64.56 mg, 0.638 mmol) and DIEA (123.74 mg, 0.957 mmol). The resulting mixture was stirred at rt for 2 h. The reaction was quenched by the addition of an aqueous solution of NH₄Cl. The mixture was extracted with CHCl₃/iPrOH (v/v, 3:1, 10 mL×3). The organic extracts were combined, dried over anhydrous sodium sulfate, filtered and concentrated. The residue was purified by Prep-HPLC using the following conditions: Column: XBridge Shield RP18 OBD Column, 30*150 mm, 5 μm; Mobile Phase A: Water (0.05% FA), Mobile Phase B: ACN; Flow rate: 60 mL/min; Gradient: 15% B to 35% B in 10 min, 35% B; Wavelength: 254 nm; RT1(min): 8.48. After lyophilization, the titled compound N-((8-fluoro-1,2,3,5,6,7-hexahydro-s-indacen-4-yl)carbamoyl)-4-((((1-hydroxycyclobutyl)methyl)amino)methyl)-5-methylfuran-2-sulfonamide Ex. 36 was obtained as a white solid (12.3 mg, 7.71%). ¹HNMR (300 MHz, DMSO-d6) δ (ppm): 6.75 (s, 1H), 3.86-3.92 (m, 2H), 2.87-2.91 (m, 2H), 2.56-2.78 (m, 8H), 2.27-2.33 (m, 3H), 1.90-2.02 (m, 9H), 1.54-1.67 (m, 1H); ¹⁹FNMR (282 MHz, DMSO-d6) δ (ppm): −127.51; LC-MS: m/z [M+H]⁺=492.1.

### Example 37: Preparation of N-((8-fluoro-1,2,3,5,6,7-hexahydro-s-indacen-4-yl)carbamoyl)-4-((((1-hydroxycyclopentyl)methyl)(methyl)amino)methyl)-5-methylfuran-2-sulfonamide

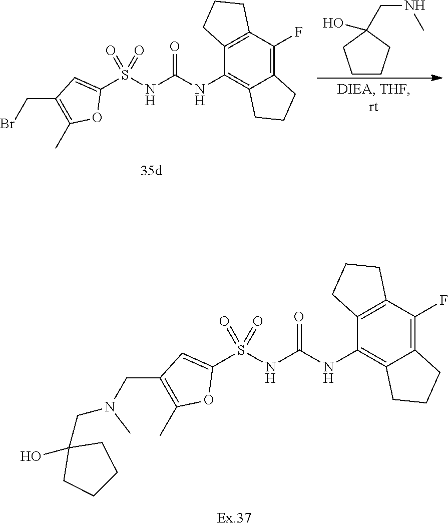

To a stirred mixture of 3-[4-(bromomethyl)-5-methylfuran-2-ylsulfonyl]-1-(8-fluoro-1,2,3,5,6,7-hexahydro-s-indacen-4-yl)urea 35d (150 mg, 0.318 mmol) in THF (2 mL) was added DIEA (123.39 mg, 0.954 mmol) and 1-[(methylamino)methyl]cyclopentane-1-ol (82.24 mg, 0.636 mmol). The reaction was continued at rt for 2 h and quenched by NH₄Cl (aq.). The aqueous mixture was extracted with CHCl₃/iPrOH (v/v, 3.1, 10 mL×3). The organic extracts were combined, dried over anhydrous sodium sulfate, filtered and concentrated. The residue was purified by Prep-HPLC using the following conditions: Column: Xselect CSH OBD Column 30*150 mm 5 μm; Mobile Phase A: Water(10 mmol/L NH₄HCO₃+0.1% NH₃·H₂O), Mobile Phase B: ACN; Flow rate: 60 mL/min; Gradient: 18% B to 38% B in 7 min, 38% B; Wavelength: 254 nm; RT1(min): 6.17. After lyophilization, the titled compound 3-(8-fluoro-1,2,3,5,6,7-hexahydro-s-indacen-4-yl)-1-[4-([[(1-hydroxycyclopentyl)methyl](methyl)amino]methyl)-5-methylfuran-2-ylsulfonyl]urea Ex. 37 was obtained as a white solid (25 mg, 15.05%). ¹HNMR (400 MHz, CD₃OD) δ (ppm): 6.94 (s, 1H), 4.17 (s, 2H), 3.18 (s, 2H), 2.70-2.88 (m, 11H), 2.40-2.43 (m, 3H), 2.04-2.15 (m, 4H), 1.55-1.80 (t, J=7.14 Hz, 3H); ¹⁹FNMR (376 MHz, CD₃OD) δ (ppm): −128.63; LC-MS: m/z [M+H]⁺=520.20.

### Example 38: Preparation of (S)—N-((8-fluoro-1,2,3,5,6,7-hexahydro-s-indacen-4-yl)carbamoyl)-4-((3-hydroxypyrrolidin-1-yl)methyl)-5-methylfuran-2-sulfonamide

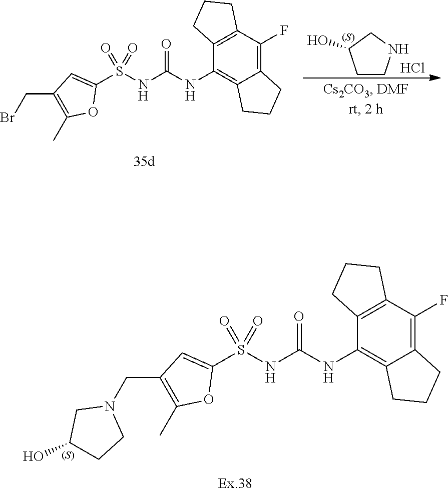

To a solution of (3S)-pyrrolidin-3-ol hydrochloride (41.95 mg, 0.340 mmol) in DMF (2 mL) was added Cs₂CO₃ (276.51 mg, 0.849 mmol). The colorless suspension was stirred at room temperature for 30 min. 3-[4-(bromomethyl)-5-methylfuran-2-ylsulfonyl]-1-(8-fluoro-1,2,3,5,6,7-hexahydro-s-indacen-4-yl)urea 35d (80.00 mg, 0.170 mmol) was added. The resulting light brown suspension was stirred at room temperature for 1.5h. Water (5 mL) was added, and the aqueous mixture was extracted with iPrOH/CHCl₃ (v/v, 1:3, 3×10 mL). The combined organic extracts were dried over anhydrous sodium sulfate, filtered and concentrated. The crude product was purified by Prep-HPLC using the following conditions: Column: XBridge Shield RP18 OBD Column, 30*150 mm, 5 μm; Mobile Phase A: Water (0.1% FA), Mobile Phase B: ACN; Flow rate: 60 mL/min; Gradient: 19% B to 39% B in 9 min, 39% B; 254 nm; RTL: 6.73 min. After lyophilization, the titled compound (S)—N-((8-fluoro-1,2,3,5,6,7-hexahydro-s-indacen-4-yl)carbamoyl)-4-((3-hydroxypyrrolidin-1-yl)methyl)-5-methylfuran-2-sulfonamide Ex. 38 was obtained as a white solid (20.2 mg, 23.74%). ¹HNMR (400 MHz, DMSO-d) δ (ppm): 6.61-7.00 (m, 1H), 4.26-4.47 (m, 1H), 3.99-4.21 (m, 2H), 2.87-3.42 (m, 4H), 2.71-2.85 (m, 4H), 2.58-2.70 (m, 4H), 2.21-2.41 (m, 3H), 2.04-2.17 (m, 1H), 1.89-2.03 (m, 4H), 1.77-1.86 (m, 1H); ¹⁹FNMR (282 MHz, DMSO-d₆) δ (ppm): −127.89; LC-MS: m/z [M+H]⁺=478.10.

### Examples 39 to 45

Following the procedures described in Preparative Example 38, by using a suitable amine precursor R₁R₂NH, Examples 39 to 45 were prepared from bromo intermediate 35d under either condition A when HCl salt was employed or condition B if free amine was employed. Results are summarized in Table 2.

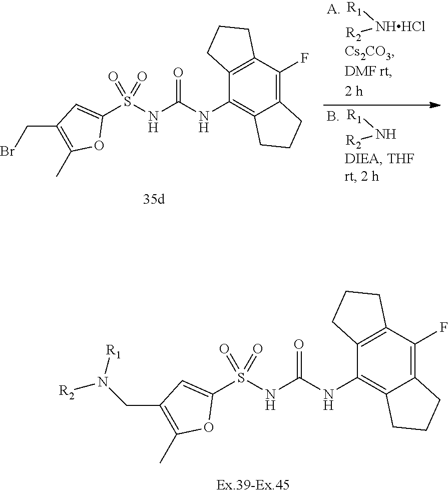

TABLE 2

| Ex. | | | |
|---|---|---|---|
| No. | R₁R₂NH | Structure/Name | LC-MS, ¹H NMR |
| 39 | 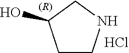 | 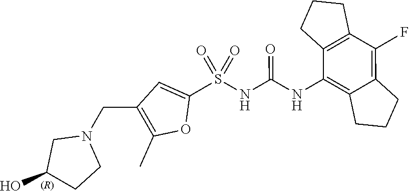 | LC-MS: m/z [M + H]⁺ = 478.15; ¹H NMR (300 MHz, DMSO-d₆) δ (ppm): 6.72 − 7.00 (m, 1H), 4.28 − 4.47 (m, 1H), 3.98 − 4.18 (m, 2H), 3.10 − 3.42 (m, 3H), 2.95 − 3.09 (m, 1H), 2.71 − 2.84 (m, 4H), 2.58 − 2.71 (m, 4H), 2.29 (s, 3H), 2.06 − 2.21 (m, 1H), 1.89 − 2.03 (m, 4H), 1.77 − 1.86 (m, 1H); ¹⁹FNMR (282 MHz, DMSO-d₆) δ (ppm): −127.75. |
| | | (R)-N-((8-fluoro-1,2,3,5,6,7- | |
| | | hexahydro-s-indacen-4- | |
| | | yl)carbamoyl)-4-((3- | |
| | | hydroxypyrrolidin-1-yl)methyl)-5- | |
| | | methylfuran-2-sulfonamide | |
| 40 | 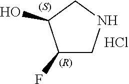 | 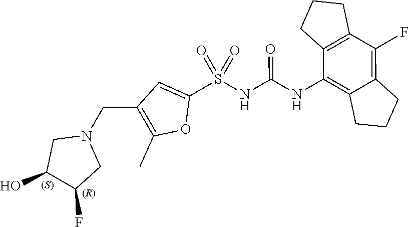 | LC-MS: m/z [M + H]⁺ = 496.20; ¹H NMR (300 MHz, DMSO-d₆) δ (ppm): ¹H NMR (300 MHz, (CD₃)₂SO) δ (ppm): 6.91 (s, 1H), 4.83, 5.02 (m, 1H), 4.10 − 4.16 (m, IH), 3.66 (s, 2H), 2.91 − 3.21 (m, 2H), 2.67 − 2.89 (m, 10H), 2.32 (s, 3H), 1.97 − 2.04 (m, 4H); ¹⁹FNMR (282 MHz, DMSO-d₆) δ (ppm): −126.33, −194.43. |
| | | N-((8-fluoro-1,2,3,5,6,7-hexahydro- | |
| | | s-indacen-4-yl)carbamoyl)-4- | |
| | | (((3R,4S)-3-fluoro-4- | |
| | | hydroxypyrrolidin-1-yl)methyl)-5- | |
| | | methylfuran-2-sulfonamide | |
| 41 | 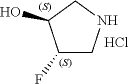 | 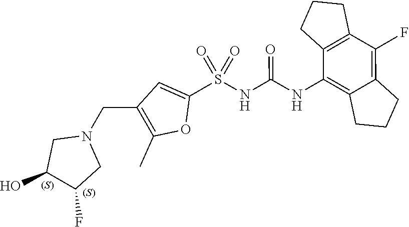 | LC-MS: m/z [M + H]⁺ = 496.20; ¹HNMR (400 MHz, DMSO-d₆) δ (ppm): 6.81 − 6.93 (m, 1H), 4.73 − 4.98 (m, 1H), 4.11 − 4.25 (m, 1H), 3.50 − 3.68 (m, 2H), 3.02 − 3.18 (m, 1H), 2.71 − 3.01 (m, 6H), 2.60 − 2.81 (m, 4H), 2.21 − 2.37 (m, 4H), 1.89 − 2.04 (m, 4H); ¹⁹FNMR (376 MHz, DMSO-d6) δ (ppm): −127.27, − 176.89. |
| | | N-((8-fluoro-1,2,3,5,6,7-hexahydro- | |
| | | s-indacen-4-yl)carbamoyl)-4- | |
| | | (((3S,4S)-3-fluoro-4- | |
| | | hydroxypyrrolidin-1-yl)methyl)-5- | |
| | | methylfuran-2-sulfonamide | |
| 42 | 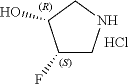 | 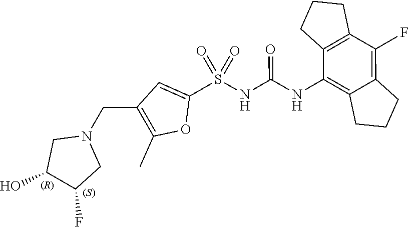 | LC-MS: m/z [M + H]⁺ = 496.20; ¹HNMR (400 MHz, DMSO-d6) δ (ppm): 6.88 (s, 1H), 4.95 − 5.08 (m, 1H), 4.17 − 4.30 (m, 1H), 3.83 − 3.99 (m, 2H), 3.19 − 3.38 (m, 1H), 3.01 − 3.17 (m, 2H), 2.75 − 2.91 (m, 5H), 2.60 − 2.68 (m, 4H), 2.32 − 2.36 (m, 3H), 1.96 − 2.08 (m, 4H). |
| | | N-((8-fluoro-1,2,3,5,6,7-hexahydro- | |
| | | s-indacen-4-yl)carbamoyl)-4- | |
| | | (((3S,4R)-3-fluoro-4- | |
| | | hydroxypyrrolidin-1-yl)methyl)-5- | |
| | | methylfuran-2-sulfonamide | |
| 43 | 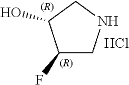 | 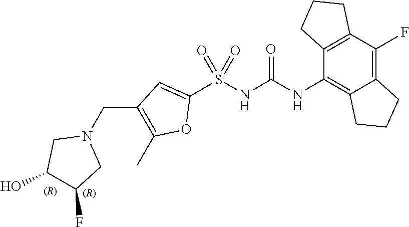 | LC-MS: m/z [M + H]⁺ = 496.20; ¹HNMR (400 MHz, DMSO-d₆) δ (ppm): 6.76 − 6.92 (m, 1H), 4.73 − 4.97 (m, 1H), 4.09 − 4.25 (m, 1H), 3.55 (s, 2H), 3.04 − 3.18 (m, 1H), 2.73 −3.02 (m, 6H), 2.57 − 2.73 (m, 4H), 2.19 − 2.41 (m, 4H), 1.84 − 2.04 (m, 4H |
| | | N-((8-fluoro-1,2,3,5,6,7-hexahydro- | |
| | | s-indacen-4-yl)carbamoyl)-4- | |
| | | (((3R,4R)-3-fluoro-4- | |
| | | hydroxypyrrolidin-1-yl)methyl)-5- | |
| | | methylfuran-2-sulfonamide | |
| 44 | 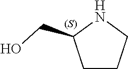 | 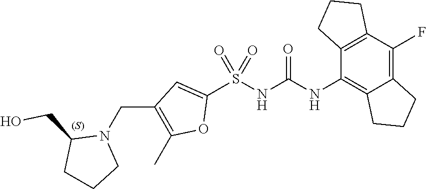 | LC-MS: m/z [M + H]⁺ = 492.20; ¹HNMR (400 MHz, DMSO-d₆) δ (ppm): 6.69 (s, 1H), 3.81 − 4.29 (m, 2H), 3.53 − 3.69 (m, 2H), 3.18 (s, 1H), 2.58 − 3.00 (m, 10H), 2.30 (s, 3H), 1.90 − 2.09 (m, 5H), 1.53 − 1.90 (m, 3H); ¹⁹FNMR (282 MHz, DMSO-d₆) δ (ppm): −127.27, −176.96. |
| | | (S)-N-((8-fluoro-1,2,3,5,6,7- | |
| | | hexahydro-s-indacen-4- | |
| | | yl)carbamoyl)-4-((2- | |
| | | (hydroxymethyl)pyrrolidin-1- | |
| | | yl)methyl)-5-methylfuran-2- | |
| | | sulfonamide | |
| 45 | 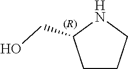 | 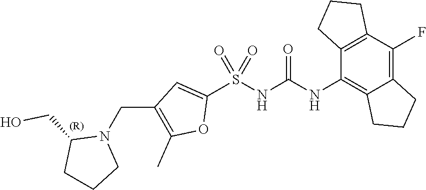 | LC-MS: m/z [M + H]⁺ = 492.20; ¹HNMR (300 MHz, DMSO-d6) δ (ppm): 6.73 (s, 1H), 4.21 − 4.25 (m, 1H), 3.94 − 3.96 (m, 1H), 3.53 − 3.59 (m, 2H), 3.24 − 3.41 (m, 2H), 2.97 (s, 3H), 2.67 − 2.82 (m, 8H), 2.29 (s, 3H), 1.71 − 2.06 (m, 8H); ¹⁹FNMR (282 MHz, DMSO-d6) δ (ppm): −127.86. |
| | | (R)-N-((8-fluoro-1,2,3,5,6,7- | |
| | | hexahydro-s-indacen-4- | |
| | | yl)carbamoyl)-4-((2- | |
| | | (hydroxymethyl)pyrrolidin-1- | |
| | | yl)methyl)-5-methylfuran-2- | |
| | | sulfonamide | |

### Example 46: Preparation of N-((8-fluoro-1,2,3,5,6,7-hexahydro-s-indacen-4-yl)carbamoyl)-4-((((4-(hydroxymethyl)tetrahydro-2H-pyran-4-yl)methyl)(methyl)amino)methyl)-5-methylfuran-2-sulfonamide

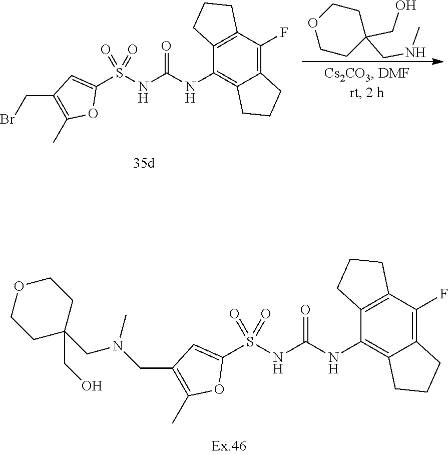

To a colorless suspension of {4-[(methylamino)methyl]oxan-4-yl}methanol (135.13 mg, 0.848 mmol) and Cs₂CO₃ (414.76 mg, 1.272 mmol) in DMF (2 mL) was added 4-(bromomethyl)-N-((8-fluoro-1,2,3,5,6,7-hexahydro-s-indacen-4-yl)carbamoyl)-5-methylfuran-2-sulfonamide 35d (200 mg, 0.424 mmol). The reaction mixture was stirred at room temperature for 2 h and filtered. The filtrate was purified by Prep-HPLC using the following condition: Column: XBridge Prep OBD C18 Column, 30*150 mm 5 μm; Mobile Phase A: Water (10 mmol/L NH₄HCO₃), Mobile Phase B: ACN; Flow rate: 60 mL/min; Gradient: 37% B to 57% B in 7 min, 57% B to B; 254 nm; RT1:6.67 min. After lyophilization, the titled compound N-((1,2,3,5,6,7-hexahydro-s-indacen-4-yl)carbamoyl)-5-((3-hydroxyazetidin-1-yl)methyl)-1-isopropyl-1H-pyrazole-3-sulfonimidamide Ex. 46 was obtained as a white solid (20.6 mg, 8.71%). ¹HNMR (400 MHz, Methanol-d4) δ (ppm): 7.00 (s, 1H), 4.03 (s, 2H), 3.47-3.78 (m, 6H), 3.05 (s, 2H), 2.84-2.91 (m, 4H), 2.75-2.84 (m, 7H), 2.42 (s, 3H), 2.03-2.16 (m, 4H), 1.52-1.57 (m, 4H); ¹⁹FNMR (376 MHz, Methanol-d4) δ (ppm): −128.38; LC-MS: m/z [M+H]⁺=550.2.

### Example 47: Preparation of N-((8-fluoro-1,2,3,5,6,7-hexahydro-s-indacen-4-yl)carbamoyl)-4-((((1-(hydroxymethyl)cyclobutylmethyl)(methyl)amino)methyl)-5-methylfuran-2-sulfonamide

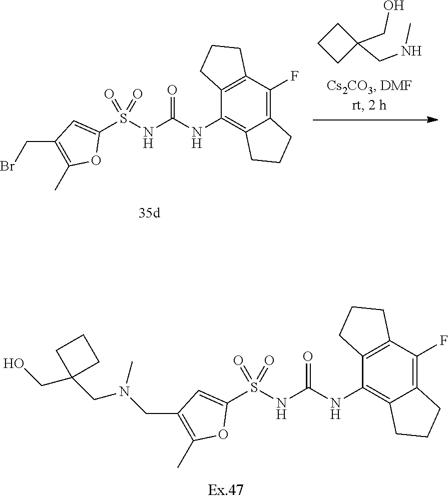

Following the procedures described in Preparative Example 46, the titled compound N-((8-fluoro-1,2,3,5,6,7-hexahydro-s-indacen-4-yl)carbamoyl)-4-((((1-(hydroxymethyl)cyclobutyl)methyl)(methyl)amino)methyl)-5-methylfuran-2-sulfonamide Ex. 47 was prepared as a colorless solid (56.8 mg). ¹HNMR (400 MHz, Methanol-d4) δ (ppm): 6.92 (s, 1H), 4.10 (s, 2H), 3.82 (s, 2H), 3.24 (s, 2H), 2.77-2.91 (m, 8H), 2.75 (s, 3H), 2.40 (s, 3H), 1.97-2.13 (m, 8H), 1.88-1.96 (m, 2H): LC-MS: m/z [M+H]⁺=520.20.

### Example 48: Preparation of N-((8-chloro-1,2,3,5,6,7-hexahydro-s-indacen-4-yl)carbamoyl)-4-((((1-hydroxycyclobutyl)methyl)(methyl)amino)methyl)-5-methylfuran-2-sulfonamide

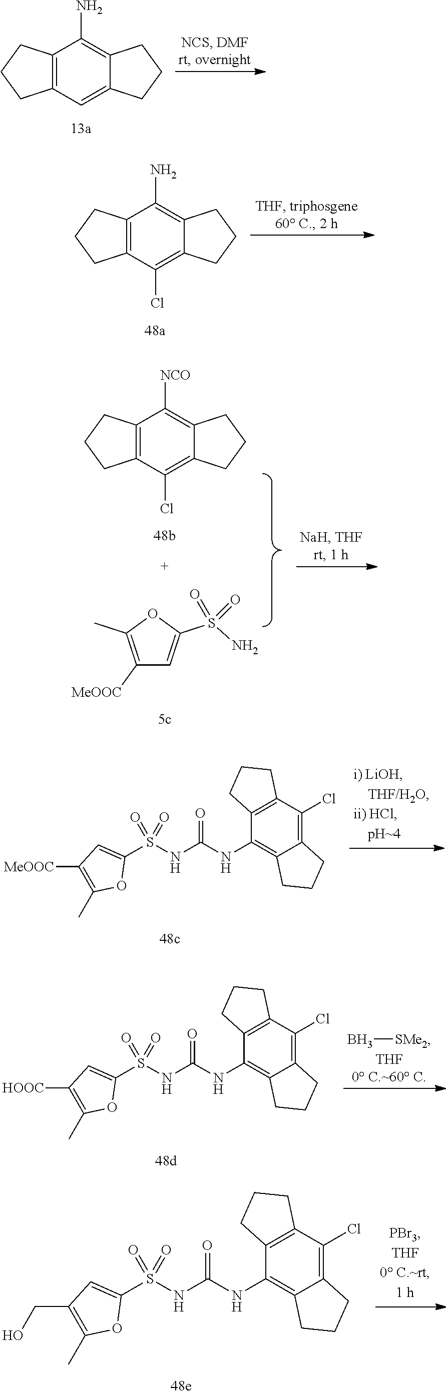

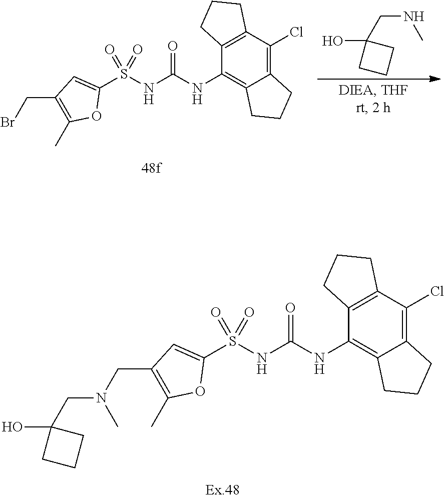

### Step 1:

A brown solution of 1,2,3,5,6,7-hexahydro-s-indacen-4-amine 13a (2.0 g, 11.561 mmol) and NCS (1.7 g, 12.717 mmol) in DMF (20 mL) was stirred at rt overnight. The mixture was added with EtOAc (40 mL) followed by water (20 mL), then separated. The aqueous phase was extracted with EtOAc (40 mL×2). The combined organic phases were washed with brine (60 mL×2), dried with Na₂SO₄, filtered and concentrated. The residue was purified by silica gel column chromatography to give 8-chloro-1,2,3,5,6,7-hexahydro-s-indacen-4-amine 48a as a white solid (1.5 g, 62.76%). LC-MS: m z [M+H]⁺=208.10.

### Step 2:

A colorless suspension of 8-chloro-1,2,3,5,6,7-hexahydro-s-indacen-4-amine 48a (100 mg, 0.481 mmol) and triphosgene (47 mg, 0.159 mmol) in THF (10 mL) was stirred at 60° C. for 2 h. After cooled down to rt, solvent was removed under vacuo to afford 4-chloro-8-isocyanato-1,2,3,5,6,7-hexahydro-s-indacene 48b as a brown solid (110 mg, 97.76%).

### Step 3:

NaH (11.3 mg, 0.471 mmol) was added to a solution of methyl 2-methyl-5-sulfamoylfuran-3-carboxylate 5c (103 mg, 0.471 mmol) in THF (3 mL) at 0° C. The resulting suspension was stirred at this temperature for 15 min. 4-chloro-8-isocyanato-1,2,3,5,6,7-hexahydro-s-indacene 48b (110 mg, 0.471 mmol) was added. Cooling was stopped, the reaction mixture was stirred at rt for 1 h. A solution of CHCl₃/iPrOH (v/v, 3/1, 10 mL) was added followed by water (5 mL). The aqueous mixture was separated, the aqueous phase was extracted with CHCl₃/CH/iPrOH (v/v, 3/1, 10 mL×2), the combined organic phases were washed with brine (5 mL×2), dried with Na₂SO₄, filtered and concentrated in vacuo to provide methyl 5-[[(8-chloro-1,2,3,5,6,7-hexahydro-s-indacen-4-yl)carbamoyl]aminosulfonyl]-2-methylfuran-3-carboxylate 48c as a white solid (200 mg, 93.82%). LC-MS: m/z [M+H]⁺=453.10.

### Step 4:

A solution of LiOH (32 mg, 1.326 mmol) in H₂O (8 mL) was added to a solution of methyl 5-[[(8-chloro-1,2,3,5,6,7-hexahydro-s-indacen-4-yl)carbamoyl]aminosulfonyl]-2-methylfuran-3-carboxylate 48c (200 mg, 0.442 mmol) in THF (8 mL). The resulting brown solution was stirred at room temperature for 3 h. The mixture was concentrated, and the residue was acidified to pH ˜4 using a 2M aqueous solution of HCl to generate a suspension. The white solids were collected by filtration and further dried in oven to give 5-[[(8-chloro-1,2,3,5,6,7-hexahydro-s-indacen-4-yl)carbamoyl]aminosulfonyl]-2-methylfuran-3-carboxylic acid 48d as a white solid (180 g, 92.88%).

### Step 5:

BH₃-Me₂S complex (0.21 mL, 10 M, 2.050 mmol) was added dropwise to a colorless suspension of 5-[[(8-chloro-1,2,3,5,6,7-hexahydro-s-indacen-4-yl)carbamoyl]aminosulfonyl]-2-methylfuran-3-carboxylic acid 48d (180 mg, 0.410 mmol) in THF (10 mL) at 0° C. under N₂. The resulting colorless solution was stirred at 60° C. for 1 h. After cooled to room temperature, the reaction was quenched with NH₄Cl (aq, 5 mL) and further diluted with water (10 mL). The mixture was extracted with CHCl₃/iPrOH (3/1, 20 mL×3). The combined organic extracts were concentrated to provide 3-(8-chloro-1,2,3,5,6,7-hexahydro-s-indacen-4-yl)-1-[4-(hydroxymethyl)-5-methylfuran-2-ylsulfonyl]urea 48e as a white solid (150 mg, 86.08%).

### Step 6:

PBr₃ (95.6 mg, 0.353 mmol) was added dropwise to a colorless suspension of 3-(8-chloro-1,2,3,5,6,7-hexahydro-s-indacen-4-yl)-1-[4-(hydroxymethyl)-5-methylfuran-2-ylsulfonyl]urea 48e (150 mg, 0.353 mmol) in THF (5 mL) at 0° C. The resulting mixture was stirred at room temperature for 1 h. The reaction mixture was diluted with water (4 mL) and extracted with iPrOH/CHCl₃ (v/v, 1/3, 10 mL×3). The combined organic extracts were washed with brine (15 mL), dried with Na₂SO₄, filtered and concentrated. The crude solid was recrystallized from petroleum ether to give 1-[4-(bromomethyl)-5-methylfuran-2-ylsulfonyl]-3-(8-chloro-1,2,3,5,6,7-hexahydro-s-indacen-4-yl)urea 48f as a white solid (150 mg, 87.11%). LC-MS: m/z [M]⁺, [M+2]=484.95, 486.95.

### Step 7:

A colorless suspension of 1-[(methylamino)methyl]cyclobutan-1-ol (70.8 mg, 0.616 mmol), DIEA (119.2 mg, 0.924 mmol) and 1-[4-(bromomethyl)-5-methylfuran-2-ylsulfonyl]-3-(8-chloro-1,2,3,5,6,7-hexahydro-s-indacen-4-yl)urea 48f (150 mg, 0.308 mmol) in THF (1.5 mL) was stirred at room temperature for 2 h. The suspension was diluted with iPrOH/CHCl₃ (v/v, 1/3, 5 mL) and water (2 mL). After separation, the aqueous phase was extracted with iPrOH/CHCl₃ (v/v, 1/3, 5 mL×2). The combined organic phases were concentrated and the residue was purified by reverse phase column (spherical C18, 80 g, 0 to 45% MeCN in water (0.05% FA)). After lyophilization, a colorless solid (50 mg) was obtained, which was further purified by Prep-HPLC using the following conditions: Column: XBridge Prep OBD C18 Column, 30*150 mm 5 μm; Mobile Phase A: Water(10 mmol/L NH₄HCO₃+0.1% NH₃·H₂O), Mobile Phase B: ACN; Flow rate: 60 mL/min; Gradient: 30% B to 60% B in 9 min, 60% B; Wavelength: 254 nm; RT1(min): 6.98. After lyophilization, the titled compound 3-(8-chloro-1,2,3,5,6,7-hexahydro-s-indacen-4-yl)-1-[4-([[(1-hydroxycyclobutyl)methyl](methyl)amino]methyl)-5-methylfuran-2-ylsulfonyl]urea Ex. 48 was obtained as a white solid (22.6 mg, 14.05%). ¹HNMR (300 MHz, CD₃OD) δ (ppm): 6.89 (s, 1H), 2.80-3.10 (m, 10H), 2.63 (s, 3H), 2.37 (s, 3H), 2.01-2.18 (m, 4H), 1.52-1.87 (m, 2H); LC-MS: m/z [M+H]⁺=522.15.

### Example 49: Preparation of (S)—N-((8-chloro-1,2,3,5,6,7-hexahydro-s-indacen-4-yl)carbamoyl)-4-((3-hydroxytropidine-1-yl)methyl)-5-methylfuran-2-sulfonamide

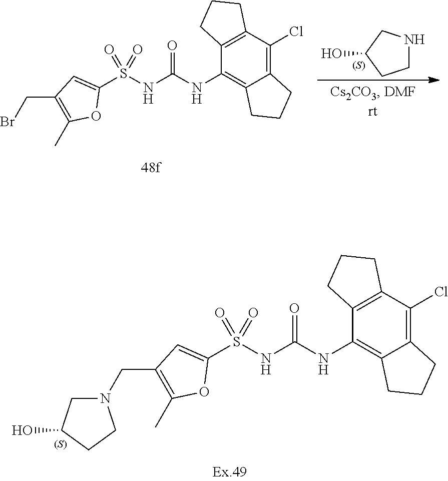

To a colorless suspension of (S)-pyrrolidin-3-ol (79 mg, 0.902 mmol) and Cs₂CO₃ (441 mg, 1.353 mmol) in DMF (2 mL) was added 4-(bromomethyl)-N-((8-chloro-1,2,3,5,6,7-hexahydro-s-indacen-4-yl)carbamoyl)-5-methylfuran-2-sulfonamide 48f (220 mg, 0.451 mmol). The resulting lightly brown suspension was stirred at rt for 2 h. The mixture was filtered, and the filtrate was purified by reverse phase column (15 to 40% MeCN in water (0.1% FA)). After lyophilization, the titled compound (S)—N-((8-chloro-1,2,3,5,6,7-hexahydro-s-indacen-4-yl)carbamoyl)-4-((3-hydroxypyrrolidin-1-yl)methyl)-5-methylfuran-2-sulfonamide Ex. 49 was obtained as an off-white solid (24.0 mg, 10.59%). ¹HNMR (300 MHz, (CD₃)₂SO) δ (ppm): 6.72 (s, 1H), 4.38 (m, 1H), 4.08 (s, 2H), 2.95-3.24 (m, 3H), 2.74-2.93 (m, 9H), 2.37 (s, 3H), 1.83-2.16 (m, 6H); LC-MS: m/z [M+H]⁺=494.15.

### Example 50: Preparation of N-((8-fluoro-1,2,3,5,6,7-hexahydro-s-indacen-4-yl)carbamoyl)-5-((((1-hydroxycyclobutyl)methyl)(methyl)amino)methyl)-4-methylfuran-2-sulfonamide

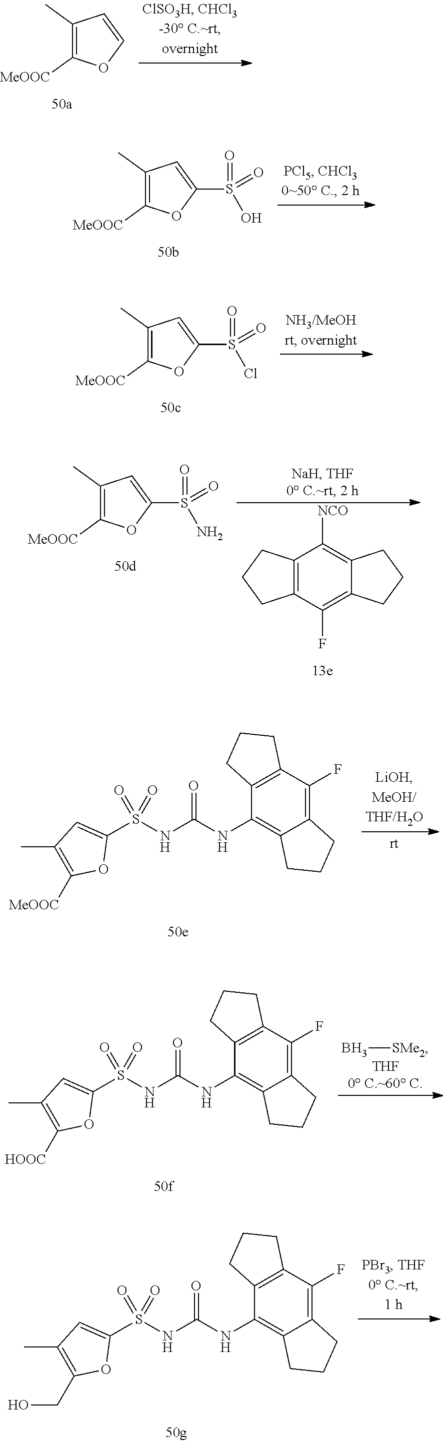

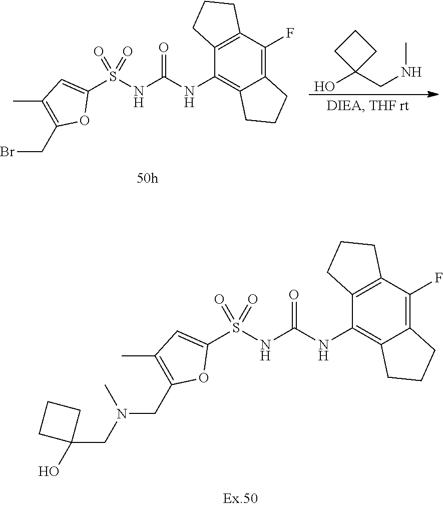

### Steps 1 and 2:

To a stirred pale yellow solution of methyl 3-methylfuran-2-carboxylate 50a (20 g, 142.716 mmol) in CHCl₃ (500 mL) at −30° C. was added dropwise a colorless solution of chlorosulfonic acid (33.26 g, 285.437 mmol). The resulting dark solution was allowed warm to rt slowly and stirred at rt overnight. The reaction progress was monitored by TLC and LC/MS. After reaction was complete, the black mixture was cooled down to −20° C. PCl₅ (74.30 g, 356.796 mmol) was added to the above reaction mixture to generate a black suspension. The reaction mixture was warmed to rt, heated to 50° C. and continued at this temperature for 2 h to afford a brown suspension. The suspension was poured onto crushed ice (1000 g) with stirring. The two phases were separated, the aqueous phase was extracted with CH₂Cl₂ (500 mL×2). The combined organic extracts were diluted with EtOAc (2000 mL), washed with cold brine (1000 mL×2), dried with Na₂SO₄, filtered and concentrated in vacuo to give methyl 5-(chlorosulfonyl)-3-methylfuran-2-carboxylate 50c as a brown oil (8 g, 23.49%). The crude product was used in the next step without further purification.

### Step 3:

To a colorless solution of NH₃ in MeOH (50 mL, 7 M, 350 mmol), methyl 5-(chlorosulfonyl)-3-methylfuran-2-carboxylate 50c (8 g, 33.523 mmol) was added. The resulting brown solution was stirred at room temperature for 4 h. N₂ gas was bubbled into the reaction mixture for 0.5 h to remove excess NH. The mixture was concentrated, and the residue was purified by flash silica gel column chromatography (0-34% EtOAc in petroleum ether) first to give a yellow solid, which was further purified by reverse phase column chromatography (15 to 40% MeCN in water (0.1% FA)) to afford methyl 3-methyl-5-sulfamoylfuran-2-carboxylate 50d as a light yellow solid (4 g, 54.43%). ¹HNMR (400 MHz, DMSO-d6) δ (ppm): 8.01 (s, 2H), 7.05 (s, 1H), 3.85 (s, 3H), 2.32 (s, 3H); LC-MS: m/z [M+H]⁺=217.90.

### Step 4:

NaH (99 mg, 4.106 mmol) was added to a solution of methyl 3-methyl-5-sulfamoylfuran-2-carboxylate 50d (900 mg, 4.106 mmol) in THF (20 mL) at 0° C. The resulting suspension was stirred at this temperature for 15 min. 4-fluoro-8-isocyanato-1,2,3,5,6,7-hexahydro-s-indacene 13e (892 mg, 4.106 mmol) was added. Cooling was stopped, and the reaction mixture was stirred at rt for 2 h. A solution of CHCl₃/iPrOH (v/v, 3/1, 40 mL) was added followed by water (20 mL). The aqueous mixture was separated, the aqueous phase was extracted with CHCl₃/iPrOH (v/v, 3/1, 40 mL×2). The combined organic phases were washed with brine (20 mL×2), dried with Na₂SO₄, filtered and concentrated in vacuo to provide methyl 5-[[(8-fluoro-1,2,3,5,6,7-hexahydro-s-indacen-4-yl)carbamoyl]aminosulfonyl]-3-methylfuran-2-carboxylate 50e as a white solid (1.6 g, 89.29%). LC-MS: m/z [M+Na]=459.05.

### Step 5:

A solution of LiOH (263 mg, 10.998 mmol) in H₂O (20 mL) was added to a solution of methyl 5-[[(8-fluoro-1,2,3,5,6,7-hexahydro-s-indacen-4-yl)carbamoyl]aminosulfonyl]-3-methylfuran-2-carboxylate 50e (1.6 g, 3.666 mmol) in THF (40 mL) and MeOH (10 mL). The resulting brown solution was stirred at room temperature for 3 h. The mixture was concentrated, and the residue was acidified to pH ˜4 using a 2M aqueous solution of HCl to generate a suspension. The white solids were collected via filtration and further dried in oven to give 5-[[(8-fluoro-1,2,3,5,6,7-hexahydro-s-indacen-4-yl)carbamoyl]aminosulfonyl]-3-methylfuran-2-carboxylic acid 50f as a white solid (1.3 g, 83.95%). LC-MS: m/z [M+H]⁺=423.05.

### Step 6:

BH₃-Me₂S complex (3.08 mL, 10 M, 30.770 mmol) was added dropwise to a stirred colorless suspension of 5-[[(8-fluoro-1,2,3,5,6,7-hexahydro-s-indacen-4-yl)carbamoyl]aminosulfonyl]-3-methylfuran-2-carboxylic acid 50f (1.3 g, 3.077 mmol) in THF (40 mL) at 0° C. under N₂. The resulting mixture was heated to 60° C. and continued at this temperature for 1 h. After cooled to room temperature, the reaction was quenched with the addition of NH₄Cl (aq, 5 mL) and diluted with water (10 mL). The aqueous mixture was extracted with CHCl₃/iPrOH (3/1, 60 mL×3). The combined organic extracts were concentrated. The residue was recrystallized from petroleum ether to give 1-(8-fluoro-1,2,3,5,6,7-hexahydro-s-indacen-4-yl)-3-[5-(hydroxymethyl)-4-methylfuran-2-ylsulfonyl]urea 50g as a white solid (1.1 g, 87.51%). LC-MS: m/z [M+H]⁺=409.10.

### Step 7:

PBr₃ (0.73 g, 2.693 mmol) was added dropwise to a colorless suspension of 1-(8-fluoro-1,2,3,5,6,7-hexahydro-s-indacen-4-yl)-3-[5-(hydroxymethyl)-4-methylfuran-2-ylsulfonyl]urea 50g (1.1 g, 2.693 mmol) in THF (20 mL) at 0° C. The resulting suspension was stirred at room temperature for 1 h. The mixture was diluted with water (20 mL) and extracted with iPrOH/CHCl₃ (v/v, 1/3, 50 mL×3). The combined organic extracts were washed with brine (50 mL), dried with Na₂SO₄, filtered and concentrated. The crude solid was recrystallized from petroleum ether to afford 3-[5-(bromomethyl)-4-methylfuran-2-ylsulfonyl]-1-(8-chloro-1,2,3,5,6,7-hexahydro-s-indacen-4-yl)urea 50h as a white solid (1.1 g, 86.66%). LC-MS: m/z [M]⁺, [M+2]⁺=471.05, 473.05.

### Step 8:

To a colorless suspension of 1-((methylamino)methyl)cyclobutan-1-ol (147 mg, 1.272 mmol) and Cs₂CO₃ (622 mg, 1.908 mmol) in DMF (3 mL) was added 5-(bromomethyl)-N-((8-fluoro-1,2,3,5,6,7-hexahydro-s-indacen-4-yl)carbamoyl)-4-methylfuran-2-sulfonamide 50h (300 mg, 0.636 mmol). The resulting suspension was stirred at rt for 2 h. After filtration, the filtrate was purified by reverse phase column (15 to 40% MeCN in water (0.1% FA)). After lyophilization, the titled compound N-((8-fluoro-1,2,3,5,6,7-hexahydro-s-indacen-4-yl)carbamoyl)-5-((((1-hydroxycyclobutyl)methyl)(methyl)amino)methyl)-4-methylfuran-2-sulfonamide Ex. 50 was obtained as a white solid (15.0 mg, 4.64%). ¹HNMR (400 MHz, DMSO-d6) δ (ppm): 6.70 (s, 1H), 4.04 (s, 2H), 2.61-2.88 (m, 10H), 2.50 (s, 3H), 1.91-2.08 (m, 11H), 1.51-1.59 (m, 1H), 1.36-1.41 (m, 1H); ¹HNMR (376 MHz, DMSO-d6) δ (ppm): −127.19; LC-MS: m/z [M+H]⁺=505.20.

### Examples 51 to 56

Following the procedures described in Preparative Example 49, by using a suitable amine precursor R₁R₂NH. Examples 51 to 56 were prepared from bromo intermediate 50h. Results are summarized in Table 3.

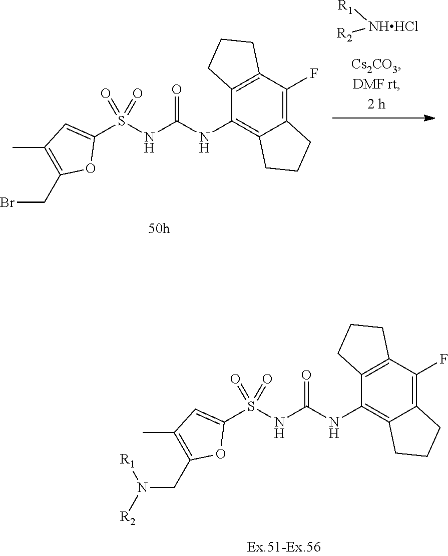

TABLE 3

| Ex. | | | |
|---|---|---|---|
| No. | R₁R₂NH | Structure/Name | LC-MS, ¹H NMR |
| 51 | 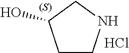 | 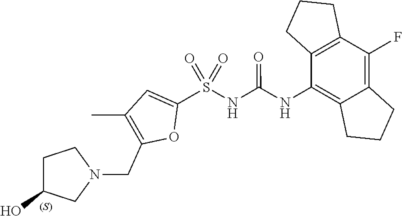 | LC-MS: m/z [M + H]⁺ = 478.15; ¹HNMR (400 MHz, DMSO-d₆) δ (ppm): 6.59 (s, 1H), 4.3 (s, 1H), 4.16 (s, 2H), 2.88 − 3.21 (m, 4H), 2.75 − 2.85 (m, 4H), 2.60 − 2.74 (m, 4H), 1.89 − 2.15 (m, 8H), 1.64 − 1.78 (m, 1H); ¹⁹FNMR (376 MHz, DMSO-d₆) δ (ppm): −127.57. |
| | | (S)-N-((8-fluoro-1,2,3,5,6,7- | |
| | | hexahydro-s-indacen-4- | |
| | | yl)carbamoyl)-5-((3- | |
| | | hydroxypyrrolidin-1-yl)methyl)-4- | |
| | | methylfuran-2-sulfonamide | |
| 52 | 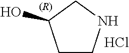 (K-144) | 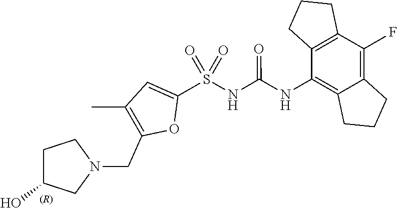 | LC-MS: m/z [M + H]⁺ = 478.20; ¹HNMR (300 MHz, (CD₃)₂SO) δ (ppm): 6.63 (s, 1H), 4.31 (m, 1H), 4.20 (s, 2H), 3.10 − 3.23 (m, 3H), 2.67 − 2.94 (m, 9H), 1.93 − 2.08 (m, 8H), 1.70 − 1.79 (m, 1H). ¹⁹F NMR (282 MHz, (CD₃)₂SO) δ (ppm): −127.45 |
| | | (R)-N-((8-fluoro-1,2,3,5,6,7- | |
| | | hexahydro-s-indacen-4- | |
| | | yl)carbamoyl)-5-((3- | |
| | | hydroxypyrrolidin-1-yl)methyl)-4- | |
| | | methylfuran-2-sulfonamide | |
| 53 | 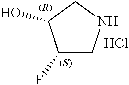 (K-145) | 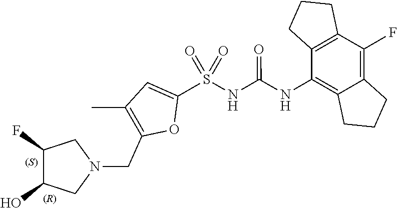 | LC-MS: m/z [M + H]⁺ = 496.20; ¹HNMR (400 MHz, (CD₃)₂SO) δ (ppm): 6.87 (s, 1H), 4.78, 4.92 (m, 1H), 4.02 − 4.11 (m, 1H), 3.74 (s, 2H), 3.05 − 3.16 (m, 1H), 2.63 − 2.95 (m, 11H), 1.94 − 2.10 (m, 7H). |
| | | N-((8-fluoro-1,2,3,5,6,7-hexahydro- | |
| | | s-indacen-4-yl)carbamoyl)-5- | |
| | | (((38,4R)-3-fluoro-4- | |
| | | hydroxypyrrolidin-1-yl)methyl)-4- | |
| | | methylfuran-2-sulfonamide | |
| 54 | 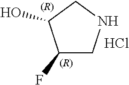 (K-146) | 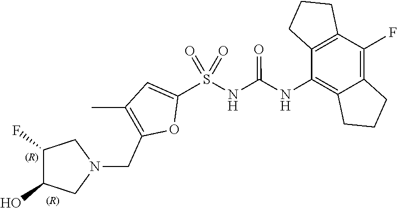 | LC-MS: m/z [M + H]⁺ = 496.20; ¹HNMR (400 MHz, (CD₃)₂SO) δ (ppm): 6.90 (s, 1H), 4.72, 4.85 (m, 1H), 4.08 − 4.17 (m, 1H), 3.68 (s, 2H), 3.05 − 3.09 (m, 1H), 2.63 − 2.90 (m, 10H), 2.22 − 2.26 (m, 1H), 1.94 − 2.06 (m, 7H). ¹⁹FNMR (376 MHz, (CD₃)₂SO) δ (ppm): −125.92, −176.67. |
| | | N-((8-fluoro-1,2,3,5,6,7-hexahydro- | |
| | | s-indacen-4-yl)carbamoyl)-5- | |
| | | (((3R,4R)-3-fluoro-4- | |
| | | hydroxypyrrolidin-1-yl)methyl)-4- | |
| | | methylfuran-2-sulfonamide | |
| 55 | 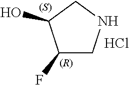 (K-147) | 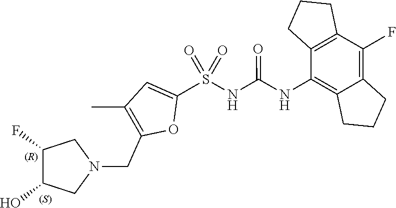 | LC-MS: m/z [M + H]⁺ = 496.20; ¹HNMR (400 MHz, (CD₃)₂SO) δ (ppm): 6.72 (s, 1H), 4.75, 4.89 (m, 1H), 4.00 − 4.08 (m, 1H), 3.65 (s, 2H), 3.00 − 3.12 (m, 1H), 2.65 − 2.90 (m, 11H), 1.94 − 2.04 (m, 7H); ¹⁹F NMR (376 MHz, (CD₃)₂SO) δ (ppm): −126.72, −193.8. |
| | | N-((8-fluoro-1,2,3,5,6,7-hexahydro- | |
| | | s-indacen-4-yl)carbamoyl)-5- | |
| | | (((3R,4S)-3-fluoro-4- | |
| | | hydroxypyrrolidin-1-yl)methyl)-4- | |
| | | methylfuran-2-sulfonamide | |
| 56 | 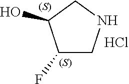 (K-148) | 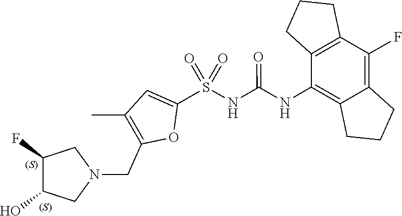 | LC-MS: m/z [M + H]⁺ = 492.20; ¹HNMR (400 MHz, (CD₃)₂SO) δ (ppm): 6.92 (s, 1H), 4.73, 4.85 (m, 1H), 4.08 − 4.16 (m, 1H), 3.69 (s, 2H), 3.06 − 3.10 (m, 1H), 2.63 − 2.90 (m, 10H), 2.23 − 2.27 (m, 1H), 1.96 − 2.06 (m, 7H); ¹⁹FNMR (376 MHz, (CD₃)₂SO) δ (ppm): −125.80, −176.71. |
| | | N-((8-fluoro-1,2,3,5,6,7-hexahydro- | |
| | | s-indacen-4-yl)carbamoyl)-5- | |
| | | (((3S,4S)-3-fluoro-4- | |
| | | hydroxypyrrolidin-1-yl)methyl)-4- | |
| | | methylfuran-2-sulfonamide | |

### Example 57: Preparation of N-((8-chloro-1,2,3,5,6,7-hexahydro-s-indacen-4-yl)carbamoyl)-5-((((1-hydroxycyclobutyl)methyl)(methyl)amino)methyl)-4-methylfuran-2-sulfonamide

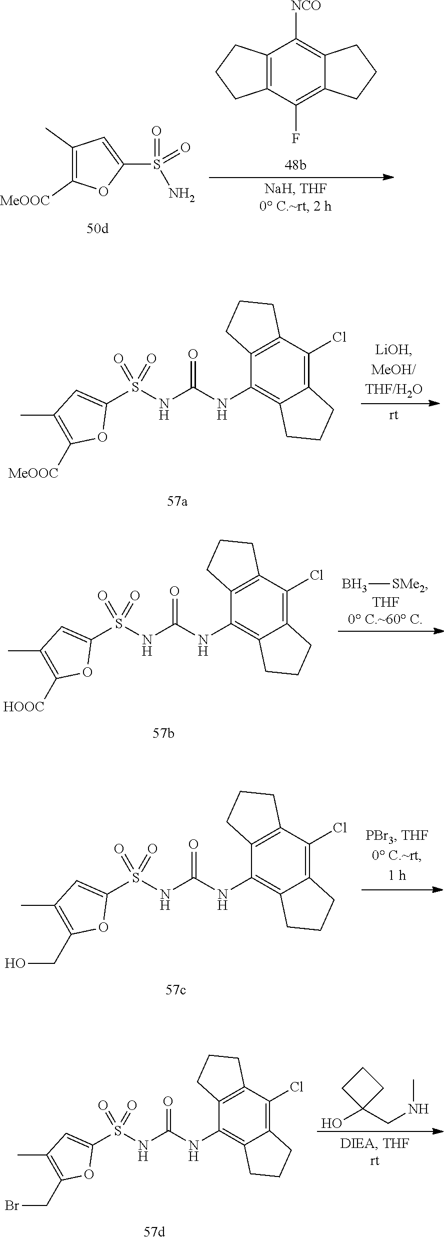

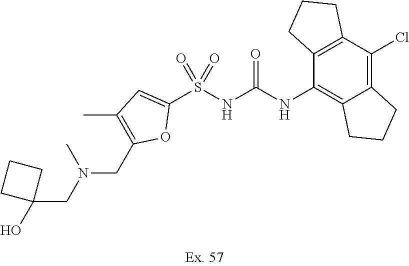 [SMILES: Cc1cc(S(=O)(=O)NC(=O)Nc2c3c(c(Cl)c4c2CCC4)CCC3)oc1CN(C)CC1(O)CCC1]

### Step 1:

NaH (13 mg, 0.547 mmol) was added to a solution of methyl 3-methyl-5-sulfamoylfuran-2-carboxylate 50d (120 mg, 0.547 mmol) in THF (3 mL) at 0° C. The resulting suspension was stirred at this temperature for 15 min. 4-chloro-8-isocyanato-1,2,3,5,6,7-hexahydro-s-indacene 48b (128 mg, 0.547 mmol) was added. Cooling was stopped, and the reaction mixture was stirred at rt for 1 h. A solution of CHCl₃/iPrOH (v/v, 3/1, 10 mL) was added followed by water (3 mL). The mixture was separated, the aqueous phase was extracted with CHCl₃/iPrOH (v/v, 3/1, 5 mL×2), and the combined organic phases were washed with brine (10 mL×2), dried with Na₂SO₄, filtered and concentrated in vacuo to give methyl 5-[[(8-chloro-1,2,3,5,6,7-hexahydro-s-indacen-4-yl)carbamoyl]aminosulfonyl]-3-methylfuran-2-carboxylate 57a as a white solid (200 mg, 80.67%).

### Step 2:

A solution of LiOH (35 mg, 1.457 mmol) in H₂O (5 mL) was added to a solution of methyl 5-[[(8-chloro-1,2,3,5,6,7-hexahydro-s-indacen-4-yl)carbamoyl]aminosulfonyl]-3-methylfuran-2-carboxylate 57a (220 mg, 0.486 mmol) in THF (10 mL) and MeOH (3 mL). The resulting brown solution was stirred at room temperature for 3 h. The mixture was concentrated and the residue was acidified to pH ˜4 using a 2M aqueous HCl solution to generate a suspension. The white solids were collected by filtration and further dried in oven to give 5-[[(8-chloro-1,2,3,5,6,7-hexahydro-s-indacen-4-yl)carbamoyl]aminosulfonyl]-3-methylfuran-2-carboxylic acid 57b as a white solid (190 mg, 89.12%).

### Step 3:

BH₃-Me₂S complex (0.41 mL, 10 M, 4.100 mmol) was added dropwise to a stirred colorless suspension of 5-[[(8-chloro-1,2,3,5,6,7-hexahydro-s-indacen-4-yl)carbamoyl]aminosulfonyl]-3-methylfuran-2-carboxylic acid 57b (180 mg, 0.410 mmol) in THF (10 mL) at 0° C. under N₂. Cooling was stopped, and the reaction mixture was heated up to 60° C. and continued at this temperature for 1 h. After cooled to room temperature, the reaction was quenched with NH₄Cl (aq, 5 mL) and diluted with water (10 mL). The aqueous mixture was extracted with CHCl₃/iPrOH (3/1, 40 mL×3). The combined organic phases were concentrated in vacuo to give 1-(8-chloro-1,2,3,5,6,7-hexahydro-s-indacen-4-yl)-3-[5-(hydroxymethyl)-4-methylfuran-2-ylsulfonyl]urea 57c as a white solid (150 mg, 86.08%).

### Step 4:

PBr₃ (96 mg, 0.353 mmol) was added dropwise into a colorless suspension of 1-(8-chloro-1,2,3,5,6,7-hexahydro-s-indacen-4-yl)-3-[5-(hydroxymethyl)-4-methylfuran-2-ylsulfonyl]urea 57c (150 mg, 0.353 mmol) in THF (5 mL) at 0° C. The resulting suspension was stirred at room temperature for 1 h. The reaction mixture was diluted with water (5 mL) and extracted with iPrOH/CHCl₃ (v/v, 1/3, 10 mL×3). The combined organic phases were washed with brine (10 mL), dried with Na₂SO₄, filtered and concentrated. The crude solid was recrystallized from petroleum ether to afford 3-[5-(bromomethyl)-4-methylfuran-2-ylsulfonyl]-1-(8-chloro-1,2,3,5,6,7-hexahydro-s-indacen-4-yl)urea 57d as a white solid (160 mg, 92.91%).

### Step 5:

A colorless suspension of 1-[(methylamino)methyl]cyclobutan-1-ol (76 mg, 0.656 mmol), DIEA (127 mg, 0.984 mmol) and 3-[5-(bromomethyl)-4-methylfuran-2-ylsulfonyl]-1-(8-chloro-1,2,3,5,6,7-hexahydro-s-indacen-4-yl)urea 57d (160 mg, 0.328 mmol) in THF (2 mL) was stirred at room temperature for 2 h. The suspension was diluted with iPrOH/CHCl₃ (v/v, 1/3, 5 mL) and water (2 mL). The aqueous mixture was separated. The aqueous phase was extracted with iPrOH/CHCl₃ (v/v, 1/3, 5 mL×2). The combined organic phases were concentrated and the residue was purified by reverse phase column (spherical C18, 80 g, 0 to 45% MeCN in water(0.05% FA)). After lyophilization, a white solid product (41 mg) was obtained, which was further purified by Prep-HPLC using the following conditions: Column: Column: XBridge Shield RP18 OBD Column, 19*250 mm, 0 μm; Mobile Phase A: Water(10 mmol/L NH₄HCO₃+0.1% NH₃·H₂O), Mobile Phase B: ACN; Flow rate: 25 mL/min; Gradient: 26% B to 41% B in 10 min, 41% B; Wavelength: 254 nm; RT1(min): 9. After lyophilization, the titled compound 3-(8-chloro-1,2,3,5,6,7-hexahydro-s-indacen-4-yl)-1-[5-([[(1-hydroxycyclobutyl)methyl](methyl)amino]methyl)-4-methylfuran-2-ylsulfonyl]urea Ex. 57 was obtained as a white solid (21.2 mg, 12.36%). ¹HNMR (400 MHz, DMSO-d6) δ (ppm): 6.61 (s, 1H), 4.19 (s, 2H), 2.95 (s, 2H), 2.73-2.89 (m, 8H), 2.53 (s, 3H), 1.95-2.03 (m, 11H), 1.49-1.68 (m, 2H); LC-MS: m/z [M+H]⁺=522.10.

### Example 58: Preparation of N-((8-fluoro-1,2,3,5,6,7-hexahydro-s-indacen-4-yl)carbamoyl)-5-((((1-hydroxycyclobutyl)methyl)(methyl)(amino)methyl)-1-methyl-1H-pyrazole-3-sulfonamide

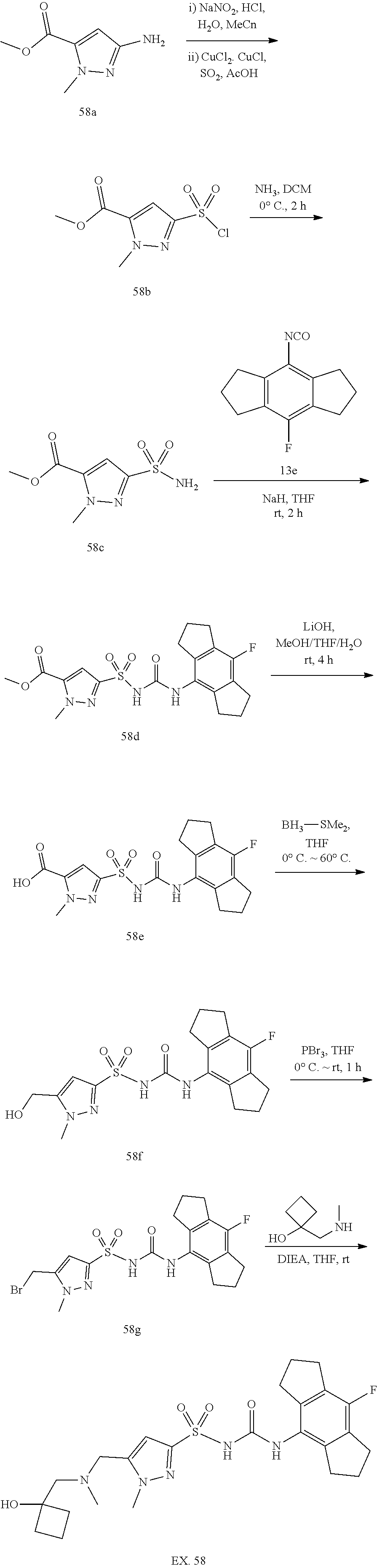

### Step 1:

A colorless solution of NaNO₂ (2.67 g, 38.670 mmol) in water (16 mL) was added dropwise to a stirred solution of methyl 3-amino-1-methyl-1H-pyrazole-5-carboxylate 58a (5 g, 32.225 mmol) in MeCN (160 mL) and HCl (aq.) (43 mL, 7.5 M, 322.25 mmol) at 0° C. to generate a yellow suspension. The mixture was stirred at this temperature for 1h. A saturated solution of SO₂ in AcOH (160 mL) was added followed by a green solution of CuCl₂ (2.17 g, 16.113 mmol) and CuCl (160 mg, 1.611 mmol) in water (2 mL) to result in a dark green solution. The reaction mixture was stirred at 0° C. for 3 h. EtOAc (300 mL) and water (100 mL) were added successively to the reaction mixture, and the mixture was separated. The aqueous phase was extracted with EtOAc (200 mL×2). The combined organic phases were washed with brine (400 mL), dried with Na₂SO₄ and filtered. Removal of solvents provided methyl 3-(chlorosulfonyl)-1-methyl-1H-pyrazole-5-carboxylate 58b as a yellow oil (2.5 g, 32.51%).

### Step 2:

Methyl 3-(chlorosulfonyl)-1-methyl-1H-pyrazole-5-carboxylate 58b (2.5 g, 10.476 mmol) was added dropwise to a saturated solution of NH₃(g) in DCM (100 mL) at 0° C. resulting in a yellow suspension. The mixture was stirred at rt for 2 h. After concentration, the residue was purified by silica gel column chromatography to give methyl 1-methyl-3-sulfamoyl-1H-pyrazole-5-carboxylate 58c as a yellow solid (2.0 g, 87.09%). ¹HNMR (300 MHz, Methanol-d₄) δ (ppm): 4.16 (s, 1H), 4.20 (s, 3H), 3.91 (s, 3H); LC-MS: m/z [M+H]⁺=220.05.

### Step 3:

NaH (120 mg, 5.018 mmol) was added to a solution of methyl 2-methyl-5-sulfamoylpyrazole-3-carboxylate 58c (1 g, 4.562 mmol) in THF (20 mL) at 0° C. and the resulting suspension was stirred at this temperature for 15 min. 4-Fluoro-8-isocyanato-1,2,3,5,6,7-hexahydro-s-indacene 13e (1.09 g, 5.018 mmol) was added. Cooling was stopped, and the reaction mixture was stirred at rt for 1 h. A solution of CHCl₂/iPrOH (v/v, 3/1, 20 mL) was added followed by water (10 mL). The mixture was separated, the aqueous phase was extracted with CHCl₃/iPrOH (v/v, 3/1, 20 mL×2), and the combined organic phases were washed with brine (30 mL×2), dried with Na₂SO₄, filtered and concentrated in vacuo to give methyl 5-[[(8-fluoro-1,2,3,5,6,7-hexahydro-s-indacen-4-yl)carbamoyl]aminosulfonyl]-2-methylpyrazole-3-carboxylate 58d as a white solid (1 0.8 g, 90.41%). LC-MS: m/z [M+H]⁺=437.15.

### Step 4:

A solution of LiOH (296 mg, 12.372 mmol) in H₂O (20 mL) was added to a solution of methyl 5-[[(8-fluoro-1,2,3,5,6,7-hexahydro-s-indacen-4-yl)carbamoyl]aminosulfonyl]-2-methylpyrazole-3-carboxylate 58d (1.8 g, 4.124 mmol) in THF (50 mL) and MeOH (10 mL). The resulting brown solution was stirred at room temperature for 4 h. The mixture was concentrated. The remaining aqueous residue was acidified to pH ˜4 using a 2M aqueous solution of HCl to produce a suspension. The white solids were collected via filtration and further dried in oven to give 5-[[(8-fluoro-1,2,3,5,6,7-hexahydro-s-indacen-4-yl)carbamoyl]aminosulfonyl]-2-methylpyrazole-3-carboxylic acid 58e as a white solid (1.6 g, 91.84%). LC-MS: m/z [M+H]⁺=423.10.

### Step 5:

BH₃-Me₂S complex (3.55 mL, 10 M, 35.510 mmol) was added dropwise to a stirred colorless suspension of 5-[[(8-fluoro-1,2,3,5,6,7-hexahydro-s-indacen-4-yl)carbamoyl]aminosulfonyl]-2-methylpyrazole-3-carboxylic acid 58e (1.5 g, 3.551 mmol) in THF (40 mL) at 0° C. under N₂. Cooling was stopped, the reaction mixture was heated to 60° C. and continued at this temperature for 2 h to yield a colorless suspension. After cooled to room temperature, the reaction was quenched with NH₄Cl (aq, 5 mL) and diluted with water (10 mL). The mixture was extracted with CHCl₃/iPrOH (3/1, 60 mL×3). The combined organic phases were concentrated to give 3-(8-fluoro-1,2,3,5,6,7-hexahydro-s-indacen-4-yl)-1-[5-(hydroxymethyl)-1-methylpyrazol-3-ylsulfonyl]urea 58f as a white solid (1.3 g, 89.63%). LC-MS: m/z [M+H]⁺=409.10.

### Step 6:

PBr₃ (860 mg, 3.183 mmol) was added dropwise to a colorless suspension of N-((8-fluoro-1,2,3,5,6,7-hexahydro-s-indacen-4-yl)carbamoyl)-5-(hydroxymethyl)-1-methyl-1H-pyrazole-3-sulfonamide 58f (1.3 g, 3.183 mmol) in THF (20 mL) at 0° C. The resulting mixture was stirred at room temperature for 1 h. The reaction mixture was diluted with water (5 mL) and extracted with iPrOH/CHCl₃ (v/v, 1/3, 40 mL×3). The combined organic phases were washed with brine (50 mL), dried with Na₂SO₄, filtered and concentrated. The crude solid was recrystallized from petroleum ether to provide 5-(bromomethyl)-N-((8-fluoro-1,2,3,5,6,7-hexahydro-s-indacen-4-yl)carbamoyl)-1-methyl-1H-pyrazole-3-sulfonamide 58g as a white solid (1.3 g, 86.66%). LC-MS: m/z [M+H]⁺=473.0.

### Step 7:

To a solution of 1-[(methylamino)methyl]cyclobutan-1-ol (63.53 mg, 0.552 mmol) in THF (2 mL) was added DIEA (0.14 mL, 0.804 mmol) and 1-[5-(bromomethyl)-1-methylpyrazol-3-ylsulfonyl]-3-(8-fluoro-1,2,3,5,6,7-hexahydro-s-indacen-4-yl)urea 58g (130.00 mg, 0.276 mmol). The resulting slightly brown suspension was stirred at room temperature for 2 h. Water (5 mL) was added, and the aqueous mixture was extracted with iPrOH/CHCl₃ (v/v, 1:3, 3×10 mL). The organic layers were combined, dried over anhydrous sodium sulfate, filtered and concentrated. The crude product was purified by Prep-HPLC using the following conditions: Column: Xselect CSH OBD Column 30*150 mm 5 μm; Mobile Phase A: Water(0.05% FA), Mobile Phase B: ACN; Flow rate: 25 mL/min; Gradient: 20% B to 40% B in 9 min, 40% B; Wavelength: 254 nm; RT1(min): 7.6. After lyophilization, the titled compound 3-(8-fluoro-1,2,3,5,6,7-hexahydro-s-indacen-4-yl)-1-[5-({[(1-hydroxycyclobutyl)methyl](methyl)amino}methyl)-1-methylpyrazol-3-ylsulfonyl]urea Ex. 58 was obtained as a white solid (21.4 mg, 15.05%). ¹HNMR (400 MHz, Methanol-d₄) δ (ppm): 6.77-6.83 (m, 1H), 3.93-4.07 (m, 3H), 3.88 (s, 2H), 2.81-2.95 (m, 4H), 2.65-2.81 (m, 6H), 2.42 (s, 3H), 1.98-2.19 (m, 8H), 1.69-1.83 (m, 1H), 1.44-1.57 (m, 1H); ¹⁹FNMR (376 MHz, Methanol-d₄) δ (ppm): −127.23; LC-MS: m/z [M+H]⁺=506.15.

### Example 59: Preparation of N-((8-fluoro-1,2,3,5,6,7-hexahydro-s-indacen-4-yl)carbamoyl)-5-((((1-hydroxycyclobutyl)methyl)amino)methyl)-1-methyl-1H-pyrazole-3-sulfonamide

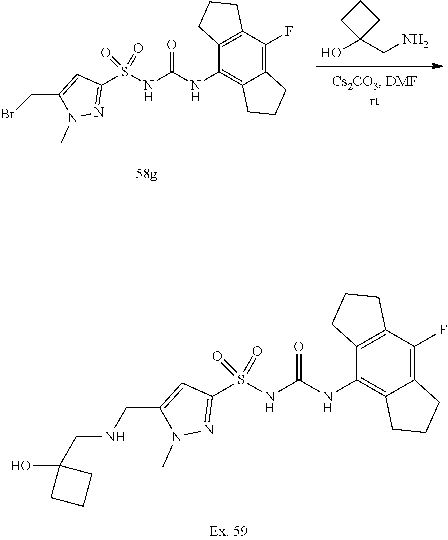 [SMILES: Cn1nc(S(=O)(=O)NC(=O)Nc2c3c(c(F)c4c2CCC4)CCC3)cc1CBr ; NCC1(O)CCC1 ; Cn1nc(S(=O)(=O)NC(=O)Nc2c3c(c(F)c4c2CCC4)CCC3)cc1CNCC1(O)CCC1]

To a colorless suspension of 1-(aminomethyl)cyclobutan-1-ol (86 mg, 0.848 mmol) and CS₂CO₃ (415 mg, 1.272 mmol) in DMF (3 mL), 5-(bromomethyl)-N-((8-fluoro-1,2,3,5,6,7-hexahydro-s-indacen-4-yl)carbamoyl)-1-methyl-1H-pyrazole-3-sulfonamide 58g (200 mg, 0.424 mmol) was added. The resulting suspension was stirred at rt for 2 h and filtered. The filtrate was purified by reverse phase column chromatography (15 to 40% MeCN in water(0.1% FA)). After lyophilization, the titled compound N-((8-fluoro-1,2,3,5,6,7-hexahydro-s-indacen-4-yl)carbamoyl)-5-((((1-hydroxycyclobutyl)methyl)amino)methyl)-1-methyl-1H-pyrazole-3-sulfonamide Ex. 59 was obtained as a white solid (22.8 mg, 10.88%). ¹HNMR (300 MHz, DMSO-d6) δ (ppm): 6.67 (s, 1H), 3.94 (s, 2H), 3.86 (s, 3H), 2.64-2.81 (m, 10H), 1.85-2.02 (m, 8H), 1.52-1.62 (m, 1H), 1.36-1.42 (m, 1H); ¹⁹FNMR (282 MHz, DMSO-d6) δ (ppm): −126.19; LC-MS: m/z [M+H]⁺=492.20.

### Examples 60 to 65

Following the procedures described in Preparative Example 59, or Preparative Example 58 step 7, by using a suitable amine precursor R₁R₂NH, Examples 60 to 65 were prepared from bromo intermediate 58g. Results are summarized in Table 4.

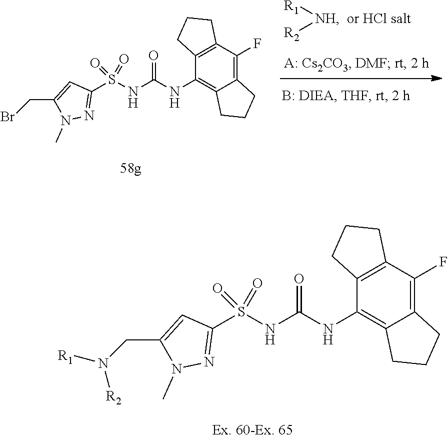

TABLE 4

| Ex. | | | |
|---|---|---|---|
| No. | R₁R₂NH | Structure/Name | LC-MS, ¹H NMR |
| 60 | 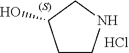 | 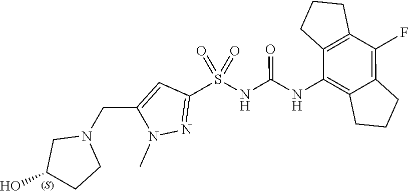 | LC-MS: m/z [M + H]⁺ = 478.15; ¹HNMR (400 MHz, DMSO-d₆) δ (ppm): 6.67 (s, 1H), 4.16 − 4.25 (m, 1H), 3.87 (s, 3H), 3.67 − 3.81 (m, 2H), 2.73 − 2.88 (m, 5H), 2.56 − 2.72 (m, 5H), 2.36 − 2.47 (m, 1H), 1.78 − 2.21 (m, 6H), 1.52 − 1.64 (m, 1H); ¹⁹FNMR (376 MHz, DMSO-d₆) δ (ppm): −125.35. |
| | | (S)-N-((8-fluoro-1,2,3,5,6,7- | |
| | | hexahydro-s-indacen-4- | |
| | | yl)carbamoyl)-5-((3- | |
| | | hydroxypyrrolidin-1-yl)methyl)-1- | |
| | | methyl-1H-pyrazole-3-sulfonamide | |
| 61 | 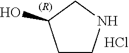 | 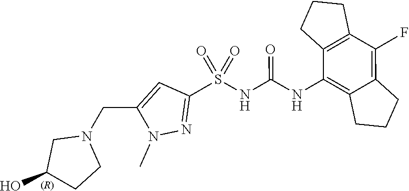 | LC-MS: m/z [M + H]⁺ = 478.15; ¹HNMR (400 MHz, DMSO-d₆) δ (ppm): 6.49 − 6.63 (m, 1H), 4.14 − 4.24 (m, 1H), 3.80 − 3.91 (m, 3H), 3.61 − 3.76 (m, 2H), 2.58 − 2.88 (m, 10H), 2.32 − 2.47 (m, 4H), 1.91 − 2.10 (m, 5H), 1.48 − 1.63 (m, 1H). |
| | | (R)-N-((8-fluoro-1,2,3,5,6,7- | |
| | | hexahydro-s-indacen-4- | |
| | | yl)carbamoyl)-5-((3- | |
| | | hydroxypyrrolidin-1-yl)methyl)-1- | |
| | | methyl-1H-pyrazole-3-sulfonamide | |
| 62 | 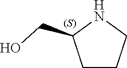 | 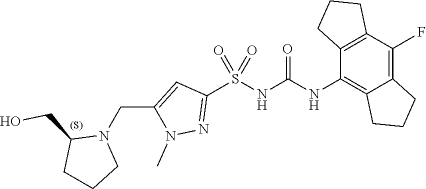 | LC-MS: m/z [M + H]⁺ = 492.20; ¹HNMR (300 MHz, DMSO-d6) δ (ppm): 6.67 (s, 1H), 4.13 − 4.21 (m, 1H), 3.74 − 3.85 (m, 3H), 3.50 − 3.64 (m, 1H), 3.35 − 3.48 (m, 2H), 2.53 − 2.90 (m, 10H), 2.21 − 2.41 (m, 1H), 1.91 − 2.08 (m, 4H), 1.77 − 1.89 (m, 1H), 1.45 − 1.72 (m, 3H). |
| | | (S)-N-((8-fluoro-1,2,3,5,6,7- | |
| | | hexahydro-s-indacen-4- | |
| | | yl)carbamoyl)-5-((2- | |
| | | (hydroxymethyl)pyrrolidin-1- | |
| | | yl)methyl)-1-methyl-1H-pyrazole- | |
| | | 3-sulfonamide | |
| 63 | 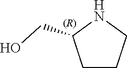 | 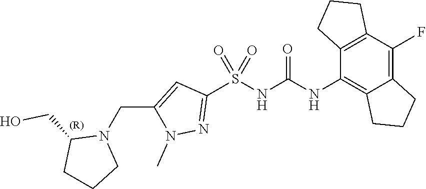 | LC-MS: m/z [M + H]⁺ = 492.20; ¹HNMR (400 MHz, DMSO-d₆) δ (ppm): 6.50 − 6.69 (m, 1H), 4.08 − 4.23 (m, 1H), 3.77 − 3.96 (m, 3H), 3.30 − 3,47 (m, 3H), 2.56 − 2.90 (m, 10H), 2.16 − 2.37 (m, 1H), 1.92 − 2.07 (m, 4H), 1.79 − 1.89 (m, 1H), 1.47 − 1.74 (m, 3H). |
| | | (R)-N-((8-fluoro-1,2,3,5,6,7- | |
| | | hexahydro-s-indacen-4- | |
| | | yl)carbamoyl)-5-((2- | |
| | | (hydroxymethyl)pyrrolidin-1- | |
| | | yl)methyl)-1-methyl-1 H-pyrazole- | |
| | | 3-sulfonamide | |
| 64 | 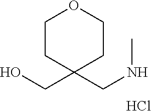 | 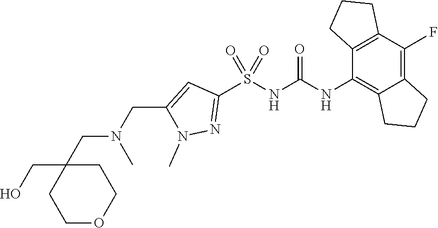 [SMILES: CN(Cc1cc(S(=O)(=O)NC(=O)Nc2c3c(c(F)c4c2CCC4)CCC3)nn1C)CC1(CO)CCOCC1] | LC-MS: m/z [M + H]⁺ = 550.20; ¹HNMR (300 MHz, (CD₃)₂SO) δ (ppm): 6.58 (s, 1H), 3.85 (s, 3H), 3.63 (m, 2H), 3.45 (m, 4H), 3.36 (s, 2H), 2.65 − 2.84 (m, 8H), 2.41 (s, 2H), 2.21 (s, 3H), 1.94 − 2.04 (m, 4H), 1.24 − 1.35 (m, 4H); ¹⁹FNMR (282 MHz, DMSO-d6) δ ppm: −125.89. |
| | | N-((8-fluoro-1,2,3,5,6,7-hexahydro- | |
| | | S-indacen-4-yl)carbamoyl)-5-((((4- | |
| | | (hydroxymethyl)tetrahydro-2H- | |
| | | pyran-4- | |
| | | yl)methyl)(methyl)amino)methyl)- | |
| | | 1-methyl-1H-pyrazole-3- | |
| | | sulfonamide | |
| 65 | 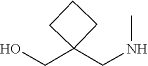 | 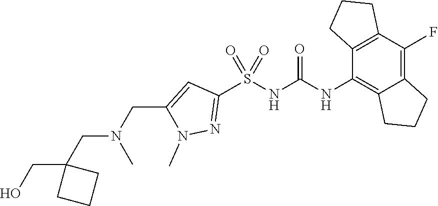 [SMILES: CN(Cc1cc(S(=O)(=O)NC(=O)Nc2c3c(c(F)c4c2CCC4)CCC3)nn1C)CC1(CO)CCC1] | LC-MS: m/z [M + H]⁺ = 520.20; ¹HNMR (400 MHz, DMSO-d₆) δ (ppm): 6.51 (s, 1H), 3.80 (s, 3H), 3.51 (s, 2H), 3.45 (s, 2H), 2.73 − 2.80 (m, 4H), 2.58 − 2.66 (m, 4H), 2.43 (s, 2H), 2.06 (s, 3H), 1.91-2.01 (m, 4H), 1.73-1.83 (m, 1H), 1.59 − 1.73 (m, 5H). |
| | | N-((8-fluoro-1,2,3,5,6,7-hexahydro- | |
| | | s-indacen-4-yl)carbamoyl)-5-((((1- | |
| | | (hydroxymethyl)cyclobutyl)methyl) | |
| | | (methyl)amino)methyl)-1-methyl- | |
| | | 1H-pyrazole-3-sulfonamide | |

### Example 66: Preparation of N-((8-fluoro-1,2,3,5,6,7-hexahydro-s-indacen-4-yl)carbamoyl)-5-((((1-hydroxycyclobutyl)methyl)(methyl)amino)methyl)-1-isopropyl-1H-pyrazole-3-sulfonamide

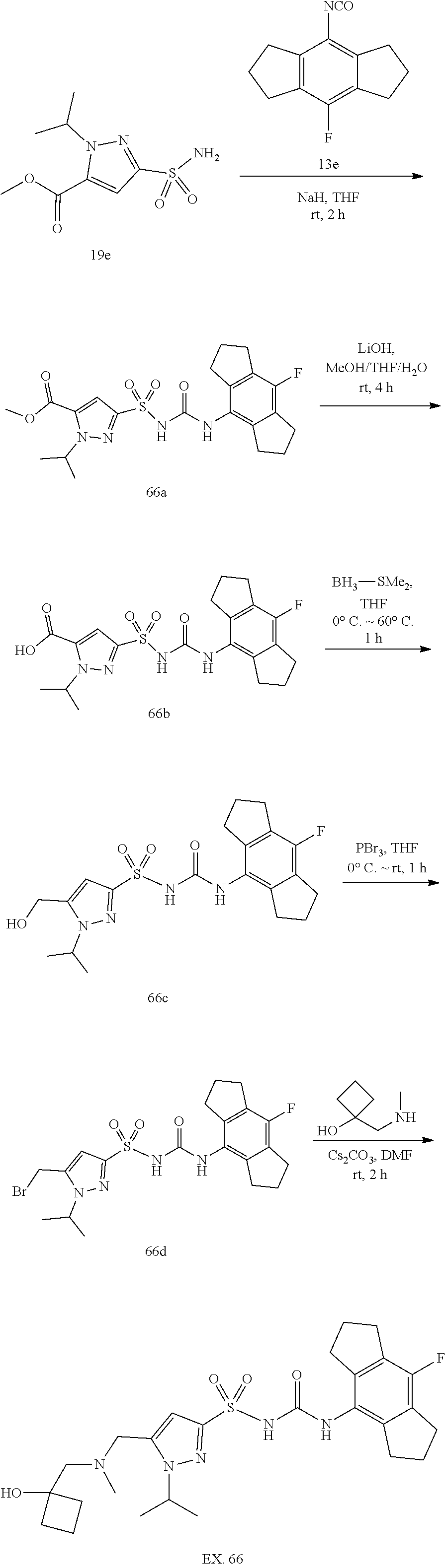

### Step 1:

NaH (97 mg, 2.420 mmol) was added to a solution of methyl 1-isopropyl-3-sulfamoyl-1H-pyrazole-5-carboxylate 19e (570 mg, 2.305 mmol) in THF (15 mL) at 0° C. The resulting suspension was stirred at rt for 0.5 h. 4-Fluoro-8-isocyanato-1,2,3,5,6,7-hexahydro-s-indacene (550 mg, 2.536 mmol) 13e was added. The reaction mixture was stirred at rt for 1.5 h. A solution of CHCl₃/iPrOH (v/v, 3/1, 10 mL) was added followed by water (10 mL). The mixture was separated, the aqueous phase was extracted with CHCl₃/iPrOH (v/v, 3/1, 10 mL×2), and the combined organic phases were washed with brine (10 mL×2), dried with Na₂SO₄, filtered and concentrated. The crude solid was washed with PE to provide methyl 3-(N-((8-fluoro-1,2,3,5,6,7-hexahydro-s-indacen-4-yl)carbamoyl)sulfamoyl)-1-isopropyl-1H-pyrazole-5-carboxylate 66a as a white solid (500 mg, 33.95%). LC-MS: m/z [M+H]⁺=465.20.

### Step 2:

A solution of LiOH (77 mg, 3.228 mmol) in H₂O (10 mL) was added into a solution of methyl 3-(N-((8-fluoro-1,2,3,5,6,7-hexahydro-s-indacen-4-yl)carbamoyl)sulfamoyl)-1-isopropyl-1H-pyrazole-5-carboxylate 66a (500 mg, 1.076 mmol) in THE (20 mL) and MeOH (5 mL) to generate a brown solution. The reaction mixture was stirred at room temperature for 3 h. The mixture was concentrated and the remainder aqueous residue was acidified to pH ˜4 using an aqueous solution of HCl (2 M) to produce a suspension. The white solids were filtered and dried in oven to give 3-(N-((8-fluoro-1,2,3,5,6,7-hexahydro-s-indacen-4-yl)carbamoyl)sulfamoyl)-1-isopropyl-1H-pyrazole-5-carboxylic acid 66b (450 mg, 92.4%) as a white solid. LC-MS: m/z [M+H]⁺=451.10.

### Step 3:

BH₃-Me₂S complex (0.47 mL, 10 M, 2.050 mmol) was added dropwise to a stirred colorless suspension of 3-(N-((8-fluoro-1,2,3,5,6,7-hexahydro-s-indacen-4-yl)carbamoyl)sulfamoyl)-1-isopropyl-1H-pyrazole-5-carboxylic acid 66b (450 mg, 0.999 mmol) in THE (20 mL) at 0° C. under N₂. Cooling was stopped and the reaction mixture was heated to 60° C. and remained at this temperature for 1 h. After cooled to room temperature, the reaction was quenched with NH₄Cl (aq, 5 mL) and diluted with water (10 mL). The mixture was extracted with CHCl₃/iPrOH (3/1, 20 mL×3). The combined organic phases were concentrated in vacuo to give N-((8-fluoro-1,2,3,5,6,7-hexahydro-s-indacen-4-yl)carbamoyl)-5-(hydroxymethyl)-1-isopropyl-1H-pyrazole-3-sulfonamide 66c as a white solid (400 mg, 77.43%). LC-MS: m/z [M+H]⁺=437.15.

### Step 4:

PBr₃ (273 mg, 1.008 mmol) was added dropwise to a colorless suspension of N-((8-fluoro-1,2,3,5,6,7-hexahydro-s-indacen-4-yl)carbamoyl)-5-(hydroxymethyl)-1-isopropyl-1H-pyrazole-3-sulfonamide 66c (400 mg, 0.916 mmol) in THE (10 mL) at 0° C. The resulting suspension was stirred at room temperature for 1 h. The reaction mixture was diluted with water (4 mL) and extracted with iPrOH/CHCl₃ (v/v, 1/3, 10 mL×3). The combined organic phases were washed with brine (15 mL), dried over Na₂SO₄, filtered and concentrated. The crude solid was recrystallized from petroleum ether to provide 5-(bromomethyl)-N-((8-fluoro-1,2,3,5,6,7-hexahydro-s-indacen-4-yl)carbamoyl)-1-isopropyl-1H-pyrazole-3-sulfonamide 66d as a white solid (400 mg, 82.56%). LC-MS: m/z [M]⁺, [M+2]=499.10, 501.10.

### Step 5:

To a colorless suspension of 1-[(methylamino)methyl]cyclobutan-1-ol (92 mg, 0.801 mmol) and Cs₂CO₃ (391 mg, 1.201 mmol) in DMF (2 mL) was added 3-[5-(bromomethyl)-1-isopropylpyrazol-3-ylsulfonyl]-1-(8-fluoro-1,2,3,5,6,7-hexahydro-s-indacen-4-yl)urea 66d (200 mg, 0.400 mmol). The resulting slightly brown suspension was stirred at room temperature for 2 h. The reaction mixture was filtered and the filtrate was purified by reverse phase column (15% to 45% CH₃CN in water (containing 0.1% FA)) to give a white solid (60 mg). This solid product was further purified by Prep-HPLC using the following conditions: Column: XBridge Prep Phenyl OBD Column, 19*150 mm, 5 μm; Mobile Phase A: Water (10 mmol/L NH₄HCO₃+0.1% NH₃·H₂O), Mobile Phase B: ACN; Flow rate: 25 mL/min; Gradient: 30% B to 38% B in 10 min, 38% B; Wavelength: 254 nm; RT1(min): 7.2. After lyophilization, the titled compound N-((8-fluoro-1,2,3,5,6,7-hexahydro-s-indacen-4-yl)carbamoyl)-5-((((1-hydroxycyclobutyl)methyl(methyl)amino)methyl)-1-isopropyl-1H-pyrazole-3-sulfonamide Ex. 66 was obtained as a white solid (20.1 mg, 9.07%). ¹HNMR (400 MHz, DMSO-d6) δ (ppm): 6.45-6.55 (m, 1H), 4.86-5.01 (m, 1H), 3.67 (s, 2H), 2.57-2.89 (m, 8H), 2.45 (s, 2H), 2.09-2.24 (m, 3H), 1.79-2.05 (m, 1H), 1.49-1.65 (m, 1H), 1.27-1.42 (m, 7H); ¹⁹F NMR (376 MHz, DMSO-d6) δ (ppm): −126.16; LC-MS: m/z [M+H]⁺=534.25.

### Example 67: Preparation of (S)—N-((8-fluoro-1,2,3,5,6,7-hexahydro-s-indacen-4-yl)carbamoyl)-5-((3-hydroxytropidine-1-yl)methyl)-1-isopropyl-1H-pyrazole-3-sulfonamide

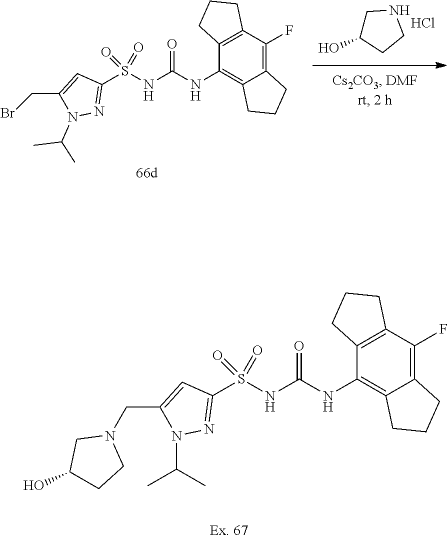 [SMILES: CC(C)n1nc(S(=O)(=O)NC(=O)Nc2c3c(c(F)c4c2CCC4)CCC3)cc1CBr ; O[C@H]1CCNC1 ; Cl ; CC(C)n1nc(S(=O)(=O)NC(=O)Nc2c3c(c(F)c4c2CCC4)CCC3)cc1CN1CC[C@H](O)C1]

Following the procedures described in Preparative Example 59, 3-[5-(bromomethyl)-1-isopropylpyrazol-3-ylsulfonyl]-1-(8-fluoro-1,2,3,5,6,7-hexahydro-s-indacen-4-yl)urea 66d (200 mg, 0.400 mmol) was reacted with (S)-pyrrolidin-3-ol hydrochloride salt to afford the titled compound (S)—N-((8-fluoro-1,2,3,5,6,7-hexahydro-s-indacen-4-yl)carbamoyl)-5-((3-hydroxypyrrolidin-1-yl)methyl)-1-isopropyl-1H-pyrazole-3-sulfonamide Ex. 67 (26.8 mg). ¹HNMR (400 MHz, DMSO-d6) δ (ppm): 6.58 (s, 1H), 4.66-4.84 (m, 1H), 4.12-4.25 (m, 1H), 3.63-3.82 (m, 2H), 2.55-2.91 (m, 10H), 2.39-2.48 (m, 1H), 2.27-2.38 (m, 1H), 1.86-2.08 (m, 5H), 1.47-1.61 (m, 1H), 1.36 (d, J=6.5 Hz, 6H); ¹⁹F NMR (376 MHz, DMSO-d6) δ (ppm): −125.58; LC-MS: m/z [M+H]⁺=506.20.

### Example 68: Preparation of N-((8-chloro-1,2,3,5,6,7-hexahydro-s-indacen-4-yl)carbamoyl)-5-((((1-hydroxycyclobutyl)methyl)(methyl)amino)methyl)-1-methyl-1H-pyrazole-3-sulfonamide

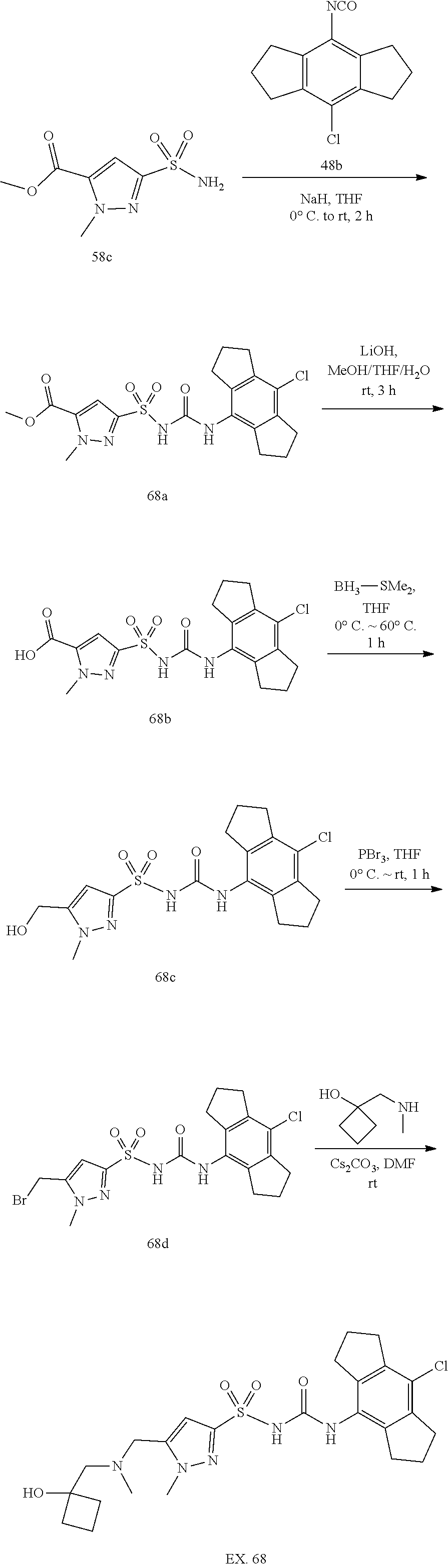

### Step 1:

NaH (46 mg, 1.150 mmol) was added to a solution of methyl 1-methyl-3-sulfamoyl-1H-pyrazole-5-carboxylate 58c (240 mg, 1.095 mmol) in THF (15 mL) at 0° C. The resulting suspension was stirred at rt for 0.5 h. 4-Chloro-8-isocyanato-1,2,3,5,6,7-hexahydro-s-indacene 48b (281 mg, 1.204 mmol) was added. The reaction mixture was stirred at rt for 1.5 h. A solution of CHCl₃/iPrOH (v/v, 3/1, 10 mL) was added followed by water (10 mL). The mixture was separated, the aqueous phase was extracted with CHCl₃/iPrOH (v/v, 3/1, 10 mL×2), and the combined organic phases were washed with brine (10 mL×2), dried with Na₂SO₄, filtered and concentrated. The crude solid was washed with PE to give methyl 3-(N-((8-fluoro-1,2,3,5,6,7-hexahydro-s-indacen-4-yl)carbamoyl)sulfamoyl)-1-isopropyl-1H-pyrazole-5-carboxylate 68a as a white solid (400 mg, 73.44%). LC-MS: m/z [M+H]⁺=453.10.

### Step 2:

A solution of LiOH (63 mg, 2.649 mmol) in H₂O (10 mL) was added into a solution of methyl 3-(N-((8-chloro-1,2,3,5,6,7-hexahydro-s-indacen-4-yl)carbamoyl)sulfamoyl)-1-methyl-1H-pyrazole-5-carboxylate 68a (400 mg, 0.883 mmol) in THF (20 mL) and MeOH (5 mL). The resulting brown solution was stirred at room temperature for 3 h. The mixture was concentrated, and the aqueous residue was acidified to pH ˜4 using a 2M aqueous solution of HCl to produce a suspension. The white solids were collected by filtration and further dried in oven to give 3-(N-((8-chloro-1,2,3,5,6,7-hexahydro-s-indacen-4-yl)carbamoyl)sulfamoyl)-1-methyl-1H-pyrazole-5-carboxylic acid 68b as a white solid (340 mg, 84.17%). LC-MS: m/z [M+H]⁺=439.15.

### Step 3:

BH₃-Me₂S complex (0.37 mL, 10 M, 3.875 mmol) was added dropwise to a stirred colorless suspension of 3-(N-((8-chloro-1,2,3,5,6,7-hexahydro-s-indacen-4-yl)carbamoyl)sulfamoyl)-1-methyl-1H-pyrazole-5-carboxylic acid 68b (340 mg, 0.802 mmol) in THF (20 mL) at 0° C. under N₂ to afford a colorless solution. Cooling was stopped, and the reaction mixture was heated to 60° C. and continued at this temperature for 1 h to yield a colorless suspension. After cooled to room temperature, the reaction was quenched with NH₄Cl (aq, 5 mL) and diluted with water (10 mL). The aqueous mixture was extracted with CHCl₃/iPrOH (3/1, 20 mL×3). The combined organic phases were concentrated in vacuo to give N-((8-chloro-1,2,3,5,6,7-hexahydro-s-indacen-4-yl)carbamoyl)-5-(hydroxymethyl)-1-methyl-1H-pyrazole-3-sulfonamide 68c as a white solid (300 mg, 81.20%). LC-MS: m/z [M+H]⁺=425.10.

### Step 4:

PBr₃ (210 mg, 0.777 mmol) was added dropwise to a colorless suspension of N-((8-chloro-1,2,3,5,6,7-hexahydro-s-indacen-4-yl)carbamoyl)-5-(hydroxymethyl)-1-methyl-1H-pyrazole-3-sulfonamide 68c (300 mg, 0.706 mmol) in THF (10 mL) at 0° C. The resulting mixture was stirred at room temperature for 1 h. The mixture was diluted with water (4 mL) and extracted with iPrOH/CHCl₃ (v/v, 1/3, 10 mL×3). The combined organic phases were washed with brine (15 mL), dried with Na₂SO₄, filtered and concentrated. The crude solid was recrystallized from petroleum ether to provide 5-(bromomethyl)-N-((8-chloro-1,2,3,5,6,7-hexahydro-s-indacen-4-yl)carbamoyl)-1-methyl-1H-pyrazole-3-sulfonamide 68d as a white solid (280 mg, 59.40%). LC-MS: m/z [M]⁺, [M+2]⁺=487.05, 489.05

### Step 5:

To a colorless suspension of 1-[(methylamino)methyl]cyclobutan-1-ol (132 mg, 1.148 mmol) and Cs₂CO₃ (561.07 mg, 1.722 mmol) in DMF (2 mL) was added 3-[5-(bromomethyl)-1-methylpyrazol-3-ylsulfonyl]-1-(8-chloro-1,2,3,5,6,7-hexahydro-s-indacen-4-yl)urea 68d (280 mg, 0.574 mmol). The resulting slightly brown suspension was stirred at room temperature for 2 h. The reaction mixture was filtered, and the filtrate was purified by reverse phase column chromatography (15-40% MeCN/water (0.1% FA)) to give a white solid (70 mg). This solid product was further purified by Prep-HPLC using the following conditions: Column: XBridge Shield RP18 OBD Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 25% B to 47% B in 7 min, 47% B; Wavelength: 254 nm; RT1(min): 4.48. After lyophilization, the titled compound N-((8-chloro-1,2,3,5,6,7-hexahydro-s-indacen-4-yl)carbamoyl)-5-((((1-hydroxycyclobutyl)methyl)(methyl)amino)methyl)-1-methyl-1H-pyrazole-3-sulfonamide Ex. 68 was obtained as a white solid (23.5 mg, 7.63%). ¹HNMR (400 MHz, DMSO-d6) δ (ppm): 6.51-6.69 (m, 1H), 3.82-3.97 (m, 3H), 3.69 (s, 2H), 2.61-2.88 (m, 8H), 2.47 (s, 2H), 2.15-2.29 (m, 3H), 1.82-2.06 (m, 8H), 1.49-1.63 (m, 1H), 1.21-1.41 (m, 1H); LC-MS: m/z [M+H]⁺=522.20.

### Example 69: Preparation of N-((8-chloro-1,2,3,5,6,7-hexahydro-s-indacen-4-yl)carbamoyl)-5-((((1-hydroxycyclobutyl)methyl)(methyl)amino)methyl)-1-isopropyl-H-pyrazole-3-sulfonamide

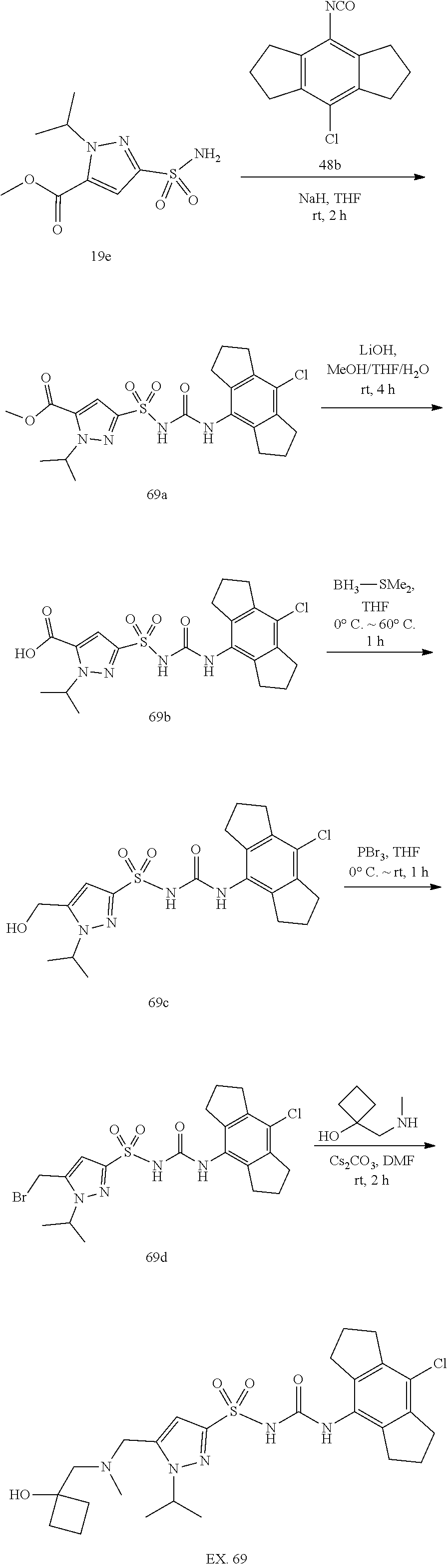

Following the procedures set forth in Preparative Example 66, by using 4-Chloro-8-isocyanato-1,2,3,5,6,7-hexahydro-s-indacene 48b in Step 1, the titled compound N-((8-chloro-1,2,3,5,6,7-hexahydro-s-indacen-4-yl)carbamoyl)-5-((((1-hydroxycyclobutyl)methyl)(methyl)amino)methyl)-1-isopropyl-1H-pyrazole-3-sulfonamide Ex. 69 was prepared (16.9 mg). ¹HNMR (400 MHz, DMSO-d6) δ (ppm): 6.55 (s, 1H), 4.97 (m, 1H), 3.67 (s, 2H), 2.80-2.83 (m, 4H), 2.65-2.69 (m, 4H), 2.46 (s, 2H), 2.17 (s, 3H), 1.85-2.04 (m, 8H), 1.53-1.59 (m, 1H), 1.30-1.37 (m, 7H); LC-MS: m/z [M+1]⁺, [M+3]⁺=550.20, 552.20.

### Example 70: Preparation of N-((1,2,3,5,6,7-hexahydro-s-indacen-4-yl)carbamoyl)-4-(2-hydroxypropan-2-yl)-5-(piperazin-1-ylmethyl)furan-2-sulfonamide

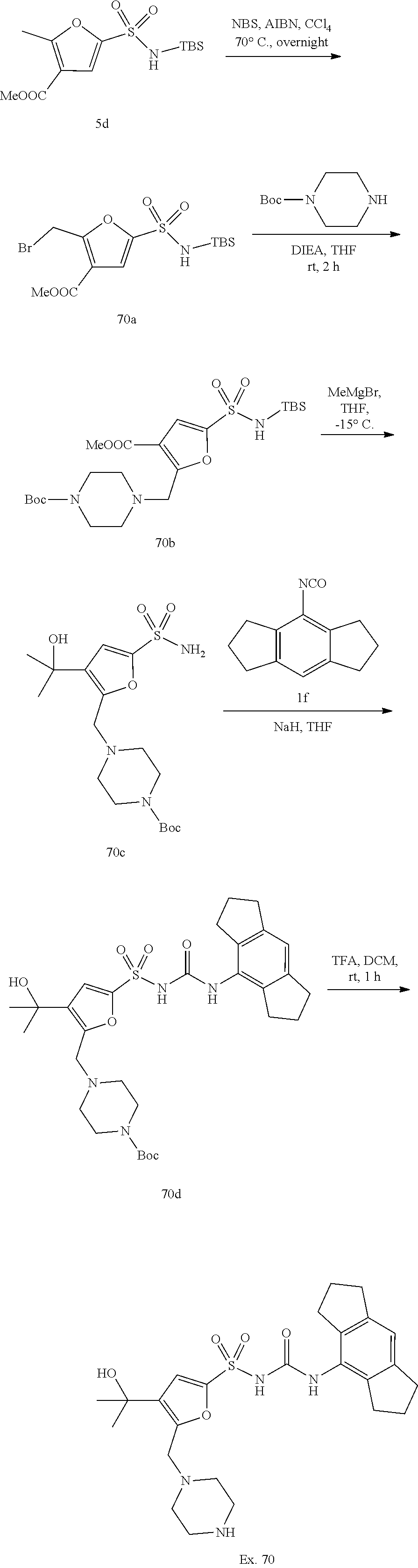

### Step 1:

A yellow stirred solution of methyl 5-[(tert-butyldimethylsilyl)sulfamoyl]-2-methylfuran-3-carboxylate 5d (1 g, 2.999 mmol), NBS (0.56 g, 3.149 mmol) and AIBN (10 mg, 0.060 mmol) in CCl₄ (16 mL) was refluxed under N₂ overnight. After cooled down to room temperature, EtOAc (20 mL) and water (10 mL) were added. The mixture was separated, the aqueous phase was extracted with EtOAc (20 mL×2), and the combined organic phases were washed with brine (20 mL×2), dried with Na₂SO₄, filtered. Removal of solvents provided methyl 2-(bromomethyl)-5-[(tert-butyldimethylsilyl)sulfamoyl]furan-3-carboxylate 70a as a white solid (600 mg, 47.67%). LC-MS: m/z [M+H]⁺=411.95.

### Step 2:

A colorless solution of tert-butyl piperazine-1-carboxylate (271 mg, 1.455 mmol), methyl 2-(bromomethyl)-5-[(tert-butyldimethylsilyl)sulfamoyl]furan-3-carboxylate 70a (300 mg, 0.728 mmol) and DIEA (282 mg, 2.183 mmol) in THF (5 mL) was stirred at room temperature for 2 h. The reaction mixture was diluted with water (10 mL) and extracted with EtOAc (20 mL×3). The combined organic phases were washed with brine (20 mL×3), dried with Na₂SO₄, filtered and concentrated. The residue was purified by silica gel column chromatography to give tert-butyl 4-([5-[(tert-butyldimethylsilyl)sulfamoyl]-3-(methoxycarbonyl)furan-2-yl]methyl)piperazine-1-carboxylate 70b (330 mg, 87.00%) as a white solid. LC-MS: m/z [M+H]⁺=518.25.

### Step 3:

A solution of MeMgBr in ether (0.97 mL, 2.895 mmol) was added dropwise to a solution of tert-butyl 4-[[3-(methoxycarbonyl)-5-sulfamoylfuran-2-yl]methyl]piperazine-1-carboxylate 70b (300 mg, 0.579 mmol) in THF (10 mL) at −15° C. The resulting mixture was allowed to warm to rt and stirred for 3 h. The reaction was quenched with the addition of a saturated NH₄C1 aqueous solution (10 mL). The aqueous mixture was extracted with EtOAc (15 mL×3). The combined organic phases were washed with brine (10 mL×2), dried with Na₂SO₄, filtered and concentrated. The residue was purified by silica gel column (0 to 100% EtOAc in PE) to provide tert-butyl 4-((3-(2-hydroxypropan-2-yl)-5-sulfamoylfuran-2-yl)methyl)piperazine-1-carboxylate 70c as a white solid (250 mg, 82.41%). LC-MS: m/z [M+H]⁺=404.20.

### Step 4:

NaH (12 mg, 0.483 mmol) was added to a solution of tert-butyl 4-((3-(2-hydroxypropan-2-yl)-5-sulfamoylfuran-2-yl)methyl)piperazine-1-carboxylate 70c (195 mg, 0.483 mmol) in THF (5 mL) at 0° C. The resulting suspension was stirred at this temperature for 15 min. 4-Isocyanato-1,2,3,5,6,7-hexahydro-s-indacene if (96 mg, 0.483 mmol) was added. Cooling was stopped, and the reaction mixture was stirred at rt for 1 h. A solution of CHCl₃/iPrOH (v/v, 3/1, 10 mL) was added followed by water (5 mL). The aqueous mixture was separated, and the aqueous phase was extracted with CHCl₃/iPrOH (v/v, 3/1, 10 mL×2). The combined organic phases were washed with brine (5 mL×2), dried with Na₂SO₄, filtered and concentrated in vacuo to give tert-butyl 4-([5-[(tert-butyldimethylsilyl)sulfamoyl]-3-(2-hydroxypropan-2-yl)furan-2-yl]methyl)piperazine-1-carboxylate 70d as a white solid (300 mg, 88.13%).

### Step 5:

A brown solution of tert-butyl 4-[(5-[[(1,2,3,5,6,7-hexahydro-s-indacen-4-yl)carbamoyl]aminosulfonyl]-3-(2-hydroxypropan-2-yl)furan-2-yl)methyl]piperazine-1-carboxylate 70d (100 mg, 0.166 mmol) in TFA (0.5 mL) and DCM (2 mL) was stirred at room temperature for 1 h. Solvents were removed through evaporation. The residue was dissolved in DMF and neutralized with NaOMe, and the resulting solution was purified by Prep-HPLC using the following conditions: Column: Kinetex EVO C18 Column, 30*150, 5 μm; Mobile Phase A: Water(0.05% FA), Mobile Phase B: ACN; Flow rate: 60 mL/min; Gradient: 18% B to 38% B in 7 min, 38% B; 254 nm; RT1: 7.40 min. After lyophilization, the titled compound 3-(1,2,3,5,6,7-hexahydro-s-indacen-4-yl)-1-[4-(2-hydroxypropan-2-yl)-5-(piperazin-1-ylmethyl)furan-2-ylsulfonyl]urea Ex. 70 was obtained as a white solid (25.0 mg, 29.05%). ¹HNMR (400 MHz, DMSO-d6) δ (ppm): 6.80 (s, 1H), 6.53 (s, 1H), 3.73 (s, 2H), 3.01-3.09 (m, 4H), 2.71-2.79 (m, 4H), 2.53-2.76 (m, 8H), 1.86-1.92 (m, 4H), 1.39 (s, 6H); LC-MS: m/z [M+H]⁺=503.25.

### Example 71: Preparation of 5-((azetidin-3-yloxy)methyl)-N-((8-fluoro-1,2,3,5,6,7-hexahydro-s-indacen-4-yl)carbamoyl)-4-(2-hydroxypropan-2-yl)furan-2-sulfonamide

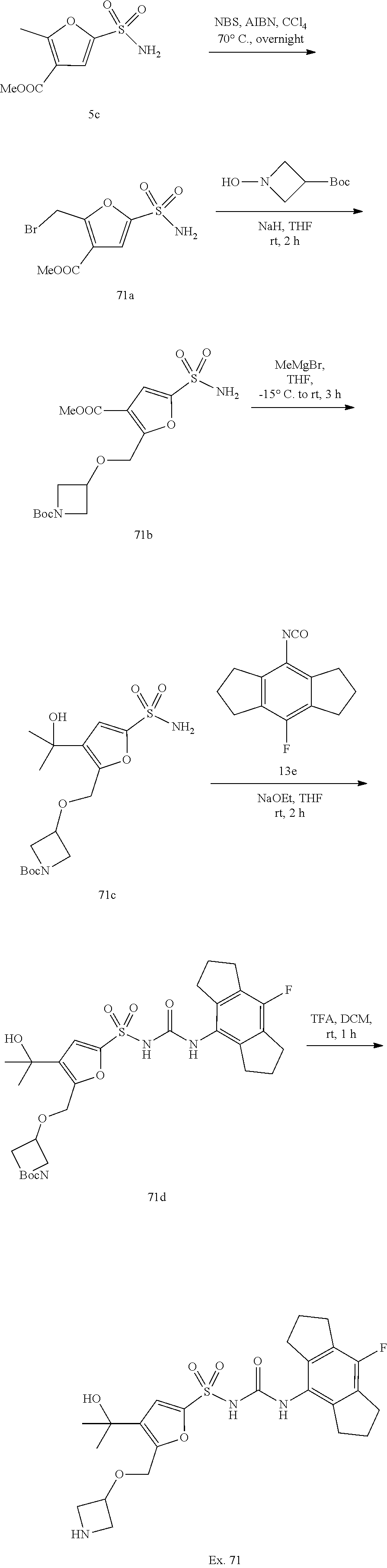

### Step 1:

To a colorless suspension of methyl 2-methyl-5-sulfamoylfuran-3-carboxylate 5c (2 g, 9.124 mmol) and AIBN (0.30 g, 1.827 mmol) in CCl₄ (50 mL) was added NBS (1.71 g, 9.608 mmol) with stirring. The resulting light brown suspension was heated to 70° C. and continued overnight. After cooling to room temperature, EtOAc (50 mL) and water (50 mL) were added. The aqueous mixture was separated. The aqueous phase was extracted with EtOAc (50 mL×2). The combined organic phases were washed with brine (50 mL×2), dried with Na₂SO₄, filtered and concentrated. The residue was submitted to silica gel column chromatography to give methyl 2-(bromomethyl)-5-sulfamoylfuran-3-carboxylate 71a as a light yellow oil (1.75 g, 48.00%).

### Step 2:

NaH (141 mg, 3.522 mmol) was added to a solution of methyl 2-(bromomethyl)-5-sulfamoylfuran-3-carboxylate 71a (700 mg, 2.348 mmol) in THF (15 mL) at 0° C., and the resulting suspension was stirred at rt for 0.5 h. tert-Butyl 3-hydroxyazetidine-1-carboxylate (610 mg, 3.522 mmol) was added. The reaction mixture was stirred at rt for 1.5 h. EtOAc (20 mL) and water (20 mL) were added. The aqueous mixture was separated. The aqueous phase was extracted with EtOAc (20 mL×2). The combined organic phases were washed with brine (20 mL×2), dried with Na₂SO₄, filtered and concentrated. The residue was purified by silica gel column chromatography to give tert-butyl 3-((3-(methoxycarbonyl)-5-sulfamoylfuran-2-yl)methoxy)azetidine-1-carboxylate 71b as a light yellow oil (400 mg, 41.04%).

### Step 3

MeMgBr (0.67 mL, 3M, 5.838 mmol) were added dropwise to a stirred colorless solution of tert-butyl 3-((3-(methoxycarbonyl)-5-sulfamoylfuran-2-yl)methoxy)azetidine-1-carboxylate 71b (380 mg, 0.973 mmol) in THF (60 mL) at −20° C. under N₂. The reaction mixture was stirred at −20° C. for 1 h and continued at rt for 2 h. EtOAc (20 mL) and water (20 mL) were added. The aqueous mixture was separated. The aqueous phase was extracted with EtOAc (20 mL×2). The combined organic phases were washed with brine (20 mL×2), dried with Na₂SO₄, filtered and concentrated. The residue was purified by silica gel column chromatography to provide tert-butyl 3-((3-(2-hydroxypropan-2-yl)-5-sulfamoylfuran-2-yl)methoxy)azetidine-1-carboxylate 71c as a light yellow oil (380 mg, 89.11%).

### Step 4:

To a colorless suspension of tert-butyl 3-((3-(2-hydroxypropan-2-yl)-5-sulfamoylfuran-2-yl)methoxy)azetidine-1-carboxylate 71c (230 mg, 0.589 mmol) and sodium ethoxide (40 mg, 0.589 mmol) in THF (3 mL) was added 4-fluoro-8-isocyanato-1,2,3,5,6,7-hexahydro-s-indacene 13e (134 mg, 0.618 mmol). The resulting light brown suspension was stirred at room temperature for 2 h. A solution of CHCl₃/iPrOH (v/v, 3/1, 10 mL) was added followed by water (10 mL). The mixture was separated, the aqueous phase was extracted with CHCl₃/iPrOH (v/v, 3/1, 20 mL×2), and the combined organic phases were washed with brine (15 mL×2), dried with Na₂SO₄, filtered and concentrated to provide tert-butyl 3-((5-(N-((8-fluoro-1,2,3,5,6,7-hexahydro-s-indacen-4-yl)carbamoyl)sulfamoyl)-3-(2-hydroxypropan-2-yl)furan-2-yl)methoxy)azetidine-1-carboxylate 71d as a white solid (270 mg, 63.78%). LC-MS: m/z [M+H]⁺=608.30.

### Step 5:

A solution of tert-butyl 3-[(5-[[(8-fluoro-1,2,3,5,6,7-hexahydro-s-indacen-4-yl)carbamoyl]aminosulfonyl]-3-(2-hydroxypropan-2-yl)furan-2-yl)methoxy]azetidine-1-carboxylate 71d (240.00 mg, 0.395 mmol) in TFA (1 mL) and DCM (5 mL) was stirred at rt for 1 h. Solvents were removed under vacuo at 0° C. The residue was dissolved in DCM and added with TEA to adjust pH to 9-10. The solution was purified by reverse phase column chromatography eluting with 15% to 40% MeCN in water(0.1% FA) to give a white solid (70 mg). This solid was further purified by Prep-HPLC using the following conditions: Column: Xselect CSH OBD Column 30*150 mm 5 μm; Mobile Phase A: Water(10 mmol/L NH₄HCO₃+0.1% NH₃·H₂O), Mobile Phase B: ACN; Flow rate: 60 m/min; Gradient: 17% B to 37% B in 8 min, 37% B; Wavelength: 254 nm; RT1(min): 7.65. After lyophilization, the titled compound 1-[5-[(azetidin-3-yloxy)methyl]-4-(2-hydroxypropan-2-yl)furan-2-ylsulfonyl]-3-(8-fluoro-1,2,3,5,6,7-hexahydro-s-indacen-4-yl)urea Ex. 71 was obtained as a white solid (18.8 mg, 9.34%). ¹HNMR (300 MHz, DMSO-d6) δ (ppm): 6.50 (s, 1H), 4.63 (s, 2H), 4.36-4.52 (m, 3H), 3.95-4.10 (m, 2H), 3.62-3.84 (m, 2H), 2.76-2.87 (m, 4H), 2.62-2.76 (m, 4H), 1.88-2.05 (m, 4H), 1.40 (s, 6H); ¹⁹F NMR (282 MHz, DMSO-d6) δ (ppm): −127.96; LC-MS: m/z [M+H]⁺=508.15.

### Example 72: Preparation of N-((8-fluoro-1,2,3,5,6,7-hexahydro-s-indacen-4-yl)carbamoyl)-4-(1-hydroxyethyl)-5-(piperazin-1-ylmethyl)furan-2-sulfonamide

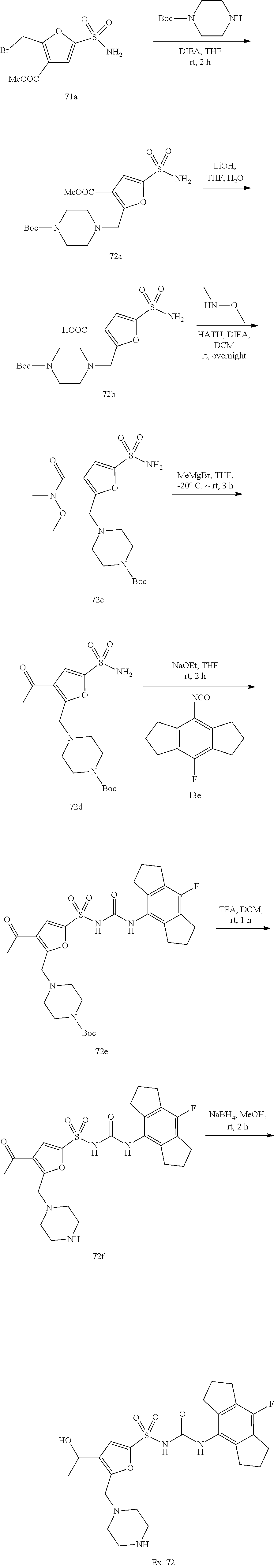

### Step 1:

To a colorless suspension of methyl 2-(bromomethyl)-5-sulfamoylfuran-3-carboxylate 71a (1 g, 3.354 mmol) and DIEA (1.75 mL, 10.047 mmol) in THF (20 mL) was added tert-butyl piperazine-1-carboxylate (1.25 g, 6.711 mmol). The resulting slightly brown suspension was stirred at room temperature for 2 h. A solution of CHCl₃/iPrOH (v/v, 3/1, 50 mL) was added followed by water (50 mL). The mixture was separated. The aqueous phase was extracted with CHCl₃/iPrOH (v/v, 3/1, 50 mL×2). The combined organic phases were washed with brine (50 mL×2), dried with Na₂SO₄, filtered and concentrated. The residue was purified by silica gel column chromatography to give tert-butyl 4-((3-(methoxycarbonyl)-5-sulfamoylfuran-2-yl)methyl)piperazine-1-carboxylate 72a as a white solid (980 mg, 63.97%). LC-MS: m/z [M+H]⁺=404.15.

### Step 2:

A solution of LiOH (174 mg, 7.287 mmol) in H₂O (10 mL) was added to a stirred solution of tert-butyl 4-((3-(methoxycarbonyl)-5-sulfamoylfuran-2-yl)methyl)piperazine-1-carboxylate 72a (980 mg, 2.429 mmol) in THE (20 mL). The resulting mixture was stirred at room temperature for 3 h and concentrated. The remainder aqueous residue was acidified to pH ˜4 using a 2M aqueous HCl solution to provide a suspension. The white solids from the suspension were collected by filtration and further dried in oven to afford 2-((4-(tert-butoxycarbonyl)piperazin-1-yl)methyl)-5-sulfamoylfuran-3-carboxylic acid 72b as a white solid (950 mg, 88.16%).

### Step 3:

A solution of 2-((4-(tert-butoxycarbonyl)piperazin-1-yl)methyl)-5-sulfamoylfuran-3-carboxylic acid 72b (950 mg, 2.440 mmol), N,O-dimethylhydroxylamine (447 mg, 7.319 mmol), HATU (1391 mg, 3.659 mmol) and DIEA (945 mg, 7.319 mmol) in DMF (5 mL) was stirred at rt overnight. The reaction mixture was diluted with water (50 mL) and extracted with iPrOH/CHCl₃ (v/v, 1/3, 20 mL×3). The combined organic phases were washed with brine (50 mL), dried with Na₂SO₄, filtered and concentrated. The crude solid product was purified by silica gel column chromatography and further by reverse phase column chromatography to give tert-butyl 4-((3-(methoxy(methyl)carbamoyl)-5-sulfamoylfuran-2-yl)methyl)piperazine-1-carboxylate 72c as a white solid (350 mg, 33.08%). LC-MS: m/z [M+H]⁺=433.15.

### Step 4:

A solution of MeMgBr in ether (0.40 mL, 3M, 3.472 mmol) was added dropwise to a stirred colorless solution of tert-butyl 4-((3-(methoxy(methyl)carbamoyl)-5-sulfamoylfuran-2-yl)methyl)piperazine-1-carboxylate 72c (300 mg, 0.694 mmol) in THE (20 mL) at −20° C. under N₂. The resulting mixture was stirred at −20° C. for 1 h and continued at rt for 2 h. EtOAc (20 mL) and water (20 mL) were added successively. The mixture was separated, the aqueous phase was extracted with EtOAc (20 mL×2), and the combined organic phases were washed with brine (20 mL×2), dried with Na₂SO₄, filtered and concentrated. The residue was purified by reverse phase column chromatography to afford tert-butyl 4-((3-acetyl-5-sulfamoylfuran-2-yl)methyl)piperazine-1-carboxylate 72d as a white solid (170 mg, 50.30%).

### Step 5:

To a colorless suspension of tert-butyl 4-((3-acetyl-5-sulfamoylfuran-2-yl)methyl)piperazine-1-carboxylate 72d (170 mg, 0.439 mmol) and sodium ethoxide (31 mg, 0.461 mmol) in THF (3 mL) was added 4-fluoro-8-isocyanato-1,2,3,5,6,7-hexahydro-s-indacene (104 mg, 0.483 mmol). The resulting slightly brown suspension was stirred at room temperature for 2 h. A solution of CHCl₃/iPrOH (v/v, 3/1, 10 mL) was added followed by water (10 mL). After separation of the biphasic mixture, the aqueous phase was extracted with CHCl₃/iPrOH (v/v, 3/1, 10 mL×2). The combined organic phases were washed with brine (10 mL×2), dried with Na₂SO₄, filtered and concentrated. The crude solid product was recrystallized from petroleum ether to give tert-butyl 4-((3-acetyl-5-(N-((8-fluoro-1,2,3,5,6,7-hexahydro-s-indacen-4-yl)carbamoyl)sulfamoyl)furan-2-yl)methyl)piperazine-1-carboxylate 72e as a white solid (180 mg, 66.87%). LC-MS: m/z [M+H]⁺=605.25.

### Step 6:

A colorless solution of tert-butyl 4-((3-acetyl-5-(N-((8-fluoro-1,2,3,5,6,7-hexahydro-s-indacen-4-yl)carbamoyl)sulfamoyl)furan-2-yl)methyl)piperazine-1-carboxylate 72e (150 mg, 0.248 mmol) in TFA (I mL) and DCM (1 mL) was stirred at rt for 1 h. Solvents were removed under vacuo to give 4-acetyl-N-((8-fluoro-1,2,3,5,6,7-hexahydro-s-indacen-4-yl)carbamoyl)-5-(piperazin-1-ylmethyl)furan-2-sulfonamide 72f as a white solid (120 mg, 92.16%). LC-MS: m % z [M+H]⁺=505.20.

### Step 7:

A brown solution of 4-acetyl-N-((8-fluoro-1,2,3,5,6,7-hexahydro-s-indacen-4-yl)carbamoyl)-5-(piperazin-1-ylmethyl)furan-2-sulfonamide 72f (120 mg, 0.238 mmol) and NaBH₄ (44.99 mg, 1.189 mmol) in MeOH (2 mL) was stirred at room temperature for 2 h. The reaction mixture was diluted with water (0.1 mL) and purified by Prep-HPLC using the following conditions: Column: Kinetex EVO C18 Column, 30*150 mm, 5 μm; Mobile Phase A: Water(0.05% FA), Mobile Phase B: ACN; Flow rate: 25 mL/min; Gradient: 14% B to 27% B in 10 min, 27% B; Wavelength: 254 nm; RT1(min): 8.72. After lyophilization, the titled compound N-((8-fluoro-1,2,3,5,6,7-hexahydro-s-indacen-4-yl)carbamoyl)-4-(1-hydroxyethyl)-5-(piperazin-1-ylmethyl)furan-2-sulfonamide Ex. 72 was obtained as a white solid (26.6 mg, 21.87%). ¹HNMR (400 MHz, DMSO-d₆) δ (ppm): 6.58 (s, 1H), 4.63-4.70 (m, 1H), 3.55 (s, 2H), 2.91-3.13 (m, 4H), 2.64-2.90 (m, 8H), 2.56 (s, 4H), 1.83-2.06 (m, 4H), 1.18-1.36 (m, 3H); ¹⁹FNMR (376 MHz, DMSO-d₆) δ (ppm): −127.92; LC-MS: m/z [M+H]⁺=507.15.

### Example 73: Preparation of N-((1,2,3,5,6,7-hexahydro-s-indacen-4-yl)carbamoyl)-4,5,6,7-tetrahydrofuro[3,2-c]pyridine-2-sulfonamide

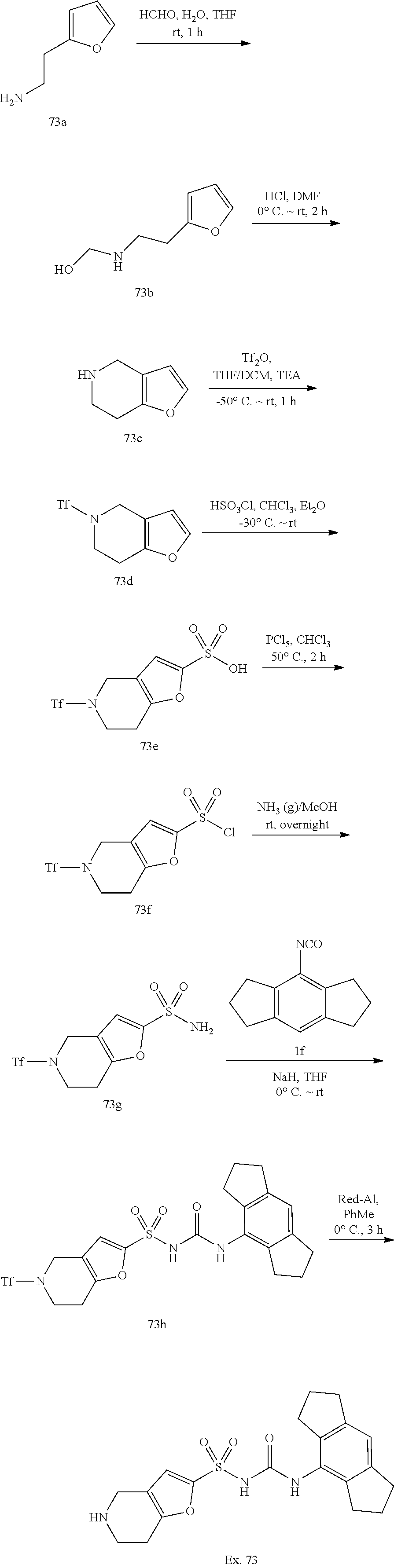

### Steps 1 and 2:

Formalin (37% aqueous formaldehyde, 1.32 mL, 35.989 mmol) was added dropwise to 2-furan-2-yl-ethylamine 73a (4 g, 35.989 mmol), and the resulting mixture was allowed to stir for 30 minutes at room temperature. The mixture was extracted with diethyl ether (3×80 mL). The diethyl ether extracts were combined, dried over sodium sulfate, filtered, and concentrated to an oily residue (73b). The oil residue 73b was dissolved in DMF (4 mL) and added to a DMF/HCl solution, which was obtained via passing hydrogen chloride gas through DMF (26 mL) for one hour. The reaction mixture was stirred at room temperature for 3 hours, then DMF was removed under high vacuum. Methyl t-butyl ether (MTBE) was added and the trace DMF was removed by extraction with water (100 mL) that had been adjusted to pH=11 using NaOH. The MTBE fraction was dried over sodium sulfate, filtered, and concentrated in vacuo to give 4H,5H,6H,7H-furo[3,2-c]pyridine 73c as a yellow oil (4.9 g, 25.34%).

### Step 3:

A colorless solution of Tf₂O (4.9 g, 17.539 mmol) in DCM (5 mL) was added dropwise to a yellow solution of 4H,5H,6H,7H-furo[3,2-c]pyridine 73c (1.8 g, 14.616 mmol) and TEA (2.2 g, 21.924 mmol) in DCM (45 mL) at −30° C. The resulting yellow solution was stirred for 1 h at this temperature and allowed to warm to rt gently. The reaction was quenched by the addition of water (40 mL). The aqueous mixture was separated, the aqueous phase was extracted with DCM (50 mL×2), and the combined organic phases were washed with brine (100 mL), dried with Na₂SO₄, filtered and concentrated. The residue was submitted to silica gel column chromatography (0 to 20% Et₂O in PE) to give 5-trifluoromethanesulfonyl-4H,6H,7H-furo[3,2-c]pyridine 73d as a colorless oil (2.6 g, 51.58%). ¹HNMR (300 MHz, CD₃Cl) δ (ppm): 7.34 (s, 1H), 6.24 (s, 1H), 4.48 (s, 2H), 3.82 (m, 2H), 2.87 (m, 2H), ¹⁹F NMR (282 MHz, CD₃Cl) δ (ppm): −76.39.

### Steps 4 and 5:

To a colorless solution of 5-trifluoromethanesulfonyl-4H,6H,7H-furo[3,2-c]pyridine 73d (600 mg, 2.351 mmol) in CHCl₃ (6 mL) and Et₂O (4 mL) at −30° C. was added a colorless solution of chlorosulfonic acid (274 mg, 2.351 mmol) in CHCl₃ (1 mL) dropwise under stirring. The resulting yellow solution was stirred at rt for 30 min. Then, PCl₅ (979 mg, 4.702 mmol) was added. The reaction mixture (a suspension) was heated at 50° C. for 2 h. The reaction mixture was purified by silica gel column chromatography eluting with EtOAc to give 5-trifluoromethanesulfonyl-4H,6H,7H-furo[3,2-c]pyridine-2-sulfonyl chloride 73f as a colorless oil (130 mg, 15.63%).

### Step 6:

A solution of NH₃ in MeOH (3 mL, 7 M, 21 mmol) was added to 5-trifluoromethanesulfonyl-4H,6H,7H-furo[3,2-c]pyridine-2-sulfonyl chloride 73f (130 mg, 0.368 mmol). The reaction mixture was stirred at rt overnight and concentrated. The residue was purified by silica gel column chromatography (EtOAc/PE) to give 5-trifluoromethanesulfonyl-4H,6H,7H-furo[3,2-c]pyridine-2-sulfonamide 73g as a white solid (120 mg, 97.67%). ¹HNMR (300 MHz, CD₃OD) δ (ppm): 6.89 (s, 1H), 4.52 (s, 2H), 3.91 (m, 2H), 2.91 (m, 2H).

### Step 7:

NaH (8.6 mg, 0.359 mmol) was added to a solution of 5-trifluoromethanesulfonyl-4H,6H,7H-furo[3,2-c]pyridine-2-sulfonamide 73g (120 mg, 0.359 mmol) in THE (5 mL) at 0° C. The resulting suspension was stirred at this temperature for 15 min. 4-Isocyanato-1,2,3,5,6,7-hexahydro-s-indacene if (71.5 mg, 0.359 mmol) was added. Cooling was stopped, and the reaction mixture was stirred at rt for 1 h. A solution of CHCl₃/iPrOH (v/v, 3/1, 20 mL) was added followed by water (10 mL). The mixture was separated, the aqueous phase was extracted with CHCl₃/iPrOH (v/v, 3/1, 20 mL×2), and the combined organic phases were washed with brine (20 mL×2), dried with Na₂SO₄, filtered and concentrated in vacuo to give 3-(1,2,3,5,6,7-hexahydro-s-indacen-4-yl)-1-[5-trifluoromethanesulfonyl-4H,6H,7H-furo[3,2-c]pyridin-2-ylsulfonyl]urea 73h as a white solid (160 mg, 83.54%).

### Step 8:

Red-Al (0.83 mL, 3.6 M, 3.000 mmol) was added dropwise to a stirred suspension of 3-(1,2,3,5,6,7-hexahydro-s-indacen-4-yl)-1-[5-trifluoromethanesulfonyl-4H,6H,7H-furo[3,2-c]pyridin-2-ylsulfonyl]urea 73h (160 mg, 0.300 mmol) in Toluene (8 mL) at 0° C. under N₂. The reaction mixture was stirred at 0° C. for 3 h. The reaction was quenched by addition of a saturated NH₄Cl aqueous solution (10 mL), and the mixture was extracted with iPrOH/CHCl₃ (v/v, 1/3, 10 mL×3). The combined organic phases was concentrated in vacuo and the residue was purified by Prep-HPLC using the following conditions: Column: YMC-Actus Triart C18 ExRS, 30*150 mm, 5 μm; Mobile Phase A: Water(10 mmol/L NH₄HCO₃+0.1% NH₃·H₂O), Mobile Phase B: ACN; Flow rate: 60 mL/min; Gradient: 18% B to 38% B in 7 min, 38% B; Wavelength: 254 nm; RT1(min): 5.5. After lyophilization, the titled compound 1-[4H,5H,6H,7H-furo[3,2-c]pyridine-2-sulfonyl]-3-(1,2,3,5,6,7-hexahydro-s-indacen-4-yl)urea Ex. 73 was obtained as a white solid (15.1 mg, 12.30%). ¹HNMR (400 MHz, DMSO-d6) δ (ppm): 6.81 (s, 1H), 6.50 (s, 1H), 3.71 (s, 2H), 3.10 (m, 2H), 2.59-2.76 (m, 10H), 1.92 (m, 4H); LC-MS: m/z [M+H]⁺=402.10.

### Example 74: Preparation of N-((1,2,3,5,6,7-hexahydro-s-indacen-4-yl)carbamoyl)-5-(3-hydroxypropyl)-4,5,6,7-tetrahydrofuro[3,2-c]pyridine-2-sulfonamide

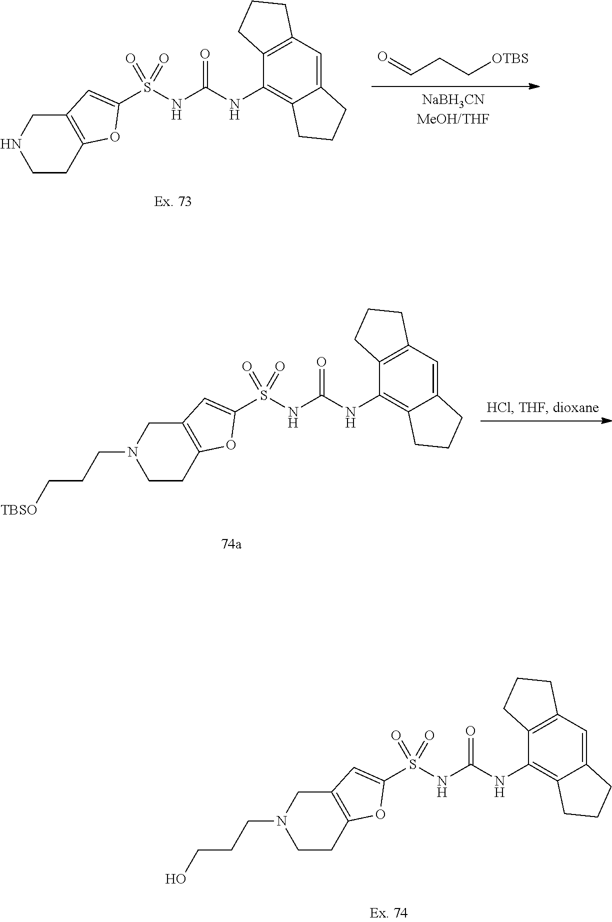 [SMILES: O=C(Nc1c2c(cc3c1CCC3)CCC2)NS(=O)(=O)c1cc2c(o1)CCNC2 ; CC(C)(C)[Si](C)(C)OCCC=O ; CC(C)(C)[Si](C)(C)OCCCN1CCc2oc(S(=O)(=O)NC(=O)Nc3c4c(cc5c3CCC5)CCC4)cc2C1 ; O=C(Nc1c2c(cc3c1CCC3)CCC2)NS(=O)(=O)c1cc2c(o1)CCN(CCCO)C2]

Step 1:

A colorless suspension of N-((1,2,3,5,6,7-hexahydro-s-indacen-4-yl)carbamoyl)-4,5,6,7-tetrahydrofuro[3,2-c]pyridine-2-sulfonamide Ex. 73 (120 mg, 0.299 mmol) and 3-((tert-butyldimethylsilyl)oxy)propanal (84 mg, 0.449 mmol) in MeOH (6 mL) and THF (2 mL) was stirred at rt for 0.5 h. NaBH₃CN (23 mg, 0.359 mmol) was added leading to the evolution of gas. The resulting colorless solution was stirred at rt for 3 h. The reaction was quenched by the addition of water (5 mL). The aqueous mixture was extracted with CHCl3/iPrOH (v/v, 3/1, 10 mL×3). The combined organic phases were washed with brine (5 mL×2), dried with Na₂SO₄, filtered and concentrated to a crude solid, which was washed with PE to give 5-(3-((tert-butyldimethylsilyl)oxy)propyl)-N-((1,2,3,5,6,7-hexahydro-s-indacen-4-yl)carbamoyl)-4,5,6,7-tetrahydrofuro[3,2-c]pyridine-2-sulfonamide 74a as a white solid (80 mg, 46.64%). LC-MS: m/z [M+H]⁺=574.30.

### Step 2:

A colorless suspension of 5-(3-((tert-butyldimethylsilyl)oxy)propyl)-N-((1,2,3,5,6,7-hexahydro-s-indacen-4-yl)carbamoyl)-4,5,6,7-tetrahydrofuro[3,2-c]pyridine-2-sulfonamide 74a (80 mg, 0.139 mmol) in DCM (1 mL) and HCl in 1,4-dioxane (1 mL, 4 M, 4 mmol) was stirred at rt for 1 h. Solvents were removed in vacuo, and the residue was dissolved in DMF and neutralized with an aqueous NaOH solution. The mixture was filtered, the filtrated was purified by reverse phase column chromatography (MeCN in water (0.05% NH₄HCO₃)). After lyophilization, the titled compound N-((1,2,3,5,6,7-hexahydro-s-indacen-4-yl)carbamoyl)-5-(3-hydroxypropyl)-4,5,6,7-tetrahydrofuro[3,2-c]pyridine-2-sulfonamide Ex. 74 was obtained as a white solid (10.2 mg, 15.30%). ¹HNMR (300 MHz, DMSO-d6) δ (ppm): 6.85 (s, 1H), 6.69 (s, 2H), 3.74 (m, 2H), 3.42 (m, 2H), 3.15 (m, 2H), 2.64-2.90 (m, 12H), 1.79-1.98 (m, 4H), 1.72-1.77 (m, 2H LC-MS: m/z [M+H]⁺=460.15.

### Examples 75 and 76: Preparation of (R)—N-((1,2,3,5,6,7-hexahydro-s-indacen-4-yl)carbamoyl)-4-((methylamino)methyl)furan-2-sulfonimidamide (Ex. 75) and (S)—N-((1,2,3,5,6,7-hexahydro-s-indacen-4-yl)carbamoyl)-4-((methylamino)methyl)furan-2-sulfonimidamide (Ex. 76)

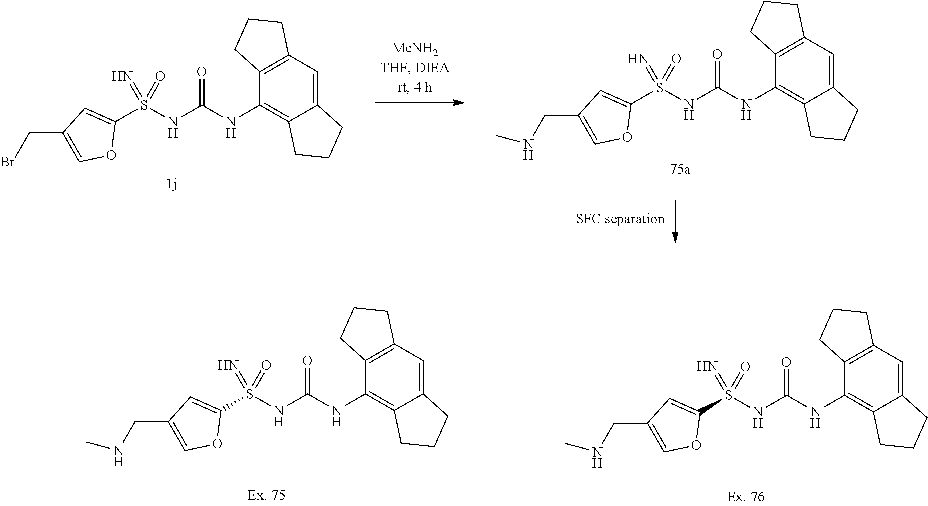 [SMILES: N=S(=O)(NC(=O)Nc1c2c(cc3c1CCC3)CCC2)c1cc(CBr)co1 ; CNCc1coc(S(=N)(=O)NC(=O)Nc2c3c(cc4c2CCC4)CCC3)c1 ; CNCc1coc([S@](=N)(=O)NC(=O)Nc2c3c(cc4c2CCC4)CCC3)c1 ; CNCc1coc([S@@](=N)(=O)NC(=O)Nc2c3c(cc4c2CCC4)CCC3)c1]

### Step 1:

A colorless suspension of 4-(bromomethyl)-N-((1,2,3,5,6,7-hexahydro-s-indacen-4-yl)carbamoyl)furan-2-sulfonimidamide 1j (300 mg, 0.684 mmol) in MeOH (5 mL) was added methylamine (5 ml). The reaction mixture was stirred at rt for 2 hours, filtered and concentrated. The residue was purified by Prep-HPLC using the following conditions: Column: CHIRALPAK IE, 2*25 cm, 5 μm; Mobile Phase A: MTBE(0.2% MSA)--HPLC, Mobile Phase B: EtOH--HPLC; Flow rate: 19 mL/min; Gradient: 20% B to 20% B in 30 min; Wavelength: 220/254 nm; RT1(min): 20; RT2(min): 25.6; Sample Solvent: MeOH; Injection Volume: 0.3 mL; Number Of Runs: 22. After lyophilization, the racemic mixture N-((1,2,3,5,6,7-hexahydro-s-indacen-4-yl)carbamoyl)-4-((methylamino)methyl)furan-2-sulfonimidamide 75a was obtained as a white solid (170 mg, 63.68%). LC-MS: m/z [M+H]⁺=389.1.

### Step 2:

3-(1,2,3,5,6,7-Hexahydro-s-indacen-4-yl)-1-[imino([4-[(methylamino)methyl]furan-2-yl])oxo-lambda6-sulfanyl]urea 75a (200 mg, 0.515 mmol) was further purified and separated by Chiral separation SFC using the following conditions: Column: CHIRALPAK IE, 2*25 cm, 5 μm; Mobile Phase A: MTBE(0.2% MSA), Mobile Phase B: EtOH; Flow rate:17 mL/min; Gradient: 30% B to 30% B in 16.2 min; 254/220 nm; RTL: 11.247 min; RT2: 13.289 min; Injection Volume: 0.3 ml; Number of Runs: 30.

The first fraction collected at RT1 (11.247 min) was neutralized with TEA and solvents were evaporated. The residue was purified again with reverse phase column (spherical C18, MeCN in water (0.05% NH₄HCO₃)). After lyophilization, the titled enantiomerically pure compound (R)—N-((1,2,3,5,6,7-hexahydro-s-indacen-4-yl)carbamoyl)-4-((methylamino)methyl)furan-2-sulfonimidamide Ex. 75 was obtained as a white solid (36.5 mg, 18.09%). The absolute stereochemistry at the S-atom was not determined, and only arbitrary assigned. ¹HNMR (300 MHz, Methanol-d4) δ (ppm): 7.68 (s, 1H), 7.06 (s, 1H), 6.90 (s, 1H), 3.65 (s, 2H), 2.70-2.88 (m, 8H), 2.41 (s, 3H), 1.93-2.14 (m, 4H); LC-MS: m/z [M+H]⁺=389.1.

The second fraction collected at RT2 (13.289 min) was neutralized with TEA and the solvents were evaporated. The residue was purified by reverse phase column (spherical C18, MeCN in water (0.05% NH₄HCO₃)). After lyophilization, the titled enantiomerically pure compound (S)—N-((1,2,3,5,6,7-hexahydro-s-indacen-4-yl)carbamoyl)-4-((methylamino)methyl)furan-2-sulfonimidamide Ex. 76 was obtained as a white solid (35.8 mg, 17.81% yield). ¹HNMR (300 MHz, Methanol-d4) δ (ppm): 7.68 (s, 1H), 7.06 (s, 1H), 6.90 (s, 1H), 3.65 (s, 2H), 2.70-2.88 (m, 8H), 2.41 (s, 3H), 1.93-2.14 (m, 4H); LC-MS: m/z [M+H]⁺=389.1.

### Example 77: Preparation of 5-(aminomethyl)-N-((1,2,3,5,6,7-hexahydro-s-indacen-4-yl)carbamoyl)-1-isopropyl-1H-pyrazole-3-sulfonimidamide

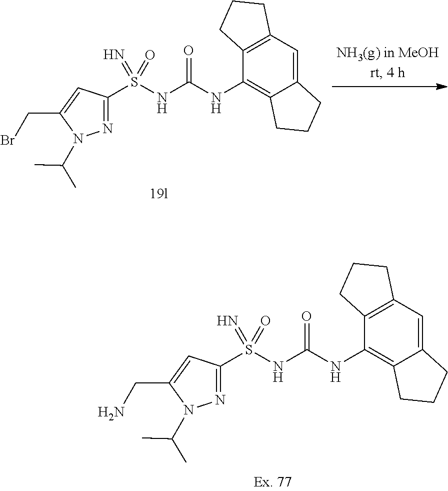 [SMILES: CC(C)n1nc(S(=N)(=O)NC(=O)Nc2c3c(cc4c2CCC4)CCC3)cc1CBr ; CC(C)n1nc(S(=N)(=O)NC(=O)Nc2c3c(cc4c2CCC4)CCC3)cc1CN]

To a solution of 5-(bromomethyl)-N-((1,2,3,5,6,7-hexahydro-s-indacen-4-yl)carbamoyl)-1-isopropyl-1H-pyrazole-3-sulfonimidamide 191 (150 mg, 0.312 mmol) in MeOH (10 ml) was added a solution of ammonia in MeOH (10 mL, 7 M) at 0° C. The resulting solution was stirred at rt for 4 h. Solvent was removed in vacuo, the residue was purified by Prep-HPLC using the following conditions: Column: XBridge Prep OBD C18 Column, 19*250 mm, 5 μm; Mobile Phase A: Water (10 mmol/L NH₄HCO₃+0.1% NH₃·H₂O), Mobile Phase B: ACN; Flow rate: 25 mL/min; Gradient: 34% B to 54% B in 7 min, 54% B to B; 254 nm; RT1: 5.42 min. After lyophilization, the titled compound 5-(aminomethyl)-N-((1,2,3,5,6,7-hexahydro-s-indacen-4-yl)carbamoyl)-1-isopropyl-1H-pyrazole-3-sulfonimidamide Ex. 77 was obtained as a white solid (25.7 mg, 19.43%). ¹HNMR (300 MHz, Methanol-d4) δ (ppm): 6.90 (s, 1H), 6.69 (s, 1H), 4.55-4.70 (m, 1H), 3.84-3.90 (m, 2H), 2.71-2.92 (m, 8H), 1.93-2.09 (m, 4H), 1.43-1.52 (m, 6H); LC-MS: m/z [M+H]⁺=417.1.

### Example 78: IC₅₀ Determination of IL-1β Release Inhibition in THP-1-ASC-GFP Cells

The compounds were tested for their ability to inhibit the IL-1β release in the THP-1-ASC-GFP cells after NLRP3 activation. THP1-ASC-GFP cells (Invivogen thp-ascgfp) were thawed following Invivogen's initial culture procedure. After the cells had been in culture for a minimum of 3 passages, they were ready to be used in assay. The growth medium was composed of RPM1640 medium (Gibco 32404-014), 10% heat inactivated (HI) serum (Gibco A3840102), Pen/Strep (Quality Biologics 118-089-721), 10 mM Hepes (Quality Biologics 118-089-721), 2 mM Glutamax (GIBCO 35050-061), 100 μg/mL normocin (Invivogen ant-nr), 100 μg/mL Zeocin (Invivogen ant-zn). The growth medium was freshly prepared once a week and sterile filtered. Assay medium consisted of RPMI 1640 medium, 10 mM Hepes, 2 mM Glutamax and Pen/Strep. To the assay medium, 1 μg/mL LPS (Invivogen tlrl-3pelps) was added. A cell suspension was prepared with assay medium and LPS at 1.25M cells per mL. With an electronic pipet, 80 μl of cell suspension was added to U bottom 96 well polystyrene plates (VWR 10062-902) leaving columns 1 and 2 empty. The plates were placed in the incubator for 4 hours. During the 4-hour priming of cells period 20 mM DMSO stock solution of the test compounds were prepared.

The compound stock plate (Plate A) was prepared as follow: in a V bottom 96 well polypropylene plate (Eppendorf 951040227), the 20 mM compound stock solution was further diluted with DMSO in a 1:10 ratio in row A to give a concentration of 2 mM; rows B to G were serially diluted in a 1:4 ratio from row A to reach the lowest concentration of 0.000488 mM, row H was solvent control (i.e. DMSO). An hour prior to the end of LPS incubation, a separate 96 deep well plate, Plate B, was prepared with 400 μL of assay media per well, then 2 μL of the stock DMSO solution from Plate A was added to each corresponding well. Plate B was covered to avoid media evaporation. In a 50 mL tube, a solution of nigericin in assay media at 50 NM/mL concentration was prepared. To a new 96 deep well plate, Plate C, 200 μL of this nigericin containing media was dispensed to each well and 1 L of the test compounds from Plate A to each corresponding well was added. Plate C was covered to avoid media evaporation. After 4 hours of priming, all the media from the LPS treated THP-1 cells in the initial U bottom plate was removed manually. To this plate of primed cells, 80 μL/well test compounds from Plate B was added in duplicate columns (5 compounds tested per plate). The plate was placed in an incubator for 30 min. After 30 min, 20 μL of the content from Plate C (assay media+nigericin+compound) was added to each corresponding well and corresponding compound columns. The plate was placed back in the incubator for 1 hour. At the end of 1 hour, the supernatant from this test plate was transferred to a new 96 well V bottom polypropylene plate which was frozen immediately at −80° C. The plate was transferred to −20° C. the next day and awaiting further determination of cytokine IL-1β level.

The release of IL-1β into the cell media was determined by MSD® Multi-Spot Assay System (V-Plex, IL-1B kit, K151KPD-4). Following the manufacture's protocol and using the Alternate Protocol 3, Dilute-In-Plate protocol specifically, the level of IL-1β in the above collected supernatant plate was measured as electrochemiluminescent signals. Using MSD DISCOVERY WORKBENCH® analysis software, the signals were converted to the concentration of IL-1β in each well. The IC₅₀ values of the compounds were calculated using the Graph Pad Prism software based on the IL-13 concentration determined. The detection range of IL-1β using MSD kit was approximately 0.15 to 600 μg/mL (based on various lots of standards purchased from MSD). The IC₅₀ values of the compounds of the present disclosure are summarized in Table 5, (“++++” means IC₅₀≤5 nM; “+++” means 5 nM<IC₅₀≤50 nM, “++” means 50 nM<IC₅₀≤500 nM; “+” means +: 500 nM<IC₅₀), derived from an average of at least three experiments.

### Example 79: IC₅₀ Determination of IL-1β Release Inhibition in U937 Cells

The compounds of the present disclosure were tested for their ability to inhibit the IL-1β release in the U937 cells after NLRP3 activation. U937 cells (U-97 CRL 1593.2) were thawed following ATCC's initial culture procedure. After the cells had been in culture for a minimum of 2 passages, they were ready to be used in assay. The growth medium was composed of RPMI1640 medium (Quality Biological 112-040-101), 10% serum (Corning 35-076-CV), Pen/Strep (GIBCO 15140-122), 10 mM Hepes (Quality Biological 118-089-721), 2 mM Glutamax (GIBCO 35050-061), and 100 μg/mL normocin (Invivogen ant-nr)-Differentiating media was prepared by adding Phorbol 12-myristate 13-acetate (PMA) (AdipoGen AG-CN2-0010-M005) to growth medium. A cell suspension was prepared with differentiating media at 500k cells/ml. With an electronic pipet, 200 ul of cell suspension was added to each well (96w white plate Corning 3903) leaving columns 1 and 2 empty. Plates were left in the biosafety cabinet for 15 min and then placed in incubator for 90 hours. Priming media was prepared with assay medium consisting of RPMI 1640 medium, 10 mM Hepes, 2 mM Glutamax and Pen/Strep plus 1 μg/mL LPS (Invivogen tlrl-3pelps). Differentiating media was manually removed and 80 ul of priming media was added to each well with an electronic pipet. The plates were placed in the incubator for 5 hours. During the 5-hour priming of cells period, a 20 mM DMSO stock solution of the test compounds were prepared. The compound stock plate (Plate A) was prepared as follow: in a V bottom 96 well polypropylene plate (Eppendorf 951040227), the 20 mM compound stock solution was further diluted with DMSO in a 1:10 ratio in row A to give a concentration of 2 mM; rows B to G were serially diluted in a 1:4 ratio from row A to reach the lowest concentration of 0.000488 mM, row H was solvent control (i.e. DMSO). An hour prior to the end of LPS incubation, a separate 96 deep well plate, Plate B, was prepared with 400 μL of assay media per well, then 2 sL of the stock DMSO solution from Plate A was added to each corresponding well. Plate B was covered to avoid media evaporation.

In a 50 mL tube, a solution of nigericin in assay media at 50 uM/mL concentration was prepared. To a new 96 deep well plate, Plate C, 200 μL of this nigericin containing media was dispensed to each well and 1 μL of the test compounds from Plate B to each corresponding well was added. Plate C was covered to avoid media evaporation. After 5 hours of priming, all the media from the LPS treated U937 cells was removed manually. To this plate of primed cells, 80 μL/well test compounds from Plate B (assay media+compound) was added in duplicate columns (5 compounds tested per plate). The plate was placed in an incubator for 30 min. After 30 min, 20 μL of the content from Plate C (assay media+nigericin+compound) was added to each corresponding well and corresponding compound columns. The plate was placed back in the incubator for 1 hour. At the end of 1 hour, the supernatant from this test plate was transferred to a new 96 well V bottom polypropylene plate which was frozen immediately at −80° C. The plate was transferred to −20° C. the next day and awaited further determination of cytokine IL-Ip level.

The release of IL-1β into the cell media was determined by MSD® Multi-Spot Assay System (V-Plex, IL-1B kit, K151KPD-4). Following the manufacture's protocol and using the Alternate Protocol 3, Dilute-In-Plate protocol specifically, the level of IL-IP in the above collected supernatant plate was measured as electrochemiluminescent signals. Using MSD DISCOVERY WORKBENCH® analysis software, the signals were converted to the concentration of IL-1β in each well. The IC₅₀ values of the compounds were calculated using the Graph Pad Prism software based on the IL-1β concentration determined. The detection range of IL-1β using MSD kit was approximately 0.15 to 600 μg/mL (based on various lots of standards purchased from MSD). The IC₅₀ values of the compounds of the present disclosure are summarized in Table 5, (“++++”: IC₅₀≤5 nM; “+++”: 5 nM<IC₅₀≤50 nM; “++”: 50 nM<IC₅₀≤500 nM; “+”: 500 nM<IC₅₀), derived from an average of at least three experiments.

TABLE 5

| | IL-1β | IL-1β |
|---|---|---|
| Ex. | IC₅₀ (nM) | IC₅₀ (nM) |
| No. | THP1 cells | U937 cells |
| 1 | ++++ | +++ |
| 2 | | ++ |
| 3 | | ++ |
| 4 | ++++ | ++++ |
| 5 | ++++ | ++++ |
| 6 | ++++ | +++ |
| 7 | ++++ | ++++ |
| 8 | ++++ | ++++ |
| 9 | ++++ | +++ |
| 10 | ++++ | +++ |
| 11 | ++++ | +++ |
| 12 | ++++ | ++++ |
| 13 | ++++ | +++ |
| 14 | ++++ | +++ |
| 15 | | +++ |
| 16 | | + |
| 17 | | ++ |
| 18 | | +++ |
| 19 | ++++ | +++ |
| 20 | ++++ | +++ |
| 21 | ++++ | ++++ |
| 22 | | +++ |
| 23 | ++++ | ++++ |
| 24 | ++++ | +++ |
| 25 | ++++ | ++++ |
| 26 | ++ | ++ |
| 27 | ++++ | ++++ |
| 28 | ++ | ++ |
| 29 | ++++ | ++++ |
| 30 | ++ | + |
| 31 | ++++ | ++++ |
| 32 | | ++ |
| 33 | +++ | +++ |
| 34 | | +++ |
| 35 | ++++ | ++++ |
| 36 | +++ | ++ |
| 37 | ++++ | +++ |
| 38 | +++ | ++ |
| 39 | +++ | ++ |
| 40 | | +++ |
| 41 | +++ | +++ |
| 42 | +++ | ++ |
| 43 | ++++ | +++ |
| 44 | +++ | +++ |
| 45 | ++++ | +++ |
| 46 | | +++ |
| 47 | | +++ |
| 48 | ++++ | +++ |
| 49 | | +++ |
| 50 | +++ | +++ |
| 51 | ++++ | +++ |
| 52 | | +++ |
| 53 | | +++ |
| 54 | | ++++ |
| 55 | | +++ |
| 56 | | +++ |
| 57 | +++ | +++ |
| 58 | ++ | +++ |
| 59 | +++ | +++ |
| 60 | +++ | ++ |
| 61 | ++ | ++ |
| 62 | +++ | +++ |
| 63 | +++ | +++ |
| 64 | | ++ |
| 65 | | +++ |
| 66 | | ++++ |
| 67 | | +++ |
| 68 | | +++ |
| 69 | | +++ |
| 70 | | +++ |
| 71 | ++++ | +++ |
| 72 | +++ | +++ |
| 73 | +++ | +++ |
| 74 | +++ | +++ |
| 75 | ++ | ++ |
| 76 | ++++ | ++++ |
| 77 | | ++ |

What is claimed is:

1. A compound represented by the structure of Formula (I): 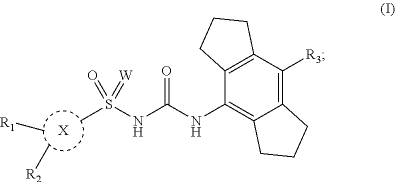
or a pharmaceutically acceptable salt, solvate, isomer, or tautomer thereof, wherein
W is selected from the group consisting of O, NH, and N—CN;
R₁ is selected from the group consisting of: 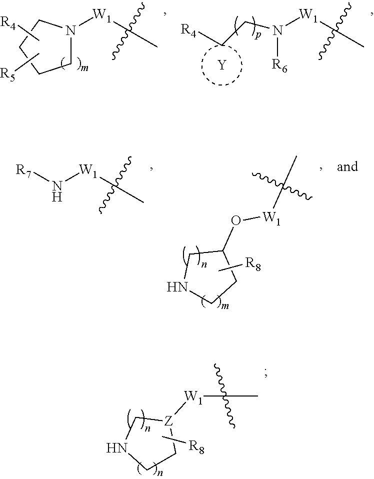
wherein
W₁ is (CH₂)_{q} or C(═O), wherein q is 1, 2, or 3;
R₄ is selected from the group consisting of halogen, CF₃, C1-C8 alkyl, and C3-C8 cycloalkyl, or absent;
R₅ is hydroxyl or hydroxyalkyl;
R₆ is selected from the group consisting of H, optionally substituted C1-C8 alkyl, and optionally substituted C3-C8 cycloalkyl; or R₆ and R₂ together form a 5-10 member optionally substituted heterocyclic ring, the heterocyclic ring having one or more heteroatoms selected from O, N, and S;
R₇ is H or C1-C8 alkyl;
R₈ is selected from the group consisting of halogen, hydroxyalkyl, and optionally substituted C1-C8 alkyl, or absent; 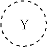
is C3-C8 cycloalkyl or 4-8 member heterocycloalkyl with one heteroatom selected from the group consisting of N, O, and S;
m is selected from the group consisting of 0, 1, 2, 3, and 4;
n is selected from the group consisting of 1, 2, 3, and 4;
p is selected from the group consisting of 1, 2, and 3; and
Z is selected from the group consisting of N, CH, and C—CH₃;
R₂ is selected from the group consisting of C1-C8 alkyl, CF, C3-C8 cycloalkyl, and optionally substituted hydroxyalkyl, or absent;
R₃ is selected from the group consisting of H, halogen, or C1-C8 alkyl; and 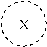
is a 5-member heteroaryl having 1 or 2 heteroatoms, each of the heteroatoms is selected from the group consisting of O, N, and S.

2. The compound of claim 1, wherein 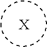
is selected from the group consisting of furan, thiophene, pyrrole, pyrazole, imidazole.

3. The compound of claim 1, wherein 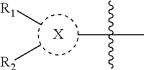
is selected from the group consisting of

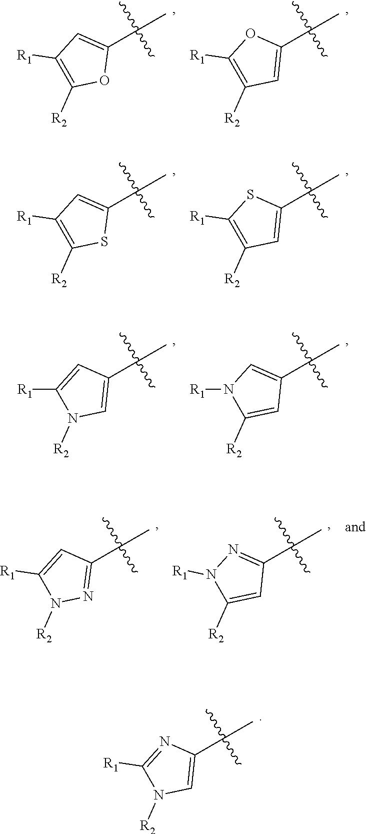

4. The compound of claim 1, wherein
R₁ is 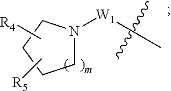
R₄ is selected from the group consisting of halogen, CF₃, C1-C8 alkyl, and C3-C8 cycloalkyl, or absent;
R₅ is hydroxyl or hydroxy(C1-C3 alkyl); and
m is 0, 1, 2, or 3.

5. The compound of claim 1, wherein
R₁ is 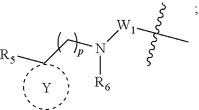
R₅ is hydroxyl or hydroxy(C1-C3 alkyl); and
R₆ is H, or optionally substituted C1-C8 alkyl; or R₆ and R₂ together form a 5-10-member optionally substituted heterocyclic ring, the heterocyclic ring having one or more heteroatom selected from O, N, and S; and 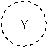
is C3-C6 cycloalkyl or 4-6-member heterocycloalkyl with one heteroatom selected from the group consisting of N, O, and S.

6. The compound of claim 1, wherein
R₁ is 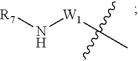
R₇ is H or C1-C8 alkyl.

7. The compound of claim 1, wherein
R₁ is 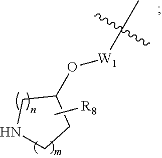
R₈ is selected from the group consisting of halogen, hydroxy, and optionally substituted alkyl, or absent;
m is selected from the group consisting of 0, 1, 2, 3, and 4; and
n is selected from the group consisting of 1, 2, 3, and 4.

8. The compound of claim 1, wherein
R₁ is 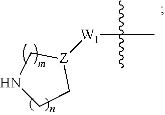
R₈ is selected from the group consisting of halogen, hydroxy, and optionally substituted alkyl, or absent;
m is selected from the group consisting of 0, 1, 2, 3, and 4; and
n is selected from the group consisting of 1, 2, 3, and 4.

9. The compound of claim 1, wherein the compound is further represented by the structure of Formula (Ia): 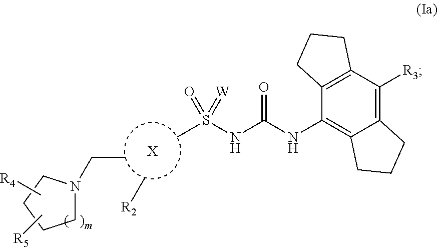
or a pharmaceutically acceptable salt, solvate, isomer, or tautomer thereof.

10. The compound of claim 9, wherein:
W is O or NH;
R₄ is halogen, CF₃, or absent;
R₅ is OH or CH₂OH;
m is 0, 1, or 3;
R₂ is H or C1-C3 alkyl;
R₃ is H, F, or Cl; and 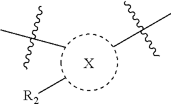
is selected from the group consisting of

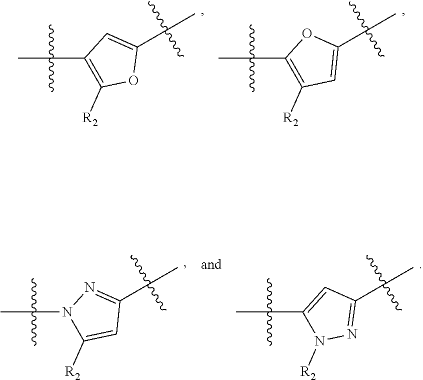

11. The compound of claim 1, wherein the compound is further represented by the structure of Formula (Ib): 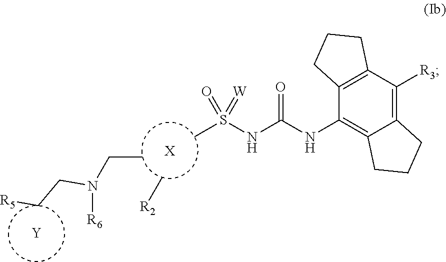
or a pharmaceutically acceptable salt, solvate, isomer, or tautomer thereof.

12. The compound of claim 11, wherein: 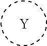
is C3-C6 cycloalkyl or a 4-6-member heterocycloalkyl having one O;
W is O or NH,
R₅ is OH or CH₂OH;
R₆ is H or C1-C3 alkyl;
p is 1 or 2;
R₂ is H or C1-C3 alkyl;
R₃ is H or halogen; and 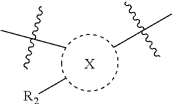
is selected from the group consisting of

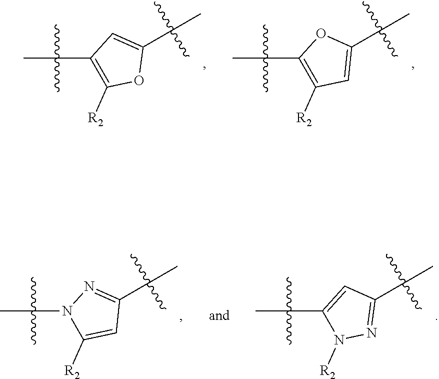

13. The compound of claim 1, wherein the compound is further represented by the structure of Formula (Ic): 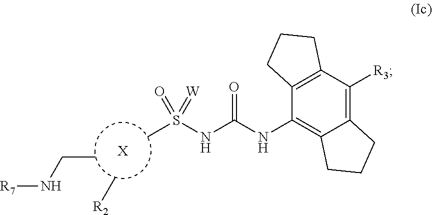
or a pharmaceutically acceptable salt, solvate, isomer, or tautomer thereof.

14. The compound of claim 13, wherein:
W is O or NH,
R₇ is H or C1-C3 alkyl;
R₂ is H or C1-C3 alkyl;
R₃ is H or halogen; and 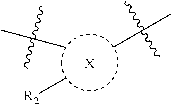
is selected from the group consisting of

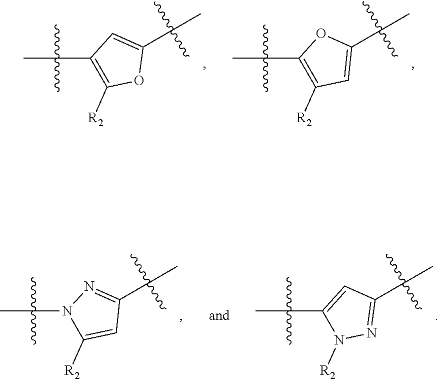

15. The compound of claim 1, wherein the compound is further represented by the structure of Formula (Id): 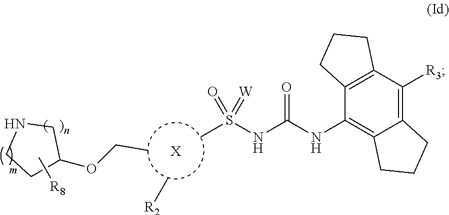
or a pharmaceutically acceptable salt, solvate, isomer, or tautomer thereof.

16. The compound of claim 15, wherein:
W is O or NH;
R₈ is absent;
m is 0, 1, or 2;
n is 1 or 2;
R₂ is hydroxy (C1-C3 alkyl);
R₃ is H or halogen; and 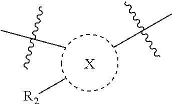
is selected from the group consisting of

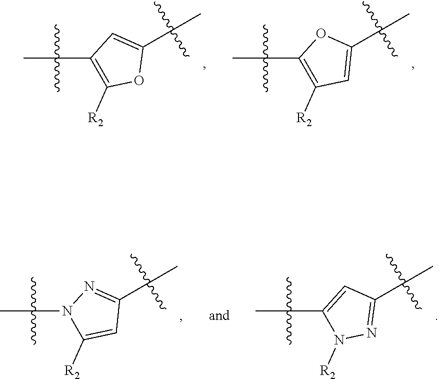

17. The compound of claim 1, wherein the compound is further represented by the structure of Formula (Ie): 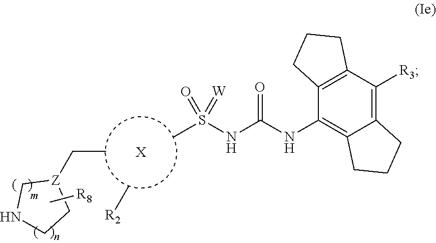
or a pharmaceutically acceptable salt, solvate, isomer, or tautomer thereof.

18. The compound of claim 17, wherein:
W is O or NH;
R₈ is absent;
mi s 0, 1, or 2;
n is 1, 2, or 3;
Z is N or CH;
R₂ is C1-C3 alkyl or hydroxy (C1-C3 alkyl);
R₃ is H or halogen; and 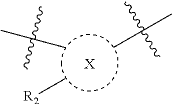
is selected from the group consisting of

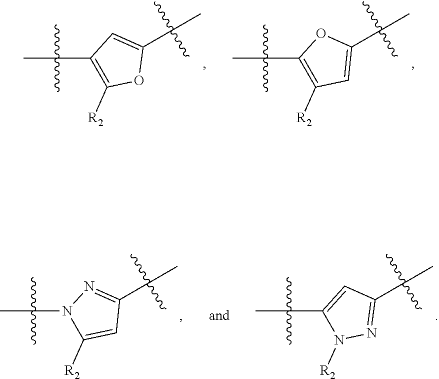

19. The compound of claim 1, further represented by any one of the following:

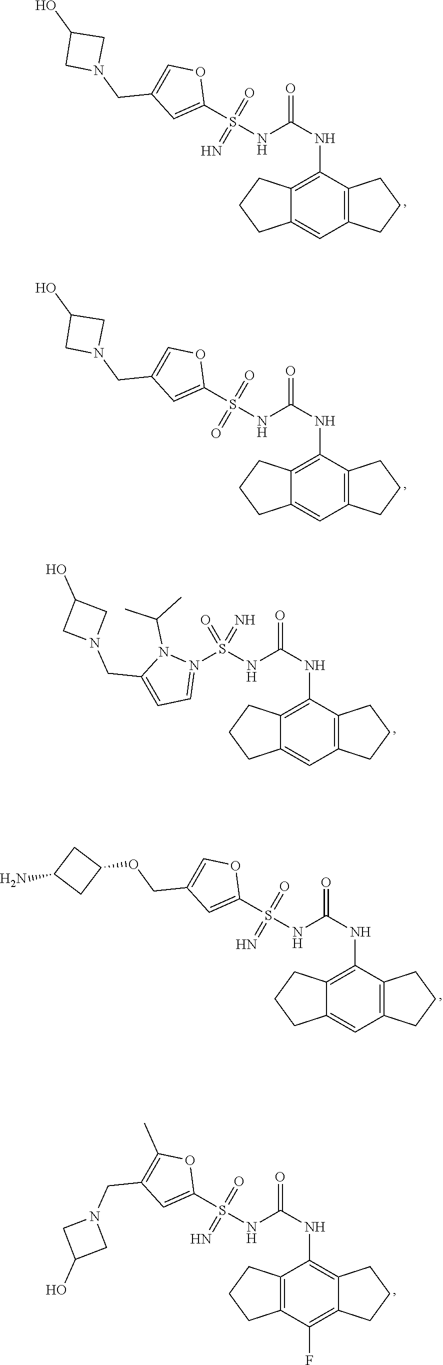

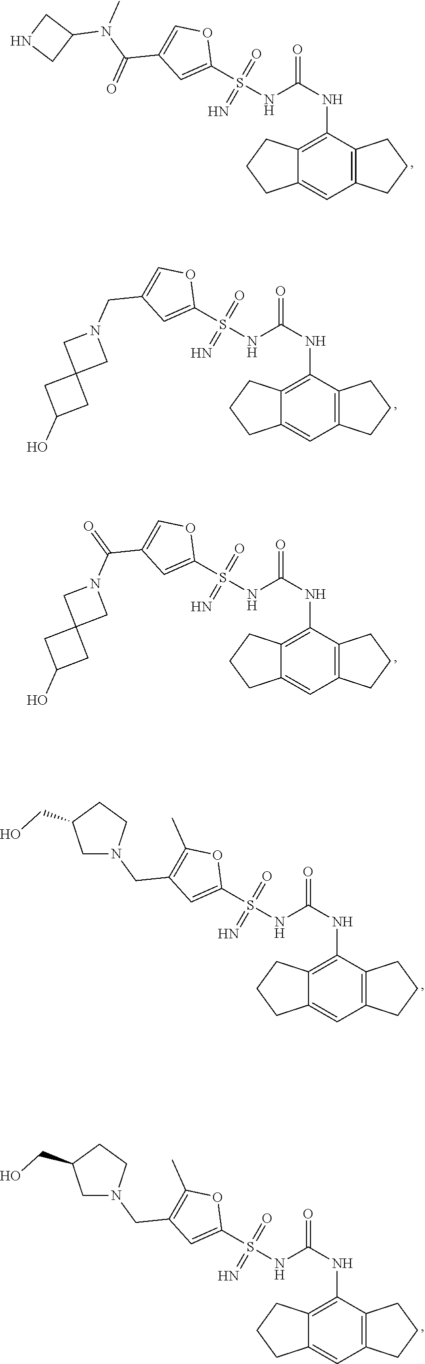 [SMILES: CN(C(=O)c1coc(S(=N)(=O)NC(=O)Nc2c3c(cc4c2CCC4)CCC3)c1)C1CNC1 ; N=S(=O)(NC(=O)Nc1c2c(cc3c1CCC3)CCC2)c1cc(CN2CC3(CC(O)C3)C2)co1 ; N=S(=O)(NC(=O)Nc1c2c(cc3c1CCC3)CCC2)c1cc(C(=O)N2CC3(CC(O)C3)C2)co1 ; Cc1oc(S(=N)(=O)NC(=O)Nc2c3c(cc4c2CCC4)CCC3)cc1CN1CC[C@@H](CO)C1 ; Cc1oc(S(=N)(=O)NC(=O)Nc2c3c(cc4c2CCC4)CCC3)cc1CN1CC[C@H](CO)C1]

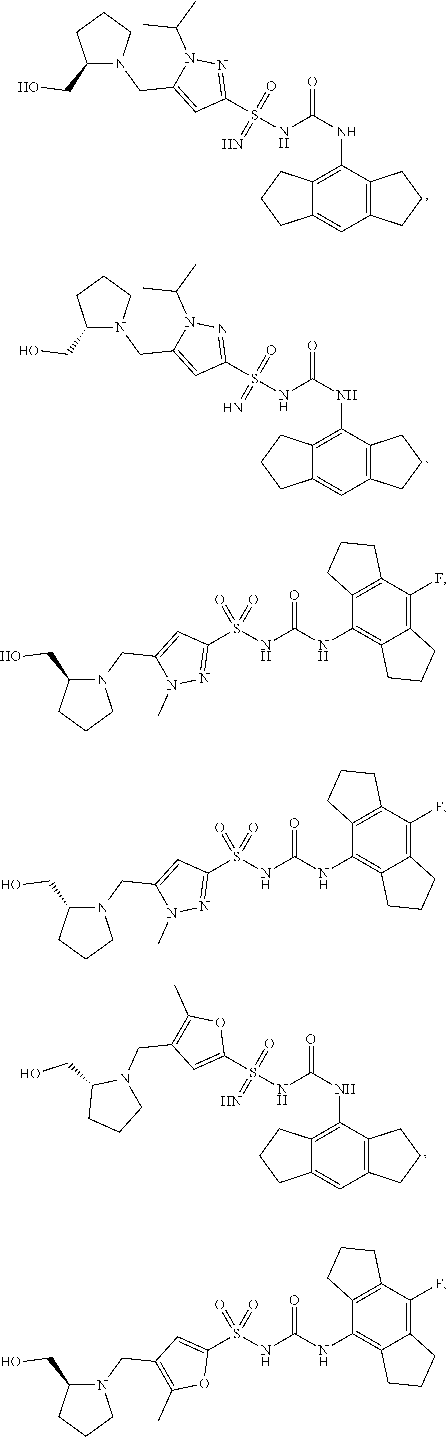

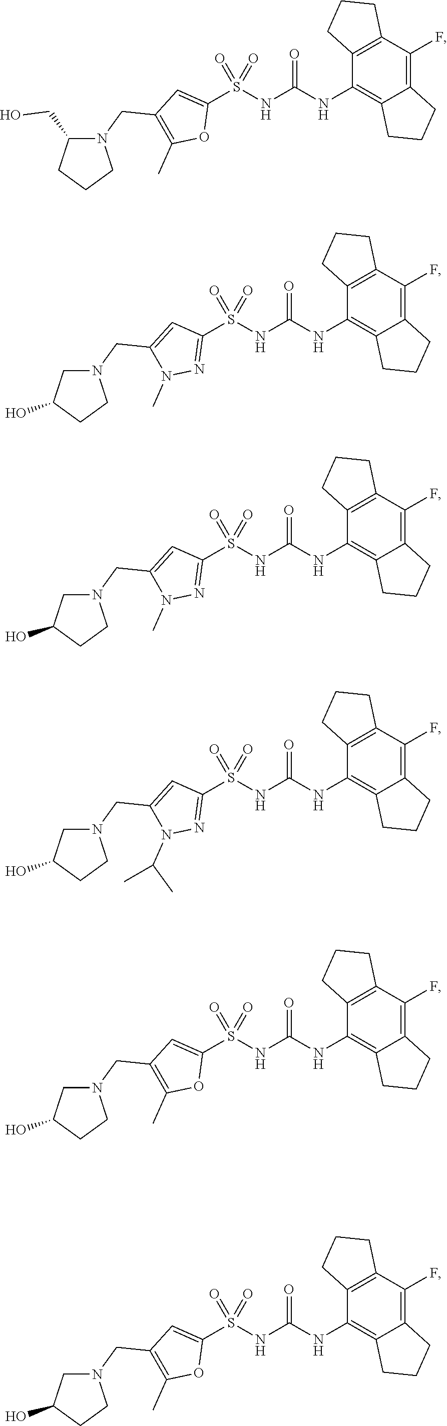 [SMILES: Cc1oc(S(=O)(=O)NC(=O)Nc2c3c(c(F)c4c2CCC4)CCC3)cc1CN1CCC[C@@H]1CO ; Cn1nc(S(=O)(=O)NC(=O)Nc2c3c(c(F)c4c2CCC4)CCC3)cc1CN1CC[C@H](O)C1 ; Cn1nc(S(=O)(=O)NC(=O)Nc2c3c(c(F)c4c2CCC4)CCC3)cc1CN1CC[C@@H](O)C1 ; CC(C)n1nc(S(=O)(=O)NC(=O)Nc2c3c(c(F)c4c2CCC4)CCC3)cc1CN1CC[C@H](O)C1 ; Cc1oc(S(=O)(=O)NC(=O)Nc2c3c(c(F)c4c2CCC4)CCC3)cc1CN1CC[C@H](O)C1 ; Cc1oc(S(=O)(=O)NC(=O)Nc2c3c(c(F)c4c2CCC4)CCC3)cc1CN1CC[C@@H](O)C1]

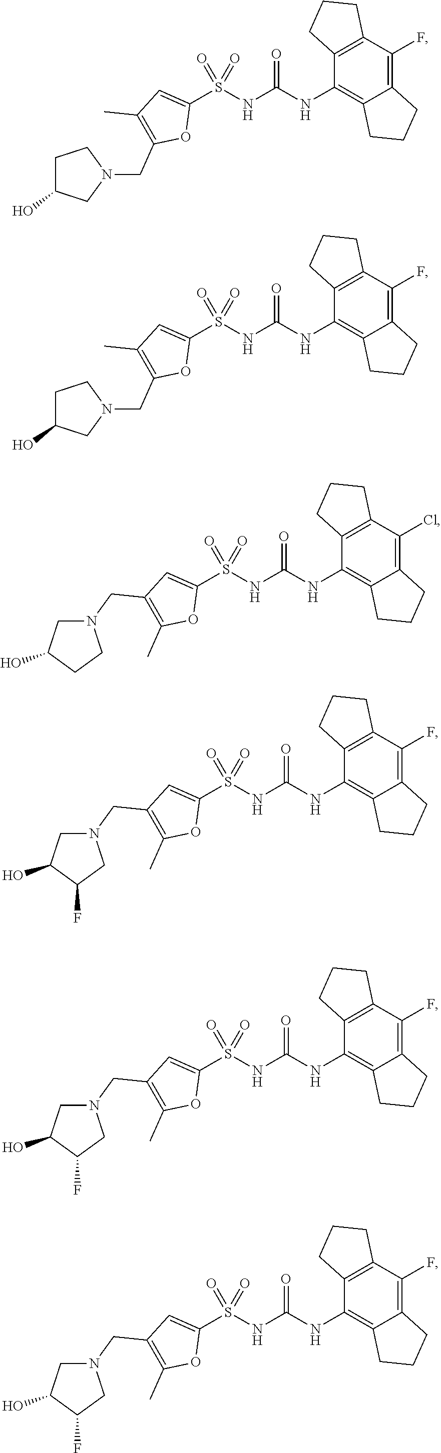 [SMILES: Cc1cc(S(=O)(=O)NC(=O)Nc2c3c(c(F)c4c2CCC4)CCC3)oc1CN1CC[C@@H](O)C1 ; Cc1cc(S(=O)(=O)NC(=O)Nc2c3c(c(F)c4c2CCC4)CCC3)oc1CN1CC[C@H](O)C1 ; Cc1oc(S(=O)(=O)NC(=O)Nc2c3c(c(Cl)c4c2CCC4)CCC3)cc1CN1CC[C@H](O)C1 ; Cc1oc(S(=O)(=O)NC(=O)Nc2c3c(c(F)c4c2CCC4)CCC3)cc1CN1C[C@@H](F)[C@@H](O)C1 ; Cc1oc(S(=O)(=O)NC(=O)Nc2c3c(c(F)c4c2CCC4)CCC3)cc1CN1C[C@H](O)[C@@H](F)C1 ; Cc1oc(S(=O)(=O)NC(=O)Nc2c3c(c(F)c4c2CCC4)CCC3)cc1CN1C[C@@H](O)[C@@H](F)C1]

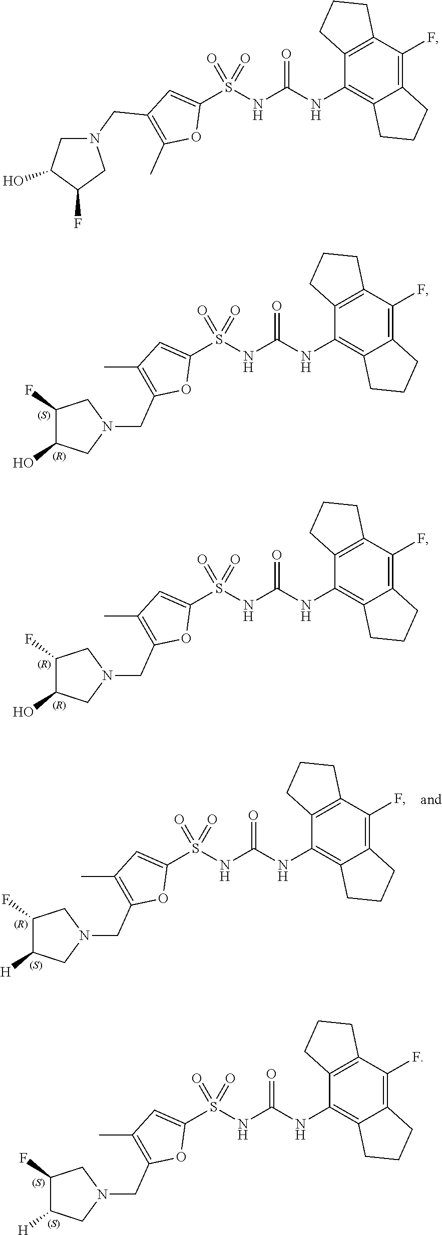

20. The compound of claim 1, further represented by any one of the following:

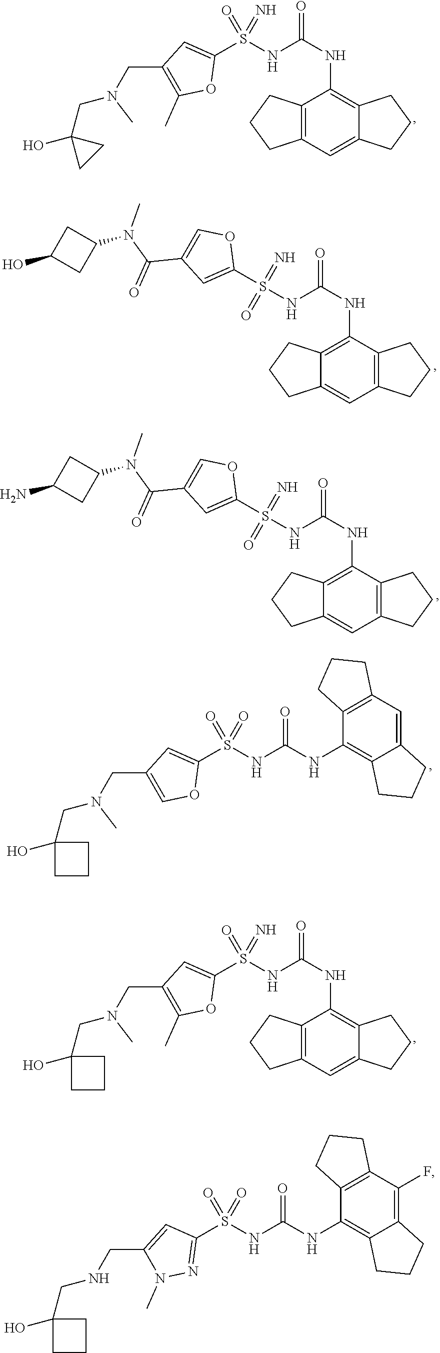

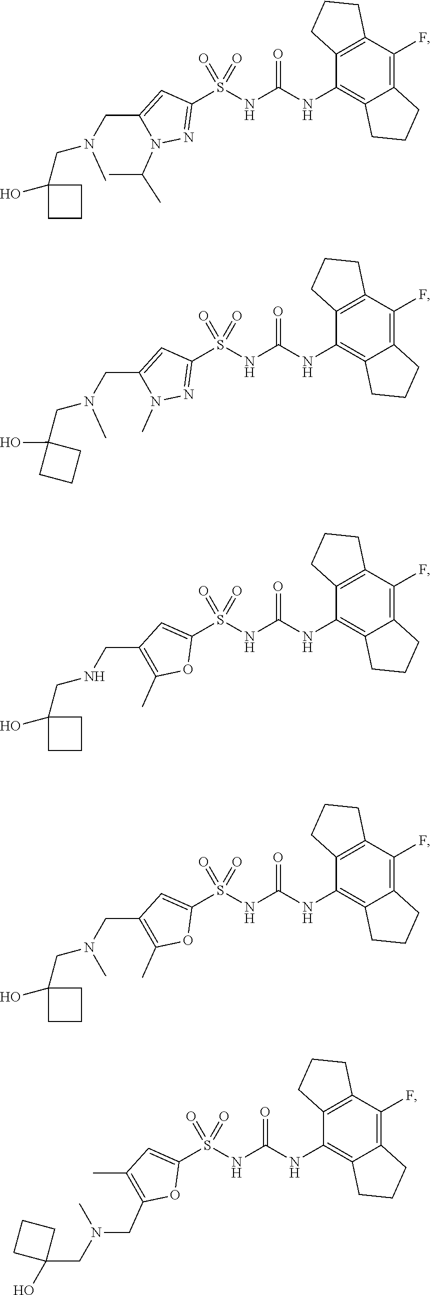 [SMILES: CC(C)n1nc(S(=O)(=O)NC(=O)Nc2c3c(c(F)c4c2CCC4)CCC3)cc1CN(C)CC1(O)CCC1 ; CN(Cc1cc(S(=O)(=O)NC(=O)Nc2c3c(c(F)c4c2CCC4)CCC3)nn1C)CC1(O)CCC1 ; Cc1oc(S(=O)(=O)NC(=O)Nc2c3c(c(F)c4c2CCC4)CCC3)cc1CNCC1(O)CCC1 ; Cc1oc(S(=O)(=O)NC(=O)Nc2c3c(c(F)c4c2CCC4)CCC3)cc1CN(C)CC1(O)CCC1 ; Cc1cc(S(=O)(=O)NC(=O)Nc2c3c(c(F)c4c2CCC4)CCC3)oc1CN(C)CC1(O)CCC1]

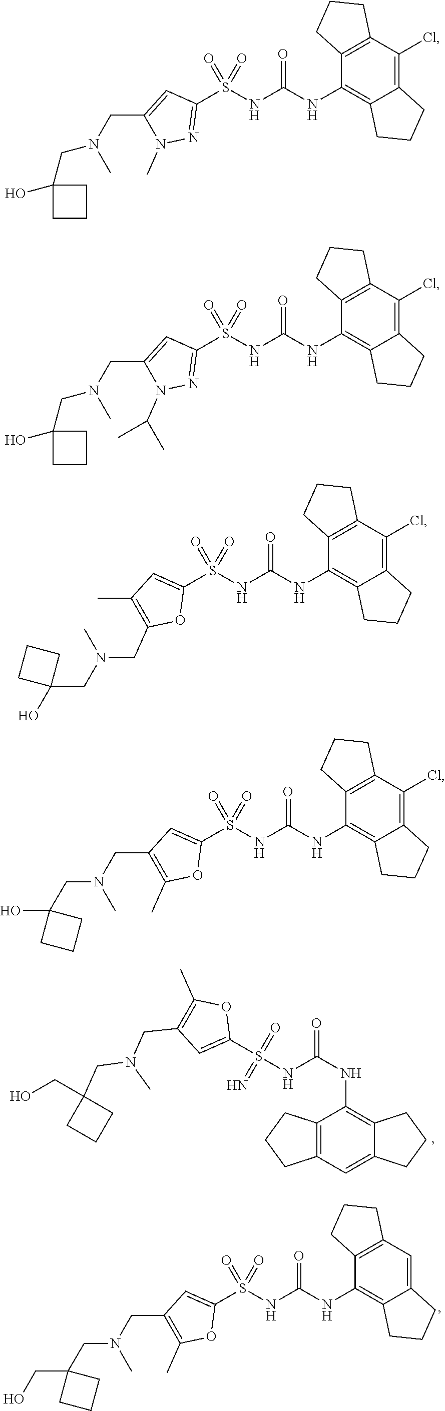

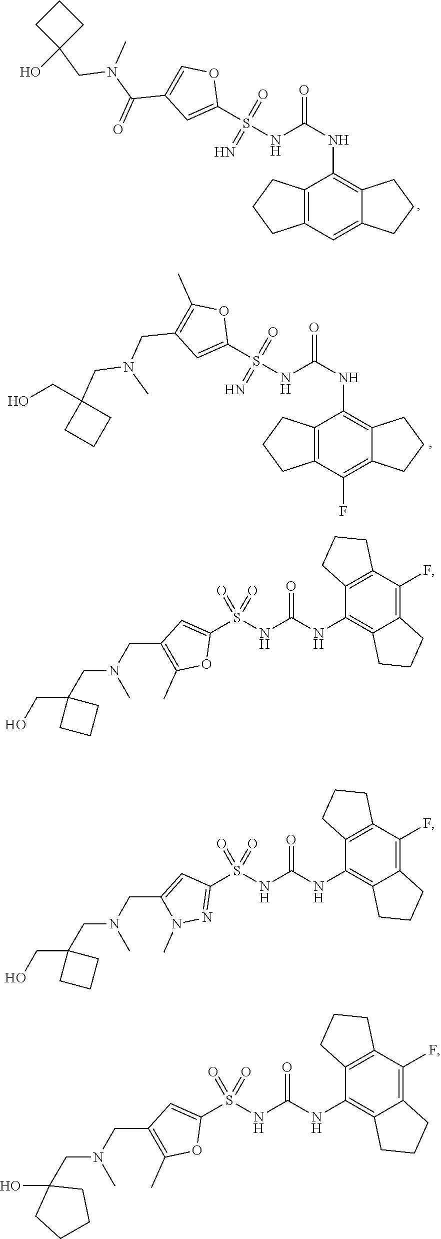

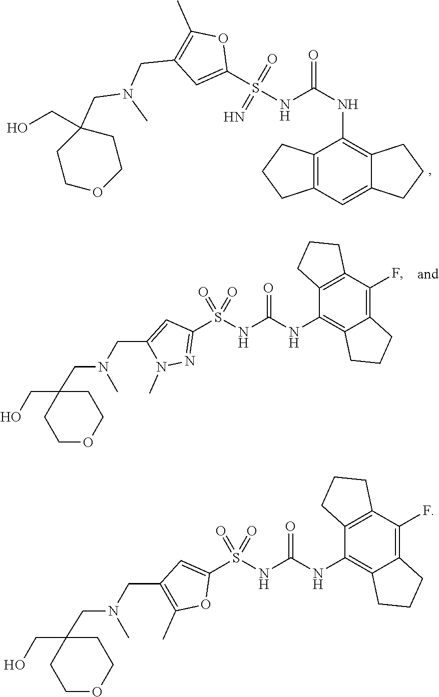

21. The compound of claim 1, further represented by any one of the following:

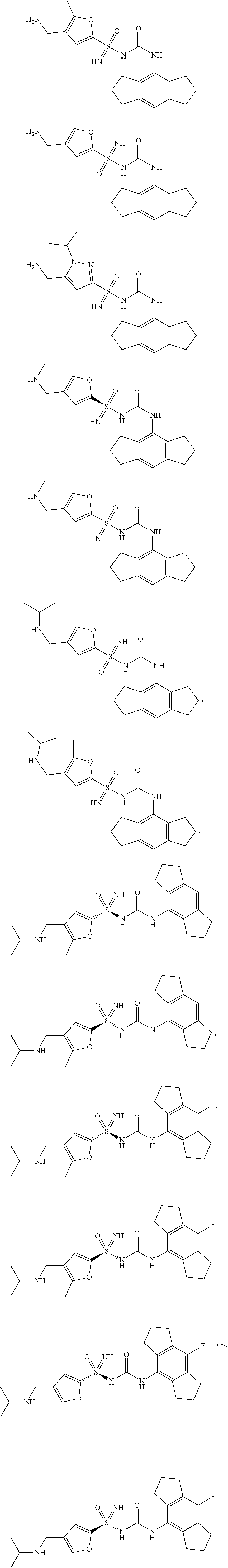

22. The compound of claim 1, further represented by the following:

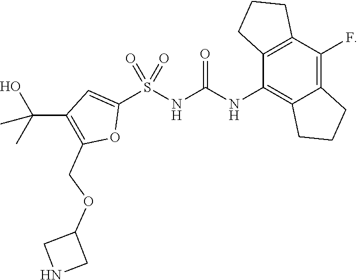 [SMILES: CC(C)(O)c1cc(S(=O)(=O)NC(=O)Nc2c3c(c(F)c4c2CCC4)CCC3)oc1COC1CNC1]

23. The compound of claim 1, further represented by any one of the following:

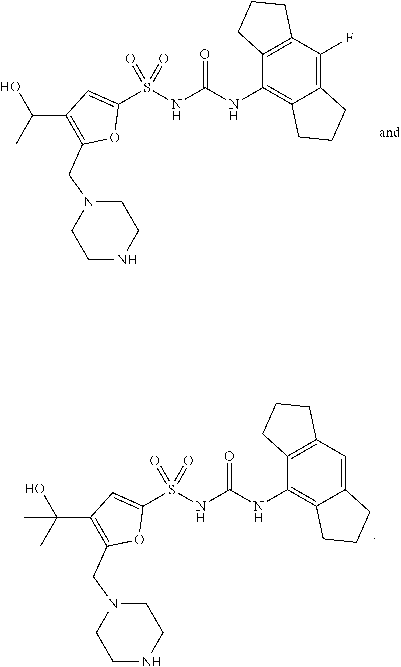 [SMILES: CC(O)c1cc(S(=O)(=O)NC(=O)Nc2c3c(c(F)c4c2CCC4)CCC3)oc1CN1CCNCC1 ; CC(C)(O)c1cc(S(=O)(=O)NC(=O)Nc2c3c(cc4c2CCC4)CCC3)oc1CN1CCNCC1]

24. A pharmaceutical composition comprising a therapeutically effective amount of a compound of any one of claims 1-23 and a pharmaceutically acceptable excipient.

25. A method of preventing, treating, or ameliorating one or more diseases in a subject, comprising administering a compound of any one of claims 1-23, or a pharmaceutically acceptable salt thereof, to a subject in need thereof.

26. The method of claim 25, wherein the disease is characterized by a disease progression that comprises the activity of IL-1β, IL-18, or both.

27. The method of claim 25, wherein the disease is selected from the group consisting of atherosclerosis, gout, acute gouty arthritis, rheumatoid arthritis, nonalcoholic steatoheptitis, inflammatory bowel disease, Parkinson's disease, Alzheimer's disease, multiple sclerosis, glaucoma, age related macula degeneration, diabetic retinopathy, and dry eye.

28. The method of claim 25, wherein the disease is at least one chronic inflammatory disorder.

29. The method of claim 28, wherein the disease is characterized by a disease progression pathology that comprises the activity of NLRP3 inflammasome.

30. The method of claim 29, wherein the NLRP3 inflammasome comprises at least one mutation.

31. The method of claim 30, wherein the disease is selected from the group consisting of cryopyrin-associated periodic syndrome, Behcet's syndrome, neonatal onset multisystem inflammatory disease, and Schnitzler's syndrome.

32. The method of claim 25, wherein the method is a monotherapy.

33. The method of claim 25, wherein the method includes administering at least one another form of treatment.

34. The method of claim 25, wherein the subject is human.
